(19) United States
(12) Patent Application Publication
(10) Pub. No.: US 2025/0320310 A1
(43) Pub. Date: Oct. 16, 2025

(54) A METHOD FOR PROGRAMMATICALLY MANAGING ANTIBODY DISULFIDE BONDS SITE-SPECIFIC MODIFICATION

(71) Applicant: SUZHOU BIOREINNO BIOTECHNOLOGY LIMITED COMPANY, SUZHOU CITY (CN)

(72) Inventors: Lili WU, SUZHOU (CN); Ao JI, SUZHOU (CN); Wenxu HE, SUZHOU (CN); Yajun RAN, SUZHOU (CN); Yaru SHAO, SUZHOU (CN); Yicheng WANG, SUZHOU (CN); Yu SHI, SUZHOU (CN)

(21) Appl. No.: 18/995,020

(22) PCT Filed: Aug. 22, 2023
(86) PCT No.: PCT/CN2023/114316

§ 371 Date: Jan. 15, 2025

(30) Foreign Application Priority Data
Aug. 22, 2022 (WO) PCT/CN2022/113992
Sep. 20, 2022 (WO) PCT/CN2022/119955
Sep. 20, 2022 (WO) PCT/CN2022/119999
Nov. 11, 2022 (WO) PCT/CN2022/131519
Nov. 11, 2022 (WO) PCT/CN2022/131521
Jan. 19, 2023 (WO) PCT/CN2023/073070

(51) Int. Cl. C07K 16/32; A61K 47/68

(52) U.S. Cl. CPC C07K 16/32; A61K 47/68031; A61K 47/6855; C07K 2317/53; C07K 2317/55

(57) ABSTRACT

The present disclosure relates to a method for programmatically managing antibody disulfide bonds site-specific modification, and the modified antibody prepared by the method and the use of the antibody modification.

### CROSS-REFERENCE TO RELATED APPLICATIONS

This application claims the priority to PCT Application No. PCT/CN2022/113992, filed on Aug. 22, 2022, PCT Application No. PCT/CN2022/119999, filed on Sep. 20, 2022, PCT Application No. PCT/CN2022/119955, filed on Sep. 20, 2022, PCT Application No. PCT/CN2022/131519, filed on Nov. 11, 2022, PCT Application No. PCT/CN2022/131521, filed on Nov. 11, 2022, and PCT Application No. PCT/CN2023/073070, filed on Jan. 19, 2023.

The contents of the prior PCT applications are considered as a part of the present disclosure and are incorporated herein in its entirety.

### TECHNICAL FIELD

The disclosure relates to a method for programmatically managing antibody disulfide bonds site-specific modification, the modified antibody prepared by the method and the use thereof.

### BACKGROUND

The statements in this section merely provide background information related to the present disclosure and do not necessarily constitute prior art.

Site-specific modification approaches have been extensively employed in the development of protein-based technologies. The modification of proteins has emerged as a valuable approach to interrogate and to intervene in biological systems. A protein modification approach, without altering the structure or activity of the protein, is crucial to the development of protein-drug conjugates, including antibody-drug conjugates (ADCs).

Generally, antibody conjugation to cytotoxic agents commonly involves conjugation to exposed residues including lysines or reduction of disulfide bonds to expose free interchain cysteines on a therapeutic IgG (Immunoglobulin G) antibody. There are other, more recent approaches that introduce conjugation sites to the mAb such as site-specific glycan conjugation, cysteine engineering, incorporation of unnatural amino acids and coupling short peptide tags to drug-linkers. There are typically 80 lysine residues on an antibody; however, less than ten residues are chemically accessible for conjugation. Cysteine conjugation eventuates in the reduction of four interchain disulfide bonds. These bonds are reduced under specific conditions and subsequently result in two, four, six or eight exposed sulfhydryl groups. Both Cys and Lys conjugation methods result in heterogeneous mixtures. (“Advances and Limitations of Antibody Drug Conjugates for Cancer”. Biomedicines. 2021 August; 9(8): 872.).

The drug-antibody ratio (DAR), or number of drug molecules conjugated to a single ADC, is very important for the determination of efficacy of ADCs. DAR widely varies and depends on other ADC variables. The DAR values are also dependent on the site of conjugation and the use of light or heavy conjugated chains. The DAR value influences the effectiveness of the medicine due to the depression in potency caused by low drug loading, while elevated drug loading can impact toxicity and pharmacokinetics (“Introduction to Antibody-Drug Conjugates”. Antibodies (Basel). 2021 December; 10(4): 42.).

A number of methods have been developed to improve the homogeneity of ADCs. For example, Synaffix's technology GlycoConnect™ (US2015/0320882, synaffix.com/platform/technology/) has been developed to covert an antibody into a stably conjugated ADC with DAR2, DAR4 or even DAR1 and DAR6, by modifying the native antibody glycan through a three-step process: enzyme digestion, enzyme mediated ligation and metal-free click chemistry

US20210040145 discloses a 14-amino acid peptide Tub-tag fused to the C-terminus of any protein of interest (POI) and catalyzes the addition of a variety of different tyrosine derivatives. Taking advantage of this enzyme, Tub-tag technology repurposed tubulin-tyrosine ligase for the attachment of functional moieties at the C-terminus of antibody to homogeneously generate antibody conjugates with DAR 2.

However, those technologies suffer from several drawbacks, such as immunogenicity risk, complicated purification, and/or high cost.

Therefore, there is still a need for site-specific modification of an antibody.

### SUMMARY

For the above-mentioned purpose, provided herein is a method for programmatically managing antibody disulfide bonds site-specific modification, comprising step,
(R1) contacting a first reductant or salt thereof and an antibody in the presence of transition metal ions, to reduce at least one of the interchain disulfide bond of the antibody.

In some embodiments, in step (R1), one of the interchain disulfide bond of the antibody is reduced, optionally, one of the interchain disulfide bond in the hinge region of the antibody is reduced.

In some embodiments, in step (R1), three of the interchain disulfide bonds of the antibody are reduced, optionally, two of the interchain disulfide bonds in the Fab region and one of the interchain disulfide bond in the hinge region of the antibody are reduced.

In some embodiments, the method also includes step (O1),
(O1) introducing an oxidant to selectively re-oxidize the reduced thiol groups resulted from step (R1).

In some embodiments, the method also includes step (R2),
(R2) incubating a second reductant or salt thereof in the buffer system to reduce one of the interchain disulfide bond of the antibody resulted from step (O1).

In some embodiments, the method further comprises the following step,
(C1) introducing an amount of metal chelators and at least equimolecular proportion of a first conjugating group to react with the reduced thiol groups resulted from step (R1), (O1) or (R2), calculated as the molar amount of antibody, wherein, the first conjugating group is a first end capping reagent, a first linker-payload or a first thiobridge reagent, optionally, the first thiobridge reagent bears the first linker-payload or reactive groups.

In some embodiments, the method further comprises the following step,
(C2) incubating at least equimolecular proportion of the first conjugating group to react with the reduced thiol groups resulted from step (R1), calculated as the molar amount of antibody; then,
optionally, introducing the metal chelators and the oxidant, or
optionally, introducing the metal chelators and the first conjugating group.

In some embodiments, the method also includes the following steps,
(R3) incubating the second reductant or salt thereof in the buffer system to reduce the interchain disulfide bonds of the antibody resulted from step (C1) or (C2), optionally, introducing the transition metal ions;
(C3) introducing at least equimolecular proportion of a second conjugating group to react with the reduced thiol groups resulted from step (R3), calculated as the molar amount of antibody, wherein, the second conjugating group is a second end capping reagent, a second linker-payload or a second thiobridge reagent, optionally, the second thiobridge reagent bears the second linker-payload or reactive groups.

The method provided herein is compatible with current thiol-reactive linker-drug technologies with minimum conformation change and intact Fc function. Meanwhile it has simple manipulation and reduced cost and is simple to operate without enzymes engineering, and it is fully compatible with current thiol-reactive linker-drug technologies.

In one aspect, provided herein is a modified antibody prepared by the method above. The modified antibody is conjugated with one, two or three kinds of conjugating groups.

In one aspect, provided herein is a pharmaceutical composition comprising the modified antibody prepared by the method above, and at least one pharmaceutically acceptable ingredient.

In one aspect, provided herein is the use of the modified antibody prepared by the method above or the pharmaceutical composition provided herein in the manufacture of a therapeutic agent for preventing, diagnosing, or treating a disease.

In one aspect, provided herein is a method of preventing or treating a disease in a subject in need thereof, comprising administrating to the subject a therapeutically effective amount of a modified antibody prepared by the method above.

### BRIEF DESCRIPTION OF THE DRAWINGS

The following is a brief description of the drawings, which are presented for the purposes of illustrating the exemplary embodiments disclosed herein and not for the purposes of limiting the same.

FIGS. 1-2 show HIC-HPLC chromatogram of Trastuzumab-[MC-VC-PAB-MMAE]₂ conjugate, prepared by example I.1 and example I.4.

FIG. 3 shows HIC-HPLC chromatogram of Trastuzumab-[Maleimide-PEG4-N3-DBCO-Cy3]₁ conjugate of example I.125.

FIG. 4 shows HIC-HPLC chromatogram of Trastuzumab-[Maleimide-PEG4-N3-DBCO-Cy3]₁ conjugate prepared of example I.126.

FIG. 5 shows HIC-HPLC chromatogram of Trastuzumab-[Maleimide]₁ conjugate of example 1.127.

FIG. 6 shows HIC-HPLC chromatogram of Trastuzumab-[Maleimide]₁[MC-GGFG-DXd]₆ conjugate of example I.127.

FIG. 7 A shows HIC-HPLC chromatogram of Trastuzumab-[Maleimide]₁ conjugate of example 1.128; B shows HIC-HPLC of Trastuzumab-[Maleimide]₁[MC-VC-PAB-MMAE]₆ conjugate of example I.128.

FIG. 8 A shows HIC-HPLC chromatogram of Trastuzumab-[MC-VC-PAB-MMAE]₂ of example I.129; B shows HIC-HPLC chromatogram of Trastuzumab-[MC-VC-PAB-MMAE]₂[MC-GGFG-DXd]₆ conjugate of example I.129.

FIG. 9 shows HIC-HPLC chromatogram of Trastuzumab-[MC-MMAF]₂ of example I.130.

FIG. 10 shows HIC-HPLC chromatogram of Trastuzumab-[MC-MMAF]₂[MC-GGFG-DXd]₆ conjugate of example I.130.

FIG. 11 A shows HIC-HPLC chromatogram of Trastuzumab-[MC-VC-PAB-MMAE]₂ conjugate prepared of Example I.131; B-C show HIC-HPLC chromatogram of Trastuzumab-[MC-VC-PAB-MMAE]₂[MC-GGFG-DXd]₂ conjugate prepared of Example I.131-I.132.

FIG. 12 shows HIC-HPLC chromatogram of Trastuzumab-[Maleimide-PEG4-N3-DBCO-Cy3]₁[MC-VC-PAB-MMAE]₂ conjugate prepared of Example I.133.

FIG. 13 A shows HIC-HPLC chromatogram of Trastuzumab-[Maleimide]₁ conjugate prepared of Example I.134; B-C shows HIC-HPLC chromatogram of Trastuzumab-[Maleimide][MC-VC-PAB-MMAE]₄ conjugate prepared of Example I.134-I.135.

FIG. 14 A shows HIC-HPLC chromatogram of Trastuzumab-[MC-GGFG-DXd]₂ conjugate prepared of Example I.136; B-C show HIC-HPLC chromatogram of Trastuzumab-[MC-GGFG-DXd]₂[MC-VC-PAB-MMAE]₄ conjugate prepared of Example I.136-I.137.

FIG. 15 shows HIC-HPLC chromatogram of Trastuzumab-[Maleimide-PEG4-N3-DBCO-Cy3]₁[MC-VC-PAB-MMAE]₄ conjugate prepared of Example I.138.

FIGS. 16-17 show HIC-HPLC chromatogram of Trastuzumab-[MC-VC-PAB-MMAE]₆, prepared by Example II.1 and Example II.4.

FIGS. 18-19 show HIC-HPLC of Trastuzumab-[Bismaleimide-DBCO]₃ conjugate prepared by example II.144 and example II.145.

FIG. 20 A shows HIC-HPLC of Trastuzumab-[MC-GGFG-DXd]₆ conjugate of example II.146; B shows HIC-HPLC of Trastuzumab-[MC-GGFG-DXd]₆[MC-VC-PAB-MMAE]₂ conjugate of example II.146.

FIG. 21 A shows HIC-HPLC of Trastuzumab-[MC-VC-PAB-MMAE]₆ conjugate of example II.147; B shows HIC-HPLC of Trastuzumab-[MC-VC-PAB-MMAE]₆ [MC-GGFG-DXd]₂ conjugate of example II.147.

FIG. 22 A shows HIC-HPLC of Trastuzumab-[MC-VC-PAB-MMAE]₆ of example II.148; B shows HIC-HPLC of Trastuzumab-[MC-VC-PAB-MMAE]₆[Maleimide-PEG4-N3-DBCO-Cy3]₁ of example II.148.

FIG. 23 A shows HIC-HPLC of Trastuzumab-[MC-GGFG-DXd]₆ conjugate of example II.149; B shows HIC-HPLC of Trastuzumab-[MC-GGFG-DXd]₆[Maleimide-PEG4-N3-DBCO-Cy3]₁ conjugate of example II.149.

FIG. 24 A shows HIC-HPLC of Trastuzumab-[Maleimide]₆ conjugate of example II.150; B shows HIC-HPLC of Trastuzumab-[Maleimide]₆[MC-VC-PAB-MMAE]₂ conjugate of example II.150.

FIG. 25 A shows HIC-HPLC of Trastuzumab-[Maleimide]₆ conjugate of example II.151; B shows HIC-HPLC of Trastuzumab-[Maleimide]₆[MC-VC-PAB-MMAE]₂ conjugate of example II.151.

FIG. 26-27 show HIC-HPLC of Trastuzumab-[Maleimide]₆[Maleimide-PEG4-N3-DBCO-Cy3]₁ conjugate of example II.152-II.153.

FIG. 28 shows HIC-HPLC of Trastuzumab-[Maleimide-PEG4-N3-DBCO-MMAE]₁ conjugate of example III.93.

FIG. 29 shows HIC-HPLC of Trastuzumab-[Maleimide-PEG4-N3-DBCO-Cy3]₁ conjugate of example III.94.

FIG. 30 shows HIC-HPLC of Trastuzumab-[Maleimide-PEG4-N3-DBCO-MMAE]₁[MC-GGFG-DXd]₆ conjugate of example III.95.

FIG. 31 shows HIC-HPLC of Trastuzumab-[Maleimide-PEG4-N3-DBCO-Cy3]1[MC-VC-PAB-MMAE]₆ conjugate of example III.96.

FIG. 32-33 show HIC-HPLC of Trastuzumab-[Maleimide-PEG4-N3-DBCO-Cy3]1[MC-VC-PAB-MMAE]₂ conjugate of example III.97-III.98.

FIG. 34 A shows HIC-HPLC of Trastuzumab-[Maleimide-PEG4-N3-DBCO-Cy3]1 conjugate of example III.99; B-C show HIC-HPLC of Trastuzumab-[Maleimide-PEG4-N3-DBCO-Cy3]1[MC-VC-PAB-MMAE]₄ conjugate of examples III.99-III.100.

FIG. 35 A shows HIC-HPLC of Trastuzumab-[Maleimide-PEG4-N3-DBCO-Cy3]1 conjugate of example III.101; B-C show HIC-HPLC of Trastuzumab-[Maleimide-PEG4-N3-DBCO-Cy3]1[MC-VC-PAB-MMAE]₄ conjugate of examples III.101-III.102.

FIG. 36 A shows HIC-HPLC of Trastuzumab-[MC-VC-PAB-MMAE]₂ conjugate of example III.103; B shows HIC-HPLC of Trastuzumab-[MC-VC-PAB-MMAE]₂[MC-GGFG-DXd]₄ conjugate of example III.103.

FIG. 37 A shows HIC-HPLC of Trastuzumab-[MC-GGFG-DXd]₂ conjugate of example III.104; B shows HIC-HPLC of Trastuzumab-[MC-GGFG-DXd]₂[MC-VC-PAB-MMAE]₄ conjugate of examples III.104.

FIG. 38 A shows HIC-HPLC of Trastuzumab-[MC-VC-PAB-MMAE]₂ conjugate of example III.105; B-C show HIC-HPLC of Trastuzumab-[MC-VC-PAB-MMAE]₂[MC-GGFG-DXd]₄ conjugate of examples III.105-III.106.

FIG. 39 shows HIC-HPLC of Trastuzumab-[MC-VC-PAB-MMAE]₄ conjugate of example V.1.

FIG. 40 A shows HIC-HPLC of Trastuzumab-[MC-VC-PAB-MMAE]₄ conjugate of example V.46; B shows HIC-HPLC of Trastuzumab-[MC-VC-PAB-MMAE]₄[MC-GGFG-DXd]₂ conjugate of examples V.46.

FIG. 41 A shows HIC-HPLC of Trastuzumab-[MC-VC-PAB-MMAE]₄ conjugate of example V.47; B shows HIC-HPLC of Trastuzumab-[MC-VC-PAB-MMAE]₄[MC-GGFG-DXd]₂ conjugate of examples V.47.

FIG. 42 A shows HIC-HPLC chromatogram of Trastuzumab-[MC-VC-PAB-MMAE]₂ conjugate of Example I.139; B shows HIC-HPLC chromatogram of Trastuzumab-[MC-VC-PAB-MMAE]₂[MC-GGFG-DXd]₂ conjugate of Example I.139 before AKTA; C shows HIC-HPLC chromatogram of Trastuzumab-[MC-VC-PAB-MMAE]₂[MC-GGFG-DXd]₂ conjugate of Example 1.139 After AKTA; D shows HIC-HPLC chromatogram of Trastuzumab-[MC-VC-PAB-MMAE]₂ [MC-GGFG-DXd]₂[MC-MMAF]₂ conjugate prepared of Example I.139.

### DETAILED DESCRIPTION

The present disclosure is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following description is intended to illustrate some particular embodiments of the invention, and not to exhaustively specify all permutations, combinations and variations thereof.

Unless defined otherwise, all technical and scientific terms used herein have the same meaning as commonly understood by one of ordinary skill in the art to which the disclosure pertains. Although any methods and materials similar or equivalent to those described herein may be used in the practice for testing of the present disclosure, the preferred materials and methods are described herein. In describing and claiming the present disclosure, the following terminology will be used.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “an antibody” includes a plurality of antibodies; reference to “a transition metal ion” includes mixtures of transition metal ions, and the like. In this application, the use of “or” means “and/or” unless stated otherwise.

Throughout this disclosure, unless the context requires otherwise, the words “comprise”, “comprises”, “comprising”, “contain”, “contains” and “containing” will be understood to imply the inclusion of a stated step or element or group of steps or elements but not the exclusion of any other step or element or group of steps or elements. By “consisting of” is meant including, and limited to, whatever follows the phrase “consisting of”. Thus, the phrase “consisting of” indicates that the listed elements are required or mandatory, and that no other elements may be present.

### Method for Antibody Modification

Provided herein is a method for programmatically managing (controlling) antibody disulfide bonds modification, which could modify a protein, such as an antibody, to covalent link a group of interest. The products may be antibody-drug conjugates (ADCs), groups of interest (e.g., cytotoxic drug) linking to a monoclonal antibody via linkers or thiobridge reagents. Particularly, provided herein is a method for programmatically managing antibody disulfide bonds site-specific modification, the site-specific modified antibody with improved homogeneity are benefit for enhancing safety and curative effect.

The present disclosure provides examples of methods for programmatically managing antibody disulfide bonds site-specific modification, the method includes the following step,
(R1) contacting a first reductant or salt thereof and an antibody in a buffer system in the presence of transition metal ions, to reduce at least one of the interchain disulfide bond of the antibody.

The reductant, optionally, with the transition metal ions, has reducibility and reduces the disulfide bond of an antibody to modify protein or antibody. Optionally, the reductant selectively reduces the interchain disulfide bonds, thus the antibody is selectively modified.

In some embodiments, provided herein is the method I or method II for programmatically managing antibody disulfide bonds site-specific modification. In some embodiments, in step (R1) of method I, one of the interchain disulfide bond of the antibody is reduced, optionally one of the interchain disulfide bond in the hinge region of the antibody is reduced. In some embodiments, in step (R1) of method II, three of the interchain disulfide bonds of the antibody are reduced, optionally two of the interchain disulfide bonds in the Fab region and one of the interchain disulfide bond in the hinge region of the antibody are reduced.

In some embodiments, the method I and/or method II includes the following steps,
(R1) incubating the first reductant or salt thereof and the antibody in the buffer system in the presence of the transition metal ions, to reduce at least one of the interchain disulfide bond of the antibody;
(C1) introducing an amount of metal chelators and at least equimolecular proportion of a first conjugating group to react with the reduced thiol groups resulted from step (R1), calculated as the molar amount of antibody, wherein, the first conjugating group is a first end capping reagent, a first linker-payload or a first thiobridge reagent, optionally, the first thiobridge reagent bears the first linker-payload or reactive groups.

In some embodiments, in step (C1) of method I, one or two of the first conjugating groups are covalently linked to the reduced thiol groups resulted from step (R1).

In some embodiments, in step (C1) of method II, three or six of the first conjugating groups are covalently linked to the reduced thiol groups resulted from step (R1).

As used herein, the term “disulfide bond” refers to a covalent bond with the structure R—S-S-R′. The amino acid cysteine comprises a thiol group that can form a disulfide bond with a second thiol group, for example from another cysteine residue. The disulfide bond can be formed between the thiol groups of two cysteine residues residing respectively on the two polypeptide chains, thereby forming an interchain bridge or interchain bond.

As used herein, the term “hinge region” refers to an antibody includes the portion of a heavy chains molecule that joins the CH1 domain to the CH2 domain. This hinge region comprises approximately 25 amino acid residues and is flexible, thus allowing the two N-terminus antigen binding regions to move independently.

As used herein, the term “Fab fragments” refers to the region of the antibody structure that can bind to antigen. It consists of a complete light chain (variable and constant regions) and part of the heavy chain structure (variable and a constant region fragment), the light and heavy chains are connected by a disulfide bond. Fab fragments can be obtained by protease digestion of full-length antibodies. Under the action of papain, human immunoglobulin G can be degraded into two Fab fragments and one Fc fragment; under the action of pepsin, IgG can be degraded into an F(ab′)2 fragment and a pFc′ fragment. The F(ab′)2 fragment can be further reduced to form two Fab′ fragments. In some embodiments, the interchain disulfide bonds connect two of the upper heavy chains in the hinge region or the interchain disulfide bonds connect the heavy chain to the light chain in Fab region.

As used herein, the term “antibody” refers to any immunoglobulin, monoclonal antibody, polyclonal antibody, multispecific antibody, or bispecific (bivalent)antibody that binds to a specific antigen. A native intact antibody comprises two heavy chains and two light chains. Each heavy chain consists of a variable region (“HCVR”) and a first, second, and third constant region (CH1, CH2 and CH3), while each light chain consists of a variable region (“LCVR”) and a constant region (CL). Mammalian heavy chains are classified as α, δ, ε, γ and μ, and mammalian light chains are classified as λ or κ. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light (L) chain CDRs including LCDR1, LCDR2, and LCDR3, heavy (H) chain CDRs including HCDR1, HCDR2, HCDR3). CDR boundaries for antibodies may be defined or identified by the conventions of Kabat, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3): 651-63 (1985); Chothia, C. and Lesk, A. M., J. Mol. Biol., 196, 901 (1987); Chothia, C. et al., Nature. December 21-28; 342(6252): 877-83 (1989); Kabat E. A. et al., National Institutes of Health, Bethesda, Md. (1991)). The three CDRs are interposed between flanking stretches known as framework regions (FRs), which are more highly conserved than the CDRs and form a scaffold to support the hypervariable loops. Each HCVR and LCVR comprises four FRs, and the CDRs and FRs are arranged from amino terminus to carboxy terminus in the order: FR1, CDR1, FR2, CDR2, FR3, CDR3, FR4. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ⁴ heavy chain), IgA1 (α1 heavy chain), or IgA2 (α2 heavy chain).

In some embodiments, the method comprises step (O1),
(O1) introducing an oxidant to selectively re-oxidize the reduced thiol groups resulted from step (R1).

In some embodiments, provided herein is the method III for programmatically managing antibody disulfide bonds site-specific modification. The method III includes the following steps,
(R1) incubating the first reductant or salt thereof and the antibody in the buffer system in the presence of the transition metal ions, to reduce three of the interchain disulfide bonds of the antibody;
(O1) introducing the oxidant to selectively re-oxidize the reduced thiol groups resulted from step (R1);
(C1) introducing an amount of the metal chelators and at least equimolecular proportion of the first conjugating group to react with reduced thiol groups resulted from step (O1), calculated as the molar amount of the antibody.

In some embodiments, the oxidant in step (O1) re-oxidizes the reduced thiol groups in Fab region of the antibody resulted from step (R1), optionally, four of the reduced thiol groups are re-oxidized to form two disulfide bonds in step (O1).

In some embodiments, in step (C1) of method III, one or two of the first conjugating groups are covalently linked to the remaining thiol groups resulted from step (O1).

In some embodiments, the method comprises step (R2),
(R2) incubating a second reductant or salt thereof in the buffer system to reduce one of the interchain disulfide bond of the antibody resulted from step (O1).

In some embodiments, provided herein is the method IV for programmatically managing antibody disulfide bonds site-specific modification, the method IV includes the following steps,
(R1) incubating the first reductant or salt thereof and the antibody in the buffer system in the presence of the transition metal ions, to reduce three of the interchain disulfide bond of the antibody;
(O1) introducing the oxidant to selectively re-oxidize the reduced thiol groups resulted from step (R1);
(R2) incubating the second reductant or salt thereof in the buffer system to reduce one of the interchain disulfide bond of the antibody resulted from step (O1);
(C1) introducing an amount of the metal chelators and at least equimolecular proportion of the first conjugating group to react with the reduced thiol groups resulted from step (R2), calculated as the molar amount of the antibody.

In some embodiments, in step (R2) of method IV, one of the interchain disulfide bond in the hinge region of the antibody is reduced.

In some embodiments, in step (C1) of the method IV, two or four of the first conjugating groups are covalently linked to the remaining thiol groups resulted from step (R2).

In some embodiments, the method further comprises the following step, (C2) incubating at least equimolecular proportion of the first conjugating group to react with the reduced thiol groups resulted from step (R1), calculated as the molar amount of antibody, then, optionally, introducing the metal chelators and the oxidant, or optionally, introducing the metal chelators and the first conjugating group.

In some embodiments, provided herein is the method V for programmatically managing antibody disulfide bonds site-specific modification, the method V includes the following steps,
(R1) incubating the first reductant or salt thereof and the antibody in the buffer system in the presence of the transition metal ions, to reduce three of the interchain disulfide bonds of the antibody;
(C2) incubating at least equimolecular proportion of the first conjugating group to react with the reduced thiol groups resulted from step (R1), calculated as the molar amount of antibody, then, optionally, introducing the metal chelators and the oxidant.

In some embodiments, provided herein is the method V for programmatically managing antibody disulfide bonds site-specific modification, the method V includes the following steps,
(R1) incubating the first reductant or salt thereof and the antibody in the buffer system in the presence of the transition metal ions, to reduce three of the interchain disulfide bonds of the antibody;
(C2) incubating at least equimolecular proportion of the first conjugating group to react with the reduced thiol groups resulted from step (R1), calculated as the molar amount of antibody, then, optionally, introducing the metal chelators and the first conjugating group.

In some embodiments, in step (C2) of the method V, the metal chelators can trap the excessive transition metal ions. Before introducing the metal chelators, the first conjugating groups could react with four of the reduced thiol groups from step (R1). After introducing the metal chelators, the first conjugating groups could react with two of the remaining reduced thiol groups again.

In some embodiments, in step (C2) of the method V, two or four of the first conjugating groups are covalently linked to the remaining thiol groups resulted from step (R1) firstly. When introducing the metal chelators and the first conjugating group, one or two of the first conjugating groups are covalently linked to the remaining thiol groups again.

In some embodiments, in step (C2) of the method V, the first conjugating groups are same or different.

In some embodiments, when the first thiobridge reagent bears the reactive groups, the step (C2) comprises the following step,
introducing the first thiobridge reagent bearing the reactive groups to re-bridge the reduced thiol groups resulted from step (R1), incubating the first linker-payload in the buffer system to react with the reactive groups of the thiobridge group, then,
optionally, introducing the metal chelators and the oxidant, or
optionally, introducing the metal chelators and the first conjugating group.

In some embodiments, when the first thiobridge reagent bears the reactive groups, the step (C1) of method I, II, III and IV comprises the following step,
introducing the metal chelators and the first thiobridge reagent bearing the reactive groups to re-bridge the reduced thiol groups resulted from step (R1), (O1) or (R2), then, incubating the first linker-payload in the buffer system to react with the reactive groups of the thiobridge group.

In some embodiments, the method I, II, III, IV and V further comprises the following steps,
(R3) incubating the second reductant or salt thereof in the buffer system to reduce the interchain disulfide bonds of the antibody resulted from step (C1) or (C2), optionally, introducing the transition metal ions;
(C3) introducing at least equimolecular proportion of a second conjugating group to react with the reduced thiol groups resulted from step (R3), calculated as the molar amount of antibody, optionally, introducing the metal chelators, wherein, the second conjugating group is a second end capping reagent, a second linker-payload or a second thiobridge reagent, optionally, the second thiobridge bears the second linker-payload or reactive groups.

In some embodiments, when the second thiobridge reagent bears the reactive groups, the step (C3) comprises the following step,
introducing the second thiobridge reagent bearing the reactive groups to re-bridge the reduced thiol groups resulted from step (R3), optionally, introducing the metal chelators, then, incubating the second linker-payload in the buffer system to react with the reactive groups of the thiobridge group.

In some embodiments, when introducing the transition metal ions in step (R3), introducing the metal chelators to trap the excess transition metal ions in step (C3).

In some embodiments, in step (R3), one, two or three of the interchain disulfide bonds of the antibody is reduced.

In some embodiments, in step (C3), one, two, three, four or six of the second conjugating groups are covalently linked to the reduced thiol groups resulted from step (R3).

In some embodiments, in step (R3) of method I and/or method III, three of the interchain disulfide bonds of the antibody are reduced. In some embodiments, in step (C3) of method I and/or method III, three or six of the second conjugating groups are covalently linked to the reduced thiol groups resulted from step (R3).

In some embodiments, in step (R3) of method I and/or method III, one or two of the interchain disulfide bonds of the antibody are reduced, when introducing the transition metal ions. In some embodiments, in step (C3) of method I and/or method III, one, two or four of the second conjugating groups are covalently linked to the reduced thiol groups resulted from step (R3), when introducing the transition metal ions.

In some embodiments, in step (R3) of method II, one of the interchain disulfide bonds of the antibody are reduced. In some embodiments, in step (C3) of method II, one or two of the second conjugating groups are covalently linked to the reduced thiol groups resulted from step (R3).

In some embodiments, in step (R3) of method IV and/or method V, two of the interchain disulfide bonds of the antibody are reduced. In some embodiments, in step (C3) of method IV and/or method V, two or four of the second conjugating groups are covalently linked to the reduced thiol groups resulted from step (R3).

In some embodiments, in step (R3) of method IV and/or method V, one of the interchain disulfide bonds of the antibody are reduced, when introducing the transition metal ions. In some embodiments, in step (C3) of method IV and/or method V, one or two of the second conjugating groups are covalently linked to the reduced thiol groups resulted from step (R3), when introducing the transition metal ions.

In some embodiments, when one of the interchain disulfide bond is reduced in step (R3) in method I and III, the method I and III further comprises the following steps,
(R4) incubating the transition metal ions and the second reductant or salt thereof in the buffer system to reduce the interchain disulfide bonds of the antibody resulted from step (C3);
(C4) introducing the metal chelators and at least equimolecular proportion of the second conjugating group to react with the reduced thiol groups resulted from step (R4), calculated as the molar amount of antibody.

In some embodiments, in step (R4) of the method I and/or method III, one of the interchain disulfide bonds of the antibody are reduced. In some embodiments, in step (C4) of the method I and/or method III, one or two of the second conjugating groups are covalently linked to the reduced thiol groups resulted from step (R4).

In some embodiments, the second conjugating groups in step (C3) is same as that in step (C4). In some embodiments, the second conjugating groups in step (C3) is different from that in step (C4).

In some embodiments, the method of the site-specific modification dose not refer to enzyme technologies and glycan modification.

In the present disclosure, in some embodiments, the reductants, including the first reductant and the second reductant, are independently selected from a group consisting of tris (2-carboxyethyl) phosphine (TCEP), or a compound having the following formula (I),

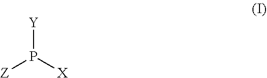

or a salt, solvate, stereoisomer thereof, wherein
X, Y and Z independently covalently connect the phosphorus atom through P-C bond, which is P-C(sp³) or P-C(sp²);
X is of formula (III):

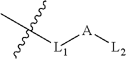

L₁ is selected from the group consisting of —CH(R₁)—, —C(CH₃)(R₁), —CH(R₁)CH(R₂)—, —CH(R₁)CH(R₂)CH(R₃)—, aryl group which is optionally independently substituted with group containing at least a coordinating atom, and heteroaryl group which is optionally independently substituted with group containing at least a coordinating atom selected from O and S;
R₁, R₂ and R₃ independently are H, C₁-C₃ alkyl group, C₁-C₃ hydroxyalkyl group, C₁-C₃ carboxy alkyl group, C₁-C₃ hydroxylamine alkyl group, C₁-C₃ N-hydroxy amide alkyl group, aryl group or heteroaryl group; or
R₂ or R₃ forms a 5-6 membered optionally substituted ring with L₂;
A is optionally present and is —C(O)—, or —C(O) J-;
J is organic group comprising amino or imino group and carbonyl group at the same time, of which the amino or imino group forms amide group with —C(O), the carboxyl group optionally covalently links to L₂;
L₂ is optionally present, L₂ works as transition metal chelator motif and is —N(R₄)(R₅) or hydroxy;
R₄ and R₅ independently are hydrogen, C₀-C₅ hydroxyalkyl group, C₁-C₅ alkyl group, C₁-C₅ alkoxy group, —(CH₂)n¹(OCH₂CH₂O)n²CH(R₆)CO(R₇), optionally substituted 5-6 membered saturated heterocyclic group, optionally substituted arylalkyl group, optionally substituted aryl alkoxy group, optionally substituted aryl group, optionally substituted heteroaryl group, optionally substituted heteroaryl alkyl group, R₄ or R₅ forms a 5-6 membered optionally substituted ring with R₂ or R₃;
R₆ is hydrogen, amino, C₁-C₅ alkyl, C₁-C₅ hydroxyalkyl group, C₁-C₅ carboxy alkyl group, aryl group, optionally substituted arylalkyl group, C₁-C₅ N-hydroxy amide alkyl group, heteroaryl group or heteroaryl alkyl group;
R₇ is hydroxy, C₁-C₅ alkoxy group, —NH(CH₂CONH)n³OH;
n¹, n² and n³ independently are the number 0, 1, 2, 3, 4;
R₄ and R₅ are not hydroxy at the same time;
Y is same as X,
Z is same as X, or
Y and Z independently are 5-6 membered optionally substituted saturated heterocyclic group, C₁-C₃ alkyl group, C₁-C₃ hydroxyalkyl group, aryl group, C₁-C₃ carboxy alkyl group, 5-6 membered optionally substituted cycloalkyl group, or

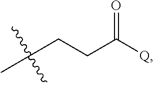

—C(O)Q is ester group, imide group or amide group,
X, Y and Z are not —CH₂CH₂C(O)OH at the same time.

In the disclosure, the term “aryl group” refers to an aromatic or hetero aromatic group, composed of one or several rings, comprising three to fourteen carbon atoms, preferentially six to ten carbon atoms. Exemplary aryl group is phenyl group.

The term “aryl group” also refers to an aromatic group, wherein one or several H atoms are replaced independently by other group, such as F, CT, Br, I, hydroxy, carboxy, sulfonyl, amino, methoxy or ethoxy, N-hydroxy formamide group, N-hydroxy acetamido group, 4-pyridyl group, 2-pyridyl group,

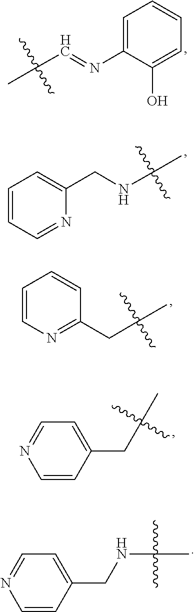

The term “C₀-C₅ alkyl group” refers to an aliphatic hydrocarbon group which having 1 to 5 carbon atoms in the chain or cyclic. Exemplary alkyl groups include methyl, ethyl, n-propyl and i-propyl.

The term “C₀-C₅ hydroxyalkyl group” refers to hydroxy group or C₁-C₅ alkyl group, wherein one or several H atoms are substituted with one, two or three hydroxy groups. Exemplary C₁-C₅ hydroxyalkyl group is hydroxy methyl group, 2-hydroxy ethyl group, 3-hydroxy propyl group.

The term “C₁-C₅ alkoxy group” refers to an oxygen atom attached to C₁-C₅ alkyl group. Exemplary C₁-C₅ alkoxy group is —OCH₃, —OCH₂CH₃, —OCH₂(CH₃)₂, —OCH₂CH₂CH₃.

The term “aryl alkoxy group” refers to an aromatic group, wherein one or several H atoms are replaced by alkoxy group. Exemplary phenyl-O-CH₂—, phenyl-O—(CH₂)₂—, phenyl-O—(CH₂)₃—, phenyl-O—(CH₂)₄—, phenyl-O—(CH₂)₅—.

The term “heteroaryl group” refers to one or several carbon on aromatic group, preferentially one, two, three or four carbon atoms are replaced by O, N, Si, Se, P or S, preferentially by O, S, N. Exemplary heteroaryl group is imidazolyl group, pyridyl group, bipyridyl group, quinolinyl group, iso-quinolinyl group.

The term “heteroaryl group” also refers to hetero aromatic group, wherein one or several H atoms are replaced independently by other group, such as F, Cl, Br, I, hydroxy, carboxy, amino, hydroxyalkyl group, carboxy alkyl group, N-hydroxy amide alkyl group, heteroaryl group.

The term “coordinating atom” refers to the atom containing lone paired electron, examples include N, O, S, P, F, Cl, Br, I.

The term “C₁-C₅ carboxy alkyl group” refers to a C₁-C₅ alkyl group which is substituted with one, two or three carboxy groups. Exemplary C₁-C₅ carboxy alkyl group is —COOH, —CH₂COOH, —CH₂CH₂COOH, —CH₂(CH₃)COOH.

The term “C₁-C₅ hydroxylamine alkyl group” refers to a C₁-C₅ alkyl group which is substituted with one, two or three hydroxylamine groups. Exemplary C₁-C₅ hydroxylamine alkyl group is —CH₂NHOH, —CH₂CH₂NHOH.

The term “C₁-C₅ N-hydroxy amide alkyl group” refers to a C₁-C₅ carboxy alkyl group, wherein one, two or three carboxy forms amide with hydroxylamine. Exemplary C₁-C₅ N-hydroxy amide alkyl group is —C(O)NHOH, —CH₂C(O)NHOH, —CH₂ CH2C(O)NHOH.

The term “heterocyclic group” refers to an aromatic or non-aromatic C₅-C₁₀ cycle composed of one or two rings, in which one or two of the ring carbon atoms are independently replaced with a heteroatom from the group of O, N, P and S. Preferable heteroatoms are O, N and S. Suitable heterocyclics are also disclosed in The Handbook of Chemistry and Physics, 76* Edition, CRC Press, Inc., 1995-1996, p 2-25 to 2-26, the disclosure of which is hereby incorporated by reference. Preferred non aromatic heterocyclic include, but are not limited to pyrrolidinyl, pyrazolidinyl, imidazolidinyl, oxiranyl, tetrahydrofuranyl, dioxolanyl, tetrahydro-pyranyl, dioxanyl.dioxolanyl, piperidyl, piperazinyl, morpholinyl, pyranyl, imidazolinyl, pyrrolinyl, pyrazolinyl, thiazolidinyl, tetrahydrothiopyranyl, dithianyl, thiomorpholinyl, dihydro-pyranyl, tetrahydropyranyl, diliydropyranyl, tetrahydro-pyridyl, dihydropyridyl, tetrahydropyrinidinyl, dihydrothiopyranyl, a/epanyl, as well as the fused systems resulting from the condensation with a phenyl group.

The term “arylalkyl group” refers to a liner, branched or cycloalkyl which is linked to at least one aryl group. Preferable the number of carbon atoms in the chain or cyclic is 1-4. Exemplary arylalkyl group is —CH₂C₆H₅, —CH₂CH₂C₆H₅, —CH₂CH₂CH₂C₆H₅, —CH₂(CH₃)CH₂C₆H₅, —CH₂(CH₃)CH₂CH₂C₆H₅,

The term “heteroaryl alkyl group” refers to a liner, branched or cycloalkyl which is linked to at least one heteroaryl group. Preferable the number of carbon atoms in the chain or cyclic is 1-4. Exemplary heteroaryl alkyl group is

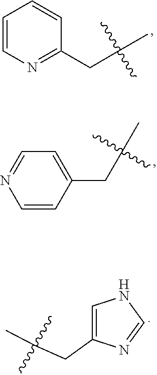 [SMILES: *Cc1ccccn1 ; *Cc1ccncc1 ; *Cc1c[nH]cn1]

The term “cycloalkyl group” refers to 3-, 4-, 5- or 6-membered saturated or unsaturated non-aromatic carbocyclic ring. Representative cycloalkyl group include, but are not limited to, cyclopropyl, cyclobutyl, cyclopentyl, cyclopentadienyl, cyclohexyl, cyclohexenyl, 1,3-cyclohexadienyl, 1,4-cyclohexadienyl. Cycloalkyl group can be unsubstituted or substituted with one or more groups including, but not limited to carboxyl, sulfonyl, amino, hydroxy, —C(O)NHOH, —CH₂C(O)NHOH, —CH₂ CH2C(O)NHOH, —COOH, —CH₂COOH, —CH₂CH₂COOH, —CH₂(CH₃)COOH, F, Cl, Br, I.

The term “halogen” refers to F, Cl, Br or I.

The term “Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted and may be straight or branched.

The term “Cyano” refers to a substituent having a carbon atom joined to a nitrogen atom by a triple bond, e.g., —CN.

In some embodiments, Li is —CH(R₁)—, —CH(R₁)CH(R₂)— or —CH(R₁)CH(R₂)CH(R₃)— in compound of formula (III).

In some embodiments, in the compound of formula (I), R₁, R₂ and R₃ independently are H, methyl group, isopropyl group, hydroxymethyl group, hydroxyethyl group, carboxy methyl group, carboxy ethyl group, N-hydroxy ethyl amide group, phenyl group, 2-pyridyl group, 4-pyridyl group or 4-imidazole group, R₂ forms a 5-6 membered optionally substituted ring with L₂.

In some embodiments, in the compound of formula (I), L₁ is —CH(R₁)CH(R₂)—, R₁ is H, and R₂ forms a 5-6 membered optionally substituted ring with R₄ of L₂. In some embodiments, R₂ forms

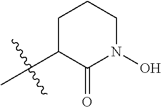 [SMILES: *C1CCCN(O)C1=O]

with L₂. In these embodiments of compound having formula (I), A is —C(O)—, L₂ is —N(R₄)(R₅), R₅ is hydroxy.

In some embodiments of compound having formula (I), L₁ is —CH(R₁)CH(R₂)—, R₁ is H, R₂ is methyl group, hydroxymethyl group, hydroxyethyl group, carboxy ethyl group, phenyl group, N-hydroxy ethyl amide group, 2-pyridyl group, 4-pyridyl group or 4-imidazole group.

In some embodiments of compound having formula (I), L₁ is —CH(R₁)CH(R₂)—, R₁ is H, R₂ is methyl group, hydroxymethyl group, hydroxyethyl group, carboxy ethyl group, phenyl group, N-hydroxy ethyl amide group, 2-pyridyl group, 4-pyridyl group or 4-imidazole group, A is —C(O)—, L₂ is —N(R₄)(R₅), R is hydrogen, optionally substituted 5-6 membered saturated heterocyclic group, R₅ is hydroxy. In these embodiments, R₄ is

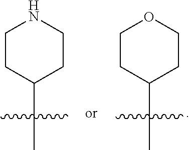 [SMILES: *C1CCNCC1 ; *C1CCOCC1]

In some embodiments of compound having formula (I), L₁ is —CH(R₁)CH(R₂)—, R₁ is H, R₂ is methyl group, hydroxymethyl group, hydroxyethyl group, carboxy ethyl group, phenyl group, N-hydroxy ethyl amide group, 2-pyridyl group, 4-pyridyl group or 4-imidazole group, A is —C(O)—, L₂ is —N(R₄)(R₅), R₄ and R₅ form a 5-6 membered optionally substituted ring. In these embodiments, L₂ is

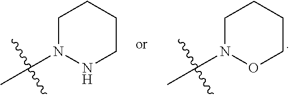 [SMILES: *N1CCCCN1 ; *N1CCCCO1]

In some embodiments of compound having formula (I), L₁ is —CH(R₁)CH(R₂)—, R₁ is methyl group, isopropyl group, carboxy ethyl group or N-hydroxy ethyl amide group, R₂ is H.

In some embodiments of compound having formula (I), L₁ is —CH(R₁)CH(R₂)—, R₁ is methyl group, isopropyl group, carboxy ethyl group or N-hydroxy ethyl amide group, R₂ is H, A is —C(O)—, L₂ is —N(R₄)(R₅), R₄ is hydrogen, and R₅ is hydroxy.

In some embodiments of compound having formula (I), L₁ is optionally substituted phenyl group connected to A in ortho, meta or para position, A is —C(O)—, L₂ is —N(R₄)(R₅) or hydroxy, R₄ is hydrogen, R₅ is hydroxy.

In some embodiments of compound having formula (I), Li is phenyl group which is optionally substituted with hydroxy, halogen, carboxyl, sulfonyl, amino, methoxy or ethoxy, in ortho, meta or para position, optionally, halogen refers to F, Cl, Br, or I. In these embodiments, A and L₂ are not present.

In some embodiments of compound having formula (I), L₁ is

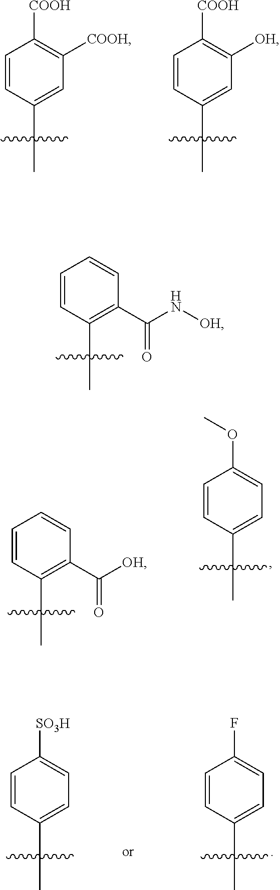

In these embodiments, A and L2 are not present.

In some embodiments of compound having formula (I), L₁ is optionally substituted 4-pyridyl group or optionally substituted 4-quinolyl group. In some embodiments of compound having formula (I), L₁ is

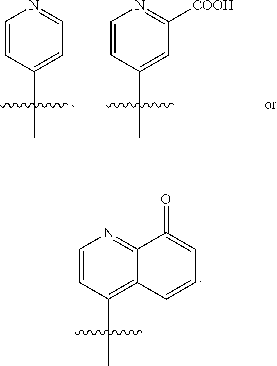

In these embodiments of compound having formula (I), A and L₂ are not present.

In some embodiments of compound having formula (I), L₁ is —CH(R₁)CH(R₂)—, R₁ and R₂ independently are H.

In some embodiments of compound having formula (I), A is —C(O)—, L₂ is —N(R₄)(R₅), R₄ is hydrogen, R₅ is hydroxy.

In some embodiments of compound having formula (I), L₂ is —N(R₄)(R₅), R₄ is hydrogen, C₁-C₅ alkyl group, —(CH₂)n¹(OCH₂CH₂O)n²CH(R₆)CO(R₇), optionally substituted 5-6 membered saturated heterocyclic group, optionally substituted arylalkyl group, optionally substituted aryl group, optionally substituted heteroaryl alkyl group, or R₄ and R₅ form a 5-6 membered optionally substituted ring; R₅ is hydroxy.

In some embodiments of compound having formula (I), L₂ is —N(R₄)(R₅), R₄ is hydrogen, C₁-C₅ alkyl group, —(CH₂)n¹(OCH₂CH₂O)n²CH(R₆)CO(R₇), optionally substituted arylalkyl group, optionally substituted aryl group; R₅ is hydroxy.

In some embodiments of compound having formula (I), R6 is hydrogen, amino, C₁-C₅ alkyl, C₁-C₅ hydroxyalkyl group, C₁-C₅ carboxy alkyl group, aryl group, C₁-C₅ N-hydroxy amide alkyl group, heteroaryl group or heteroaryl alkyl group.

In some embodiments of compound having formula (I), R₆ is hydrogen, C₁-C₅ alkyl, C₁-C₅ hydroxyalkyl group, or heteroaryl alkyl group.

In some embodiments of compound having formula (I), R6 is hydrogen, methyl group, hydroxymethyl group amino, benzyl group, carboxy ethyl group, N-hydroxy ethyl amide group,

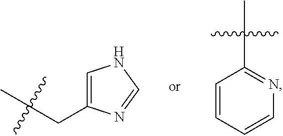 [SMILES: *Cc1c[nH]cn1 ; *c1ccccn1]

optionally, R₆ is hydrogen.

In some embodiments of compound having formula (I), R₇ is hydroxy, C₁-C₅ alkoxy group, —NH(CH₂CONH)n³OH, optionally, R₇ is hydroxy or C₁-C₅ alkoxy group. In some embodiments, R₇ is hydroxy, methoxy group, —NH(CH₂CONH)n³, optionally, R₇ is hydroxy or methoxy group.

In the compound of formula (I), n¹, n² and n³ independently are the number 0, 1, 2, 3, 4.

In some embodiments of compound having formula (I), R₄ is

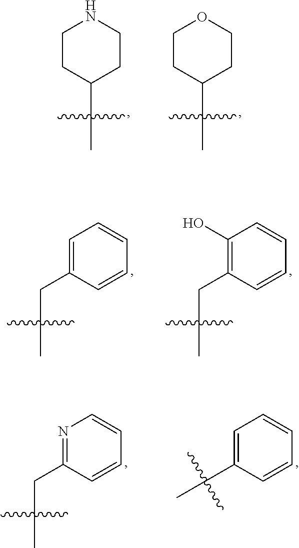 [SMILES: *C1CCNCC1 ; *C1CCOCC1 ; *Cc1ccccc1 ; *Cc1ccccc1O ; *Cc1ccccn1 ; *c1ccccc1]

hydrogen or —(CH₂)n¹(OCH₂CH₂O)n²CH(R₆)CO(R₇); R₅ is hydroxy; R₆ is hydrogen, methyl group, hydroxymethyl group or

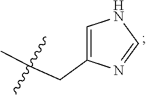 [SMILES: *Cc1c[nH]cn1]

R₇ is hydroxy or —NH(CH₂CONH)n³OH; n¹, n² and n³ independently are the number 0.

In some embodiments, R₄ is

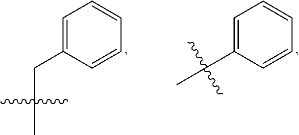 [SMILES: *Cc1ccccc1 ; *c1ccccc1]

hydrogen or —CH(R₆)CO(R₇), R₅ is hydroxy, R₆ is hydrogen, R₇ is hydroxy.

In some embodiments of compound having formula (I), L₂ is —N(R₄)(R₅);
R₄ and R₅ are independently —(CH₂)n¹(OCH₂CH₂O)n²CH(R₆)CO(R₇) or optionally substituted heteroaryl alkyl group;
R₆ is hydrogen, amino, C₁-C₅ alkyl, C₁-C₅ hydroxyalkyl group, C₁-C₅ carboxy alkyl group, aryl group, C₁-C₅ N-hydroxy amide alkyl group, heteroaryl group or heteroaryl alkyl group;
R₇ is hydroxy, C₁-C₅ alkoxy group, —NH(CH₂CONH)n³OH;
n¹, n² and n³ independently are the number 0, 1, 2, 3, 4.

In some embodiments of compound having formula (I), R₄ and R₅ are independently —(CH₂)n¹(OCH₂CH₂O)n²CH(R₆)CO(R₇) or 6 membered heteroaryl alkyl group;
R₆ is hydrogen;
R₇ is hydroxy, C₁-C₅ alkoxy group, —NH(CH₂CONH)n³OH;
n¹, n² and n³ independently are the number 0, 1, 2, 3, 4.

In some embodiments of compound having formula (I), R₄ and R₅ are independently —(CH₂)n¹(OCH₂CH₂O)n²CH(R₆)CO(R⁷) or

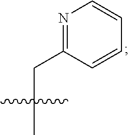 [SMILES: *Cc1ccccn1]

R₆ is hydrogen;
R₇ is hydroxy or —NH(CH₂CONH)n³OH;
n¹, n² and n³ independently are the number 0.

In some embodiments, R₄ and R₅ are independently —(CH₂)n¹(OCH₂CH₂O)n²CH(R₆)CO(R₇),
R₆ is hydrogen,
R₇ is hydroxy or —NH(CH₂CONH)n³OH;
n¹, n² and n³ independently are the number 0.

In some embodiments of compound having formula (I), L₂ is —N(R₄)(R₅);
R₄ is hydrogen, C₀-C₅ hydroxy alkyl group, C₁-C₅ alkyl group, optionally substituted C₁-C₅ alkoxy group, —(CH₂)n¹(OCH₂CH₂O)n²CH(R₆)CO(R₇), optionally substituted arylalkyl group, optionally substituted aryl alkoxy group, optionally substituted aryl group, optionally substituted heteroaryl group, optionally substituted heteroaryl alkyl group;
R₅ is hydrogen;
R₆ is hydrogen, amino, C₁-C₅ alkyl, C₁-C₅ hydroxyalkyl group, C₁-C₅ carboxy alkyl group, aryl group, optionally substituted arylalkyl group, C₁-C₅ N-hydroxy amide alkyl group, heteroaryl group or heteroaryl alkyl group;
R₇ is hydroxy, C₁-C₅ alkoxy group, —NH(CH₂CONH)n³OH;
n¹, n² and n³ independently are the number 0, 1, 2, 3, 4.

In some embodiments of compound having formula (I), R₄ is hydrogen, C₀-C₃ hydroxyalkyl group, C₁-C₃ alkoxy group, —(CH₂)n¹(OCH₂CH₂O)n²CH(R₆)CO(R₇), phenyl group which is substituted with carboxy, hydroxy, amino, halogen, pyridyl group, amino which is substituted with 2-methylpyridine, benzyl group which is substituted with carboxy, hydroxy, amino or halogen, aryl alkoxy group, pyridyl group which is substituted with carboxy, bipyridyl group, or

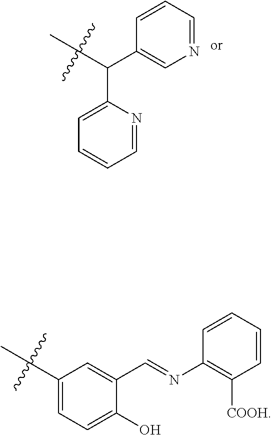 [SMILES: *C(c1cccnc1)c1ccccn1 ; *c1ccc(O)c(/C=N/c2ccccc2C(=O)O)c1]

In some embodiments of compound having formula (I), R₄ is hydrogen, hydroxy, methyl hydroxyl group, ethyl hydroxyl group, propyl hydroxyl group, methoxy group, ethyoxy group,

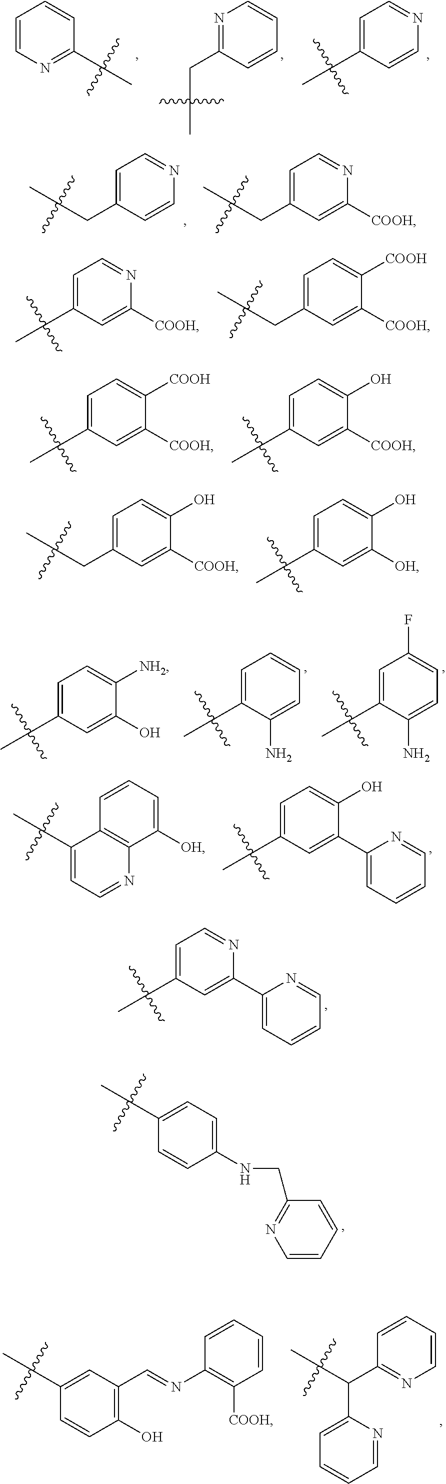

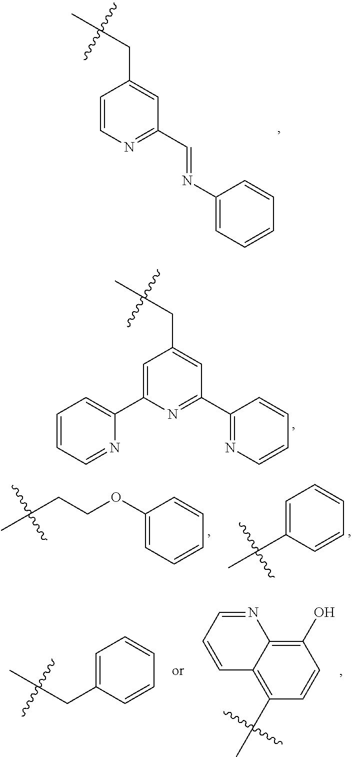

and R₅ is hydrogen.

In some embodiments, R₄ is hydroxy, methoxy group, or

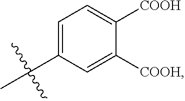 [SMILES: *c1ccc(C(=O)O)c(C(=O)O)c1]

and R₅ is hydrogen.

In some embodiments of compound having formula (I), R₄ is —(CH₂)n¹(OCH₂CH₂O)n²CH(R₆)CO(R₇);
R₅ is hydrogen;
R₆ is hydrogen, amino, C₁-C₃ alkyl, C₁-C₃ hydroxyalkyl group. C₁-C₃ carboxy alkyl group, aryl group, arylalkyl group which is optionally substituted with hydroxyl group, halogen, cyano group or nitro group, C₁-C₅ N-hydroxy amide alkyl group, heteroaryl group or heteroaryl alkyl group;
R₇ is hydroxy, C₁-C₅ alkoxy group, —NH(CH₂CONH)n³OH;
n¹, n² and n³ independently are the number 0, 1, 2, 3, 4.

In some embodiments, R₄ is —(CH₂)n¹(OCH₂CH₂O)n²CH(R₆)CO(R₇),
R₅ is hydrogen,
R₆ is hydrogen, amino, methyl, hydroxymethyl group, benzyl group, benzyl group substituted with hydroxyl group, halogen, cyano group or nitro group, halogen, carboxy ethyl group, N-hydroxy ethyl amide group,

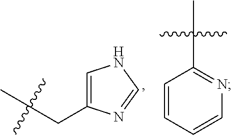

R7 is hydroxyl, —NH(CH₂CONH)n³OH;
n¹ and n³ independently are the number 0, 1, 2, 3, 4; n² is the number 0.

In some embodiments of compound having formula (I), R₄ is —(CH₂)n¹(OCH₂CH₂O)n²CH(R₆)CO(R₇); R₅ is hydrogen; R₆ is hydrogen, amino, methyl, hydroxymethyl group, benzyl group, carboxy ethyl group,

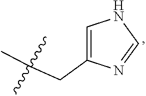 [SMILES: *Cc1c[nH]cn1]

N-hydroxy ethyl amide group,

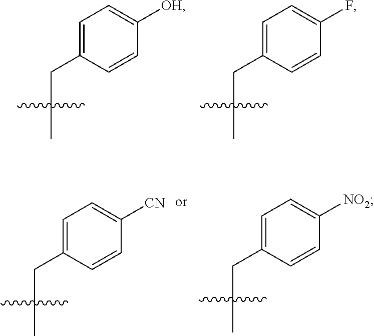 [SMILES: *Cc1ccc(O)cc1 ; *Cc1ccc(F)cc1 ; *Cc1ccc(C#N)cc1 ; *Cc1ccc([N+](=O)[O-])cc1]

R₇ is hydroxyl or —NH(CH₂CONH)n³OH;
n¹ is the number 2; n² is the number 1; n³ is the number 0.

In some embodiments, R₄ is —(CH₂)n¹(OCH₂CH₂O)n²CH(R₆)CO(R₇), R₅ is hydrogen, R₆ is hydrogen; R₇ is —NH(CH₂CONH)n³OH; n¹, n² and n³ are the number 0.

In some embodiments of compound having formula (I), A is —C(O)J-, J is peptide residue, comprising mono amino acid residue, dipeptide, tripeptide, tetrapeptide, pentapeptide, aminopropionic acid, aminobutyric acid, amino valeric acid, aminoacid, aminoheptanoic acid, aminooctanoic acid, or NH₂(OCH₂CH₂O)n⁴CH₂COOH, n⁴ is the number of 2-10.

The amino acid is selected from the group consisting of glycine (Gly), alanine (Ala), serine (Ser), arginine (Arg), asparagine (Asn), aspartic acid (Asp), cysteine (Cys), glutamine (Gln), glutamic acid (Glu), histidine (His), isoleucine (Ile), leucine (Leu), lysine (Lys), methionine (Met), phenylalanine (Phe), proline (Pro), threonine (Thr), tryptophan (Trp), tyrosine (Tyr) and valine (Val).

In some embodiments of compound having formula (I), J is the residue of histidine, serine, alanine, glycine, phenylalanine, asparagine, tyrosine, or asparagine.

In some embodiments of compound having formula (I), A is —C(O) J-, J is the residue of histidine, serine, alanine, glycine, phenylalanine, asparagine, tyrosine or asparagine, L₂ is —N(R₄)(R₅), R₄ is hydrogen, R₅ is hydroxy.

In some embodiments of compound having formula (I), Y is same as X.

In some embodiments of compound having formula (I), Z is same as X.

In some embodiments of compound having formula (I), Y and Z independently are

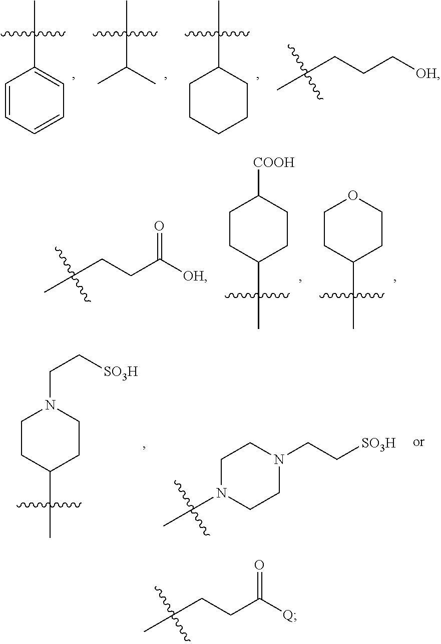

Q is —NHOH, —NHCH₂CH₂SO₃H, —N(CH₂CH₂OH)₂, —NHCH₂COOH, —NHCH₂(CH₃)COOH, —NH(CH₂CH₂O)₃CH₃.

In the compound of formula (II), in some embodiments, R₁ is H, and R2 is H.

In some embodiments, the reductants, including the first reductant and the second reductant, are independently selected from a group consisting of tris (2-carboxyethyl) phosphine (TCEP), or the compound having the following formula (II):

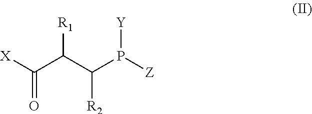

or a salt, solvate, stereoisomer thereof, wherein,
R₁ is H, —NH₂, —C(O)(R₃R₄), optionally substituted C₁-C₅ alkyl group, optionally substituted C₁-C₅ hydroxyalkyl group, or optionally substituted aryl group;
R₃ is N, NH or O;
R₄ is H, optionally substituted C₁-C₅ alkyl group, optionally substituted C₁-C₅ hydroxyalkyl group, or optionally substituted aryl group;
R₂ is H, optionally substituted C₁-C₅ alkyl group, or optionally substituted C₁-C₅ hydroxyalkyl group;
X is OH, optionally substituted C₁-C₅ alkoxy group or —NR₅R₆,
R₅ and R₆ independently are H, C₀-C₅ hydroxyalkyl group, optionally substituted C₁-C₅ alkyl group, optionally substituted C₂-C₈ carboxy alkyl group, or optionally substituted C₁-C₅ alkoxy group, optionally substituted heteroaryl alkyl group, optionally substituted aryl alkoxy group, optionally substituted arylalkyl group, optionally substituted aryl group, C₁-C₅ alkyl sulfonyl group, —(CH₂)n¹(OCH₂CH₂O)n²CH(R₈)CO(R₇),
R₇ C₀-C₅ hydroxyalkyl group, —NHOH,
R₅ is H, optionally substituted arylalkyl group,
n¹ and n² independently are the number 0, 1, 2, 3, 4,
Y is the same as X, or Y is an ester or amide of X,
Z is the same as X or Y, or
Y and Z independently are selected from the group consisting of

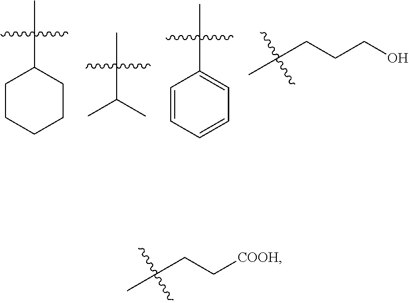

X, Y and Z are not

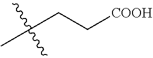 [SMILES: *CCC(=O)O]

at the same time.

In some embodiments of compound having formula (II), R₁ is H, and R₂ is H.

In some embodiments of compound having formula (II), X is —OCH₃, —OCH₂CH₃, or —O(CH₃)₂.

In some embodiments of compound having formula (II), X is —NR₅R₆, R₅ is H, and
R₆ is H, C₀-C₅ hydroxyalkyl group, C₁-C₅ alkoxy group, optionally substituted heteroaryl alkyl group, optionally substituted aryl alkoxy group, optionally substituted aryl group, optionally substituted arylalkyl group, C₁-C₅ alkyl sulfonyl group or —(CH₂)n¹(OCH₂CH₂O)n²CH(R₅)CO(R₇),
R₇ is C₀-C₃ hydroxyalkyl group or —NHOH,
R₈ is H or optionally substituted arylalkyl group,
n¹ and n² independently are the number 0.

In some embodiments of compound having formula (IT), R₆ is H, C₀-C₂ hydroxyalkyl group, C₁-C₃ alkoxy group, C₁-C₃ alkyl sulfonyl group, bipyridyl group, benzyl group, aryl alkoxy group, phenyl group which is optionally substituted with OH, carboxy or pyridyl group, or —CH(R₈)CO(R₇),
R₇ is OH or —NHOH.
R8 is H or benzyl group which is optionally substituted with OH, halogen, cyano group or nitro group.

In some embodiments of compound having formula (II), X is —NR₅R₆, R₅ is H, and
R₆ is H, OH, C₁ hydroxyalkyl group, C₂ hydroxyalkyl group, C₃ hydroxyalkyl group, C₄ hydroxyalkyl group, C₅ hydroxyalkyl group, C₁ alkoxy group, C₂ alkoxy group, C₃ alkoxy group, C₄ alkoxy group, C₅ alkoxy group, heteroaryl alkyl group optionally substituted with heteroaryl group, aryl methoxy group, aryl ethoxy group, aryl propoxy group, aryl butoxy group, aryl group optionally substituted with OH, carboxy or pyridyl group, benzyl group, aryl ethyl group, aryl propyl group, C₁ alkyl sulfonyl group, C₂ alkyl sulfonyl group, C₃ alkyl sulfonyl group, C₅ alkyl sulfonyl group, C₅ alkyl sulfonyl group or —(CH₂)n¹(OCH₂CH₂O)n²CH(R₅)CO(R₇),
R₇ is OH, C₁ hydroxyalkyl group, C₂ hydroxyalkyl group, C₃ hydroxyalkyl group or —NHOH,
R₈ is H or arylalkyl group which is optionally substituted with OH, halogen, cyano group or nitro group, wherein, halogen is selected from F, Cl, Br or I,
n¹ and n² independently are the number 0.

In some embodiments of compound having formula (II), X is —NR₅R₆, R₅ is H, and
R₆ is H, OH, —CH₂OH, —(CH₂)₂OH, —CH₃, —CH₂CH₃, —CH₂COOH, —(CH₂)₂COOH, —(CH₂)₃COOH, —(CH₂)₄COOH, —(CH₂)₅COOH, —OCH₃, —OCH₂CH₃, —CH₂CONHOH, —OC(C₆H₅)₃, —(CH₂)₃S(O)₂OH,

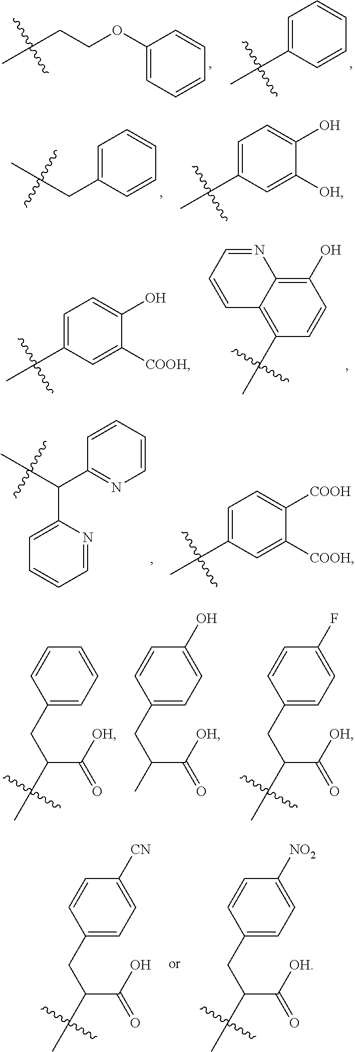

In some embodiments of compound having formula (II), X is —NR₅R₆, R₅ is H, and
R₆ is H, OH, —CH₂COOH, —CH₂CONHOH, —OC(C₆H₅)₃,

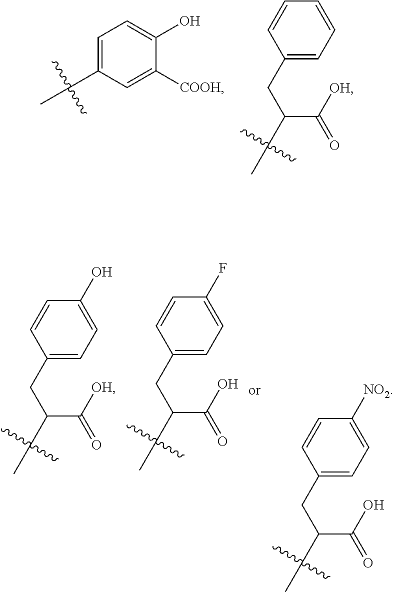 [SMILES: *c1ccc(O)c(C(=O)O)c1 ; *C(Cc1ccccc1)C(=O)O ; *C(Cc1ccc(O)cc1)C(=O)O ; *C(Cc1ccc(F)cc1)C(=O)O ; *C(Cc1ccc([N+](=O)[O-])cc1)C(=O)O]

In some embodiments of compound having formula (II), X is —NR₅R6, R₅ is H, and R₆ is OH, —CH₂COOH, —(CH₂)₂COOH, —(CH₂)₃COOH, —(CH₂)₄COOH, —(CH₂)₅COOH, —OCH₃, —OCH₂CH₃, or —OC(C₆H₅)₃.

In some embodiments of compound having formula (II), X is —NR₅R₆, R₅ is OH,
R₆ is C₁-C₅ alkyl group, optionally substituted heteroaryl alkyl group, optionally substituted arylalkyl group, optionally substituted aryl group, or —(CH₂)n¹(OCH₂CH₂O)n²CH(R₈)CO(R₇),
R7 is C₀-C₅ hydroxyalkyl group,
R8 is H,
n¹ and n² independently are the number 0, 1, 2, 3, 4.

In some embodiments of compound having formula (II), R₆ is C₁-C₃ alkyl group, heteroaryl alkyl group which comprises a heteroatom N, optionally substituted benzyl group, optionally substituted phenyl group, or —CH(R₈)CO(R₇),
R₇ is C₀-C₃ hydroxyalkyl group,
R₈ is H.

In some embodiments of compound having formula (II), R6 is C₁ alkyl group, C₂ alkyl group, C₃ alkyl group, C₄ alkyl group, C₅ alkyl group, heteroaryl methyl group, heteroaryl ethyl group, heteroaryl propyl group, benzyl group, aryl ethyl group, aryl propyl group, or —CH(R₈)CO(R₇), R₇ is hydroxy, C₁ hydroxyalkyl group, C₂ hydroxyalkyl group, C₃ hydroxyalkyl group, C₄ hydroxyalkyl group or C₅ hydroxyalkyl group, R₅ is H.

In some embodiments of compound having formula (II), R₆ is —CH₃, —CH₂COCH₃, —CH₂COOH,

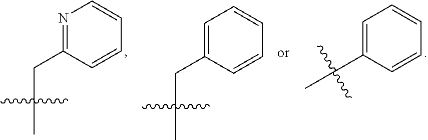 [SMILES: *Cc1ccccn1 ; *Cc1ccccc1 ; *c1ccccc1]

In some embodiments of compound having formula (ID), R₆ is —CH₂COOH.

In some embodiments of compound having formula (II), X is —NR₅R₆,
R₅ and R₆ independently are C₁-C₅ alkyl group, C₀-C₅ hydroxyalkyl group, optionally substituted heteroaryl alkyl group or —(CH₂)n¹(OCH₂CH₂O)n²CH(R₈)CO(R₇),
R₇ is C₀₋₅ hydroxyalkyl group or —NHOH,
R₈ is H,
n¹ and n² independently are the number 0, 1, 2, 3, 4.

In some embodiments of compound having formula (II), X is —NR₅R₆,
R₅ and R₆ independently are C₁ alkyl group, C₂ alkyl group, C₃ alkyl group, C₄ alkyl group, C₅ alkyl group, OH, C₁ hydroxyalkyl group, C₂ hydroxyalkyl group, C₃ hydroxyalkyl group, C₄ hydroxyalkyl group, C₅ hydroxyalkyl group, heteroaryl methyl group, heteroaryl ethyl group, heteroaryl propyl group or —(CH₂)n¹(OCH₂CH₂O)n²CH(R₈)CO(R₇),
R₇ is hydroxy, C₁ hydroxyalkyl group, C₂ hydroxyalkyl group, C₃ hydroxyalkyl group, C₄ hydroxyalkyl group, C₅ hydroxyalkyl group or —NHOH,
R₈ is H,
n¹ and n² independently are the number 0, 1, 2, 3, 4.

In some embodiments of compound having formula (II), X is —NR₅R₆, R₅ and R₆ independently are methyl, ethyl group, —(CH₂)₂OH, —CH₂COOH, —CH₂CONHOH or

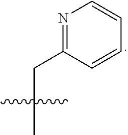 [SMILES: *Cc1ccccn1]

In some embodiments of compound having formula (II), Y is

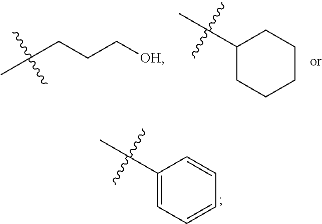 [SMILES: *CCCO ; *C1CCCCC1 ; *c1ccccc1]

### Z is

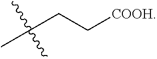 [SMILES: *CCC(=O)O]

In some embodiments, the first reductant and the second reductant are independently selected from the group consisting of

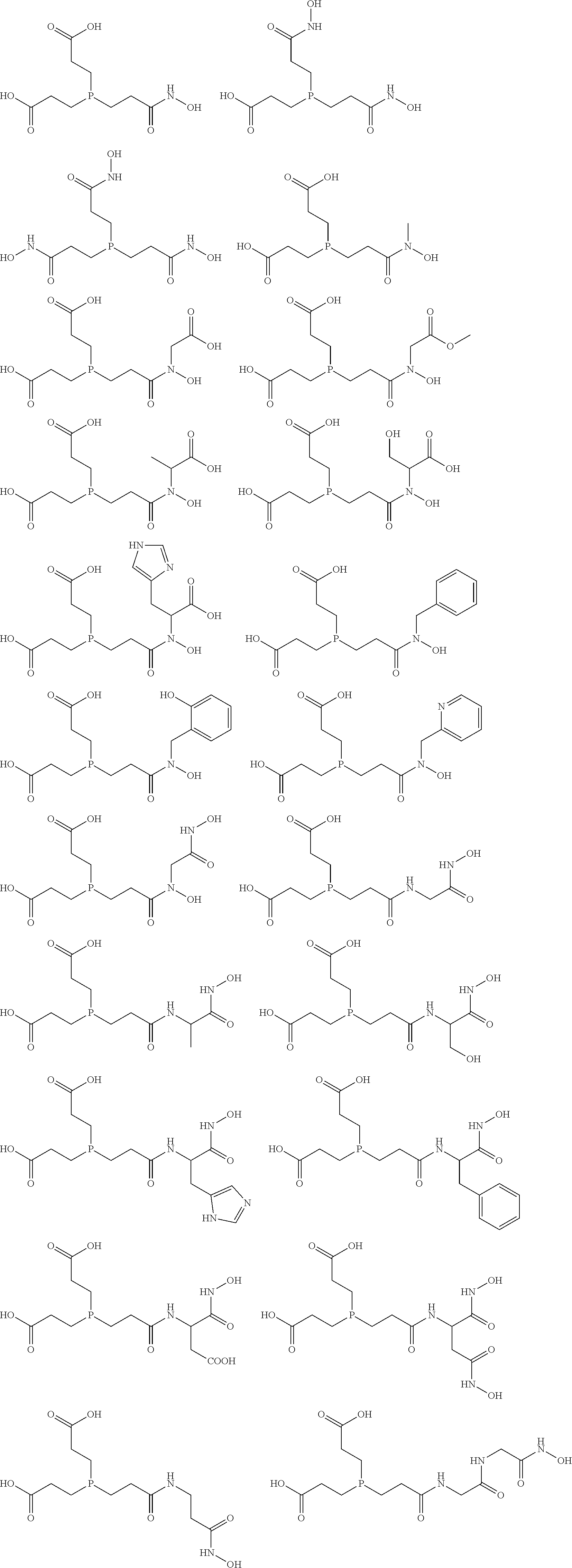

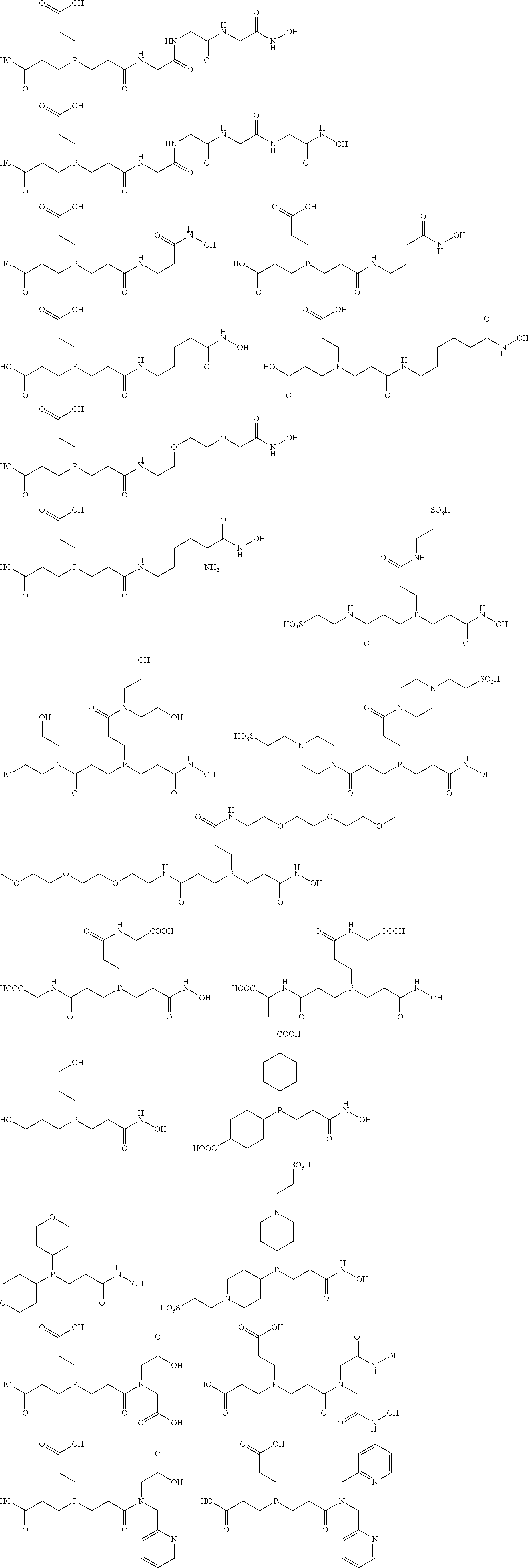

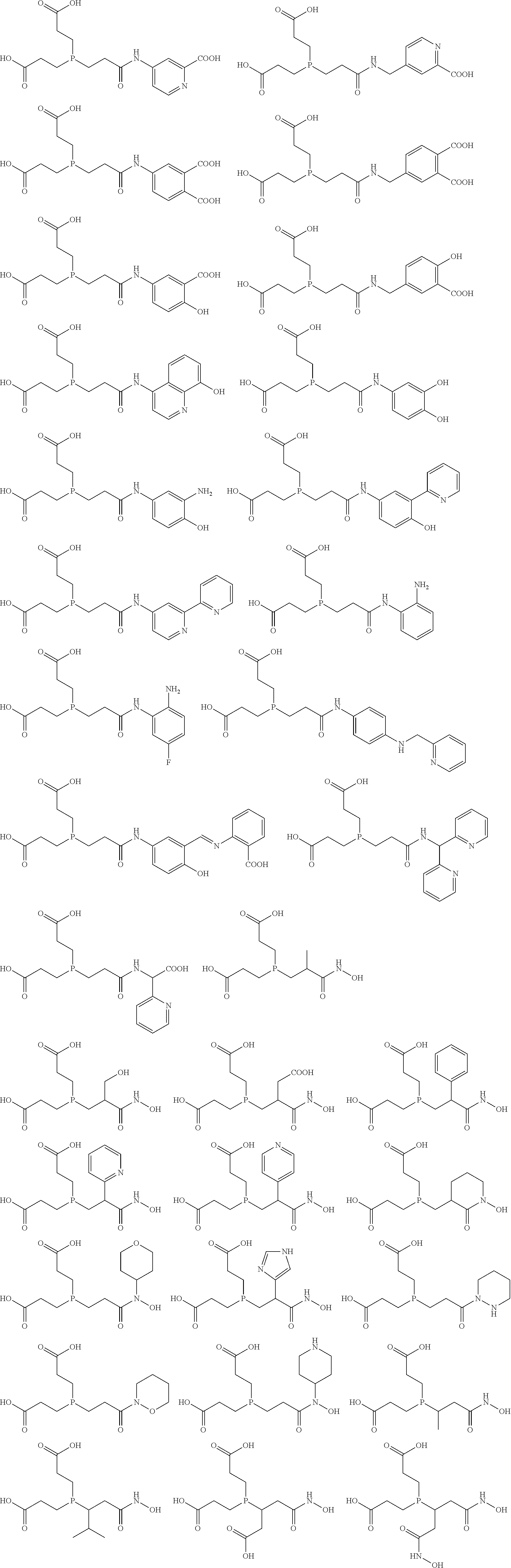

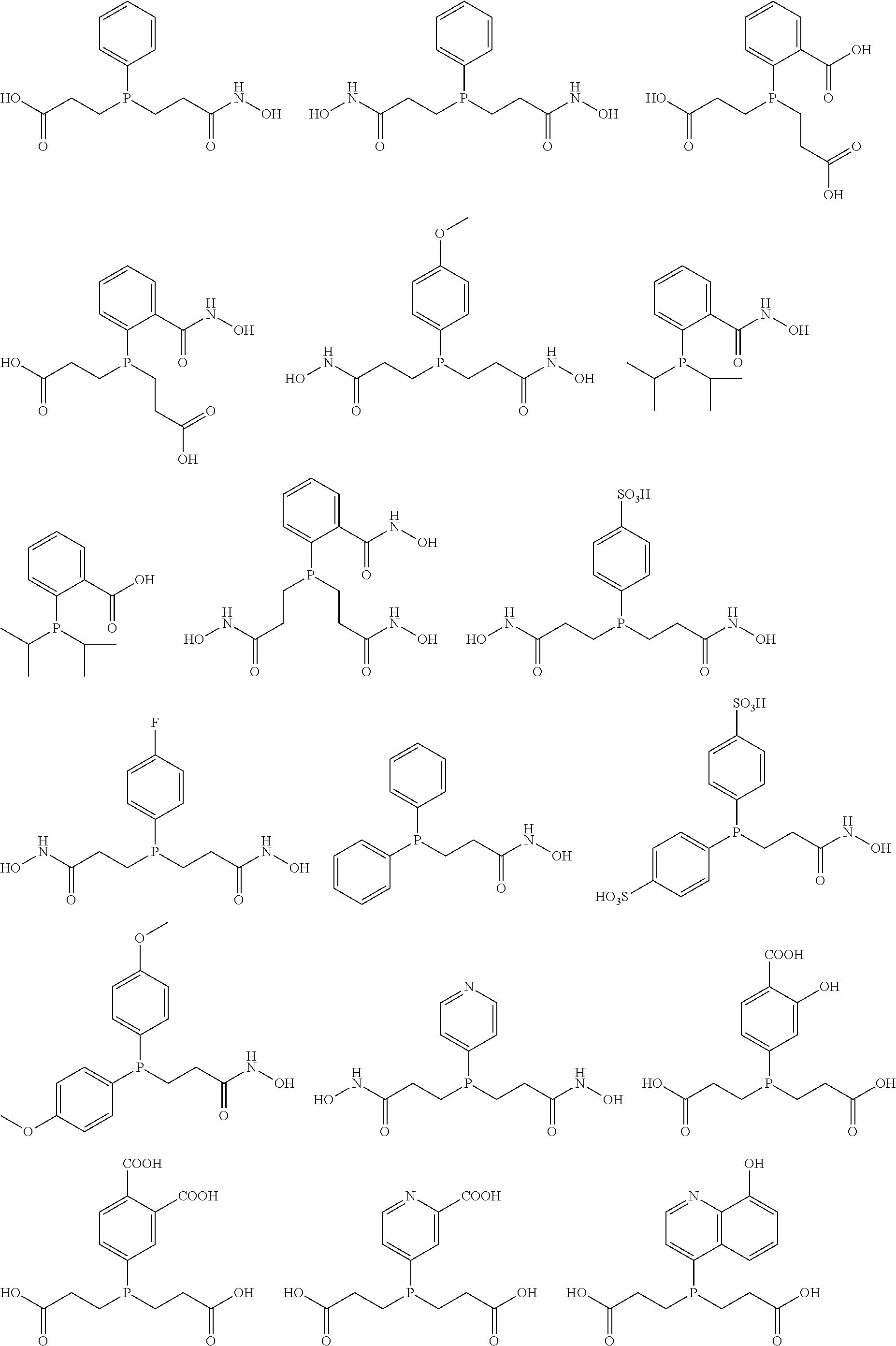

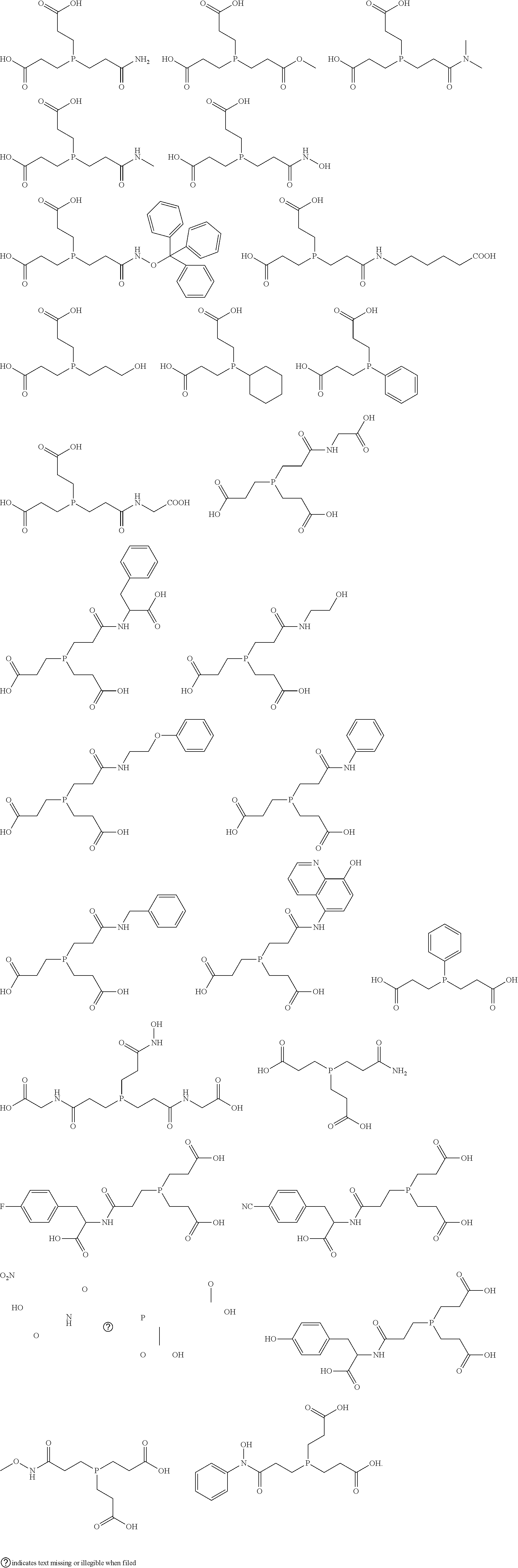

In some embodiments, the first reductant and the second reductant independently are TCEP, Tris(3-hydroxypropyl)phosphine (THPP), or Dithiothreitol (DTT).

In some embodiments, the first reductant and the second reductant independently are

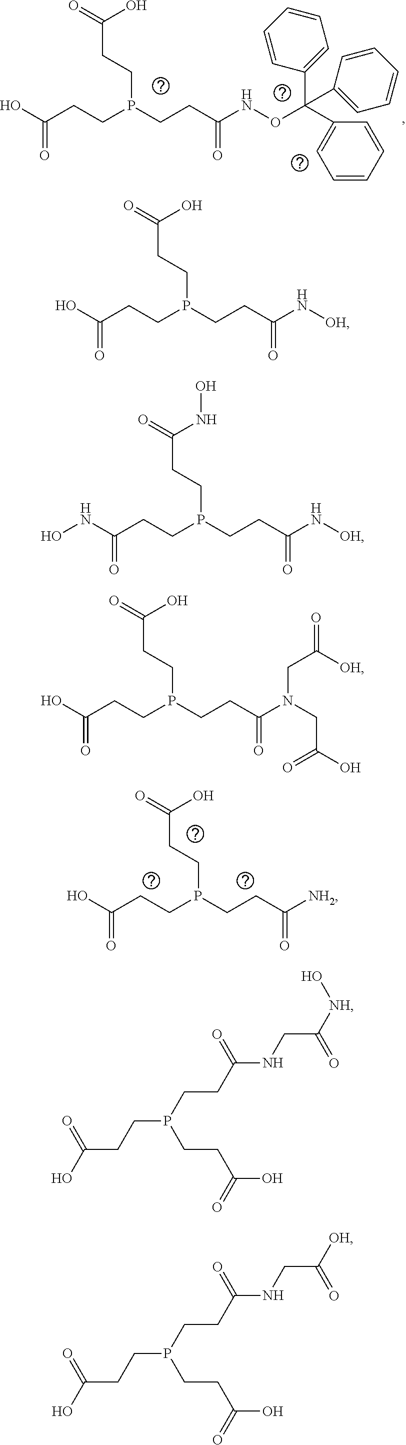

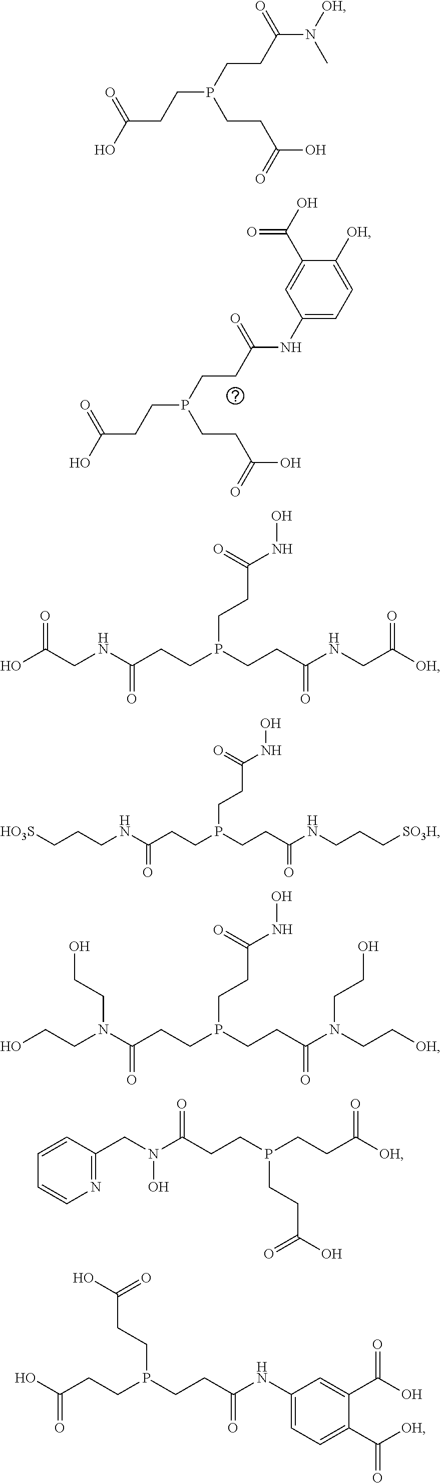

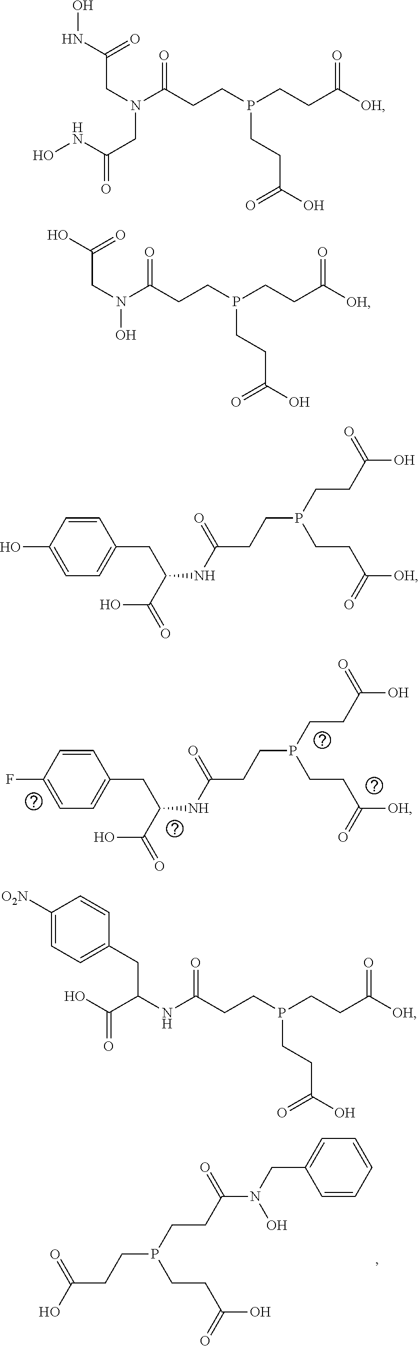

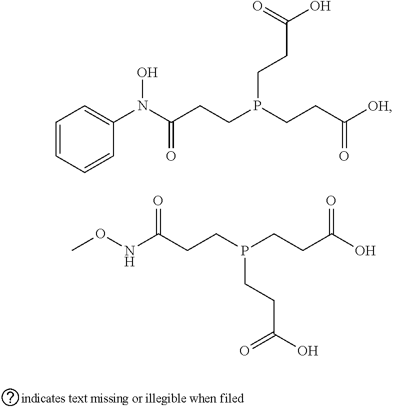

### or TCEP.

In some embodiments, the first reductant is TCEP.

In some embodiments, the first reductant is

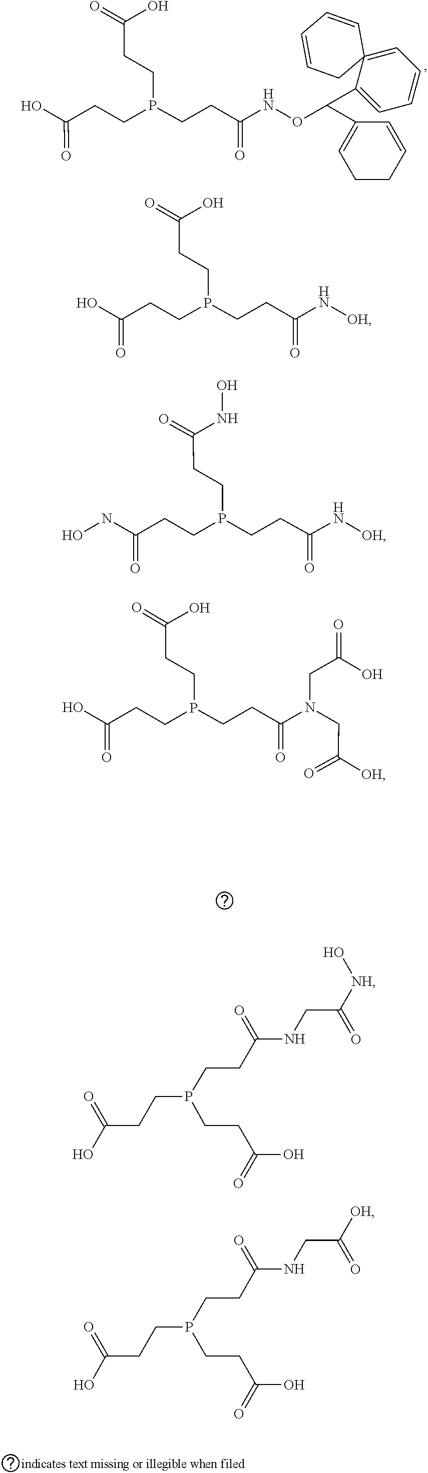

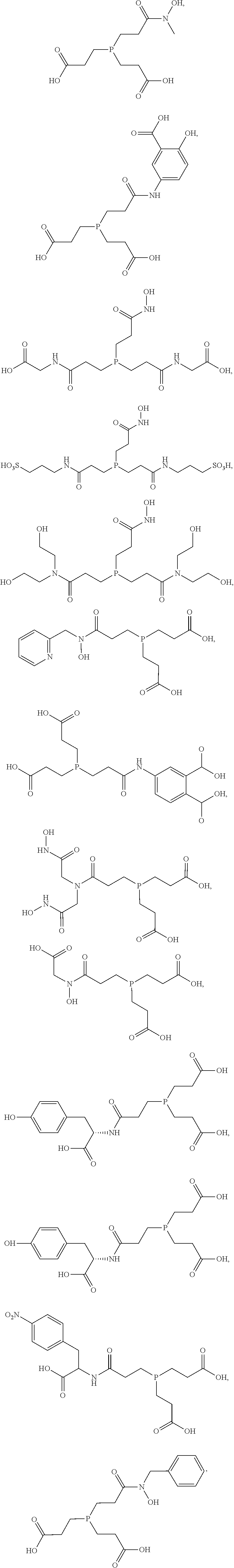

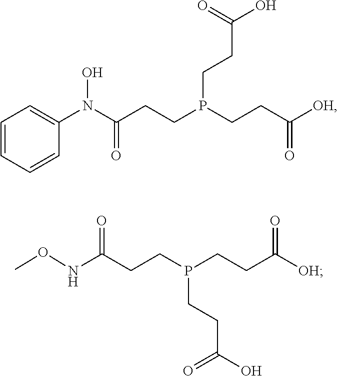 [SMILES: O=C(O)CCP(CCC(=O)O)CCC(=O)N(O)c1ccccc1 ; CONC(=O)CCP(CCC(=O)O)CCC(=O)O]

the second reductant is TCEP.

For antibody-drug conjugates, a mixture of ADCs will be generated by the conventional conjugation processes or the bio-conjugation process of the present disclosure. In general, one antibody molecule belonging to IgG1 or IgG4 subclass has 4 inter-chain disulfide bonds, each of which is formed with two —SH groups. The antibody molecule can be subjected to partial or complete reduction of one or more interchain disulfide bonds to form 2n (n is an integer selected from 1, 2, 3 or 4) reactive —SH groups, and thus, the number of drugs (or payloads) coupling to a single antibody molecule is 1, 2, 3, 4, 5, 6, 7 or 8. In accordance with the number of drugs coupling to a single antibody molecule, the different conjugates containing different number of drug molecules are denominated as D0, D1, D2, D3, D4, D6, D1+D3, D1+D6, D1+D2, D1+D4, D2+D3, D2+D6, D0+D3, D0+D6, D3+D1, D3+D2, D6+D2, D6+D1, D0+D1, D0+D3, D4+D1 or D4+D2. And thus, the “homogeneity” of antibody-drug conjugates is used to describe the property of dominance of one specific type of antibody-drug conjugate (i.e., one type selected from DO, D1, D2, D3, D4, D6, D1+D3, D1+D6, D1+D2, D1+D4, D2+D3, D2+D6, D0+D3, D0+D6, D3+D1, D3+D2, D6+D2, D6+D1, D0+D1, D0+D3, D4+D1 or D4+D2 conjugates) in one given mixture of antibody-drug conjugates.

Drug to Antibody Ratio (DAR) of ADC is the average number of drugs linked to each antibody. DAR is a key property used to measures the quality of ADC because it can significantly affect ADC efficacy. The DAR distribution (DO, D1, D2, D3, D4, D6, D1+D3, D1+D6, D1+D2, D1+D4, D2+D3, D2+D6, D0+D3, D0+D6, D3+D1, D3+D2, D6+D2, D6+D1, D0+D1, D0+D3, D4+D1 or D4+D2) could reflect the homogeneity of the ADC.

Drug loading is represented by the number of drug moieties per antibody in a molecule of ADC. For some antibody-drug conjugates, the drug loading may be limited by the number of attachment sites on the antibody. For example, where the attachment is a cysteine thiol, as in certain exemplary embodiments described herein, the drug loading may range from 0 to 8 drug moieties per antibody. In certain embodiments, the average drug loading for an antibody-drug conjugate ranges from 1 to about 8; from about 2 to about 6; or from about 3 to about 5.

As used herein, the term “DO” or “the ADC with DO” refers to the ADC in which the average number of drugs coupling to a single antibody molecule is about zero.

As used herein, the term “D1” or “the ADC with D1” refers to DAR about 1, it means about one drug molecules (e.g., 0.5, 1.0, 1.4 molecules) are coupled to one single antibody molecule on average. Drug molecules may be coupled to —SH groups generated by reduction of the disulfide bond between heavy and light chains or heavy and heavy chains via linkers. In some embodiments, D1 refers to the ADC in which one of the first thiobridge group bearing the linker-payload re-bridges two thiol groups of one single antibody molecule.

As used herein, the term “D2” or “the ADC with D2” refers to DAR about 2, it means about two drug molecules (e.g., 1.5, 2.0, 2.4 molecules) are coupled to one single antibody molecule on average. Drug molecules may be coupled to —SH groups generated by reduction of the interchain disulfide bonds between heavy and light chains and/or heavy and heavy chains via linkers. In some embodiments, D2 refers to the ADC in which two of the linker-payloads are coupled to one single antibody molecule.

As used herein, the term “D3” or “the ADC with D3” refers to DAR about 3, it means about three drug molecules (e.g., 2.5, 3.0, 3.4 molecules) are coupled to one single antibody molecule on average. Drug molecules may be coupled to —SH groups generated by reduction of the interchain disulfide bonds between heavy and light chains and heavy and heavy chains via linkers. In some embodiments, D3 refers to the ADC in which three of the thiobridge group bearing the linker-payload re-bridge six thiol groups of one single antibody molecule.

As used herein, the term “D4” or “the ADC with D4” refers to the ADC in which about four drug molecules (e.g., 3.5, 4.0, 4.4 molecules) are coupled to one single antibody molecule on average, where the drug molecules may be coupled to —SH groups generated by reduction of the interchain disulfide bonds. In some embodiments, D4 refers to the ADC in which four of the linker-payloads are coupled to one single antibody molecule.

As used herein, the term “D6” or “the ADC with D6” refers to the ADC in which about six drug molecules (e.g., 5.5, 6.0, 6.4 molecules) are coupled to one single antibody molecule on average, where the drug molecules may be coupled to six —SH groups generated by reduction of three disulfide bond. In some embodiments, D6 refers to the ADC in which six of the linker-payloads are coupled to one single antibody molecule.

As used herein, the term “D8” refers to the ADC in which about eight drug molecules (e.g., 7.5, 8.0, 8.4 molecules) are coupled to one single antibody molecule on average, where the drug molecules may be coupled to eight —SH groups generated by reduction of four disulfide bond. In some embodiments, D8 refers to the ADC in which eight of the linker-payloads are coupled to one single antibody molecule.

As used herein, the term “about” refers to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In particular embodiments, the terms “about” when preceding a numerical value indicates the value plus or minus a range of 50%, 30%, 15%, 10%, 5%, or 1%.

In some embodiments, by conjugating two different linker-payloads to a single antibody to form a bi-payload ADC. According to the DAR value of each linker-payload, the bi-payload ADC may be with two DAR values, such as D6 for first linker-payload and D2 for second linker-payload. The DAR value of di-payload ADC in present disclosure is referred to as DN+DM, of which N denotes the average number of the first linker-payload coupled to one single antibody molecule on average, and M denotes the average number of the second linker-payload coupled to one single antibody molecule on average.

In some embodiments, “D1+D6” or the “the ADC with D1+D6” refers to the ADC in which one of the first thiobridge group bearing the first linker-payload re-bridging two thiol groups and six of the second linker-payloads are coupled to one single antibody molecule.

In some embodiments, “D2+D6” or “the ADC with D2+D6” refers to the ADC in which two of the first linker-payloads and six of the second linker-payloads are coupled to one single antibody molecule.

In some embodiments, “D0+D2” or “the ADC with D0+D2” refers to the ADC in which one, two or three of the first thiobridge group re-bridge six thiol groups and two of the second linker-payloads are coupled to one single antibody molecule, or refers to the ADC in which two, four or six of the first end capping reagents and two of the second linker-payloads are coupled to one single antibody molecule.

In some embodiments, the salt refers to acid addition salts or base addition salts.

In some embodiments, acid addition salts can be formed with inorganic acids and organic acids. The inorganic acids from which salts can be derived include, for example, hydrochloric acid, hydrobromic acid, sulfuric acid, phosphoric acid, and the like. The organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

In some embodiments, base addition salts can be formed with inorganic bases and organic bases. The inorganic bases from which salts can be derived include groups 1 to 2 of the periodic table. In certain embodiments, the salts are derived from lithium, sodium, potassium, calcium, magnesium and the like. The organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In some embodiments, in step (R1) of the method I, the molar ratio of the first reductant and the transition metal ions is 1:0.4 to 1:250, 1:0.4 to 1:200, 1:1 to 1:70, 1:0.4 to 1:60, 1:0.1 to 1:20, 1:6 to 1:16, 1:0.2 to 1:8, 1:0.5 to 1:8, 1:0.25 to 1:7.5, or 1:0.25 to 1:7.

In some embodiments, in method I, the first reductant and the transition metal ions with specific molar ratio selectively reduce one of four interchain disulfide bonds within antibody, optionally one interchain disulfide bond in the hinge region of the antibody is reduced.

In the disclosure, the term “transition metal ions” refers to the elements of groups 4-12, justified by their typical chemistry, i.e., a large range of complex ions in various oxidation states, colored complexes, and catalytic properties either as the element or as ions (or both). Sc and Y in Group 3 are also generally recognized as transition metals.

In some embodiments, the transition metal ions are selected from the group consisting of Zn²⁺, Cd²⁺, Hg²⁺, Ni²⁺, Co²⁺, or the combination thereof.

In some embodiments, the transition metal ions are Zn²⁺.

In some embodiments, there is no specific limitation to the salts of the transition metal ions, as long as the transition metal ions are soluble in the reaction solution so that free transition metal ions can be released in the reaction solution. In some embodiments, the salts of the transition metal ions are chloride, nitrate, sulfate, acetate, iodide, bromine, formate or tetrafluorborate.

In some embodiments, the salts of Zn²⁺ are ZnCl₂, Zn(NO₃)₂, ZnSO₄, Zn(CH₃COO)₂, ZnI₂, ZnBr₂, Zinc formate, or zinc tetrafluoroborate. In some embodiments, the salts of Zn²⁺ are ZnCl₂.

In some embodiments, in step (R1) of the method I, when the first reductant is TCEP, the molar ratio of the first reductant and the transition metal ions is 1:0.4 to 1:200, 1:0.4 to 1:70, 1:1 to 1:16, or 1:2 to 1:16. In some embodiments, in step (R1) of the method I, when the first reductant is TCEP, the molar ratio of the first reductant and the transition metal ions is 1:0.5, 1:2, 1:3, 1:4, 1:6, 1:8, 1:10, 1:12, 1:14, 1:16, 1:20, 1:25, 1:30, 1:35, 1:40, 1:45, 1:50, 1:55, 1:60, 1:65, 1:70, 1:75, 1:80, 1:85, 1:90, 1:95, 1:100, 1:110, 1:120, 1:130, 1:140, 1:150, 1:160, 1:170, 1:180, 1:190 or 1:200.

In some embodiments, in step (R1) of the method I, when the first reductant is the compound having formular (I) or (II), the molar ratio of the first reductant and the transition metal ions is 1:0.4 to 1:250, 1:0.4 to 1:60, 1:2 to 1:60, 1:4 to 1:60, 1:4 to 1:24, 1:2 to 1:12, or 1:6 to 1:16, optionally, the first reductant is TCEP, TCEPA or TCEP-NO-Trtyl. In some embodiments, in step (R1) of the method 1, when the first reductant is the compound having formular (I) or (II), the molar ratio of the first reductant and the transition metal ions is 1:1 to 1:180, 1:1 to 1:150, 1:1 to 1:130, 1:1 to 1:100, 1:1 to 1:80, 1:1 to 1:70, 1:60. In some embodiments, in step (R1) of the method I, when the first reductant is the compound having formular (I) or (II), the molar ratio of the first reductant and the transition metal ions is 1:0.5, 1:2, 1:3, 1:4, 1:6, 1:8, 1:10, 1:12, 1:14, 1:16, 1:20, 1:25, 1:30, 1:35, 1:40, 1:45, 1:50, 1:55, 1:60, 1:65, 1:70, 1:75, 1:80, 1:85, 1:90, 1:95, 1:100, 1:110, 1:120, 1:130, 1:140, 1:150, 1:160, 1:170, 1:180, 1:190, 1:200, 1:220, 1:240 or 1:250.

In some embodiments, the molar ratio of the first reductant and the antibody in step (R1) is 1:1 to 5:1, 2:1 to 5:1, 4:1 to 5:1, 1:1 to 3:1, 3.8:1 to 4.6:1, 1:1 to 2:1 or 3:1 to 5:1.

In some embodiments, in step (R1) of the method I, the molar ratio of the first reductant and the antibody is 3:1 to 0.5:1, optionally, the molar ratio of the first reductant and the antibody is 3:1 to 1:1, more optionally, the molar ratio of the first reductant and the antibody is 2:1 to 1:1.

In some embodiments, in step (R1) of the method I, the molar ratio of the first reductant and the antibody is 0.6:1 to 1:1, 1.5:1 to 1:1, 1.2:1 to 1:1. In some embodiments, in step (R1) of the method I, the molar ratio of the first reductant and the antibody is 2.9:1, 2.8:1, 2.7:1, 2.6:1, 2.5:1, 2.4:1, 2.3:1, 2.2:1, 2.1:1, 2:1, 1.9:1, 1.8:1, 1.5:1, 1.4:1, 1.2:1, 1:1, 0.8:1 or 0.5:1.

In some embodiments, in step (R1) of the method I, there is no specific limitation to the concentration of the first reductant, as long as scaling up or down the concentration of the transition metal ions and the antibody in equal proportions. In some embodiment, the concentration of the first reductant is 0.01 mM to 0.2 mM, 0.02 mM to 0.15 mM or 0.05 mM to 0.1 mM. In some embodiments, the concentration of the first reductant is 0.01 mM, 0.02 mM, 0.03 mM, 0.04 mM, 0.05 mM, 0.06 mM, 0.07 mM, 0.08 mM, 0.09 mM, 0.10 mM, 0.11 mM, 0.12 mM, 0.13 mM, 0.14 mM, 0.15 mM, 0.16 mM, 0.17 mM, 0.18 mM, 0.19 mM or 0.20 mM.

In some embodiment, there is no specific limitation to the concentration of the transition metal ions in step (R1) of method I, as long as scaling up or down the concentration of the first reductant and the antibody in equal proportions.

In some embodiments, there is no specific limitation to the concentration of the antibody in step (R1) in method I, as long as scaling up or down the concentration of the first reductant and the transition metal ions in equal proportions.

In some embodiment, the concentration of the first reductant, the transition metal ions and the antibody in step (R1) of the method I applies to that in step (R1) of the method II, III, IV and V.

In some embodiments, in step (R1) of the method I, the incubation temperature is 0° C. to 37° C., 0° C. to 25° C., 0° C. to 15° C., 0° C. to 10° C., or 0° C. to 5° C. In some embodiments, in step (R1) of the method I, incubation temperature is 4° C., 8° C., 12° C., 15° C., 18° C., 24° C., 30° C., 35° C. or 37° C. In some embodiments, the incubation temperature in step (R1) also applies to that in step (R1) of method II, III, IV and V.

In some embodiments, in step (R1) of the method I, the incubation time is 0.2 h to 24 h, optionally, the incubation time is 2 h to 16 h. In some embodiments, in step (R1) of the method I, the incubation time is 0.5 h to 24 h, 0.5 h to 20 h, 0.5 h to 16 h, 0.5 h to 12 h, 0.5 h to 8 h or 0.5 h to 6 h. In some embodiments, in step (R1) of the method I, the incubation time is 0.25 h, 0.3 h, 0.5 h, 0.7 h, 1 h, 1.5 h, 2 h, 3 h, 4 h, 5 h, 6 h, 7 h, 10 h, 12 h, 14 h, 16 h, 18 h, 20 h, 22 h or 24 h.

In some embodiments, in step (R1) of the method I, the molar ratio of the first reductant and the antibody is 2:1 to 3.0:1, and the incubation time is 0.5 h to 9 h. In some embodiments, in step (R1) of the method I, the molar ratio of the first reductant and the antibody is 2.8:1 to 3.0:1, and the incubation time is 0.5 h to 9 h. In some embodiments, in step (R1) of the method I, the molar ratio of the first reductant and the antibody is 2.1:1, 2.2:1, 2.3:1, 2.4:1, 2.5:1, 2.6:1, 2.7:1, 2.8:1, 2.9:1 or 3:1, the incubation time is 0.5, 1 h, 2 h, 3 h, 4 h,5 h, 6 h, 7 h, 8 h, 9 h, 9.5 h or 9.9 h.

In some embodiments, in step (R3) of method I, there is no specific limitation to the second reductant, as long as the second reductant could reduce the interchain disulfide bonds within the antibody. In some embodiments, the second reductant is the same as the first reductant. In some embodiments, the second reductant is different from the first reductant. In some embodiments, the second reductant is the compound having the formula (I) or (II), TCEP, Tris(3-hydroxypropyl)phosphine (THPP), or Dithiothreitol (DTT). In some embodiments, the second reductant is TCEP. In some embodiments, the second reductant of the method I also applies to that of the method II, III, IV and V.

In some embodiments, in step (R3), there is no specific limitation to concentration of the second reductant, as long as the second reductant could reduce the interchain disulfide bonds within the antibody. In some embodiments, the molar ratio of the second reductant and the antibody in step (R3) is 10:1 to 25:1, 10:1 to 23:1, 1:1 to 20:1, 5:1 to 20:1, 10:1 to 19:1, 10:1 to 18:1, 15:1 to 25:1, 10:1 to 20:1, 15:1 to 20:1, 1:1 to 8:1, 1:1 to 5:1, 1:1 to 3:1, or 1:1 to 2:1, when the second reductant is TCEP or the compound having formula (I) or (IT).

In some embodiments, in step (R3), the molar ratio of the second reductant and the antibody in step (R3) is 1:1 to 10:1, 1:1 to 8:1, 1:1 to 5:1, 1:1 to 3:1, or 1:1 to 2:1, when the second reductant is TCEP, TCEP-NO or TCEPA.

In some embodiments, in step (R3) of the method I, three of the interchain disulfide bonds in the product prepared from step (C1) are reduced completely without the transition metal ions. In some embodiments, one interchain disulfide bond or two interchain disulfide bonds in the product prepared from step (C1) is(are) reduced with the transition metal ions.

In some embodiments, in step (R3) of the method I, without the transition metal ions, the molar ratio of the second reductant and the antibody is 3:1 to 20:1, 3:1 to 10:1, 4:1 to 10:1, 5:1 to 9:1, 6:1 to 9:1, 6:1 to 8:1. In some embodiments, the molar ratio of the second reductant and the antibody is 20:3. In some embodiments, the molar ratio of the second reductant and the antibody of the method I also applies to that in step (R3) of the method III, IV, and V.

In some embodiments, in step (R3) of method I, the incubation temperature of the second reductant is 0° C. to 37° C., or 5° C. to 30° C. In some embodiments, in step (R3) of method I, the incubation temperature of the second reductant is 10° C. to 30° C., 15° C. to 30° C., 20° C. to 30C, or 25° C. to 30° C.. In some embodiments, in step (R3) of method I, the incubation temperature of the second reductant is 2° C., 4° C., 6° C., 8° C., 10° C., 12° C., 14° C., 16° C., 18° C., 20° C., 22° C., 25° C. In some embodiments, the incubation temperature in step (R3) of the method I also applies to that in step (R2) of the method IV and that in step (R3) of the method II, III, IV and V.

In some embodiments, in step (R3) of the method I, the incubation time of the second reductant is 0.5 h to 24 h, or 5 h to 20 h. In some embodiments, in step (R3) of the method I, the incubation time of the second reductant is 6 h to 18 h, 8 h to 18 h, 8 h to 15 h, or 8 h to 12 h. In some embodiments, in step (R3) of the method I, the incubation time of the second reductant is 8 h or 12 h. In some embodiments, the incubation time in step (R3) of method I also applies to that in step (R3) of the method III, IV and V.

In some embodiments, in step (R3) of method I, introducing the transition metal ions, two of the interchain disulfide bonds are selectively reduced. In some embodiments, in step (R3) of method I, the molar ratio of the second reductant and the transition metal ions is 1:0.05 to 1:40, and/or the molar ratio of the second reductant and the antibody is 2.5:1 to 20:1, and/or the incubation time is 1 h to 24 h.

In some embodiments, in step (R3) of method I, the molar ratio of the second reductant and the transition metal ions is 1:0.05, 1:0.08, 1:0.1, 1:0.2, 1:0.3, 1:0.4, 1:0.5, 1:0.6, 1:0.7, 1:0.8, 1:0.9, 1:1, 1:2, 1:4, 1:6, 1:8, 1:10, 1:12, 1:14, 1:16, 1:18 or 1:20. In some embodiments, in step (R3) of method I, the molar ratio of the second reductant and the antibody is 2.5:1, 3:1, 5:1, 7:1, 9:1, 11:1, 13:1, 15:1, 17:1, 19:1 or 20:1. In some embodiments, in step (R3) of method I, the incubation time is 1 h, 2 h, 4 h, 6 h, 8 h, 10 h, 12 h, 14 h, 16 h, 18 h, 20 h, 22 or 24 h. In some embodiments, in step (R3) of method I, the molar ratio of the second reductant and the transition metal ions is 1:0.05 to 1:40, and/or the molar ratio of the second reductant and the antibody is 3:1 to 15:1, and the incubation time is 1 h to 12 h. In some embodiments, in step (R3) of method I, the molar ratio of the second reductant and the transition metal ions is 1:0.05 to 1:40, and/or the molar ratio of the second reductant and the antibody is 2.5:1 to 15:1, and the incubation time is 12 to 24 h. In some embodiments, the molar ratio of the second reductant and the transition metal ions, the molar ratio of the second reductant and the antibody, and the incubation time in step (R3) of the method I also applies to that in step (R3) of the method III.

In some embodiments, in step (R3) of method I, introducing the transition metal ions, one of the interchain disulfide bonds are selectively reduced. In some embodiments, in step (R3) of method I, the molar ratio of the second reductant and the transition metal ions is 1:0.4 to 1:100, and/or the molar ratio of the second reductant and the antibody is 0.8:1 to 2.5:1, and/or the incubation time is 0.5 h to 24 h. In some embodiments, in step (R3) of method I, the molar ratio of the second reductant and the transition metal ions is 1:0.5, 1:1, 1:4, 1:8, 1:12, 1:24, 1:30, 1:40, 1:50, 1:50, 1:70, 1:80, 1:90, 1:100. In some embodiments, in step (R3) of method I, the molar ratio of the second reductant and the antibody is 0.5:1, 1:1, 1.2:1, 1.4:1, 1.6:1, 1.8:1, 2:1, 2.2:1, 2.4:1 or 2.5:1. In some embodiments, in step (R3) of method I, the incubation time is 0.5 h, 1 h, 2 h, 4 h, 6 h, 8 h, 10 h, 12 h, 14 h, 16 h, 18 h, 20 h, 22 or 24 h. In some embodiments, in step (R3) of method I, the molar ratio of the second reductant and the transition metal ions is 1:0.5 to 1:100, and/or the molar ratio of the second reductant and the antibody is 0.8:1 to 2:1, and the incubation time is 0.5 h to 24 h. In some embodiments, in step (R3), the molar ratio of the second reductant and the transition metal ions is 1:0.4 to 1:100, and/or the molar ratio of the second reductant and the antibody is 2:1 to 2.5:1, and the incubation time is 1 h to 9 h. In some embodiments, the molar ratio of the second reductant and the transition metal ions, the molar ratio of the second reductant and the antibody, and the incubation time in step (R3) of the method I also applies to that in step (R3) of the method III, IV and V.

In some embodiments, in step (C1) and in step (C3) of method I, the reaction temperature with the reduced thiol groups is 4° C. to 40° C., 10° C. to 40° C., 10° C. to 35° C., 10° C. to 30° C., 10° C. to 25° C., 15° C. to 35° C., 20° C. to 30° C., 4° C. to 37° C., 20° C. to 30° C. or 20° C. to 25° C. In some embodiments, in step (C1) and in step (C3), the reaction temperature with the reduced thiol groups is 24° C.. In some embodiments, the reaction temperature with the reduced thiol groups in step (C1) and in step (C3) of method I also applies to that in step (C2) and/or (C3) of method V and that in step (C1) and/or (C3) of method II. III and IV.

In some embodiments, in step (C1) and in step (C3) of method I, the reaction time with the reduced thiol groups is 0.5 h to 10 h, 0.5 h to 6 h, 0.5 h to 5 h, 0.5 h to 4 h, 0.5 h to 3 h, 0.5 h to 3 h, 0.5 h to 2 h, 0.5 h to 1 h. In some embodiments, in step (C1) and in step (C3) of method I, the reaction time with the reduced thiol groups is 0.5 h, 1 h, 2 h, 3 h, 4 h, 5 h, 6 h, 7 h, 8 h, 9 h or 10 h. In some embodiments, the reaction time with the reduced thiol groups in step (C1) and in step (C3) of method I also applies to that in step (C2) and/or (C3) of method V and that in step (C1) and/or (C3) of method II, III and IV.

In some embodiments, the reactive temperature and time with the reduced thiol groups in step (C1) and step (C3) are independent.

In some embodiments, in step (C1) and in step (C3) of method I, the reaction temperature with the reactive groups is 4° C. to 37° C., 10° C. to 37° C., 20° C. to 30C, 10° C. to 30° C., 15C to 30° C. or 25° C. to 30° C. In some embodiments, in step (C1) and in step (C3) of method I, the reaction temperature with the reactive groups is 4° C., 6° C., 8° C., 10C, 13° C., 17° C., 20° C., 23C, 27C, 30C, 34C, 35° cor 37° C. In some embodiments, the reaction temperature with the reactive groups in step (C1) and in step (C3) of method I also applies to that in step (C2) and/or (C3) of method V and that in step (C1) and/or (C3) of method II, III and IV.

In some embodiments, in step (C1) and in step (C3) of method I, the reaction time with the reactive groups is 2 h to 12 h, 2 h to 10 h, 4 h to 10 h, 6 h to 10 h, or 8 h to 10 h. In some embodiments, in step (C1) and in step (C3) of method I, the reaction time with the reactive groups is 2 h, 3 h, 4 h, 5 h, 6 h, 7 h, 8 h, 9 h, 10 h, 11 h, 12 h. In some embodiments, the reaction time with the reactive groups in step (C1) and in step (C3) of method I also applies to that in step (C2) and/or (C3) of method V and that in step (C1) and/or (C3) of method II, III and IV.

In some embodiments, the reactive temperature and time with the reactive groups in step (C1) and step (C3) are independent.

In some embodiments, in step (C1), according to the amount of the antibody, the first conjugating group is excess.

In some embodiments, the molar ratio of the first conjugating group and the antibody in step (C1) is 1:1 to 50:1, 1:1 to 20:1, 1:1 to 10:1, 1:1 to 8:1, 1:1 to 6:1, 1:1 to 5:1, 2:1 to 8:1, or 2:1 to 6:1.

In some embodiments, in step (C1) of the method I, the molar ratio of the first thiobridge reagent and the antibody is 10:1 to 1:1, 5:1 to 1:1, 2:1 to 1:1, 1.5:1 to 1:1, 1.2:1 to 1:1 or 1.1:1 to 1:1. In some embodiment, in step (C1) of the method I, the molar ratio of the first thiobrige reagent and the antibody is 1:1, 1.5:1, 2:1, 3:1, 3.8:1, 4:1, 4.8:1 or 5:1.

In some embodiments, in step (C1) of the method I, when the first linker-payload reacts with the reactive groups in the first thiobridge reagent, the molar ratio of the first linker-payload and the antibody is 10:1 to 1:1, 5:1 to 1:1, 4:1 to 1:1.1, 3:1 to 1:1 or 2:1 to 1:1. In some embodiments, in the step (C1) of the method I, the molar ratio of the first linker-payload and the antibody is 5:3.

In some embodiments, in step (C1) of the method I, when the first linker-payload reacts with the reduced thiol groups, the molar ratio of the first linker-payload and the antibody is 2:1 to 20:1, 2:1 to 10:1, 3:1 to 10:1, 4:1 to 9:1 or 5:1 to 7:1. In some embodiments, in step (C1) of the method I, when the first linker-payload reacts with the reduced thiol groups, the molar ratio of the first linker-payload and the antibody is 2:1, 3:1, 4:1, 5:1, 6:1, 7:1, 8:1, 9:1, 10:1, 12:1, 13:1, 14:1, 15:1, 16:1, 17:1, 18:1, 19:1 or 20:1.

In some embodiments, the molar ratio of the first conjugating group and the antibody in step (C1) of method I also applies to that in step (C1) of method III.

In some embodiments, in step (C3), according to the amount of the antibody, the second conjugating group is excess.

In some embodiments, the molar ratio of the second conjugating group and the antibody in step (C3) is 1:1 to 30:1, 1:1 to 20:1, 1:1 to 10:1, 1:1 to 8:1, 1:1 to 6:1, 1:1 to 5:1, 2:1 to 8:1, or 2:1 to 6:1.

In some embodiments, in step (C3) of the method I, the molar ratio of the second thiobridge reagent and the antibody is 10:1 to 1:1, 5:1 to 1:1, 5:1 to 3:1, 4:1 to 3:1, 4:1 to 3.2:1 or 4:1 to 3.5:1. In some embodiments, in step (C3) of the method I, the molar ratio of the second thiobridge reagent and the antibody is 5:1, 4.5:1, 4:1, 3.8:1, 3.5:1 or 3.2:1.

In some embodiments, in step (C3) of the method I, when the second linker-payload reacts with the reactive groups in the second thiobridge reagent, the molar ratio of the second linker-payload and the antibody is 10:1 to 1:1, 10:1 to 3:1, 9:1 to 3:1, 8:1 to 3:1, 7:1 to 3:1, 6:1 to 3:1, 5:1 to 3:1 or 4:1 to 3:1.

In some embodiments, in step (C3) of the method I, when the second linker-payload reacts with the reduced thiol groups, the molar ratio of the second linker-payload and the antibody is 20:1 to 1:1, 20:1 to 6:1, 18:1 to 8:1, 16:1 to 8:1, 14:1 to 8:1, 12:1 to 10:1. In some embodiments, in step (C3) of the method I, when the second linker-payload reacts with the reduced thiol groups, the molar ratio of the second linker-payload and the antibody is 35:3.

In some embodiments, the molar ratio of the second conjugating group and the antibody in step (C3) of method I also applies to that in step (C3) of method III, IV and V.

In some embodiments, in step (R4) of the method I, introducing the transition metal ions, one of the interchain disulfide bonds are selectively reduced. In some embodiments, in step (R4) of method I, the molar ratio of the second reductant and the transition metal ions is 1:0.4 to 1:100, and/or the molar ratio of the second reductant and the antibody is 0.8:1 to 2.5:1, and/or the incubation time is 0.5 h to 24 h. In some embodiments, in step (R4) of method I, the molar ratio of the second reductant and the transition metal ions is 1:0.5, 1:1, 1:4, 1:8, 1:12, 1:24, 1:30, 1:40, 1:50, 1:50, 1:70, 1:80, 1:90, 1:100. In some embodiments, in step (R4) of method I, the molar ratio of the second reductant and the antibody is 0.5:1, 1:1, 1.2:1, 1.4:1, 1.6:1, 1.8:1, 2:1, 2.2:1, 2.4:1 or 2.5:1. In some embodiments, in step (R4) of method I, the incubation time is 0.5 h, 1 h, 2 h, 4 h, 6 h, 8 h, 10 h, 12 h, 14 h, 16 h, 18 h, 20 h, 22 or 24 h. In some embodiments, in step (R4) of method I, the molar ratio of the second reductant and the transition metal ions is 1:0.5 to 1:100, and/or the molar ratio of the second reductant and the antibody is 0.8:1 to 2:1, and the incubation time is 0.5 h to 24 h. In some embodiments, in step (R4), the molar ratio of the second reductant and the transition metal ions is 1:0.4 to 1:100, and/or the molar ratio of the second reductant and the antibody is 2:1 to 2.5:1, and the incubation time is 1 h to 9 h. In some embodiments, the molar ratio of the second reductant and the transition metal ions, the molar ratio of the second reductant and the antibody, the incubation time in step (R4) of the method I also applies to that in step (R4) of the method III.

In some embodiments, the temperature in step (R4) of method I is same as that in step (R3) of the method I.

In some embodiments, the parameters in step (C4) of the method I are same as that in step (C1) of the method I. In some embodiments, the time and the temperature, and the molar ratio of the second conjugating group and the antibody in step (C4) of the method I are same as that in step (C1) of method I.

In some embodiments, the second conjugating groups in step (C4) of the method I and the second conjugating groups in step (C3) of the method I are same or different.

In some embodiments, the DAR value of conjugates prepared by method I is 2 (or D2), in this case, two first linker-payloads are linked to the antibody. In some embodiments, the DAR value of conjugates prepared by method I is 1 (or D1), in this case, one of the first thiobridge reagent bearing the first linker-payloads are linked to an antibody. In some embodiments, in method I, with step (R3) and (C3), bi-payload conjugates are prepared, which includes D2+D3, D2+D6, D2+D4, D2+D2, D1+D3, D1+D6, D1+D4, D1+D2, D0+D3 or D0+D6. In some embodiments, in method I, with step (R3), (C3), (R4) and (C4), D1+D2+D2, D2+D1+D2, D2+D2+D1, D2+D1+D1, D1+D1+D2, D1+D1+D1, D2+D2+D2 are prepared.

In some embodiments, in step (R1) of the method II, three of the interchain disulfide bonds of the antibody are reduced, optionally two of the interchain disulfide bonds in the Fab region and one of the interchain disulfide bond in the hinge region of the antibody are reduced.

In some embodiments, in step (R1) of the method II, the molar ratio of the first reductant and the transition metal ions is 1:0.05 to 1:40, 1:0.08 to 1:30, 1:0.1 to 1:30, 1:0.1 to 1:20, 1:0.5 to 8:1 or 1:0.25 to 1:7.5, optionally, the first reductant is TCEP or the compound having formula (I) or formula (II).

In some embodiments, in step (R1) of the method H, the molar ratio of the first reductant and the transition metal ions is 1:0.1 to 1:15, 1:0.1 to 1:10, 1:0.25 to 1:15, 1:0.25 to 1:12, 1:0.25 to 1:10, 1:0.25 to 1:8, 1:0.25 to 1:7.5, 1:0.25 to 1:7, 1:0.25 to 1:5, 1:0.25 to 1:4 or 1:0.5 to 1:4. In some embodiments, in step (R1) of the method II, the molar ratio of the first reductant and the transition metal ions is 1:0.08, 1:0.2, 1:0.5, 1:0.8, 1:1, 1:2, 1:4, 1:8, 1:10, 1:12, 1:14, 1:16, 1:18, 1:20, 1:23, 1:2, 1:27, 1:29, 1:32, 1:34, 1:36, 1:38 or 1:40.

In some embodiments, the molar ratio of the first reductant and the transition metal ions in step (R1) of method II also applies to that in step (R1) of method III, IV and V.

In some embodiments, in step (R1) of the method II, the molar ratio of the first reductant and the antibody is 2.8:1 to 20:1, 3:1 to 15:1, 3:1 to 6:1, 3.5:1 to 5:1, 4:1 to 10:1, 5:1 to 13:1.

In some embodiments, in step (R1) of the method II, the molar ratio of the first reductant and the antibody is 3.5:1 to 5:1, 4:1 to 10:1 or 5:1 to 13:1, 3:1 to 5:1, 4:1 to 5:1, or 3.8:1 to 4.6:1. In some embodiments, in step (R1) of the method II, the molar ratio of the first reductant and the antibody is 2.8:1, 3:1, 4:1, 5:1, 6:1, 7:1, 8:1, 9:1, 10:1, 11:1, 12:1, 13:1, 14:1, 15:1, 16:1, 17:1, 18:1, 19:1 or 20:1.

In some embodiments, the molar ratio of the first reductant and the antibody in step (R1) of method II also applies to that in step (R1) of method III, IV and V.

In some embodiments, in step (R1) of the method II, the incubation time is 1 h to 24 h, 14 h to 24 h, 16 h to 20 h, or 16 h to 18 h. In some embodiments, in step (R1) of the method II, the incubation time is 2 h, 4 h, 6 h, 8 h, 10 h, 12 h, 14 h, 16 h, 18 h, 20 h, 22 h or 24 h. In some embodiments, the incubation time in step (R1) of method II also applies to that in step (R1) of method III, IV and V.

In some embodiments, in step (R1) of the method II, the molar ratio of the first reductant and the antibody is 3:1 to 6:1, the incubation time is 10 h to 24 h. In some embodiments, in step (R1) of the method II, the molar ratio of the first reductant and the antibody is 2.8:1 to 3:1, the incubation time is 10 h to 24 h. In some embodiments, in step (R1) of the method II, the molar ratio of the first reductant and the antibody is 2.8:1, 2.9:1 or 3:1, the incubation time is 10 h, 12 h, 16 h, 18 h, 20 h, 22 h or 24 h. In some embodiments, in step (R1) of the method II, the molar ratio of the first reductant and the antibody is 3:1, 3.2:1, 3.5:1, 3.8:1, 4:1, 4.5:1, 5:1, 5.5:1 or 5:8:1, the incubation time is 10 h, 11 h, 12 h, 13 h, 14 h, 15 h, 16 h, 17 h, 18 h, 19 h, 20 h, 21 h, 22 h, 23 h or 24 h.

In some embodiments, the incubation time in step (R1) of the method II is shortened with increasing the molar ratio of the first reductant and the antibody. In some embodiments, in step (R1) of the method II, the molar ratio of the first reductant and the antibody is 6:1 to 20:1, and the incubation time is 4 h to 16 h. In some embodiments, in step (R1) of the method II, the molar ratio of the first reductant and the antibody is 7:1, 8:1, 9:1, 10:1, 11:1, 12:1, 13:1, 14:1 or 15:1, and the incubation time is 4 h, 5 h, 6 h, 7 h, 8 h, 9 h, 10, 11 h, 12 h, 13 h, 14 h, 15 h or 16 h.

In some embodiments, in step (R1) of the method II, the molar ratio of the transition metal ions and the antibody is 1:1 to 50:1, 1:1 to 30:1, 1:1 to 20:1, 1:1 to 15:1, 8:1 to 30:1, 12:1 to 30:1, 12:1 to 30:1, 8:1 to 16:1, 4:1 to 30:1, 4:1 to 16:1, 8:1 to 16:1, 1:1 to 10:1, 1:1 to 8:1, 1:1 to 6:1, 1:1 to 5:1, 1:1 to 4:1, 1:1 to 3:1, 1:1 to 2:1, 2:1 to 6:1, 2:1 to 4:1.

In some embodiments, in step (R3) of the method II, one of the interchain disulfide bond in the product prepared with step (C1) is reduced completely without the transition metal ions.

In some embodiments, in step (R3) of the method II, the molar ratio of the second reductant and the antibody is 1:1 to 20:1. In some embodiments, in step (R3) of the method II, the molar ratio of the second reductant and the antibody is 1:1, 2:1, 4:1, 6:1, 8:1, 10:1, 12:1, 14:1, 16:1, 18:1 or 20:1.

In some embodiments, in step (R3) of the method U, the incubation temperature of the second reductant is 0° C. to 37° C., the incubation time is 0.5 h to 24 h.

In some embodiments, in step (R3) of the method U, the incubation temperature of the second reductant is 5° C. to 30° C., 10° C. to 30° C., 15° C. to 30° C., 20° C. to 30° C. or 25° C. to 30° C. In some embodiments, in step (R3) of the method II, the incubation temperature of the second reductant is 25° C.

In some embodiments, in step (R3) of the method II, the incubation time is 1 h to 20 h, 5 h to 20 h, 6 h to 18 h, 8 h to 18 h, 8 h to 15 h or 8 h to 12 h. In some embodiments, in step (R3) of the method II, the incubation time is 1 h, 2 h, 3 h, 4 h, 5 h, 6 h, 7 h, 8 h or 12 h.

In some embodiments, in step (C1) of the method II, the molar ratio of the first thiobridge reagent and the antibody is 3:1 to 10:1. In some embodiment, in step (C1) of the method II, the molar ratio of the first thiobrige reagent and the antibody is 3:1, 3.3:1, 4:1, 5:1, 6:1, 7:1, 8:1, 9:1 or 10:1.

In some embodiments, in step (C1) of the method H, when the first linker-payload reacts with the reactive groups in the first thiobridge reagent, the molar ratio of the first linker-payload and the antibody is 3:1 to 10:1. In some embodiments, in step (C1) of the method II, the molar ratio of the first linker-payload and the antibody is 3:1, 4:1, 5:1, 6:1, 7:1, 8:1, 9:1 or 10:1.

In some embodiments, in step (C1) of the method H, when the first linker-payload reacts with the reduced thiol groups, the molar ratio of the first linker-payload and the antibody is 6:1 to 20:1. In some embodiments, in step (C1) of the method H, when the first linker-payload reacts with the reduced thiol groups, the molar ratio of the first linker-payload and the antibody is 6:1, 20:3, 7:1, 8:1, 9:1, 10:1, 15:1 or 20:1.

In some embodiments, in step (C3) of the method II, according to the amount of the antibody, the second conjugating group is excess.

In some embodiments, in step (C3) of the method II, the molar ratio of the second thiobridge reagent and the antibody is 1:1 to 3:1. In some embodiments, in step (C3) of the method II, the molar ratio of the second thiobridge reagent and the antibody is 1:1, 1.5:1, 2:1 or 3:1.

In some embodiments, in step (C3) of the method H, when the second linker-payload reacts with the reactive groups in the second thiobridge reagent, the molar ratio of the second linker-payload and the antibody is 1:1 to 8:1. In some embodiments, in step (C3) of the method II, when the second linker-payload reacts with the reactive groups in the second thiobridge reagent, the molar ratio of the second linker-payload and the antibody is 1:1, 2:1, 3:1, 4:1, 5:1, 6:1, 7:1 or 8:1.

In some embodiments, in step (C3) of the method I, when the second linker-payload reacts with the reduced thiol groups, the molar ratio of the second linker-payload and the antibody is 2:1 to 20:1.

In some embodiments, in step (C3) of the method IT, when the second linker-payload reacts with the reduced thiol groups, the molar ratio of the second linker-payload and the antibody is 2:1, 3:1, 4:1, 5:1, 6:1, 7:1, 8:1, 10:1, 12:1, 14:1, 16:1 or 18:1.

In some embodiments, the DAR value of conjugates prepared by method II is 6 (or D6), in this case, six of the first linker-payloads are linked to the antibody. In some embodiments, the DAR value of conjugates prepared by method I is 3 (or D3), in this case, three of the first thiobridge reagents bearing the first a linker-payloads are linked to the antibody. In some embodiments, in method II, with step (R3) and (C3), bi-payload conjugates are prepared, which includes D6+D1, D6+D2, D3+D1, D3+D2, D0+D1 and D0+D2.

In some embodiments, the parameters in step (R1) of method III are same as that in step (R1) of method II. In some embodiments, the molar ratio of the first reductant and the transition metal ions, the molar ratio of the first reductant and the antibody, the incubation time and temperature in step (R1) of method III are same as that in step (R1) of method II.

In some embodiments, in step (R1) of the method HI, the molar ratio of the first reductant and the antibody is 4:1 to 15:1, and the incubation time is 1 h to 16 h. In some embodiments, in step (R1) of the method III, the molar ratio of the first reductant and the antibody is 4.5:1, 5:1, 5:1, 7:1, 8:1, 9:1, 10:1, 11:1, 12:1, 13:1, 14:1 or 15:1, the incubation time is 1 h, 2 h, 3 h, 4 h, 5 h, 6 h, 7 h, 8 h, 9 h, 10, 11 h, 12 h, 13 h, 14 h, 15 h or 16 h.

In the disclosure, there is no specific limitation to the oxidant of step (O1), as long as the oxidant can re-oxidize the reduced thiol groups. In some embodiments, the oxidant is Dehydroascorbic acid (DHAA) in step (O1).

In some embodiments, in step (O1) of the method III, the molar ratio of oxidant and the antibody is 2:1 to 25:1, 2:1 to 20:1, 4:1 to 22:1, 4:1 to 15:1, or 6:1 to 14:1. In some embodiments, in step (O1) of the method III, the molar ratio of oxidant and the antibody is 1:1 to 20:1, 3:1 to 18:1, 6:1 to 18:1, 8:1 to 14:1, 6:1 to 12:1, 5:1 to 20:1, 4:1 to 15:1, or 6:1 to 14:1. In some embodiments, in step (O1) of the method III, the molar ratio of oxidant and the antibody is 1:1 to 15:1, 3:1 to 15:1, 6:1 to 15:1, 8:1 to 14:1, 6:1 to 10:1, 8:1 to 12:1, 6:1 to 10:1, 6:1 to 12:1, 3:1 to 8:1, 3:1 to 6:1, 5:1 to 15:1, 5:1 to 10:1, 5:1 to 8:1, 2:1 to 7:1, 4:1 to 9:1, 1:1 to 5:1, 2:1 to 4:1, or 2:1 to 6:1 in step (O1).

In some embodiments, in step (O1) of the method III, the oxidation temperature is 0° C. to 37° C., 0° C. to 30° C., 0° C. to 25° C., 0° C. to 20° C., 0° C. to 15° C., 0° C. to 10° C., or 0° C. to 5° C., 5° C. to 30° C., 10° C. to 30° C., 15° C. to 30° C., 20° C. to 30° C., 0° C. to 25° C., 0° C. to 20° C., 10° C. to 25° C., 15° C. to 30° C., 5° C. to 25° C., 10° C. to 20° C. or 0° C. to 15° C.

In some embodiments, in step (O1) of the method III, the oxidation temperature is 10° C. to 40° C., 10° C. to 35° C., 10° C. to 30° C., 10° C. to 25° C., 15° C. to 35° C., 20° C. to 30° C. In some embodiments, in step (O1) of the method III, the oxidation temperature is room temperature. As used herein, the term “room temperature” refers to 23° C.±2° C., 25° C.±5° C. or 20° C.±5° C.

In some embodiments, in step (O1) of the method III, the oxidation time is 1 h to 48 h, 0.5 h to 15 h, 1 h to 10 h, 1 h to 5 h, 0.5 h to 5 h, 0.5 h to 3 h, 1 h to 3 h, 2 h to 5 h, 2 h to 4 h or 2 h to 3 h.

In some embodiments, in step (O1) of the method III, the oxidation time is 0.5 h to 4 h, 0.5 h to 3 h, 0.5 h to 2 h, 0.5 h to 1 h, 1 h to 4 h, 1 h to 3 h, 1 h to 2 h, or 2 h to 4 h.

In some embodiments, the oxidation reaction in step (O1) of the method III is performing, optionally in darkness, at temperature of 0° C. to 37° C., 0° C. to 30° C., 15° C. to 30° C., or 20° C. to 30° C., and/or the oxidation time is 1 h to 48 h, 1 h to 5 h, or 1 h to 3 h.

In some embodiments, in step (O1) of the method III, the oxidation temperature is 0° C. to 25° C., 0° C. to 15° C., 0° C. to 10° C., or 0° C. to 5° C., and/or the oxidation time is 0.5 h to 12 h, 1 h to 10 h, 1 h to 8 h, 2 h to 5 h, or 3 h to 8 h.

In some embodiments, in step (O1) of the method III, the oxidation temperature is 15° C. to 25° C., and the oxidation time is 1 h to 3 h.

In some embodiments, in step (O1) of the method III, the thiol groups in Fab region are re-oxidized. In some embodiments, four thiol groups in Fab region are re-oxidized.

In some embodiments, the parameters in step (C1), (R3), (C3), (R4) and (C4) of method III are same as that in step (C1), (R3), (C3), (R4) and (C4) of method I.

In some embodiments, the DAR value of conjugates prepared by method III is 2 (or D2), in this case, two of the first linker-payloads are linked to the antibody. In some embodiments, the DAR value of conjugates prepared by method III is 1 (or D1), in this case, one of the first thiobridge reagent bearing the first linker-payloads are linked to an antibody. In some embodiments, in method III, with step (R3) and (C3), bi-payload conjugates are prepared, which includes D2+D3, D2+D6, D2+D4, D2+D2, D1+D3, D1+D6, D1+D4, D1+D2, D0+D3 or D0+D6. In some embodiments, in method III, with step (R3), (C3), (R4) and (C4), D1+D2+D2, D2+D1+D2, D2+D2+D1, D2+D1+D1, D1+D1+D2, D1+D1+D1, D2+D2+D2 are prepared.

In some embodiments, the parameters in step (R1) and (O1) of method IV are same as that in step (R1) and (O1) of method III.

In some embodiments, in step (R2) of the method IV, introducing the metal chelators.

In some embodiments, in step (R2) of the method IV, the molar ratio of the metal chelators and the antibody is 2:1 to 120:1, 2:1 to 100:1, 2:1 to 80:1, 5:1 to 60:1, 10:1 to 60:1, 20:1 to 60:1, 30:1 to 60:1, 40:1 to 60:1 or 50:1 to 60:1.

In some embodiments, in step (R2) of the method IV, the molar ratio of the second reductant and the antibody is 10:1 to 25:1, 10:1 to 23:1, 10:1 to 20:1, 10:1 to 19:1, 10:1 to 18:1, 15:1 to 25:1, 15:1 to 20:1, 1:1 to 8:1, 1:1 to 5:1, 1:1 to 3:1 or 1:1 to 2:1. In some embodiments, in step (R2) of the method IV, the molar ratio of the second reductant and the antibody is 1:1, 1.2:1, 1.4:1, 1.6:1, 1.8:1, 2:1, 2.2:1, 2.4:1, 2.6:1, 2.8:1 or 3:1.

In some embodiments, in step (R2) of the method IV, the reduction temperature is 0° C. to 30° C., the reduction time is 1 h to 8 h.

In some embodiments, in step (R2) of the method IV, the reduction temperature is 0° C. to 37° C., 5° C. to 25° C., 1° C. to 20° C. or 1° C. to 15° C.

In some embodiments, in step (R2) of the method IV, the reduction time is 1 h to 8 h, 1 h to 7 h, 1 h to 6 h, 1 h to 5 h, 2 h to 5 h, 2 h to 4 h or 2 h to 3 h.

In some embodiments, in step (R2) of the method IV, one of the interchain disulfides of the product prepared from step (O1) of the method IV is reduced.

In some embodiments, in step (R3) of the method IV, two of the interchain disulfide bonds in the product prepared from step (C1) are reduced completely without the transition metal ions. In some embodiments, one of the interchain disulfide bond in the product prepared from step (C1) is selectively reduced with the transition metal ions.

In some embodiments, the incubation temperature and time in step (R3) of method IV are same as that in step (R3) of method I.

In some embodiments, without the transition metal ions, the molar ratio of the second reductant and the antibody in step (R3) of the method IV is same as that in step (R3) of method I.

In some embodiments, in step (R3) of the method IV, introducing the transition metal ions, one of the interchain disulfide bond is selectively reduced, and the parameters in step (R3) of the method IV are same as that in step (R3) of method I.

In some embodiments, the reaction temperature and time in step (C1) and (C3) of method IV are same as that in step (C1) and (C3) of method I.

In some embodiments, in step (C1) of the method IV, according to the amount of the antibody, the first conjugating group is excess.

In some embodiments, in step (C1) of the method IV the molar ratio of the first thiobridge reagent and the antibody is 2:1 to 10:1. In some embodiment, in step (C1) of the method IV, the molar ratio of the first thiobrige reagent and the antibody is 2:1, 3:1, 3.3:1, 4:1, 5:1, 6:1, 7:1, 8:1, 9:1 or 10:1.

In some embodiments, in step (C1) of the method IV, when the first linker-payload reacts with the reactive groups in the first thiobridge reagent, the molar ratio of the first linker-payload and the antibody is 2:1 to 10:1. In some embodiments, in step (C1) of the method IV, the molar ratio of the first linker-payload and the antibody is 2:1, 3:1, 4:1, 5:1, 6:1, 7:1, 8:1, 9:1 or 10:1.

In some embodiments, in step (C1) of the method IV, when the first linker-payload reacts with the reduced thiol groups, the molar ratio of the first linker-payload and the antibody is 4:1 to 20:1. In some embodiments, in step (C1) of the method IV, when the first linker-payload reacts with the reduced thiol groups, the molar ratio of the first linker-payload and the antibody is 4:1, 5:1, 6:1, 20:3, 7:1, 8:1, 9:1, 10:1, 15:1 or 20:1.

In some embodiments, in step (C3) of the method IV, according to the amount of the antibody, the second conjugating group is excess. In some embodiments, the molar ratio of the second conjugating group and the antibody in step (C3) of the method IV is same as that in step (C3) of the method I.

In some embodiments, the DAR value of conjugates prepared by method IV is 4 (or D4), in this case, four of the first linker-payloads are linked to the antibody. In some embodiments, in method IV, with step (R3) and (C3), bi-payload conjugates are prepared, which includes D4+D4 or D4+D2.

In some embodiments, the parameters in step (R1) of the method V are same as that in step (R1) of the method II. In some embodiments, the molar ratio of the first reductant and the transition metal ions, the molar ratio of the first reductant and the antibody, the incubation time and temperature in step (R1) of the method V are same as that in step (R1) of the method II.

In some embodiments, in step (R1) of the method V, the molar ratio of the first reductant and the transition metal ions is 1:0.05 to 1:40 or 1:0.5 to 1:10. In some embodiments, in step (R1) of the method II, the molar ratio of the first reductant and the transition metal ions is 1:0.1, 1:0.2, 1:0.3, 1:0.4, 1:0.5, 1:0.6, 1:0.7, 1:0.8, 1:1, 1:2, 1:3, 1:4, 1:5, 1:6, 1:7, 1:8, 1:9 or 1:10.

In some embodiments, in step (R1) of the method V, the molar ratio of the first reductant and the antibody is 2.8:1 to 20:1 or 3.5:1 to 10:1. In some embodiments, in step (R1) of the method V, the molar ratio of the first reductant and the antibody is 3.5:1, 4:1, 4.5:1, 5:1, 6:1, 7:1, 8:1, 9:1 or 10:1.

In some embodiments, the parameters in step (C2) of the method V are same as that in step (C1) of the method IV.

In some embodiments, in step (C2) of the method V, the reactive temperature is 20° C.-37° C. In some embodiments, in step (C2) pf the method V, the reactive temperature is 20° C., 24° C., 26° C., 28° C., 30° C., 32° C., 34° C. or 36° C. In some embodiments, improve the reactive temperature in step (C2) are useful to increase the content of the ADC with D4.

In some embodiments, in step (C2) of the method V, the molar ratio of the first linker-payload and the antibody is 4:1 to 30:1. In some embodiments, in step (C2) of the method V, the molar ratio of the first linker-payload and the antibody is 4:1, 8:1, 12:1, 16:1, 20:1, 24:1 or 28:1. In some embodiments, improve the molar ratio of the first linker-payload and the antibody in step (C2) are useful to increase the content of the ADC with D4.

In some embodiments, in step (R3) of the method V, two of the interchain disulfide bonds in the product prepared from step (C2) are reduced completely without the transition metal ions. In some embodiments, in step (R3) of the method V, one of the interchain disulfide bond in the product prepared from step (C2) is selectively reduced with the transition metal ions.

In some embodiments, the incubation temperature and time in step (R3) of method V are same as that in step (R3) of method I.

In some embodiments, without the transition metal ions, the molar ratio of the second reductant and the antibody in step (R3) of the method V is same as that in step (R3) of method I.

In some embodiments, in step (R3) of the method V, introducing the transition metal ions, one of the interchain disulfide bond is selectively reduced, and the parameters in step (R3) of the method V are same as that in step (R3) of method I.

In some embodiments, the parameters in step (C3) of the method V are same as that in step (C3) of the method IV.

In some embodiments, the DAR value of conjugates prepared by method V is 4 (or D4), in this case, four of the first linker-payloads are linked to the antibody. In some embodiments, in method V, with step (R3) and (C3), bi-payload conjugates are prepared, which includes D4+D4 or D4+D2.

Without to bound by any theory, the reductant selectively reduces disulfide bonds in buffer system.

The buffer system of the method I, II, III, IV and V independently is MES buffer, Bis-Tris buffer, PIPES buffer, MOPS buffer, BES buffer, HEPES buffer, DIPSO buffer, MOBS buffer, MOPSO buffer, TES buffer, ACES buffer, TAPSO buffer, PBS, ADA buffer, PB, Acetate buffer, BTP buffer, HEPPSO buffer, POPSO buffer, EPPS buffer or Tris buffer.

As used herein, the term “ADA buffer” refers to N-(Carbamoylmethyl) iminodiacetic acid buffer.

As used herein, the term “MES buffer” refers to 2-(N-morpholino) ethane sulfonic acid buffer.

As used herein, the term “Bis-Tris buffer” refers to Bis(2-hydroxyethyl) amino-tris(hydroxymethyl)methane buffer.

As used herein, the term “PIPES buffer” refers to piperazine-1,4-bisethanesulfonic acid buffer.

As used herein, the term “MOPS buffer” refers to 3-morpholinopropanesulfonic Acid buffer.

As used herein, the term “BES buffer” refers to N, N-Bis (2-hydroxyethyl)-2-aminoethanesulphonic acid buffer.

As used herein, the term “HEPES buffer” refers to 4-hydroxyethyl piperazine ethane sulfonic acid buffer.

As used herein, the term “DIPSO buffer” refers to 3-[bis(2-hydroxyethyl) amino]-2-hydroxypropanesulphonic acid buffer.

As used herein, the term “MOBS buffer” refers to 3-morpholinopropanesulfonic Acid buffer.

As used herein, the term “MOPSO buffer” refers to 3-(N-morpholino)-2-hydroxy-1-propanesulfonic acid buffer.

As used herein, the term “TES buffer” refers to 2-[tris(hydroxymethyl)methylamino]-1-ethanesulfonic acid buffer.

As used herein, the term “ACES buffer” refers to N-(carbamoylmethyl)taurine buffer.

As used herein, the term “TAPSO buffer” refers to 3-[N-tris-(hydroxymethyl) methylamino]-2-hydroxypropanesulphonic acid buffer.

As used herein, the term “PBS” refers to phosphate buffer saline.

As used herein, the term “ADA buffer” refers to N-(Carbamoylmethyl) iminodiacetic acid buffer.

As used herein, the term “PB buffer” refers to refers to phosphate buffer.

As used herein, the term “BTP buffer” refers to Bis-tris propane buffer.

As used herein, the term “Heppso buffer” refers to N-(Hydroxyethyl) piperazine-N′-2-hydroxypropanesulfonic acid buffer.

As used herein, the term “POPSO buffer” refers to piperazine-N, N′-bis(2-hydroxy-propane sulfonic) acid buffer.

As used herein, the term “EPPS buffer” refers to 4-(2-Hydroxyethyl)-1-piperazinepropanesulfonic acid buffer.

As used herein, the term “Tris buffer” refers to tris(hydroxymethyl)aminomethane buffer.

In some embodiments, the buffer system of method I, II, III and IV independently is Bis-Tris buffer, MOPS buffer, BES buffer, HEPES buffer, DIPSO buffer, MOBS buffer, MOPSO buffer, TES buffer, ACES buffer, TAPSO buffer or MES buffer.

In some embodiments, the buffer system of method I, II, III and IV independently is Bis-Tris buffer, MOPS buffer, BES buffer, HEPES buffer, DIPSO buffer, MOBS buffer, MOPSO buffer, TES buffer, ACES buffer or TAPSO buffer.

In some embodiments, the buffer system of method V is selected from the group consisting of Bis-Tris buffer, MOPS buffer, BES buffer, HEPES buffer, DIPSO buffer, MOBS buffer, MOPSO buffer, TES buffer, ACES buffer, TAPSO buffer, PIPES buffer or MES buffer.

In some embodiments, the buffer system of method I, II, III, IV and V is BES buffer.

In some embodiments, the buffer system of step (R1) and (R3) is same. In some embodiments, the buffer system of step (R1) and (R3) is different. In some embodiments, the buffer system of step (R1) and (R2) is same. In some embodiments, the buffer system of step (R1) and (R2) is different.

In some embodiments, the pH value of the buffer system is 5.8 to 8.0. In some embodiments, the pH value of the buffer system is 6.0 to 7.4. In some embodiments, the pH value of the buffer system is 6.4 to 7.4. In some embodiments, the pH value of the buffer system is 6.7 to 7.4. In some embodiments, the pH value of the buffer system is 6.0, 6.2, 6.5, 6.8, 7.0, 7.2 or 7.4.

The concentration of the buffer system independently is ranging from 10 mM to 100 mM (mmol/L), optionally, the concertation of the buffer system is 20 mM to 100 mM, 20 mM to 80 mM, 20 mM to 60 mM, 20 mM to 40 mM, 40 mM to 80 mM, 40 mM to 60 mM, 30 mM to 80 mM, 30 mM to 60 mM, 50 mM to 80 mM, or 30 mM to 70 mM.

In some embodiments, the concentration of buffer system of step (R1) and (R3) is same. In some embodiments, the concentration of buffer system of step (R1) and (R3) is different. In some embodiments, the concentration of buffer system of step (R1) and (R2) is same. In some embodiments, the concentration of buffer system of step (R1) and (R2) is different.

In the disclosure, the metal chelators can trap excessive the transition metal ions. There is no specific limitation to the metal chelators, as long as the metal chelators can trap the excessive transition metal ions and do not affect the reduction of the disulfide bonds within the antibody. In some embodiments, the metal chelators are selected from a group consisting of ethylene diamine tetraacetic acid (EDTA), nitrilotriacetic acid (NTA), diethylenetriaminepentaacetic acid (DTPA), citric Acid (CA), tartaric acid (TA), gluconic acid (GA) or N-(2-hydroxyethyl) ethylenediamine-N, N′, N′-triacetic acid (HEDTA).

In some embodiments, the metal chelators are selected from a group consisting of EDTA, NTA and DTPA, or their sodium salt. In some embodiments, the metal chelators in the method are Ethylenediaminetetraacetic acid disodium salt (EDTA-2Na).

In some embodiments, the molar ratio of the metal chelators and the antibody in step (C1) of method I, II, III or IV, or in step (C2) in method V is 1:1 to 100:1, 10:1 to 100:1, 20:1 to 100:1, 20:1 to 80:1, 20:1 to 70:1, 30:1 to 60:1, 40:1 to 50:1, 35:1 to 60:1, 40:1 to 55:1.

In some embodiments, the molar ratio of the metal chelators and the antibody in step (C3) of method I, II, III, IV and V is 1:1 to 100:1, 1:1 to 60:1, 1:1 to 50:1, 1:1 to 20:1, 1:1 to 10:1, 1:1 to 8:1, 1:1 to 6:1, 1:1 to 5:1, 2:1 to 8:1, 2:1 to 6:1.

In some embodiments, the method further comprises a step of purification after step (O1), (C1), (C2) and/or (C3).

In some embodiments, the products prepared from step (O1), (C1), (C2) and/or (C3) are recovered by any suitable purification method, such as using a de-salting column, size exclusion chromatography, ultrafiltration, dialysis, ultrafiltration (UF)-diafiltration (DF), and the like. If needed, further product enrichment (e.g., D2) may be applied in some case using hydrophobic interaction chromatography (HIC). In some embodiments, the products prepared from step (O1), (C1), (C2) and/or (C3) are purified by a desalting column, size exclusion chromatography, ultrafiltration, dialysis and/or the like. In some embodiments, the products prepared from step (O1), (C1), (C2) and/or (C3) are purified by a desalting column.

In some embodiments, the excess amount of metal chelators and a complex of the metal chelators and the transition metal ions are filtered out in dialysis, ultrafiltration or gel filtration.

In some embodiments, the method further comprises the following steps, introducing a compound that contains at least one thiol group to consume excessive the first conjugating group in step (C1) and/or (C2), and/or the second conjugating group in step (C3) there is no specific limitation to the compound to consume excessive the first conjugating group and/or the second conjugating group, as long as the compound contains at least one thiol group. In some embodiments, the compound is cysteine.

In some embodiments, A linker-payload includes a linker and a payload, which are covalently linked.

In some embodiments, a linker of the first linker-payload and the second liner payload is selected from any one of which the one terminal can be connected to the reduced thiol group of the antibody or the reactive groups of the thiobridge reagent, and the other terminal can be connected to a payload of the payload.

As used herein, the term “linker” refers to a molecule which contains at least two substituted groups, one of which can covalently bond a drug molecule and the other of which can covalently couple to an antibody or the reactive groups of the thiobridge reagent.

In some embodiments, when the first linker-payload and/or the second linker-payload react(s) with the reduced thiol groups, the linker of the first linker-payload and the second linker-payload independently includes a cleavable linker or a noncleavable linker. Cleavable linkers optionally are chemically labile and enzyme-labile linkers. Due to the high plasma stability and good intracellular cleaving selectivity and efficiency, enzyme-labile linkers (such as cathepsin B) are broadly selected as cleavable linker candidates in antibody-drug conjugates (ADCs). In some embodiments, enzyme-labile linkers include a peptide unit (-AAs-), -maleimidocaproyl-(-MC-), -maleimidocaproyl-peptide moiety-(-MC-peptide moiety-). -p-aminobenzyl alcohol-(-PAB-). In some embodiments, the peptide unit is dipeptides, tripeptides, tetrapeptides or pentapeptides.

In some embodiments, without the limitation, the dipeptides may be valine-alanine (VA), valine-citrulline (VC), alanine-asparagine (AD), alanine-phenylalanine (AF), phenylalanine-lysine (FK), alanine-lysine (AK), alanine-valine (AV), valine-lysine (VK), lysine-lysine (KK), phenylalanine-citrulline (FC), leucine-citrulline (LC), isoleucine-citrulline (IC), tryptophan-citrulline (WC) or phenylalanine-alanine (FA). In some embodiments, the dipeptides can be valline-citruline- (-Val-Cit-), -valline-lysine- (-Val-Lys-), -valline-arginine- (-Val-Arg-), -phenylalanine-citruline- (-Phe-Cit-), -phenylalanine-lysine-(-Phe-Lys-), and -phenylalanine-arginine- (-Phe-Arg-). Typical enzyme-labile linkers include -Val-Cit- and -Phe-Lys-, which can be recognized by cathepsin B.

In some embodiments, without the limitation, the tripeptides may be alanine-alanine-asparagine (AAD), glycine-valine-citrulline (GVC), glycine-glycine-glycine (GGG), phenylalanine-phenylalanine-lysine (FFK), glutamic acid-valine-citrulline (EVC), or glycine-phenylalanine-lysine (GFK).

In some embodiments, without the limitation, the tetrapeptides may be glycine-glycine-phenylalanine-glycine (GGFG).

In some embodiments, without the limitation, when the first linker-payload and/or the second linker-payload react(s) with the reduced thiol groups, the linker of the first linker-payload and the second linker-payload can be MC-VA-PAB, MC-VC-PAB, MC-AD-PAB, MC-AF-PAB, MC-FK-PAB, MC-AK-PAB, MC-AV-PAB, MC-VK-PAB, MC-KK-PAB, MC-FC-PAB, MC-LC-PAB, MC-IC-PAB, MC-WC-PAB or MC-FA-PAB independently. In some embodiments, without the limitation, when the first linker-payload and/or the second linker-payload react(s) with the reduced thiol groups, the linker of the first linker-payload and the second linker-payload can be MC-AAD-PAB, MC-GVC-PAB, MC-GGG-PAB, MC-FFK-PAB, MC-EVC-PAB, or MC-GFK-PAB independently. In some embodiments, without the limitation, when the first linker-payload and/or the second linker-payload react(s) with the reduced thiol groups, the linker of the first linker-payload and the second linker-payload can be MC-GGFG.

In some embodiments, the linker comprises a maleimide bearing a drug, an organic chloride bearing a drug, an organic bromide bearing a drug, an organic iodide bearing a drug and/or vinylpyrimidine bearing a drug.

In some embodiments, the linker includes a maleimide bearing a drug, an organic chloride bearing a drug, an organic bromide bearing a drug, an organic iodide bearing a drug and/or vinylpyrimidine bearing a drug.

In some embodiments, when the first linker-payload and/or the second linker-payload react(s) with the reactive groups in the thiobridgee reagent, the linker of the first linker-payload and/or the second linker-payload further include(s) azido and/or dibenzocyclooctyne (DBCO). In some embodiments, when the linker of the first linker-payload and/or the second linker-payload contains azido, the reactive groups of the thiobridge group contain DBCO. In some embodiments, when the linker of the first linker-payload and/or the second linker-payload contains DBCO, the reactive groups of the thiobridge group contain azido.

In some embodiments, when the linker of the first linker-payload and/or the second linker-payload react(s) with the reactive groups in the thiobridge reagent, the linker of the first linker-payload and the second linker-payload is independently selected from any one of the groups consisting of

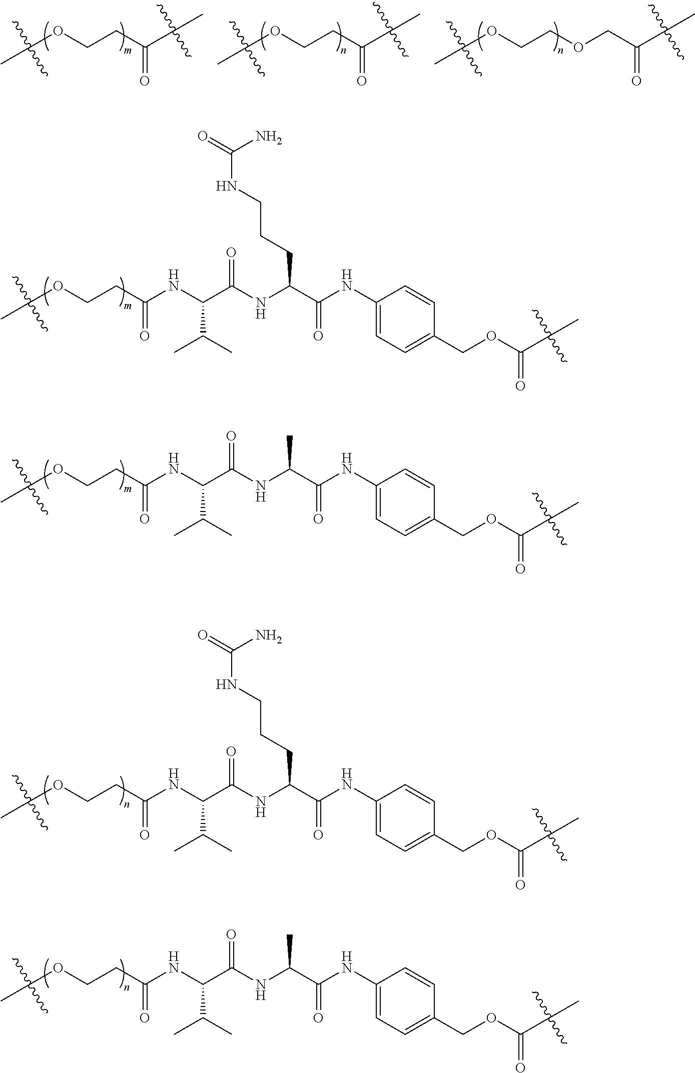

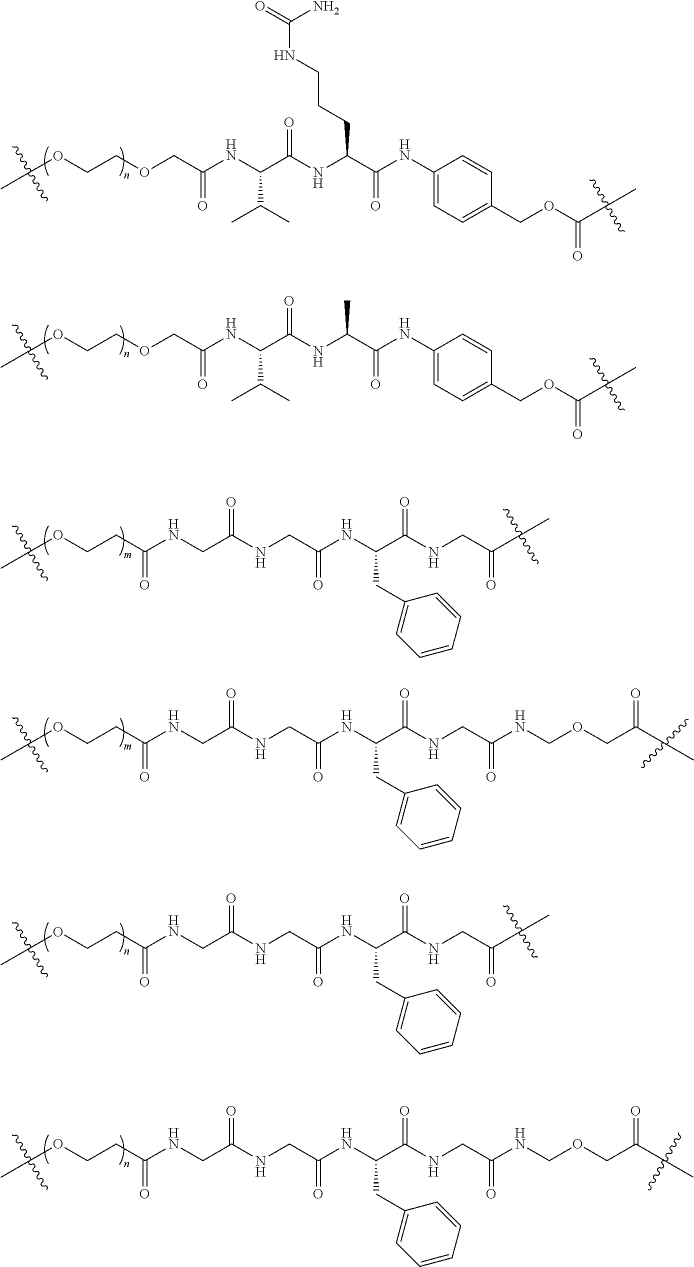

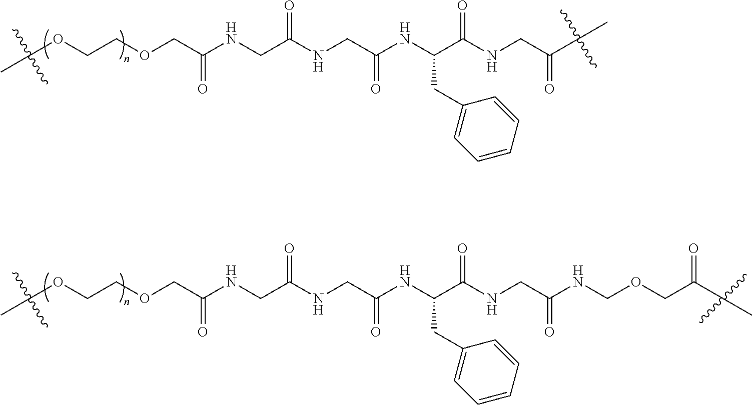

wherein, n is integer of 0-20, and m is integer of 0-20 in the linker. In some embodiments, n is 0, 1, 2, 3, 4, 5, 6, 7, 8, 9, 10; and m is 0, 1, 2, 3, 4, 5, 6, 7, 8, 9, 10 in the linker.

In the method, the linker optionally bears an azido and/or dibenzocyclooctyne (DBCO), so that the linker links to the antibody.

As used herein, the term “end capping reagent”, including the first end capping reagents and the second end capping reagent, refers to a compound which does not bear a drug and contains at least one substituted group which can covalently couple to an antibody.

In some embodiments, the end capping reagent is the cleavable linker or the noncleavable linker.

In some embodiments, the end capping reagent is (2-Aminoethyl) maleimide.

As used herein, the term “payload” refers to any cytotoxic molecule at least one substituted group or a partial structure allowing connection to a linker structure. The payload may kill cancer cells and/or inhibit growth, proliferation, or metastasis of cancer cells, thereby reducing, alleviating, or eliminating one or more symptoms of a disease or disorder.

In some embodiments, the payload is a cytotoxic agent, a cytokine, a nucleic acid, a radionuclide, a chemokine, an immuno(co)-stimulatory molecule, an immunosuppressive molecule, a kinase, a prodrug-converting enzyme, a RNase, a growth factor, a hormone, a coagulation factor, a fibrinolytic protein, peptides mimicking these, and fragments, fusion proteins, or derivatives thereof.

In some embodiments, the payload is a cytotoxic molecule at least one substituted group or a partial structure allowing connection to a linker structure. The payload may kill cancer cells and/or inhibit growth, proliferation, or metastasis of cancer cells, thereby reducing, alleviating, or eliminating one or more symptoms of a disease or disorder. The payload includes but not limited to topoisomerases inhibitor and tubulin inhibitors.

Exemplary payloads are monomethyl auristatin E (MMAE), monomethyl auristatin D (MMAD), monomethyl auristatin EF(MMAF), calicheamicins (CLM), mertansine (DM1), maytansinoids, duocarmycins, anthracyclines, pyrrolobenzodiazepine dimers, amatoxin, quinolinealkaloid, Dxd, doxorubicin hydrochloride, methotrexate, erlotinib, bortezomib, fulvestrant, sunitib imatinib mesylate, letrozole, finasunate, platins such as oxaliplatin, carboplatin, and cisplatin, finasunate, fluorouracil, rapamycin, leucovorin, lapatinib, lonafamib, sorafenib, gefitinib, capmtothecin, topotecan, bryostatin, adezelesin, anthracyclin, carzelesin, bizelesin, dolastatin, auristatins, duocarmycin, eleutherobin, taxols such as paclitaxel or docetaxel, cyclophasphamide, doxorubicin, vincristine, prednisone or prednisolone, other alkylating agents such as mechlorethamine, chlorambucil, and ifosfamide, antimetabolites such as azathioprine or mercaptopurine, vincristine, vinblastine, vinorelbine, vindesine, etoposide, teniposide, etoposide phosphate, epipodophyllotoxins, actinomycin, daunorubicin, valrubicin, idarubicin, edrecolomab, epirubicin bleomycin, plicamycin or mitomycin, and salts thereof.

In some embodiments, the payload is deruxtecan (DXd), cyanine 3 (Cy3), MMAE, MMAD or MMAF. In some embodiments of the present application, the payload is MMAE, DXd or Cy3.

The linker-payload is a chemical moiety, which is synthesized by connecting the linker to the payload. Depending on the desired payload and selected linker, those skilled in the art can select suitable method for coupling them together. For example, some conventional coupling methods, such as amine coupling methods, may be used to form the desired linker-payload which still contains reactive groups for conjugating to the antibodies through covalent linkage. A drug-maleimide complex (i.e., maleimide linking drug) is taken as an example of the payload bearing reactive group in the present disclosure. Most common reactive group capable of bonding to thiol group is maleimide.

Additionally, organic chloride, bromides, iodides also are frequently used.

The linker-payload could be any physical active compound, or any compound used to diagnose, prevent or treat a disease, such as MC-GGFG-DXd, MC-VC-PAB-MMAE, MC-VC-PAB-MMAD, and MC-VC-PAB-MMAF.

In some embodiments, the first linker-payload and the second linker-payload are same. In some embodiments, the first linker-payload and the second linker-payload are different.

The first thiobridge reagent and the second thio-bridging reagent independently contain at least two substituted groups a allowing re-bridging of the thiol groups.

In some embodiments, without the limitation, the first thiobridge reagent and the second thiobridge reagent are independently selected from the group consisting of

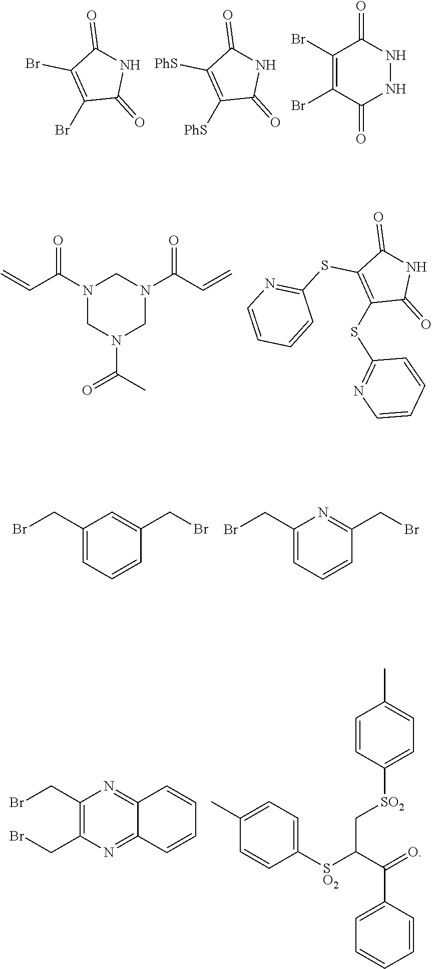

In some embodiments, the reactive groups contain an azido and/or dibenzocyclooctyne (DBCO).

In some embodiments, the thiobridge reagent and the reactive groups are connected by alkyl group or polyethylene glycol (PEG).

In some embodiments, without the limitation, the first thiobridge reagent bearing reactive groups and the second thiobridge reagent bearing reactive groups are independently selected from the group consisting of

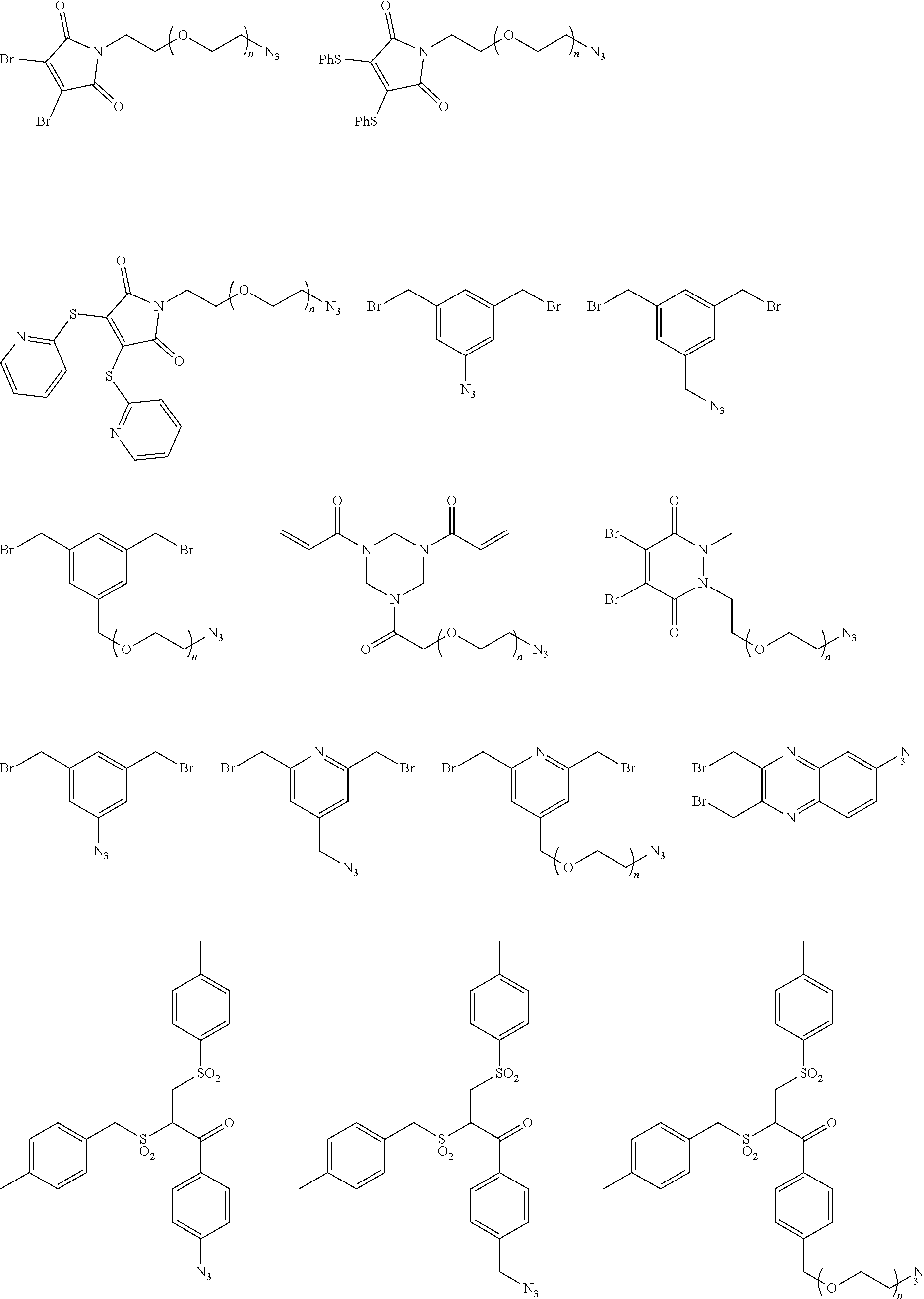

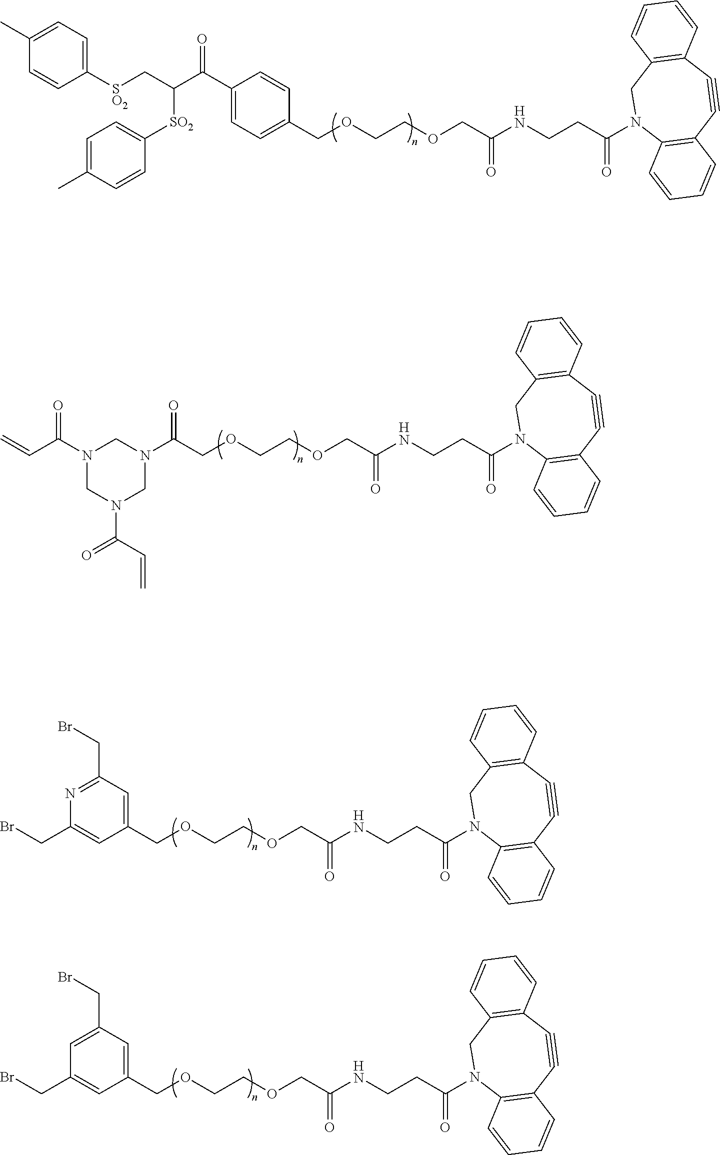

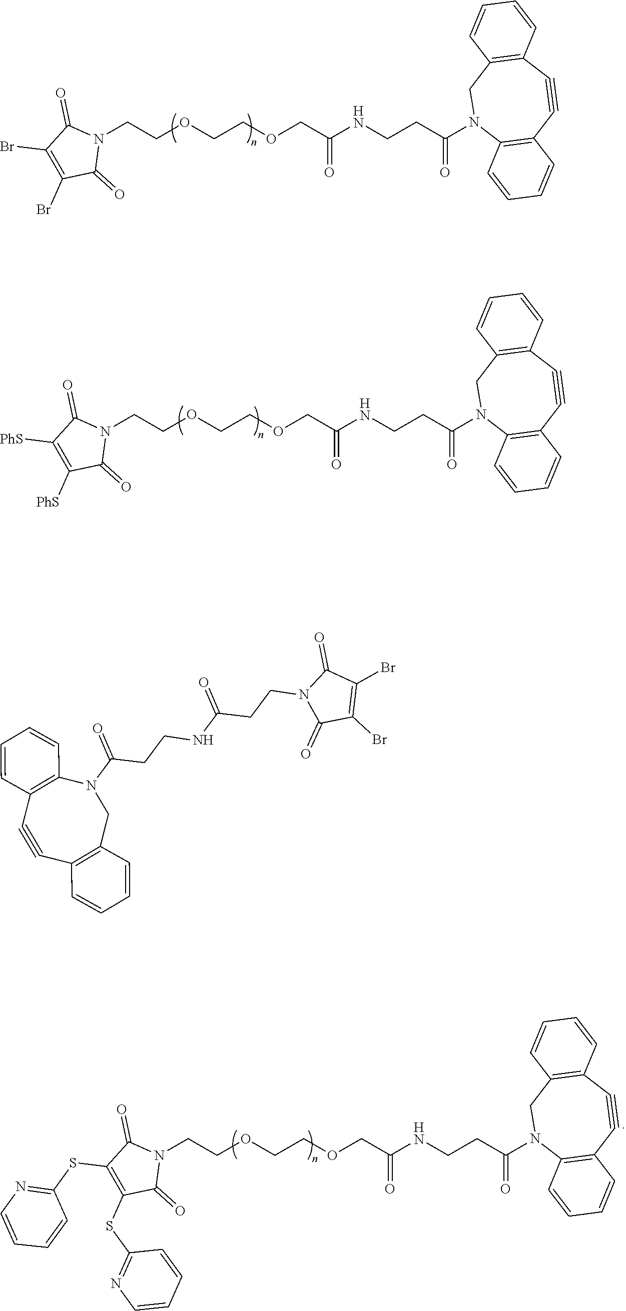

Wherein n is an integer of 0-20, optionally, n is 0, 1, 2, 3, 4, 5, 6, 7 8, 9, 10 in the first thiobridge reagent bearing reactive groups and in the second thiobridge reagent bearing reactive groups.

In some embodiments, the first thiobridge reagent bearing reactive groups and the second thiobridge reagent bearing reactive groups are dibromomaleimide-PEG4-N3 having the following formula

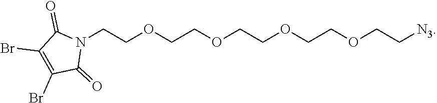 [SMILES: [N-]=[N+]=NCCOCCOCCOCCOCCN1C(=O)C(Br)=C(Br)C1=O]

On appropriate conditions, the reactive groups could react with the linker-payloads, and the linker-payload is connected to thiobridge reagent by covalence. With the change of reactive groups or linker-payloads, the reaction products maybe change. In the disclosure, the products of different reactive groups and linker-payloads are collectively referred to as thiobridge reagent bearing linker-payload.

In some embodiments, the first thiobridge reagent bearing the first linker-payload and the second thiobridge reagent bearing the second linker-payload have the following formula:

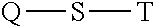

Wherein, Q is selected from the groups consisting of

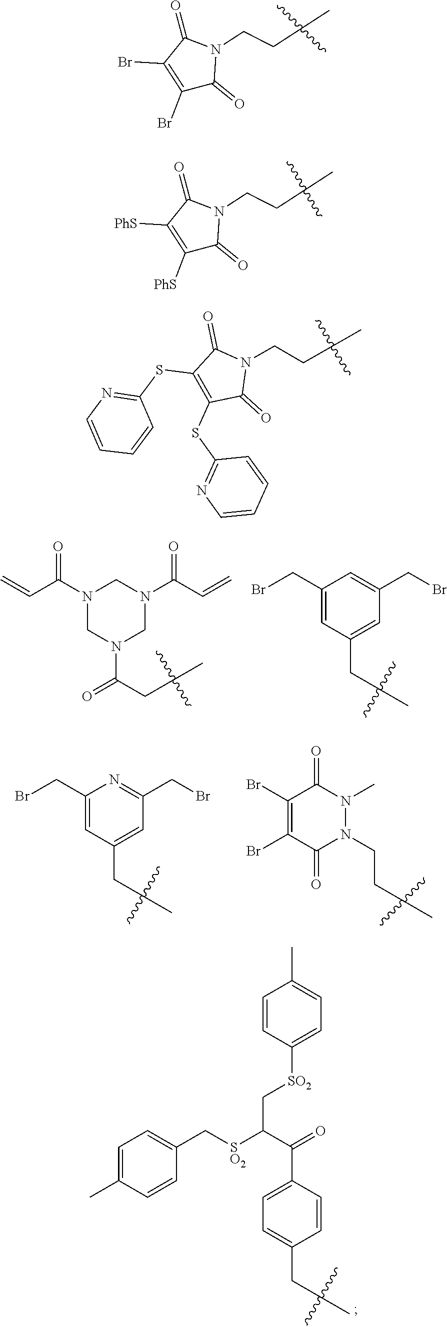

S is selected from a cleavable linker or a non-cleavable linker, without the limitation, S is selected from the groups consisting of

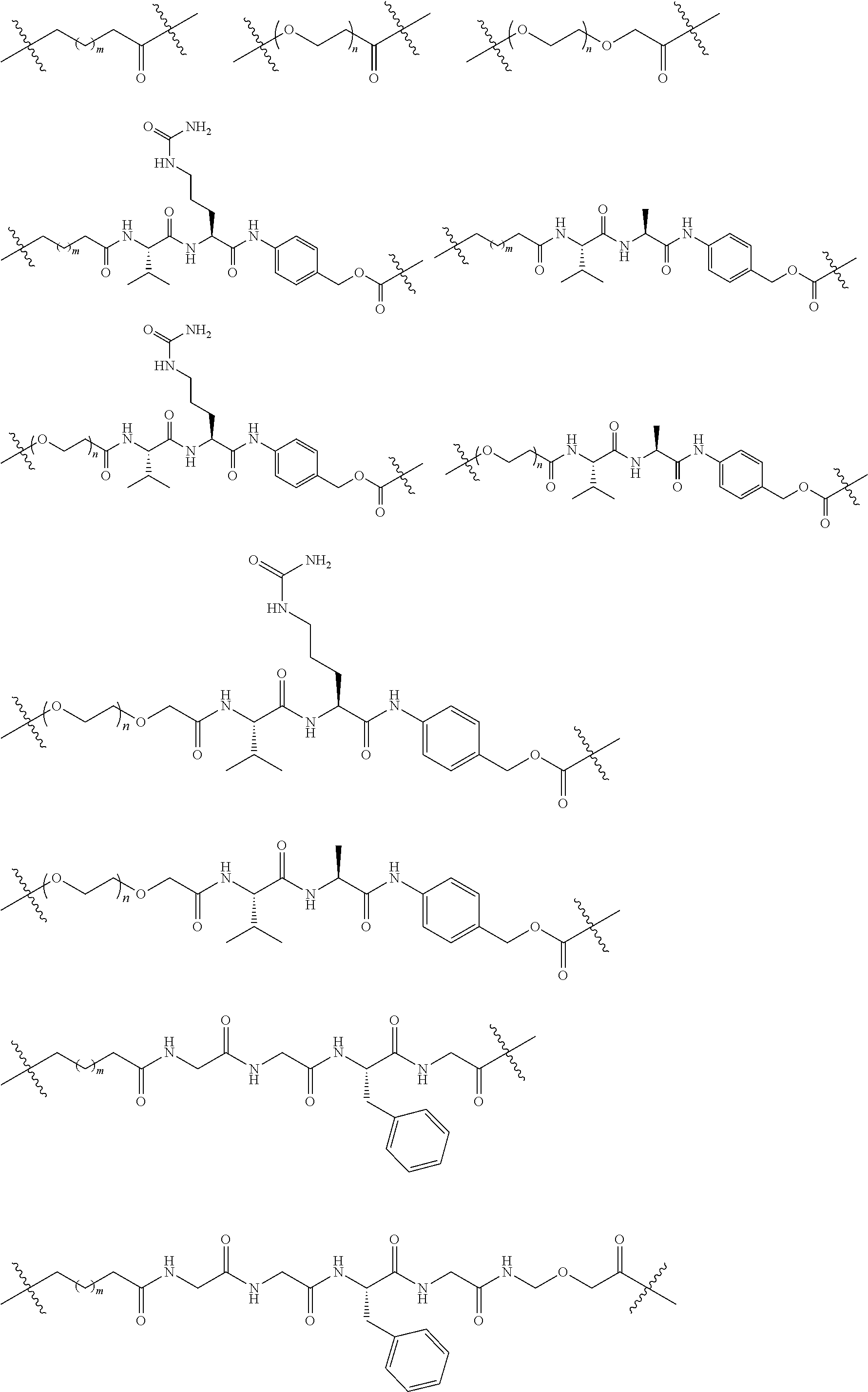

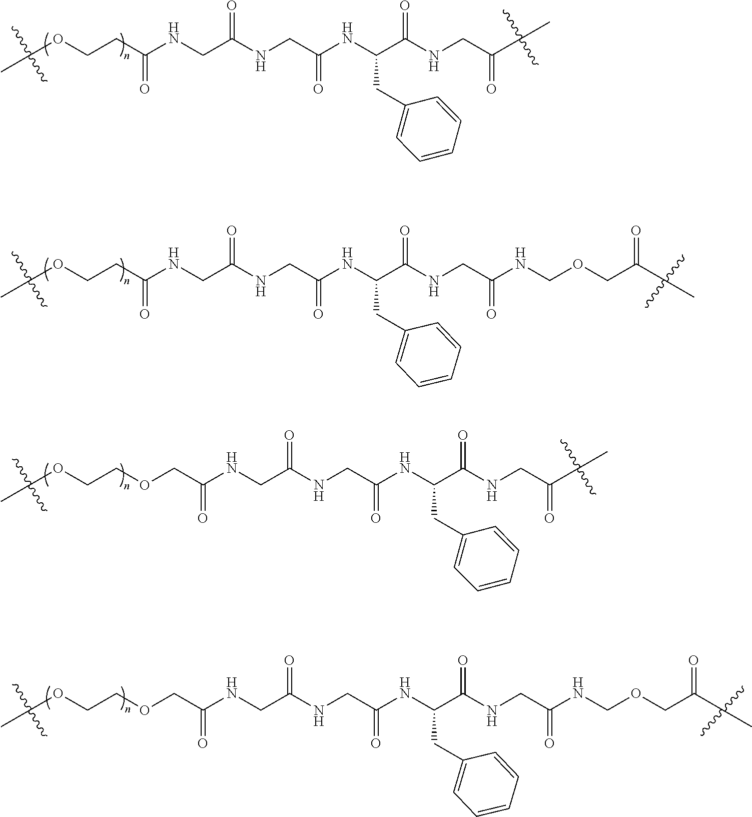

wherein, n is 0-20, m is 0-20, optionally, n is 0, 1, 2, 3, 4, 5, 6, 7, 8, 9 or 10, m is 0, 1, 2, 3, 4, 5, 6, 7, 8, 9 or 10.

T is payload.

In some embodiments, the first thiobridge reagent bearing the first linker-payload and the second thiobridge bearing the second linker-payload, are independently selected from the group consisting of

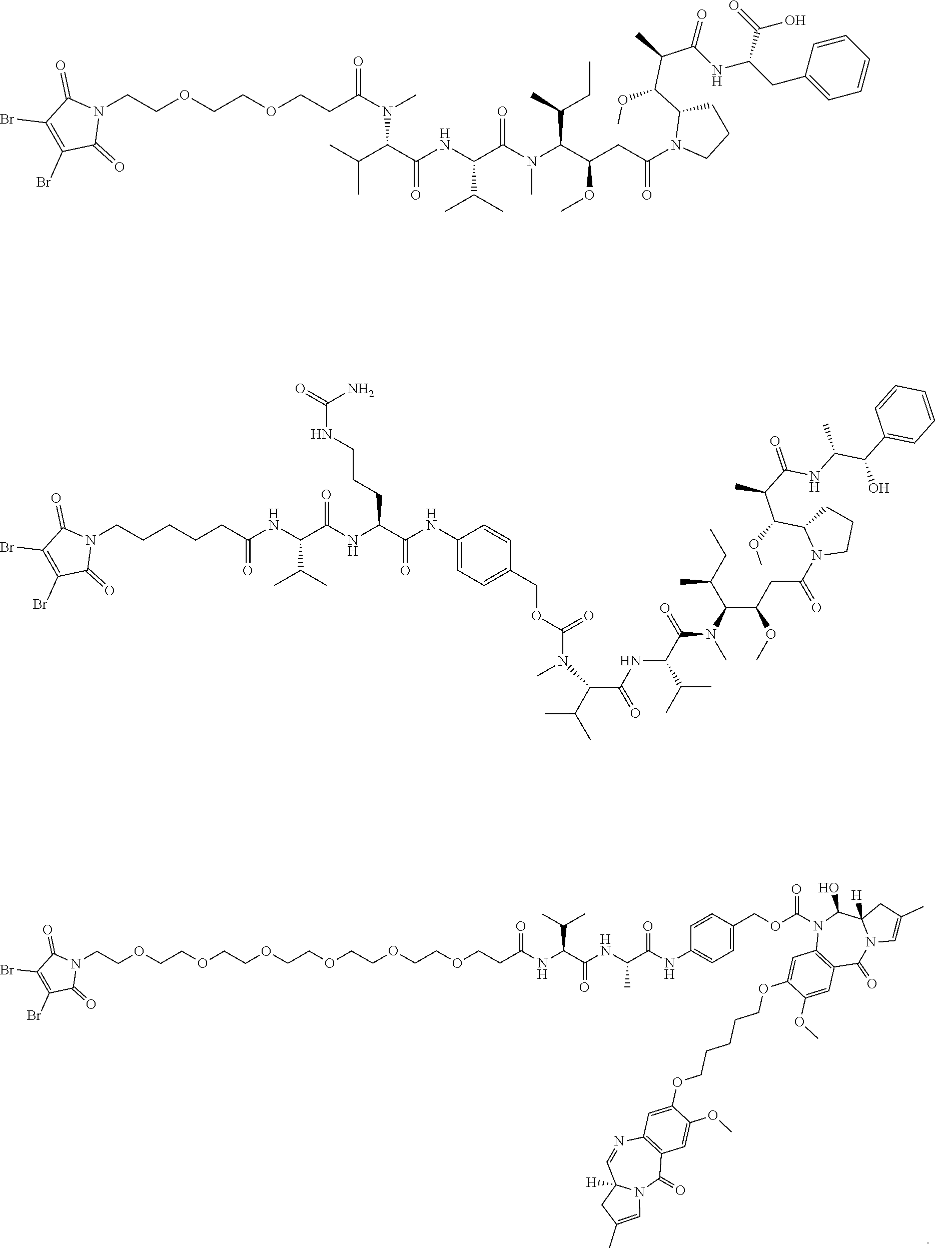

In some embodiments, the first conjugating group is the first linker-payload. In some embodiments, the first conjugating group is the first thiobridge reagent, optimally, the first thiobridge reagent bearing the reactive groups or the first liker-payload.

In some embodiments, the second conjugating group is the second linker-payload. In some embodiments, the second conjugating group is the second thiobridge reagent, optimally, the second thiobridge reagent bearing the reactive groups or the second liker-payload.

In some embodiments, the first conjugating group and the second conjugating group include Maleimide-PEG4-N3-DBCO-Cy3, Maleimide-VC-PAB-MMAE or MC-GGFG-DXd independently.

In some embodiments, there is no specific limitation to the antibody. According to the antigens associated with the disease, those skilled in the art can select suitable antibody useful in the bio-conjugation process of the present application. In some embodiments, the antibody is a monoclonal antibody, a polyclonal antibody, a mono-specific antibody or a multi-specific antibody.

In some embodiments, the antibody is a human antibody, a humanized antibody, a chimeric antibody, or an antigen-binding moiety thereof.

As used herein, the term “human antibody” refers to one which possesses an amino acid sequence which corresponds to that of an antibody produced by a human or a human cell or derived from anon-human source that utilizes human antibody repertoires or other human antibody-encoding sequences. This definition of a human antibody specifically excludes a humanized antibody comprising non-human antigen-binding residues.

As used herein, the term “humanized antibody” refers to a chimeric antibody comprising amino acid residues from non-human heavy chain variable regions (HVRs) and amino acid residues from human FRs. In certain embodiments, a humanized antibody will include substantially all or at least one, and typically two, variable domains, in which all or substantially all of the HVRs (e.g., CDRs) correspond to those of a non-human antibody, and all or substantially all of the FRs correspond to those of a human antibody. A humanized antibody optionally may include at least a portion of an antibody constant region derived from a human antibody. A “humanized form” of an antibody, e.g., a non-human antibody, refers to an antibody that has undergone humanization.

As used herein, the term “chimeric antibody” refers to an antibody in which a portion of the heavy and/or light chain is derived from a particular source or species, while the remainder of the heavy and/or light chain is derived from a different source or species.

In some embodiments, the antibody means an immunoglobulin and is a molecule containing an antigen-binding site immunospecifically binding to an antigen. In some embodiments of the present application, the class of the antibody is IgG, IgE, IgM, IgD, IgA, or IgY. In some embodiments of the present application, the class of the antibody is IgG.

In some embodiments, the class of the antibody is IgG1, IgG2, IgG3 or IgG4. In some embodiments, the antibody is IgG1 or IgG4.

In some embodiment, the antibody is wild type. As use herein, the term “wild type” refers to naturally occurring and without mutation.

In some embodiments, the antibody is an engineered antibody having two amino acid substitutions of two interchain cysteines forming one interchain disulfide bond in the hinge region.

In some embodiments, the amino acid substitutions are selected from the following, cysteine to alanine, to leucine, to arginine, to lysine, to asparagines, to methionine, to aspartic acid, to phenylalanine, to praline, to glutamine, to serine, to glutamic acid, to threonine, to glycine, to tryptophan, to histidine, to tyrosine, to isoleucine or to valine, respectively.

In some embodiments, the amino acid substitutions are selected from the following, cysteine to asparagines, to glutamine, to serine, to threonine or to tyrosine, respectively.

In some embodiments, the amino acid substitutions are selected from the following, cysteine to serine.

In some embodiments, the antibody includes at least one mutation in the Fc region. In some embodiments, the at least one mutation modulates effector function, or attenuates or eliminates Fc-g receptor binding.

In some embodiments, the one or more mutations are to stabilize the antibody and/or to increase half-life. In some instances, the one or more mutations are to modulate Fc receptor interactions, to reduce or eliminate Fc effector functions such as FcyR, antibody- dependent cell-mediated cytotoxicity (ADCC) or complement-dependent cytotoxicity (CDC). In additional instances, the one or more mutations are to modulate glycosylation.

In some embodiments, the one or more mutations are located in the Fc region. In some instances, the Fc region includes a mutation at residue position L234, L235, or a combination thereof. In some instances, the mutations include L234 and L235. In some instances, the mutations include L234A and L235A. In some cases, the residue positions are in reference to IgGI.

In some embodiments, the Fc region includes a mutation at residue position L234, L235, D265, N21, K46, L52, or P53, or a combination thereof. In some instances, the mutations include L234 and L235 in combination with a mutation at residue position K46, L52, or P53. In some cases, the residue positions are in reference to IgG1.

In some embodiments, the Fc region includes mutations at L234, L235, and K46. In some cases, the Fc region includes mutations at L234, L235, and L52. In some cases, the Fc region includes mutations at L234, L235, and P53. In some cases, the Fc region includes mutations at D265 and N21.

In some cases, the residue position is in reference to IgG1.

In some instances, the Fc region includes L234A, L235A, D265A, N21G, K46G, L52R, or P53G, or a combination thereof. In some instances, the Fc region includes L234A and L235A in combination with K46G, L52R, or P53G. In some cases, the Fc region includes L234A, L235A, and K46G. In some cases, the Fc region includes L234A, L235A, and L52R. In some cases, the Fc region includes L234A, L235A, and P53G. In some cases, the Fc region includes D265A and N21G. In some cases, the residue position is in reference to IgG1.

In some embodiments, the Fc region includes a mutation at residue position L233, L234, D264, N20, K45, L51, or P52. In some instances, the Fc region includes mutations at L233 and L234 in combination with a mutation at residue position K45, L51, or P52. In some cases, the Fc region includes mutations at L233, L234, and K45. In some cases, the Fc region includes mutations at L233, L234, and L51. In some cases, the Fc region includes mutations at L233, L234, and K45. In some cases, the Fc region includes mutations at L233, L234, and P52. In some instances, the Fc region includes mutations at D264 and N20. In some cases, equivalent positions to residue L²³³, L234, D264, N20, K45, L51, or P52 in an IgG1, IgG2, IgG3, or IgG4 framework are contemplated.

In some embodiments, the Fc region includes L233A, L234A, D264A, N20G, K45G, L51R, or P52G. In some instances, the Fc region includes L233A and L234A. In some instances, the Fc region includes L233A and L234A in combination with K45G, L51R, or P52G. In some cases, the Fc region includes L233A, L234A, and K45G. In some cases, the Fc region includes L233A, L234A, and L51R.

In some cases, the Fc region includes L233A, L234A, and K45G. In some cases, the Fc region includes L233A, L234A, and P52G. In some instances, the Fc region includes D264A and N20G. In some cases, the residue position is in reference to IgG1.

In some embodiments, the human IgG constant region is modified to alter antibody- dependent cellular cytotoxicity (ADCC) and/or complement-dependent cytotoxicity (CDC), e.g., with an amino acid modification described in Natsume et al., 2008 Cancer Res, 68(10): 3863-72; Idusogie et al., 2001 J Immunol, 166(4): 2571-5; Moore et al., 2010 mAbs, 2(2): 181-189; Lazar etal, 2006 PNAS, 103(11): 4005-4010, Shields etal, 2001 JBC, 276(9): 6591-6604; Stavenhagen etal., 2007 Cancer Res, 67(18): 8882-8890; Stavenhagen etal., 2008 Advan. Enzyme Regul., 48: 152-164; Alegre et al, 1992 J Immunol, 148: 3461-3468; Reviewed in Kaneko and Niwa, 2011 Biodrugs, 25(1): 1-11.

In some embodiments, the antibody of IgG1, IgG2, IgG3 or IgG4 is human or humanized antibody. The information of IgG1, IgG2, IgG3 or IgG4 can be obtained on NCBI or UniProt (https://www.uniprot.org/).

In some embodiments of the present application, the antibody is bispecific antibodies. In some embodiments of the present application, the antibody is IgG1 like bispecific antibodies.

In some embodiments of the present application, those skilled in the art can select suitable method to prepare the bispecific antibodies. In some embodiments of the present application, the bispecific antibodies can be obtained by Knobs-in-holes technology (Ridgway J B B, Presta L G, Paul C. ‘Knobs-into-holes’ engineering of antibody CH₃ domains for heavy chain heterodimerization[J]. Protein Engineering (7):617(2023-08-11), format chain exchange (FORCE) technology, a common light chain format technology (De Nardis C, Hendriks L J A, Poirier E, et al. A new approach for generating bispecific antibodies based on a common light chain format and the stable architecture of human immunoglobulin G1[J]. Journal of Biological Chemistry, 2017:jbc.M117.793497.), controlled Fab arm exchange technology (Yanakieva De, Pekar L, Evers A, et al. Beyond bispecificity: Controlled Fab arm exchange for the generation of antibodies with multiple specificities[J]. MABS, 2022, 14(1), e2018960), CrossMAb technology (Klein C, Schaefer W, Regula J T. The use of CrossMAb technology for the generation of bi- and multispecific antibodies[J]. MABS, 2016, 8(6), P1010-P1020.) or their combination.

As used herein, the term “knobs-into-holes” is used in its broadest sense and encompasses various situations, such as the CH₁ domain of one heavy chain with the knob mutations and the CH₁ domain of the other heavy chain with the hole mutations, the CH₂ domain of one heavy chain with the knob mutations and the CH₂ domain of the other heavy chain with the hole mutations, and/or the CH₃ domain of one heavy chain with the knob mutations and the CH₃ domain of the other heavy chain with the hole mutations. For example, and generally, “knobs-into-holes” may refer to an intra-interface modification between two antibody heavy chains in the CH₃ domains: i) in the CH₃ domain of one heavy chain (first CH₃ domain), an amino acid residue is substituted with another amino acid residue bearing a large side chain, thereby creating a protrusion (“knob”) in the interface in the first CH₃ domain; ii) in the CH₃ domain of the other heavy chain (second CH₃ domain), an amino acid residue is substituted with another amino acid residue bearing a smaller side chain, thereby creating a cavity (“hole”) within the interface in the second CH₃ domain, in which a protrusion (“knob”) in the first CH₃ domain can be placed.

In some embodiment, the antibody is selected from any one of cytotoxic antibodies, inhibitors of cell proliferation, regulators of cell activation and interaction, regulators of the human immune system, neutralizations of antigens, antibodies that are immunospecific for viral antigens or antibodies that are immunospecific for microbial antigens.

In some embodiment, the antibody can be target-specific antibodies, In some embodiments of the present application, without the limitation, the antibody can be anti-HER2 antibody, anti-FAP antibody, anti-OX-40 antibody, anti-41BB antibody, anti-Angiopoietin-2 antibody, anti-anti-IL-4Rα antibody, anti-BCMA antibody, anti-Blys antibody, anti-BTNO2 antibody, anti-C5 antibody, anti-CD122 antibody, anti-CD13 antibody, anti-CD133 antibody, anti-CD137 antibody, anti-CD138 antibody, anti-CD16a antibody, anti-CD19 antibody, anti-CD20 antibody, anti-CD22 antibody, anti-CD27 antibody, anti-CD28 antibody, anti-CD3 antibody, anti-CD30 antibody, anti-CD33 antibody, anti-CD38 antibody, anti-CD40 antibody, anti-CD47 antibody, anti-CD-8 antibody, anti-CD79 antibody, anti-CEA antibody, anti-CGPR/CGRPR antibody, anti-CSPGs antibody, anti-CTLA4 antibody, anti-CTLA-4 domains antibody, anti-DLL-4 antibody, anti-EGFR antibody, anti-EpCAM antibody, anti-factor IXa antibody, anti-factor X antibody, anti-GITR antibody, anti-GP130 antibody, anti-Her3 antibody, anti-HSG antibody, anti-ICOS antibody, anti-IGF1 antibody, anti-IGF1/2 antibody, anti-IGF-J R antibody, anti-IGF2 antibody, anti-IGFR antibody, anti-IL-1 antibody, anti-IL-12 antibody, anti-IL-12p40 antibody, anti-IL-13 antibody, anti-IL-17A antibody, anti-IL-1β antibody, anti-IL-23 antibody, anti-IL-5 antibody, anti-IL-6 antibody, anti-IL-6R antibody, anti-Lag-3 antibody, anti-LAG3 antibody, anti-MAG antibody, anti-Met antibody, anti-NgR antibody, anti-NogoA antibody, anti-OMGp antibody, anti-OX40 antibody, anti-PD-1 antibody, anti-PDGFR antibody, anti-PDL-1 antibody, anti-PSMA antibody, anti-RGMA antibody, anti-RGMB antibody, anti-SARS-CoV-2 antibody, anti-Te38 antibody, anti-TIM-3 antibody, anti-TNF antibody, anti-TNFα antibody, anti-TROP-2 antibody, anti-TWEAK antibody, anti-VEGF antibody, or anti-VEGFR antibody.

In some embodiments, the antibody is target-specific, which is targeted to, HER2 (Human Epidermal GrowthFactor Receptor 2), TROP2 (TACSTD2, tumor associated calcium signal transducer 2), BCMA (TNFRSF17, TNF receptor superfamily member 17).

In some embodiments, the antibody is Trastuzumab, Sacituzumab or Belantamab.

In some embodiments of the present application, the antibody can be obtained commercially or produced by any method known to those skilled in the art.

### Modified Antibody

The present application provides a modified antibody prepared from the method described above.

In some embodiments, the modified antibody is conjugated with one, two or three kinds of conjugating groups.

In some embodiments, the modified antibody is an antibody drug conjugate (ADC) with D1, D2, D3, D4, D6, D1+D3, D1+D6, D1+D2, D1+D4, D2+D3, D2+D6, D0+D3, D0+D6, D3+D1, D3+D2, D6+D2, D6+D1, D0+D1, D0+D3, D4+D1 or D4+D2.

In some embodiments, the ADC is Trastuzumab-[MC-VC-PAB-MMAE]₂, Sacituzumab-[MC-VC-PAB-MMAE]₂, Belantamab-[MC-VC-PAB-MMAE]₂, Trastuzumab-[MC-GGFG-DXd]₂, Trastuzumab-[Maleimide-PEG4-N3-DBCO-Cy3], Trastuzumab-[MC-VC-PAB-MMAE]₆, Sacituzumab-[MC-VC-PAB-MMAE]₆, Belantamab-[MC-VC-PAB-MMAE]₆, Trastuzumab-[MC-VC-PAB-MMAE]₆[MC-GGFG-DXd]₂, Trastuzumab-[MC-VC-PAB-MMAE]₆[Maleimide-PEG4-N3-DBCO-Cy3]₁, Trastuzumab-[Maleimide]₃[MC-VC-PAB-MMAE]₂, Trastuzumab-[Maleimide]₃[Maleimide-PEG4-N3-DBCO-Cy3]I, Trastuzumab-[Maleimide-PEG4-N3-DBCO-Cy3]₁[MC-VC-PAB-MMAE]₆, Trastuzumab-[Maleimide-PEG4-N3-DBCO-Cy3]₁[MC-VC-PAB-MMAE]₂, Trastuzumab-[Maleimide-PEG4-N3-DBCO-Cy3]₁[MC-VC-PAB-MMAE]₄, Trastuzumab-[MC-GGFG-DXd]₂[MC-VC-PAB-MMAE]₄, Trastuzumab-[MC-VC-MMAE]₄ or Trastuzumab-[MC-VC-MMAE]₄[MV-GGFG DXd]₂.

In some embodiments, the antibody with site-specific modification (ADC with D6) is prepared by the method II including steps (R1) and (CI), wherein the first conjugating group is the first linker-payload.

In some embodiments, the antibody with site-specific modification (ADC with D3) is prepared by the method II including steps (R1) and (C1), wherein the first conjugating group is the first thiobridge reagent bearing the first linker-payload, or the first conjugating group is the thiobridge reagent bearing reactive groups which reacts with the first linker-payload.

In some embodiments, the antibody with site-specific modification (ADC with D2) is prepared by the method I including steps (R1) and (C1), wherein the first conjugating group is the first linker-payload.

In some embodiments, the antibody with site-specific modification (ADC with D1) prepared by the method I includings step (R1) and (C1), wherein the first conjugating group is the first thiobridge reagent bearing the first linker-payload, or the first conjugating group is the thiobridge reagent bearing reactive groups which reacts with the first linker-payload.

In some embodiments, the antibody with site-specific modification (ADC with D2) is prepared by the method III including the steps (R1), (O1) and (C1), wherein the first conjugating group is the first linker-payload.

In some embodiments, the antibody with site-specific modification (ADC with D1) is prepared by the method III including the steps (R1), (O1) and (C1), wherein the first conjugating group is the first thiobridge reagent bearing the first linker-payload, or the first conjugating group is the thiobridge reagent bearing reactive groups which reacts with the first linker-payload.

In some embodiments, the antibody with site-specific modification (ADC with D4) is prepared by the method IV including the steps (R1), (O1), (R2) and (C1), wherein the first conjugating group is the first linker-payload.

In some embodiments, the antibody with site-specific modification (ADC with D4) is prepared by the method V including the steps (R1) and (C2), wherein the first conjugating group is the first linker-payload.

In some embodiments, the antibody with site-specific modification (ADC with D2+D6) is prepared by the method I or method III, which further comprises the steps (R3) and (C3), wherein the first conjugating group and the second conjugating group are the linker-payloads.

In some embodiments, the antibody with site-specific modification (ADC with D2+D3) is prepared by the method I or method III, which further comprises the steps (R3) and (C3), wherein the first conjugating group is the first linker-payload, and the second conjugating group is the second thiobridge reagent bearing the second linker-payload, or the second conjugating group is the second thiobridge reagent bearing reactive groups which reacts with the second linker-payload.

In some embodiments, the antibody with site-specific modification (ADC with D1+D6) is prepared by the method I or method III, which further comprises the steps (R3) and (C3), wherein the first conjugating group is the first thiobridge reagent bearing the first linker-payload, or the first conjugating group is the first thiobridge reagent bearing reactive groups which reacts with the first linker-payload, and the second conjugating group is the second linker-payload.

In some embodiments, the antibody with site-specific modification (ADC with D1+D3) is prepared by the method I or method III, which further comprises the steps (R3) and (C3), wherein the first conjugating group is the first thiobridge reagent bearing the first linker-payload, or the first conjugating group is the first thiobridge reagent bearing reactive groups which reacts with the first linker-payload, and the second conjugating group is the second thiobridge reagent bearing the second linker-payload, or the second conjugating group is the second thiobridge reagent bearing reactive groups which reacts with the second linker-payload.

In some embodiments, the antibody with site-specific modification (ADC with D0+D6) is prepared by the method I or method III, which further comprises the steps (R3) and (C3), wherein the first conjugating group is the first thiobridging reagent or the first end capping reagent, and the second conjugating group is the second linker-payload.

In some embodiments, the antibody with site-specific modification (ADC with D0+D3) is prepared by the method I or method III, which further comprises the steps (R3) and (C3), wherein the first conjugating group is the first thiobridging reagent or the first end capping reagent, and the second conjugating group is the second thiobridge reagent bearing the second linker-payload, or the second conjugating group is the second thiobridge reagent bearing reactive groups which reacts with the second linker-payload.

In some embodiments, the antibody with site-specific modification (ADC with D2+D2, ADC with D2+D4) is prepared by the method I or method III, which further comprises the steps (R3) and (C3) and introducing the transition metal ions in step (R3), wherein the first conjugating group and the second conjugating group are the linker-payloads.

In some embodiments, the antibody with site-specific modification (ADC with D1+D2, D1+D4) is prepared by the method I or method III, which further comprises the steps (R3) and (C3) and introducing the transition metal ions in step (R3), wherein the first conjugating group is the first thiobridge reagent bearing the first linker-payload, or the first conjugating group is the first thiobridge reagent bearing reactive groups which reacts with the first linker-payload, and the second conjugating group is the second linker-payload.

In some embodiments, the antibody with site-specific modification (ADC with D6+D2) is prepared by the method II which furthers comprised the steps (R3) and (C3), wherein the first conjugating group and the second conjugating group are linker-payloads.

In some embodiments, the antibody with site-specific modification (ADC with D6+D1) is prepared by the method II which further comprises the steps (R3) and (C3), wherein the first conjugating group is the first linker-payload, and the second conjugating group is the second thiobridge reagent bearing the second linker-payload, or the second conjugating group is the second thiobridge reagent bearing reactive groups which reacts with the second linker-payload.

In some embodiments, the antibody with site-specific modification (ADC with D3+D2) is prepared by the method II which further comprises the steps (R3) and (C3), wherein the first conjugating group is the first thiobridge reagent bearing the first linker-payload, or the first conjugating group is the first thiobridge reagent bearing reactive groups which reacts with the first linker-payload, and the second conjugating group is the second linker-payload.

In some embodiments, the antibody with site-specific modification (ADC with D3+D1) is prepared by the method II which further comprises the steps (R3) and (C3), wherein the first conjugating group is the first thiobridge reagent bearing the first linker-payload, or the first conjugating group is the first thiobridge reagent bearing reactive groups which reacts with the first linker-payload, and the second conjugating group is the second thiobridge reagent bearing the second linker-payload, or the second conjugating group is the second thiobridge reagent bearing reactive groups which reacts with the second linker-payload.

In some embodiments, the antibody with site-specific modification (ADC with D0+D2) is prepared by the method II which further comprises the steps (R3) and (C3), wherein the first conjugating group is the first thiobridge reagent or the first end capping reagent, and the second conjugating group is the second linker-payload.

In some embodiments, the antibody with site-specific modification (ADC with D0+D3) is prepared by the method II which further comprises the steps (R3) and (C3), wherein the first conjugating group is the first thiobridge reagent or the end capping reagent, and the second conjugating group is the second thiobridge reagent bearing the second linker-payload, or the second conjugating group is the second thiobridge reagent bearing reactive groups which reacts with the second linker-payload.

In some embodiments, the antibody with site-specific modification (ADC with D4+D4) is prepared by the method IV or method V, which furthers comprised the steps (R3) and (C3), wherein the first conjugating group and the second conjugating group are linker-payloads.

In some embodiments, the antibody with site-specific modification (ADC with D4+D2) is prepared by the method IV or method V, which furthers comprises the step (R3) and (C3) and introducing the transition metal ions in step (R3), wherein the first conjugating group and the second conjugating group are linker-payloads.

Various analytical methods can be used to determine the yields and isomeric mixtures of the antibody with site-specific modification. In some embodiments of the present application, the analytical method is HIC-HPLC (Hydrophobic interaction chromatography-High performance liquid chromatography). HIC-HPLC can separate the antibodies loaded with various numbers of linker-payload. The loading level of payload can be determined based on the ratio of absorbances, e.g., at 250 nm and 280 nm. For example, if a payload (e.g., drug) can absorb at 250 nm while the antibody absorbs at 280 nm. The 250/280 ratio therefore increases with drug loading.

The process of generating antibodies with site-specific modification bypasses any need of protein engineering or enzyme catalysis, but is based on native inter-chain disulfide bonds, and only needs reductants and transition metal ions. Therefore, the process of the disclosure is less complicate, the homogeneity of the resultant antibodies with site-specific modification (antibody-drug conjugate) is dramatically improved.

In some embodiments, the homogeneity of the ADC with D6 is at least up to 50% of the total weight of DO, D2, D4, D6 and D8 combined. In some embodiments, the homogeneity of the ADC with D6 is up to 60%, 65%, 70%, 75%, 80%, 81%, 82%, 83%, 84%, 85%, 86%, 87%, 89%, 91%, 92%, 93%, 94% or 95%.

In some embodiments, the homogeneity of the ADC with D2 is at least up to 50% of the total weight of DO, D2, D4, D6 and D8 combined. In some embodiments, the homogeneity of the ADC with D2 is up to 55%, 60%, 70%, 75%, 80%, 81%, 82%, 83%, 84%, 85%, 86%, 87%, 88%, 89%, 90%, 91%, 92%, 93%, 94%, or 95% of the total weight of DO, D2, D4, D6 and D8 combined. In some embodiments, the homogeneity of the ADC with D1 is at least up to 65%. In some embodiments, the homogeneity of the ADC with D1 is up to 70%, 75%, 76%, 77%, 78%, 79%, 80%, 81%, 82%, 83%, 90%.

In some embodiments, the homogeneity of the ADC with D3 is at least up to 75%. In some embodiments, the homogeneity of the ADC with D3 is up to 75%, 76%, 77%, 78%, 79%, 80%, 81%, 82%, 83%, 85% or 87%.

In some embodiments, the homogeneity of the ADC with D2+D6 is at least up to 65%. In some embodiments, the homogeneity of the ADC includes D1 is up to 75%, 76%, 77%, 78%, 79%, 80%, 81%, 900/.

In some embodiments, the homogeneity of the ADC with D0+D6 is at least up to 65%. In some embodiments, the homogeneity of the ADC includes D1 is up to 75%, 76%, 77%, 78%, 79%, 80%, 81%, 84% or 86%.

In some embodiments, the homogeneity of the ADC with D6+D2 is at least up to 65%. In some embodiments, the homogeneity of the ADC includes D1 is up to 75%, 76%, 77%, 78%, 79%, 80%, 81%, 84%, 86% or 88%.

In some embodiments, the homogeneity of the ADC with D6+D1 is at least up to 65%. In some embodiments, the homogeneity of the ADC includes D1 is up to 75%, 76%, 77%, 78%, 79%, 80%, 81%, 84%, 86% or 90%.

In some embodiments, the homogeneity of the ADC with D1+D2 is at least up to 65%. In some embodiments, the homogeneity of the ADC includes D1 is up to 66%, 68%, 69/c, 70%, 73% or 75%.

In some embodiments, the homogeneity of the ADC with D1+D4 is at least up to 60%. In some embodiments, the homogeneity of the ADC includes D1 is up to 62%, 65%, 67%, 70%, 75%, 76%, 77%, 78%, 79%, 80%, 81%, 84% or 86%.

In some embodiments, the homogeneity of the ADC with D2+D4 is at least up to 65%. In some embodiments, the homogeneity of the ADC includes D1 is up to 66%, 68%, 69%, 70%, 73%, 75%, 78%, 80%, 82% or 84%.

In some embodiments, the homogeneity of the ADC with D4 is at least up to 70%. In some embodiments, the homogeneity of the ADC includes D4 is up to 75%, 80%, 85%, 90% or 95%.

In some embodiments, the homogeneity of the ADC with D4+D2 is at least up to 65%. In some embodiments, the homogeneity of the ADC includes D4+D2 is up to 68% or 70%.

The method of the present application is compatible with current thiol-reactive linker-drug technologies with minimum conformation change and intact Fc function. Meanwhile it has simple manipulation and reduced cost. The method provided herein is simple to operate, and it is fully compatible with current thiol-reactive linker-drug technologies.

### Pharmaceutical Composition

The disclosure also provides a pharmaceutical composition comprising the modified antibody prepared by the method described above and at least a pharmaceutically acceptable ingredient.

In some embodiments, the modified antibody is the antibody with site-specific modification. In some embodiments, the modified antibody is the ADC.

As use herein, the term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

As use herein, the term “pharmaceutically acceptable ingredient” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22nd Ed. Pharmaceutical Press, 2013, pp. 1049-1070).

Pharmaceutical compositions provided herein may be formulated in any manner known in the art, such as, pharmaceutical compositions provided herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions are formulated to be compatible with their intended route of administration (e.g., intravenous, intraarterial, intramuscular, intradermal, subcutaneous, or intraperitoneal).

Pharmaceutical acceptable ingredient for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxyanisole, butylated hydroxytoluene, and/or propyl gallate. As disclosed herein, inclusion of one or more antioxidants such as methionine in a composition comprising an antibody or antigen-binding fragment thereof and conjugates provided herein decreases oxidation of the antibody or antigen-binding fragment thereof. This reduction in oxidation prevents or reduces loss of binding affinity, thereby improving antibody stability and maximizing shelf-life. Therefore, in certain embodiments, pharmaceutical compositions are provided that include one or more antibodies or antigen-binding fragments thereof as disclosed herein and one or more antioxidants such as methionine.

In some embodiments, the pharmaceutical compositions can be a liquid solution, suspension, or emulsion. In some embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In some embodiments, there is the pH regulator in the pharmaceutical compositions, and the pH regulator is sodium dihydrogen phosphate, disodium hydrogen phosphate, potassium dihydrogen phosphate, potassium dihydrogen phosphate, boric acid, acetic acid, sodium acetate, citric acid, sodium citrate, tartaric acid, sodium tartrate, sodium carbonate, potassium carbonate, sodium bicarbonate, potassium bicarbonate, sodium hydroxide, potassium hydroxide, hydrochloric acid, phosphoric acid or the like.

In some embodiments, there is stabilizer in the pharmaceutical compositions, and the stabilizer is disodium edilate, calcium disodium edilate, dipotassium edilate, diamine edilate, α-lipoic acid, ethylene glycol dimethacrylate, sodium oleate, anhydrous sodium sulfite, sodium ascorbate, desferric amine, malate, citric acid, succinate, sodium, calcium and magnesium salts of malate, citric acid, succinate or the like.

In some embodiments, the pharmaceutical composition is combined with other therapeutic agents. There is no specific limitation to the other therapeutic agents, as long as the other therapeutic agents can reduce the side effects of the pharmaceutical composition or increase the efficacy of the pharmaceutical composition. The other therapeutic agents are anti-cancer agents, anti-autoimmune disease agent, anti-emetics, anti-allergic and the like.

In some embodiments, the anti-cancer agents can include, but not limited to, erlotinib, bortezomib, fulvestrant, sunitib imatinib, mesylate, letrozole, finasunate, platins such as oxaliplatin, carboplatin, and cisplatin, finasunate, fluorouracil, rapamycin, leucovorin, lapatinib, lonafamib, sorafenib, gefitinib, capmtothecin, topotecan, bryostatin, adezelesin, anthracyclin, carzelesin, bizelesin, dolastatin, auristatins, duocarmycin, eleutherobin, taxols such as paclitaxel or docetaxel, cyclophasphamide, doxorubicin, vincristine, prednisone or prednisolone, other alkylating agents such as mechlorethamine, chlorambucil, and ifosfamide, antimetabolites such as azathioprine or mercaptopurine, other microtubule inhibitors (vinca alkaloids like vincristine, vinblastine, vinorelbine and vindesine, as well as taxanes), podophyllotoxins (etoposide, teniposide, etoposide phosphate, and epipodophyllotoxins), topoisomerase inhibitors, other cytotoxins such as actinomycin, daunorubicin, valrubicin, idarubicin, epirubicin, bleomycin, plicamycin, mitomycin and the like.

The disclosure provides the use of the modified antibody provided herein or the pharmaceutical composition provided herein in the manufacture of a therapeutic agent for preventing, diagnosing or treating a disease.

In some embodiments, the modified antibody is the antibody with site-specific modification. In some embodiments, the modified antibody is the ADC.

As use herein, the term “treat” of any disease refers to alleviating or ameliorating the disease (i.e., slowing or arresting the development of the disease or at least one of the clinical symptoms thereof); or alleviating or ameliorating at least one physical parameter or biomarker associated with the disease, including those which may not be discernible to the patient. For cancer, “treating” may refer to dampen or slow the tumor or malignant cell growth, proliferation, or metastasis, or some combination thereof. For tumors, “treatment” includes removal of all or part of the tumor, inhibiting or slowing tumor growth and metastasis, delaying the development of a tumor, or some combination thereof.

As used herein, the term “prevent” of any disease refers to the prophylactic treatment of the disease; or delaying the onset or progression of the disease.

In some embodiments, the disease is a tumor or cancer. In some embodiments, the disease is an autoimmune disease and the like.

In some embodiments, the cancer can include, but not limited to, carcinoma, lymphoma, blastema, sarcoma, and leukemia or lymphoid malignancies. More particular examples of the cancer include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

In some embodiments, the pharmaceutical composition is combined with other therapeutic agents. There is no specific limitation to the other therapeutic agents, as long as the other therapeutic agents can reduce the side effects of the pharmaceutical composition or increase the efficacy of the pharmaceutical composition. The other therapeutic agents are anti-cancer agents, anti-autoimmune disease agent, anti-emetics, anti-allergic and the like.

In some embodiments, the anti-cancer agents include, but not limited to, erlotinib, bortezomib, fulvestrant, sunitib imatinib, mesylate, letrozole, finasunate, platins such as oxaliplatin, carboplatin, and cisplatin, finasunate, fluorouracil, rapamycin, leucovorin, lapatinib, lonafamib, sorafenib, gefitinib, capmtothecin, topotecan, bryostatin, adezelesin, anthracyclin, carzelesin, bizelesin, dolastatin, auristatins, duocarmycin, eleutherobin, taxols such as paclitaxel or docetaxel, cyclophasphamide, doxorubicin, vincristine, prednisone or prednisolone, other alkylating agents such as mechlorethamine, chlorambucil, and ifosfamide, antimetabolites such as azathioprine or mercaptopurine, other microtubule inhibitors (vinca alkaloids like vincristine, vinblastine, vinorelbine and vindesine, as well as taxanes), podophyllotoxins (etoposide, teniposide, etoposide phosphate, and epipodophyllotoxins), topoisomerase inhibitors, other cytotoxins such as actinomycin, daunorubicin, valrubicin, idarubicin, epirubicin, bleomycin, plicamycin, mitomycin and the like.

In some embodiments, the anti-autoimmune disease agent includes, but not limited to, ibuprofen, loxoprofen, naproxen, diclofenac, indomethacin, meloxicam, lornoxicam, nabumetone, celecoxib, paracetamol, glucocorticoids, azathioprine, cyclophosphamide and the like.

In some embodiments, patients may experience nausea during and after administration of the ADCs of the present application. Therefore, anti-emetics may be administered in preventing nausea (upper stomach) and vomiting. The anti-emetics can include, but not limited to, aprepitant, ondansetron, granisetron HCl, lorazepam, dexamethasone, prochlorperazine, casopitant and the like.

In some embodiments, patients may experience allergic reactions during and after administration of the ADCs of the present application. Therefore, anti-allergic agents may be administered to minimize the risk of an allergic reaction. The anti-allergic agents include dexamethasone, beclomethasone, hydrocortisone, prednisolone, prednisone, methylprednisolone, hydroxyzine, cyproheptadine, bronchodilators, terbutaline and the like.

The disclosure provides the method of preventing, diagnosing or treating a disease in a subject in need thereof, comprising administrating to the subject a therapeutically effective amount of the modified antibody prepared by the method described above.

In some embodiments, the modified antibody is the antibody with site-specific modification. In some embodiments, the modified antibody is the ADC.

As use herein, the term “subject” refers to mammals, primates (e.g., humans, male or female), dogs, rabbits, guinea pigs, pigs, rats and mice. In certain embodiments, the subject is a primate. In yet other embodiments, the subject is a human.

As used herein, the term “a therapeutically effective amount” refers to an amount of the ADC of the present application that will elicit the biological or medical response of a subject, for example, ameliorate symptoms, alleviate conditions, slow or delay disease progression, or prevent a disease, etc. The therapeutically effective amount will vary with the type and severity of the condition to be alleviated. It is to be further understood that for any particular subject, specific dosage regimens should be adjusted over time according to the individual need and the professional judgment of the person administering or supervising the administration of the compositions. In some embodiments of the present application, the therapeutically effective amount is based on a variety of factors, such as the type of disease, the age, weight, sex, medical condition of the patient, the severity, of the condition, the route of administration, and the particular antibody employed. In some embodiments of the present application, the therapeutically effective amount can vary widely, but can be determined routinely using standard methods. In some embodiments of the present application, the therapeutically effective amount can be adjusted based on the pharmacokinetic or pharmacodynamic parameters, which may include clinical effects such as toxic effects and/or laboratory values.

It will be readily apparent to those skilled in the art that other suitable modifications and adaptations of the present application described herein are obvious and may be made using suitable equivalents without departing from the scope of the disclosure or the embodiments disclosed herein. Having now described the disclosure in detail, the same will be more clearly understood by reference to the following examples, which are included for purposes of illustration only and are not intended to be limiting. Further, unless specifically described otherwise, the reagent and the solvent described in the description can be easily obtained from a commercial supplier.

### EXAMPLES

The invention is further described in the following examples, which do not limit the scope of the invention described in the claims.

### Reagent and Manufacturer

Trastuzumab is commercially available from Roche.

Sacituzumab and Belantamab are commercially available from MedChemExpress.

MC-VC-PAB-MMAE is commercially available from Levena biopharma.

MC-GGFG-DXd is commercially available from Levena biopharma.

Desalting column (type: 40K, 0.5 mL, REF:87766, Lot SJ251704) is commercially available from Thermo Scientific.

DBCO-Cy3 is commercially available from Confluore.

TCEP is commercially available from Bidepharm.

Dibromomaleimide, EDTA, cysteine is commercially available from Aladdin.

Azido-PEG4-Amine is commercially available from Xi'an Confluore Biological Technology Co., Ltd.

The reagents used in examples, include but not limited to 1-Hydroxybenzotriazole (HOBT), Dimethylacetamide (DMA), 1-(3-Dimethylaminopropyl)-3-ethylcarbodiimide hydrochloride (EDC), 1-Hydroxybenzotriazole (HOBt), N,N-Diisopropylethylamine (DIPEA), ethyl acetate (EtOAc), N,N-Dimethylformamide (DMF), Bicyclic amidine (DBU), 2-(7-Azabenzotriazol-1-yl)-N,N,N′,N′-tetramethyluronium hexafluorophosphate (HATU), N-(3-dimethylaminopropyl)-N′-ethylcarbodiimide hydrochloride (EDCI), trifluoroacetic acid (TFA), dichloromethane (DCM), tert-butylchlorodiphenylsilane (TBDPSCl) are commercially available.

### Synthesis Procedure A of the Compound According to the Present Application

To a solution of TCEP (286.6 mg, 1.0 mmol, 2.0 eq.) in DMF (3 mL) was added HATU (190 mg, 0.5 mmol, 1 eq) followed by DIPEA (2.0 mmol, 4.0 eq) under N₂ atmosphere. The mixture was stirred for 30 min and the amine reagent (0.5 mmol, depending on the structure of the compound) was added. The reaction was stirred at room temperature for 1 h. The reaction mixture was purified by RP-HPLC using a C18 column yielding the desired product.

### Synthesis Procedure A-1 of the Compound According to the Present Application

The product prepared by the synthesis procedure A was dissolved in DCM (3 mL) and TFA (0.3 mL) was added. The mixture was stirred for 1 h, LCMS showed reaction was completed. The mixture was concentrated and the residue was taken up by distilled water, washed with EtOAc twice. The aqueous layer was lyophilized to give corresponding product.

### Synthesis Procedure B of the Compound According to the Present Application

To a solution of TCEP (286.6 mg, 1.0 mmol, 2.0 eq.) in DMF (3 mL) was added HOBt (67.5 mg, 0.5 mmol, 1 eq) and EDCI (95.5 mg, 0.5 mmol, 1.0 eq), followed by DIPEA (2.0 mmol, 4.0 eq) under N₂ atmosphere. The mixture was stirred for 30 min and the amine reagent (0.5 mmo, depending on the structure of the compound) was added. The reaction was stirred at room temperature for 1 h. The reaction mixture was purified by RP-HPLC using a C18 column yielding the corresponding product.

### Synthesis Procedure B-1 of the Compound According to the Present Application

The product prepared by the synthesis procedure B was dissolved in DCM (3 mL) and TFA (0.3 mL) was added. The mixture was stirred for 1 h, LCMS showed reaction was completed. The mixture was concentrated and the residue was taken up by distilled water, washed with EtOAc twice. The aqueous layer was lyophilized to give corresponding product.

### Homogeneity Assays I

The drug/antibody ratio (DAR) and product distribution were analyzed using HIC-HPLC (Agilent1200) with a TSK gel Butyl-NPR column (4.6 mm ID×3.5 cm) (commercially available from Tosoh Biosciences) at a flow rate of 0.5 mL/min at 30° C. Solvent A was 1.5M (NH₄)₂SO₄ and 50 mM potassium phosphate pH 7. Solvent B was 75% v/v 50 mM potassium phosphate pH 7 and 25% v/v isopropanol. The washout procedure is as follows:

| Time [min] | Solvent A [%] | Solvent B [%] |
|---|---|---|
| 0.00 | 100.0 | 0.0 |
| 2.00 | 100.0 | 0.0 |
| 15.00 | 0.0 | 100.0 |
| 17.00 | 0.0 | 100.0 |
| 18.00 | 100.0 | 0.0 |
| 20.00 | 100.0 | 0.0 |

Homogeneity assay I was performed with the method of examples I.1-I.139 and comparative examples I.1-I.22.

### Homogeneity Assays II:

The ADCs distribution were analyzed using HIC-HPLC (Agilent1200) with a TSK gel Butyl-NPR column (2.5 μm, 4.6 mm*35 mm) (commercially available from Tosoh Biosciences) at a flow rate of 0.5 mL/min at 25T. Solvent A was 50 mM K₂HPO₄.3H₂O and 1.5M (NH₄)₂SO₄. Solvent B was 75% v/v 21.3 mM KH₂PO₄, 28.6 mM K₂HPO₄ and 25% v/v isopropanol. The sampler temperature is 4° C. The washout procedure is as follows:

| Time [min] | Solvent A [%] | Solvent B [%] |
|---|---|---|
| 0.00 | 100.0 | 0.0 |
| 2.00 | 100.0 | 0.0 |
| 15.00 | 0.0 | 100.0 |
| 17.00 | 0.0 | 100.0 |
| 18.00 | 100.0 | 0.0 |
| 20.00 | 100.0 | 0.0 |

Homogeneity assay II was performed with the method of examples II.1-II.153, comparative examples II.1-II.11, examples III.1-III.107, comparative example III.1-III.5, examples V.1-V.47 and the comparative example V.1.

Akta with HIC Chromatography:

The ADCs purification were performed using AKTA explorer with a polar MC30-HIC butyl column (4.2 mL, 800 Å, 30 μm) (commercially available from Sepax Technologies). Solvent A was 50 mM PB and 1 M (NH₄)₂SO₄. Solvent B was 50 mM PB and 20% v/v isopropanol. Solvent C was 50 mM PB and 2 M (NH₄)₂SO₄. ADC sample was mixed with solvent C in 1:1 volume ratio, filtered and then loaded at a flow rate of 2 mL/min. At a flow rate of 3 mL/min at 25T, the target component was washed out with solvent A/solvent B between v/v 65%/35% and v/v 0%/100%, and the collected product solution was concentrated and exchanged into His buffer (20 mM, pH5.5) through ultracentrifugation.

### Example 1: Synthesis of TCEP-NO-Trtyl and TCEP-NO

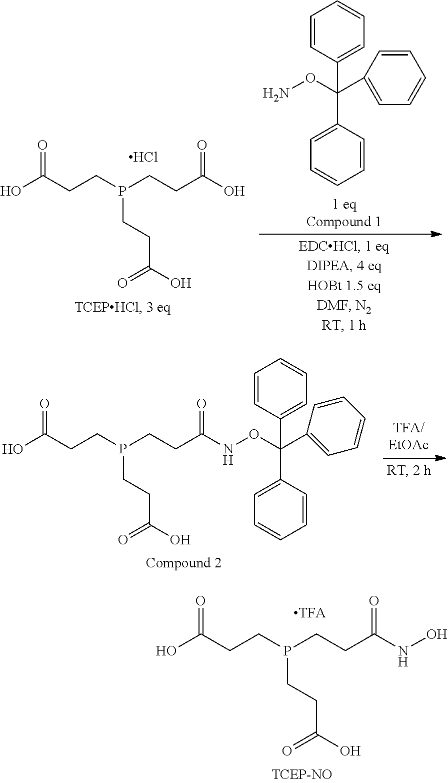 [SMILES: O=C(O)CCP(CCC(=O)O)CCC(=O)O ; Cl ; NOC(c1ccccc1)(c1ccccc1)c1ccccc1 ; O=C(O)CCP(CCC(=O)O)CCC(=O)NOC(c1ccccc1)(c1ccccc1)c1ccccc1 ; O=C(O)CCP(CCC(=O)O)CCC(=O)NO]

Compound 1 O-trithylhydroxylamine (NH₂—O-Trt, 176 μmol, 48.5 mg, 1 eq.), EDC (176 μmol 33.7 mg, 1 eq.) and HOBt (352 μmol, 53.8 mg, 2 eq.) were dissolved in 1.5 mL DMF (degassed) under an inert atmosphere. This solution was added to TCEP (528 μmol, 150 mg, 3 eq.) dissolved in 1.5 mL degassed DMF containing DIPEA (704 μmol, 123 μL, 4 eq.) under an inert atmosphere. The reaction was stirred at room temperature for 60 min. After that, DMF was removed in vacuo and the residue was added CH₃COOH (0.1 N, 5 ml) and EtOAc (2 ml). The resulting mixture was stirred for 5 min and filtered to yield Compound 2 (TCEP-NO-Trtyl) as a white solid (61.2 mg, 12.2%). For Compound 2 (also called TCEP-NO-Trtyl, or TCEP-19-int1), MS[M−H]⁻=506.2, Exact mass calc. for C₂₈H₃₀NO₆P is 507.18. ¹H NMR (400 MHz, DMSO-d6): δ 7.40-7.15 (m, 15H), 2.48-2.38 (m, 4H), 2.22 (s, 1H), 2.12-1.52 (m, 7H).

Without further purification, Compound 2 was added EtOAc (2 ml), 5% TFA and 5% TIPS. The reaction was continued for another 2 h. Then H₂O (5 ml) and EtOAc (10 ml) were introduced. The aqueous phase was further washed with EtOAc (10 ml) twice and concentrated to afford TCEP-NO (13 mg). For TCEP-NO, MS[M−H]⁻=263.94, exact mass calc. for C₉H₁₆NO₆P is 265.07. ¹H-NMR (400 MHz, Deuterium Oxide): δ 2.93-2.85 (m, 4H), 2.73-2.56 (m, 8H).

### Example 2: Synthesis of TCEP-3N0

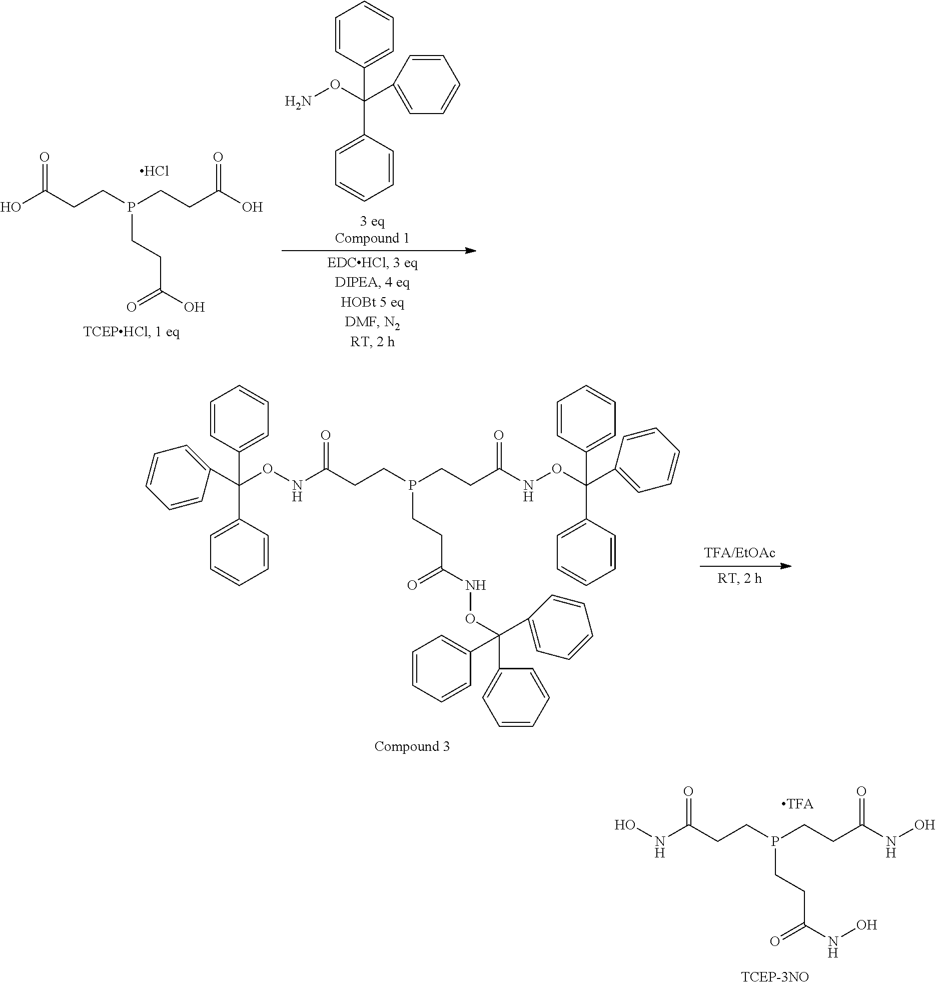 [SMILES: O=C(O)CCP(CCC(=O)O)CCC(=O)O ; Cl ; NOC(c1ccccc1)(c1ccccc1)c1ccccc1 ; O=C(CCP(CCC(=O)NOC(c1ccccc1)(c1ccccc1)c1ccccc1)CCC(=O)NOC(c1ccccc1)(c1ccccc1)c1ccccc1)NOC(c1ccccc1)(c1ccccc1)c1ccccc1 ; O=C(CCP(CCC(=O)NO)CCC(=O)NO)NO]

Compound 1 O-Trithylhydroxylamine (NH₂—O-Trt, 528 μmol, 145.5 mg, 3 eq.), EDC (528 μmol 101 mg, 3 eq.) and HOBt (880 μmol, 134.5 mg, 5 eq.) were dissolved in 4 mL DMF (degassed) under an inert atmosphere. This solution was added to TCEP (176 μmol, 50 mg, 1 eq.) dissolved in 4 mL degassed DMF containing DIPEA (704 μmol, 123 μl, 4 eq.) under an inert atmosphere. The reaction was stirred at room temperature for 60 min. After that, DMF was removed in vacuo and the residue was added CH₃COOH (0.1 N, 5 ml) and EtOAc (5 ml). The resulting mixture was stirred for 5 min and filtered to yield Compound 3 as a white solid, without further purification which was added EtOAc (2 ml), 5% TFA and 5% TIPS. The reaction was continued for another 2 h. Then H₂O (5 ml) and EtOAc (10 ml) were introduced. The aqueous phase was further washed with EtOAc (10 ml) twice and concentrated to afford TCEP-NO (32 mg). For TCEP-3NO, MS[M+H]⁺=295.87, exact mass calc. for C₉H₁₈N₃O₆P is 295.09. ¹H NMR (400 MHz, Deuterium Oxide): δ 3.08-2.38 (m, 10H), 2.24-2.17 (m, 5.6 Hz, 2H).

### Example 3: Synthesis of TCEP-CO

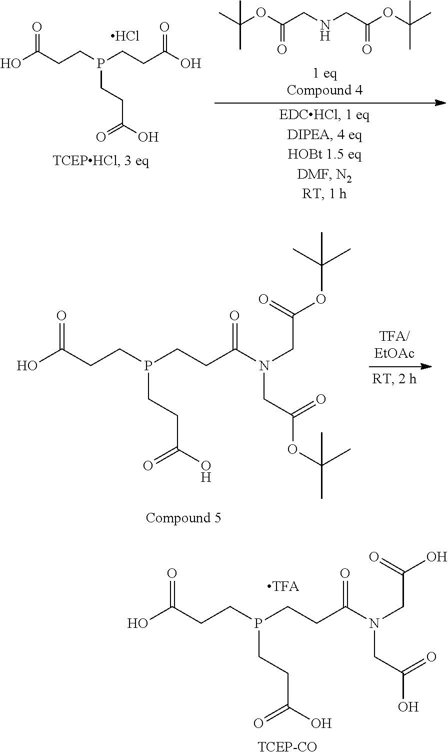 [SMILES: O=C(O)CCP(CCC(=O)O)CCC(=O)O ; Cl ; CC(C)(C)OC(=O)CNCC(=O)OC(C)(C)C ; CC(C)(C)OC(=O)CN(CC(=O)OC(C)(C)C)C(=O)CCP(CCC(=O)O)CCC(=O)O ; O=C(O)CCP(CCC(=O)O)CCC(=O)N(CC(=O)O)CC(=O)O]

Compound 4 (176 μmol, 43.2 mg, 1 eq.), EDC (176 μmol 33.7 mg, 1 eq.) and HOBt (352 μmol, 53.8 mg, 2 eq.) were dissolved in 1.5 mL DMF (degassed) under an inert atmosphere. This solution was added to TCEP (528 μmol, 150 mg, 3 eq.) dissolved in 1.5 mL degassed DMF containing DIPEA (704 μmol, 123 ul, 4 eq.) under an inert atmosphere. The reaction was stirred at room temperature for 60 min. After that, DMF was removed in vacuo and the residue was added CH₃COOH (0.1 N, 5 ml) and EtOAc (2 ml). The resulting mixture was stirred for 5 min and filtered to yield Compound 5 as a white solid, without further purification which was added EtOAc (2 ml), 5% TFA and 5% TIPS. The reaction was continued for another 2 h. Then H₂O (5 ml) and EtOAc (10 ml) were introduced. The aqueous phase was further washed with EtOAc (10 ml) twice and concentrated to afford TCEP-CO (8 mg). For TCEP-CO, MS[M−H]⁻=363.86, exact mass calc. for C9H₁₆NO₆P is 365.09. ¹H NMR (400 MHz, Deuterium Oxide): δ 4.28 (s, 2H), 4.13 (s, 2H), 2.97 (dt, J=20.0, 6.4 Hz, 2H), 2.83 (dt, J=18.3, 6.9 Hz, 4H), 2.57-2.48 (m, 6H).

### Example 4: Synthesis of TCEPA

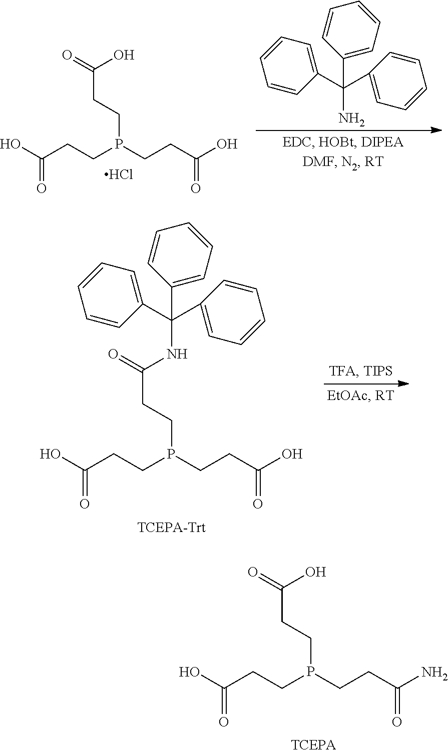

Tritylamine (176 μmol, 45.6 mg, 1 eq.), EDC (176 μmol 33.7 mg, 1 eq.) and HOBt (352 μmol, 53.8 mg, 2 eq.) were dissolved in 1.5 mL DMF (degassed) under an inert atmosphere. This solution was added to TCEP (528 μmol, 150 mg, 3 eq.) dissolved in 1.5 mL degassed DMF containing DIPEA (704 μmol, 123 ul, 4 eq.) under an inert atmosphere. The reaction was stirred at room temperature for 60 min. After that, DMF was removed in vacuo and the residue was added CH₃COOH (0.1 N, 5 ml) and EtOAc (20 ml). The resulting mixture was washed with H2O three times and the organic phase was concentrated followed by petroleum ether precipitation to yield TCEPA-Trt as a white solid, without further purification which was added EtOAc (2 ml), 5% TFA and 5% TIPS. The reaction was continued for another 2 h. Then H₂O (5 ml) and EtOAc (10 ml) were introduced. The aqueous phase was further washed with EtOAc (10 ml) twice and concentrated to afford TCEPA (8 mg). MS[M+H]⁺=250.18, exact mass calc. for C₉H₁₆NO₅P is 249.08. ¹H NMR (400 MHz, Deuterium Oxide) δ 2.85-2.70 (m, 4H), 2.61-2.43 (m, 6H), 2.15-2.06 (m, 2H).

### Example 5: Synthesis of TCEP-1

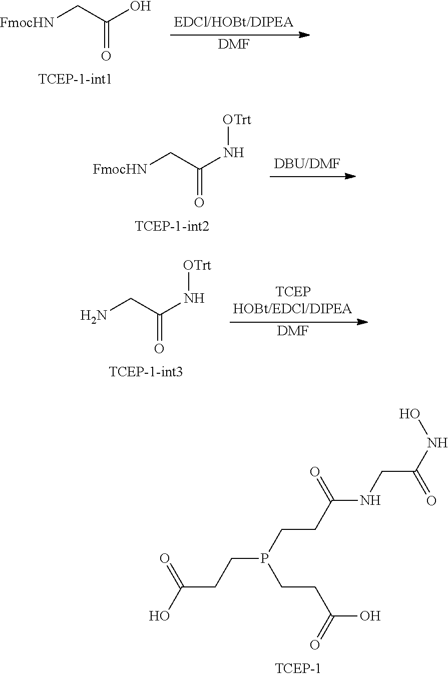

1. TCEP-1-int2

To a solution of TCEP-1-int1 (3.0 g, 10.0 mmol, 1.0 eq, Fmoc-Glycine, Bidepharm) in DMF (30 mL) was added HOBt (1.64 g, 12.0 mmol, 1.2 eq) and EDCI (2.32 g, 12 mmol, 1.2 eq), followed by DIPEA (4.4 mL, 25.2 mmol, 2.5 eq) under N2 atmosphere. The mixture was stirred for 30 min and the Compound 1 O-Trithylhydroxylamine (2.78 g, 10 mmol, 1.0 eq, Bidepharm) was added. The reaction mixture was stirred at room temperature for 1 h and poured into ice-water. The precipitate was collected by filtration and washed with water. The filter cake was dried over vacuum to give the crude product TCEP-1-int2 (4.5 g, 80% yield, white solid), which was used in next step directly without further purification.

2. TCEP-1-int3

To a solution of TCEP-1-int2 (4.5 g, 8.1 mmol, 1.0 eq) in DMF (25 mL) was added DBU (5 mL). The resulting mixture was stirred for 0.5 h at room temperature, LCMS showed reaction was completed. The mixture was poured into ice-water, and extracted with EtOAc, dried over vacuum and purified with flash column (EtOAc/petroleum ether=0˜50%) to give product TCEP-1-int3 (2.0 g, 77% yield, white solid).

### 3. TCEP-1

TCEP-1 was synthesized as the synthesis procedure B-1 wherein TCEP-1-int3 was the amine reagent, yielding TCEP-1 (45.1 mg, 28%) as white solid. MS[M−H]⁻=321.15, exact mass calc. for C₁₁H₁₉N₂O₇P is 322.25. ¹H-NMR (400 MHz, Deuterium Oxide): δ 3.99 (s, 0.64H), 3.87 (s, 1.34 H), 2.96-2.81 (m, 6H), 2.63-2.56 (m, 6H).

### Example 6: Synthesis of TCEP-2

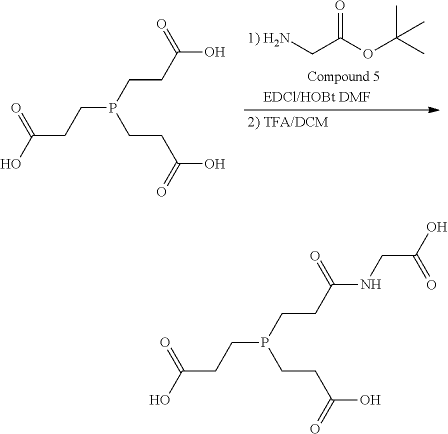

TCEP-2 was synthesized as the synthesis procedure B-1 wherein the Compound 5 (tert-Butyl glycinate, Bidepharm) was the amine reagent, yielding TCEP-2 (52.3 mg, 34%) as white solid. MS[M−H]⁻=306.18, exact mass calc. for C₁₁H₁₈NO₇P is 307.24. ¹H NMR (400 MHz, Deuterium Oxide): δ 3.99 (s, 2H), 3.00-2.76 (m, 6H), 2.60 (dtd, J=14.0, 7.0, 3.8 Hz, 6H).

### Example 7: Synthesis of TCEP-3

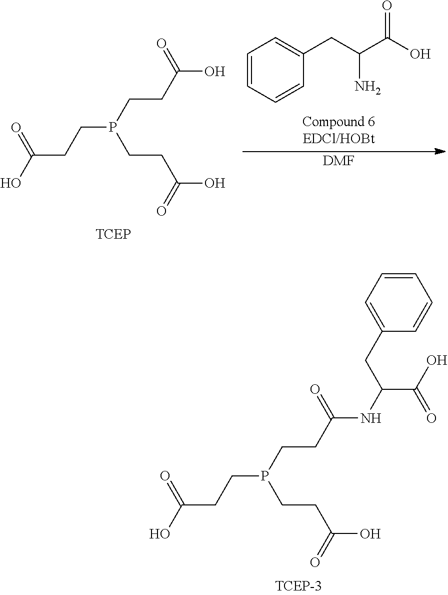

TCEP-3 was synthesized as the procedure B wherein the compound 6 (DL-Phenylalanine, Adamas) is amine reagent, yielding TCEP-3 (65.0 mg, 33%) as white solid. MS[M−H]⁻=396.24, exact mass calc. for C₁₈H₂₄NO₇P is 397.13. ¹H NMR (400 MHz, Deuterium Oxide): δ 7.43-7.25 (m, 5H), 4.71 (dd, J=10.3, 4.8 Hz, 1H), 3.31 (dd, J=13.9, 4.8 Hz, 1H), 2.91 (dd, J=13.9, 10.3 Hz, 1H), 2.86-2.59 (m, 6H), 2.53-2.27 (m, 6H).

### Example 8: Synthesis of TCEP-4

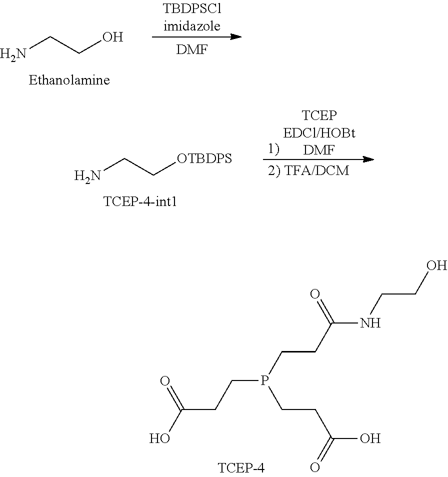

1. TCEP-4-int1

To a solution of Ethanolamine (610 mg, 10 mmol, 1.0 eq, Adamas) in DMC (20 mL) was added imidazole (15 mmol, 1.5 eq) followed by TBDPSCl (10 mmol, 1.0 eq, Adamas) at 0° C. The mixture was stirred for 2 h at room temperature. TLC showed reaction was completed, the reaction mixture was washed with water and brine, organic layer was dried over Na₂SO₄ and filtered. The filtrate was concentrated to give the crude product, which was used in next step directly without further purification.

### 2. TCEP-4

TCEP-4 was synthesized as the synthesis procedure B-1 wherein TCEP-4-int1 was amine reagent, yielding TCEP-4 (60.0 mg, 40.8%) as white solid. MS[M−H]⁻=292.25, exact mass calc. for C₁₁H₂₀NO₆P is 293.10. ¹H NMR (400 MHz, Deuterium Oxide): δ 4.23 (t, J=5.4 Hz, 1H), 3.64 (t, J=5.4 Hz, 1H), 3.48 (t, J=5.3 Hz, 1H), 3.32 (t, J=5.5 Hz, 1H), 2.98-2.78 (m, 6H), 2.60 (dp, J=13.4, 6.8 Hz, 6H).

### Example 9: Synthesis of TCEP-5

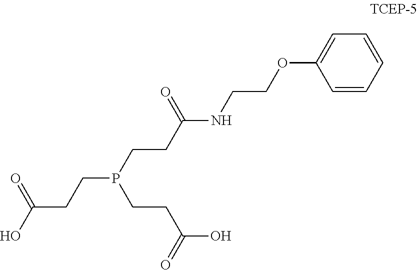 [SMILES: O=C(O)CCP(CCC(=O)O)CCC(=O)NCCOc1ccccc1]

TCEP-5 was synthesized as the procedure B wherein (2-phenoxy-ethylamine, Bidepharm) was amine reagent, yielding TCEP-5 (105.0 mg, 56.8%) as white solid. MS[M−H]⁻=368.24, exact mass calc. for C₁₇H₂₄NO₆P is 369.13. ¹H NMR (400 MHz, Deuterium Oxide): δ 7.34 (dd, J=8.5, 7.2 Hz, 2H), 7.00 (dd, J=19.2, 7.7 Hz, 3H), 4.14 (t, J=5.1 Hz, 2H), 3.56 (t, J=5.1 Hz, 2H), 2.78 (ddt, J=41.6, 20.1, 6.9 Hz, 6H), 2.60-2.43 (m, 6H).

### Example 10: Synthesis of TCEP-6

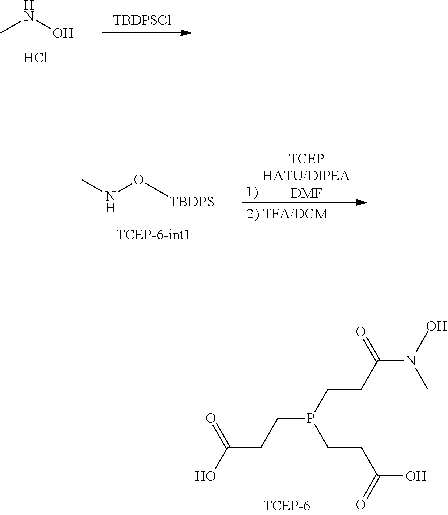

1. TCEP-6-int1

To a solution of N-Methylhydroxylamine hydrochloride (830 mg, 10 mmol, 1.0 eq, Bidepharm) in DMC (20 mL) was added imidazole (15 mmol, 1.5 eq) followed by TBDPSCl (10 mmol, 1.0 eq, Adamas) at 0° C. The mixture was stirred for 2 h at room temperature. TLC showed reaction was completed, the reaction mixture was washed with water and brine, organic layer was dried over Na₂SO₄ and filtered. The filtrate was concentrated to give the crude product, which was used in next step directly without further purification.

### 2. TCEP-6

TCEP-6 was synthesized as the procedure A-1 wherein TCEP-6-int1 was amine reagent, yielding TCEP-6 (13.0 mg, 9.3%) as white solid. MS[M+H]⁺=280.22, exact mass calc. for C₁₀H₁₈NO₆P is 279.09. ¹H NMR (400 MHz, Deuterium Oxide): δ 3.15 (s, 3H), 3.01-2.80 (m, 4H), 2.64-2.45 (m, 6H), 2.17-2.08 (m, 2H).

### Example 11: Synthesis of TCEP-7

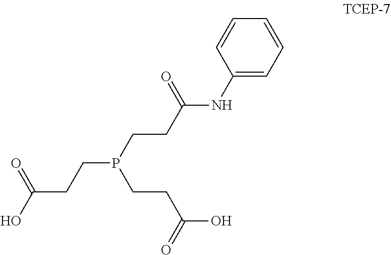 [SMILES: O=C(O)CCP(CCC(=O)O)CCC(=O)Nc1ccccc1]

TCEP-7 was synthesized as the synthesis procedure B wherein (Phenylamine, Adamas) was amine reagent, yielding TCEP-7 (73.0 mg, 45.0% yield) as white solid. MS[M−H]⁻=324.21, exact mass calc. for C₁₅H₂₀NO₅P is 325.11. ¹H NMR (400 MHz, Deuterium Oxide): δ 7.42 (d, J=4.3 Hz, 4H), 7.25 (p, J=4.5 Hz, 1H), 2.96 (ddt, J=32.1, 18.3, 6.9 Hz, 6H), 2.75-2.50 (m, 6H).

### Example 12: Synthesis of TCEP-8

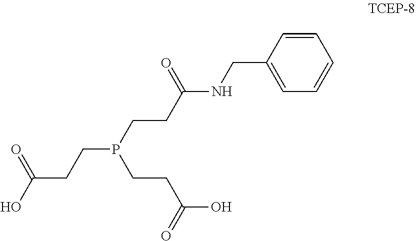 [SMILES: O=C(O)CCP(CCC(=O)O)CCC(=O)NCc1ccccc1]

TCEP-8 was synthesized as the synthesis procedure B wherein (Benzylamine, Adamas) was amine reagent, yielding TCEP-8 (85.6 mg, 50.5% yield) as white solid. MS[M−H]⁻=338.23, exact mass calc. for C₁₆H₂₂NO₅P is 339.12. ¹H NMR (400 MHz, Deuterium Oxide): δ 7.43-7.27 (m, 5H), 4.36 (s, 2H), 2.93-2.77 (m, 6H), 2.63-2.45 (m, 6H).

### Example 13: Synthesis of TCEP-9

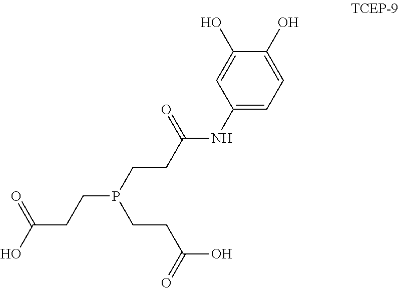 [SMILES: O=C(O)CCP(CCC(=O)O)CCC(=O)Nc1ccc(O)c(O)c1]

TCEP-9 was synthesized as the synthesis procedure A wherein (4-Aminobenzene-1,2-diol, Bidepharm) was amine reagent, yielding TCEP-9 (63.8 mg, 35.7%) as white solid. MS[M−H]⁻=356.20, exact mass calc. for C₁₅H₂₀NO₇P is 357.10. ¹H NMR (400 MHz, Deuterium Oxide): δ 6.97 (d, J=2.4 Hz, 1H), 6.87 (d, J=8.5 Hz, 1H), 6.78 (dd, J=8.5, 2.5 Hz, 1H), 2.92 (ddt, J=17.8, 10.0, 7.0 Hz, 6H), 2.61 (dq, J=13.9, 6.7 Hz, 6H).

### Example 14: Synthesis of TCEP-10

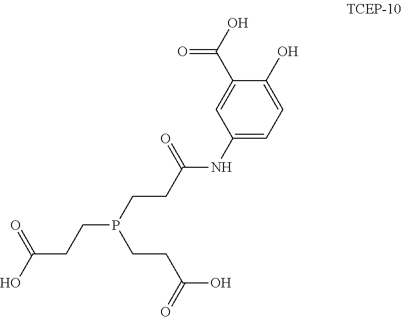 [SMILES: O=C(O)CCP(CCC(=O)O)CCC(=O)Nc1ccc(O)c(C(=O)O)c1]

TCEP-10 was synthesized as the synthesis procedure A wherein (5-Amino-2-hydroxybenzoic acid, Bidepharm) was amine reagent, yielding TCEP-10 (53.7 mg, 27.9%) as white solid. MS[M−H]⁻=384.20, exact mass calc. for C₁₆H₂₀NO₈P is 385.09. ¹H NMR (400 MHz, Deuterium Oxide) δ 7.76 (d, J=2.7 Hz, 1H), 7.41 (dd, J=8.9, 2.7 Hz, 1H), 6.89 (d, J=8.9 Hz, 1H), 3.00-2.83 (m, 6H), 2.61 (dq, J=13.9, 6.7 Hz, 6H).

### Example 15: Synthesis of TCEP-11

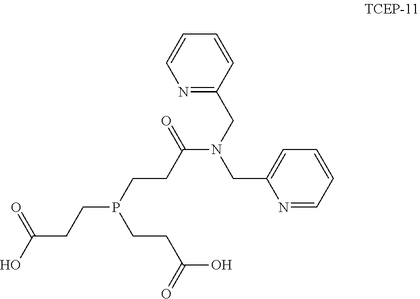 [SMILES: O=C(O)CCP(CCC(=O)O)CCC(=O)N(Cc1ccccn1)Cc1ccccn1]

TCEP-11 was synthesized as the synthesis procedure B wherein (Bis(pyridin-2-ylmethyl)amine, Shanghai Acmec Biochemical Co., Ltd) was amine reagent, yielding TCEP-11 (10.5 mg, 4.9%) as brown solid. MS[M+H]⁺=432.24, exact mass calc. for C₂₁H₂₆N₃O₅P is 431.16. ¹H NMR (400 MHz, Deuterium Oxide) δ 8.82-8.68 (m, 2H), 8.58-8.36 (m, 2H), 8.01-7.81 (m, 4H), 5.34 (s, 1H), 5.29 (s, 1H), 5.04 (d, J=2.9 Hz, 2H), 4.48 (s, 1H), 3.16 (dt, J=19.2, 6.5 Hz, 1H), 2.95-2.79 (m, 3H), 2.73-2.48 (m, 5H), 2.20 (ddt, J=36.1, 11.6, 7.5 Hz, 2H).

### Example 16: Synthesis of TCEP-12

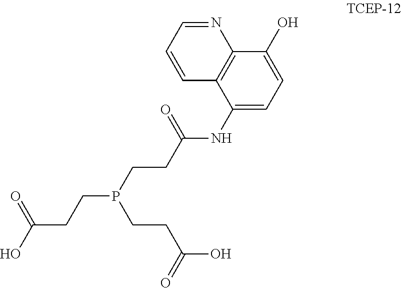 [SMILES: O=C(O)CCP(CCC(=O)O)CCC(=O)Nc1ccc(O)c2ncccc12]

TCEP-12 was synthesized as the synthesis procedure A wherein (5-Amino-8-hydroxyquinoline, Bidepharm) was amine reagent, yielding TCEP-12 (33.2 mg, 16.9%) as white solid. MS[M−H]⁻=391.24, exact mass calc. for C₁₈H₂₁N₂O₆P is 392.11. ¹H NMR (400 MHz, Deuterium Oxide) δ 9.02-8.96 (m, 2H), 8.05-7.98 (m, 1H), 7.84 (s, 1H), 7.64 (d, J=8.4 Hz, 1H), 7.44 (d, J=8.4 Hz, 1H), 2.90-2.80 (m, 2H), 2.67-2.56 (m, 6H), 2.26-2.17 (m, 4H).

### Example 17: Synthesis of TCEP-15

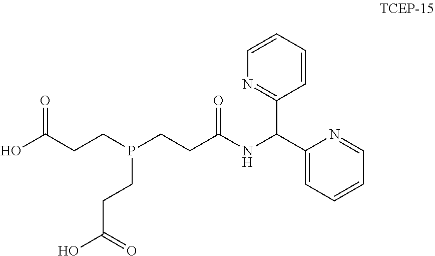 [SMILES: O=C(O)CCP(CCC(=O)O)CCC(=O)NC(c1ccccn1)c1ccccn1]

TCEP-15 was synthesized as the synthesis procedure B wherein (Bis(pyridin-2-yl) methanamine, Bidepharm) was amine reagent, yielding TCEP-15 (21.7 mg, 10.4%) as white solid. MS[M+H]⁺=418.26, exact mass calc. for C₂₀H₂₄N₃O₅P is 417.15. ¹H NMR (400 MHz, Deuterium Oxide) δ 8.69 (td, J=6.2, 1.6 Hz, 2H), 8.37 (dtd, J=15.8, 7.9, 1.7 Hz, 2H), 7.93-7.79 (m, 4H), 3.09-2.72 (m, 6H), 2.70-2.52 (m, 6H).

### Example 18: Synthesis of TCEP-18

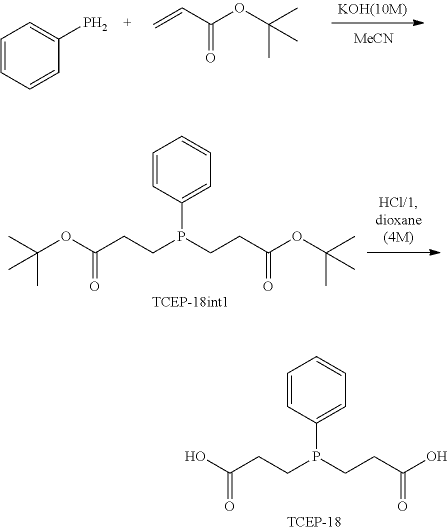

1. TCEP-18-int1

Phenyl phosphine (110 mg, 1.0 mmol, Adamas) was dissolved in acetonitrile (5 ml, degassed) in a flame-dried, round bottom flask under N₂(g). Potassium hydroxide (10N, 10 ul) was added to this mixture, and the resulting solution was cooled to 0° C. Tert-Butyl acrylate (0.44 ml, 3.0 mmol, Adamas) was added. Upon complete addition of Tert-Butyl acrylate, the reaction was heated at 50° C. and stirred for 8 hours. The reaction mixture was taken up by EtOAc (10 mL), then washed with brine (2×5 ml). The organic layer was dried over Na₂SO₄, filtered, and concentrated in vacuo. The residue was purified by flash column (EtOAc/petroleum ether=0˜20% (v/v)) to give product TCEP-18-int1 as a clear liquid (254 mg, 69.4%).

### 2. TCEP-18

The solution of TCEP-18-int1 (254 mg, 0.69 mmol) in HCl/1,4-dioxane (4M, Adamas) was stirred for 2 h at room temperature under N₂ atmosphere. LCMS showed reaction was completed and the mixture was concentrated to remove 1,4-dioxane, the resulting residue was taken up by water and lyophilized to give TCEP-18 (152.7 mg, 88.2%) as white solid. MS[M−H]⁻=253.19, exact mass calc. for C₁₂H₁₅O₄P is 254.07. ¹H NMR (400 MHz, DMSO-d₆): δ7.74 (dd, J=10.9, 7.3 Hz, 2H), 7.62-7.49 (m, 3H), 2.46-2.35 (m, 2H), 2.33-2.00 (m, 6H).

### Example 19: Synthesis of TCEP-19

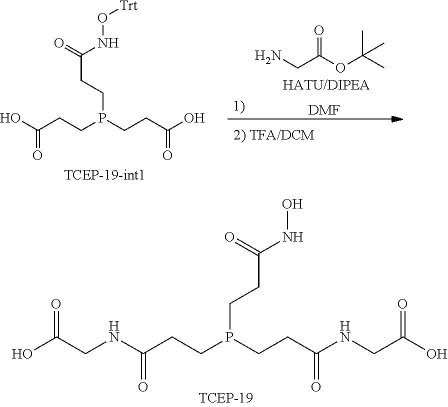

To a solution of TCEP-19-int1 (200 mg, 0.39 mmol, 1.0 eq.) in DMF (3 mL) was added HATU (380 mg, 1.0 mmol, 2.5 eq) followed by DIPEA (174 μL, 1.0 mmol, 2.5 eq) at 0° C. under N₂ atmosphere. The mixture was stirred for 30 min, tert-Butyl glycinate (1 mmol, Adamas) was added. The reaction was stirred at room temperature for 1 h. The reaction mixture was purified by RP-HPLC using a C18 column yielding the protected product. The product was dissolved in DCM (3 mL) and TFA (0.5 mL) was added. The mixture was stirred for 1 h, LCMS showed reaction was completed. The mixture was concentrated and the residue was taken up by distilled water (10 mL), washed with EtOAc (2*5 mL). The aqueous layer was lyophilized to give TCEP-19 (10.2 mg, 6.8%) as brown solid. MS[M+H]⁺=380.24, exact mass calc. for C₁₃H₂₂N₃O₈P is 379.31. ¹H-NMR (400 MHz, Deuterium Oxide): δ 3.99 (s, 4H), 2.93-2.84 (m, 4H), 2.76-2.53 (m, 8H).

### Example 20: Synthesis of TCEP-20

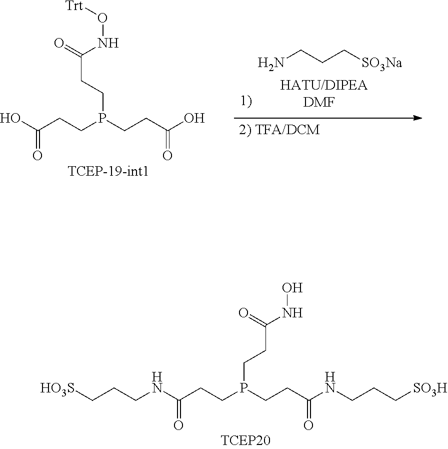

To a solution of TCEP-19-int1 (200 mg, 0.39 mmol, 1.0 eq.) in DMF (3 mL) was added HATU (380 mg, 1.0 mmol, 2.5 eq) followed by DIPEA (174 μL, 1.0 mmol, 2.5 eq) at 0° C. under N₂ atmosphere. The mixture was stirred for 30 min, Sodium 3-Aminopropane-1-Sulfonate (1 mmol, Adamas) was added. The reaction was stirred at room temperature for 1 h. The reaction mixture was purified by RP-HPLC using a C18 column yielding the protected product. The product was dissolved in DCM (3 mL) and TFA (0.5 mL) was added. The mixture was stirred for 1 h, LCMS showed reaction was completed. The mixture was concentrated and the residue was taken up by distilled water (10 mL), washed with EtOAc (2*5 mL). The aqueous layer was lyophilized to give TCEP-20 (23.7 mg, 11.7%) as brown solid. MS[M−H]⁻=506.22, exact mass calc. for C₁₅H₃₀N₃O₁₀PS₂ is 507.51. ¹H NMR (400 MHz, Deuterium Oxide): δ 3.27 (t, J=6.8 Hz, 4H), 2.91-2.85 (m, 4H), 2.80-2.71 (m, 4H), 2.69-2.61 (m, 2H), 2.58-2.49 (m, 6H), 1.94-1.85 (m, 4H).

### Example 21: Synthesis of TCEP-21

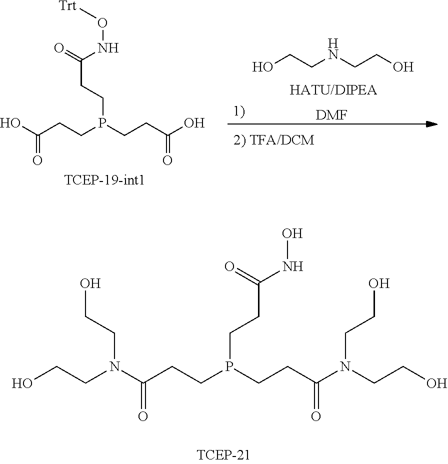

To a solution of TCEP-19-int1 (200 mg, 0.39 mmol, 1.0 eq.) in DMF (3 mL) was added HATU (380 mg, 1.0 mmol, 2.5 eq) followed by DIPEA (174 μL, 1.0 mmol, 2.5 eq) at 0° C. under N₂ atmosphere. The mixture was stirred for 30 min, Diethanolamine (1 mmol, Bidepharm) was added. The reaction was stirred at room temperature for 1 h. The reaction mixture was purified by RP-HPLC using a C18 column yielding the protected product. The product was dissolved in DCM (3 mL) and TFA (0.5 mL) was added. The mixture was stirred for 1 h, LCMS showed reaction was completed. The mixture was concentrated and the residue was taken up by distilled water (10 mL), washed with EtOAc (2*5 mL). The aqueous layer was lyophilized to give TCEP-20 (13.8 mg, 7.8 yield) as white solid. MS[M+H])=440.27, exact mass calc. for C₁₇H₃₄N₃O₈P is 439.45. ¹H NMR (400 MHz, Deuterium Oxide): δ 4.50-4.28 (m, 4H), 3.78 (t, J=5.2 Hz, 4H), 3.38 (t, J=5.1 Hz, 4H), 3.17 (q, J=5.2 Hz, 4H), 2.96-2.80 (m, 4H), 2.68-2.51 (m, 8H).

### Example 22: Synthesis of TCEP-23

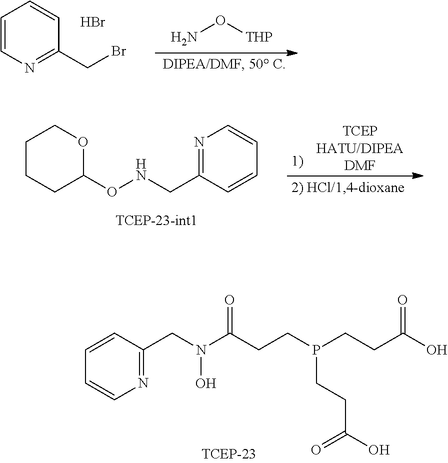

1. TCEP-23-int1

To a solution of 2-(Aminooxy)tetrahydro-2H-pyran (1.17 g, 10 mmol, 2.0 eq, Bidepharm) in DMF (15 mL) was added DIPEA (3.5 mL, 20 mmol, 4 .eq) followed by 2-(Bromomethyl)pyridine hydrobromide (1.3 g, 5.0 mmol, 1.0 eq, Adamas). The mixture was stirred for 16 h at 50° C. The reaction mixture was poured into water (100 mL), extracted with EtOAc (30Ml*3). The organic layer was washed with brine (30 mL), dried over Na₂SO₄ and filtered. The filtrate was concentrated and purified by flash column, to give TCEP-23-int1 (800 mg, 80%), as colorless oil.

### 2. TCEP-23

To a solution of TCEP (286.6 mg, 1.0 mmol, 2.0 eq.) in DMF (3 mL) was added HATU (190 mg, 0.5 mmol, 1 eq) followed by DIPEA (2.0 mmol, 4.0 eq) under N₂ atmosphere. The mixture was stirred for 30 min and TCEP-23-int1 (100 mg, 0.5 mmol, 1.0 eq) was added. The reaction was stirred at room temperature for 4 h. The reaction mixture was purified by RP-HPLC using a C18 column yielding the desired product. The product was dissolved in HCl/1,4-dioxane (3 mL). The mixture was stirred for 1 h, LCMS showed reaction was completed. The mixture was concentrated and the residue was taken up by distilled water, lyophilized to give TCEP-23 (17.9 mg, 10.0%) as white solid. MS[M+H]+=357.19, exact mass calc. for C₁₅H₂₁N₂O₆P is 356.31. ¹H-NMR (400 MHz, Deuterium Oxide): δ 8.73 (dd, J=6.3, 1.7 Hz, 1H), 8.59 (td, J=7.9, 1.6 Hz, 1H), 8.02 (dd, J=6.4, 3.4 Hz, 2H), 5.21 (s, 2H), 3.21-2.97 (m, 2H), 2.95-2.80 (m, 4H), 2.69-2.56 (m, 6H).

### Example 23: Synthesis of TCEP-24

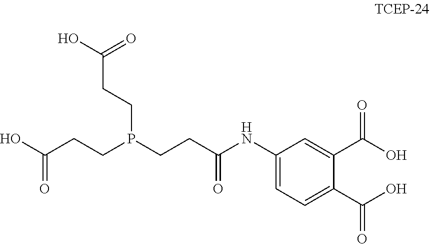 [SMILES: O=C(O)CCP(CCC(=O)O)CCC(=O)Nc1ccc(C(=O)O)c(C(=O)O)c1]

TCEP-24 was synthesized as the synthesis procedure A wherein (4-Aminophthalic acid, Bidepharm) was amine reagent, yielding TCEP-24 (21.5 mg, 10.4% yield) as white solid. MS[M−H]⁻=412.22, exact mass calc. for C₁₇H₂₀NO₉P is 413.09. ¹H NMR (400 MHz, Deuterium Oxide): δ 7.87 (d, J=8.5 Hz, 1H), 7.77 (d, J=2.1 Hz, 1H), 7.71-7.64 (m, 1H), 3.08-3.00 (m, 2H), 2.96-2.86 (m, 4H), 2.70-2.59 (m, 6H).

### Example 24: Synthesis of TCEP-25

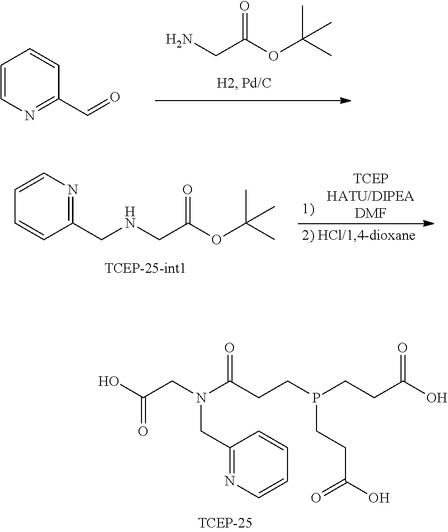

1. TCEP-25-int1

To a solution of 2-Pyridinecarboxaldehyde (1.0 g, 10 mmol, 1.0 eq. Adamas) and tert-Butyl glycinate (1.3 g, 10.0 mmol, 1.0 eq) in MeOH (25 mL) was added Pd/C (150 mg) and two drops of AcOH. The mixture was degassed 3 times and purged with H₂, then stirred for 16 h at room temperature under H₂ atmosphere. The reaction mixture was filtered through a Celite pad and the filtrate was concentrate then purified by flash column, to give TCEP-25-int1 (1.6 g, 72.0%) as yellow oil.

### 2. TCEP-25

To a solution of TCEP (286.6 mg, 1.0 mmol, 2.0 eq.) in DMF (3 mL) was added HATU (190 mg, 0.5 mmol, 1 eq) followed by DIPEA (2.0 mmol, 4.0 eq) under N₂ atmosphere. The mixture was stirred for 30 min and TCEP-25-int1 (111 mg, 0.5 mmol, 1.0 eq) was added. The reaction was stirred at room temperature for 4 h. The reaction mixture was purified by RP-HPLC using a C18 column yielding the desired product. The product was dissolved in HCl/1,4-dioxane (3 mL). The mixture was stirred for 1 h, LCMS showed reaction was completed. The mixture was concentrated and the residue was taken up by distilled water, lyophilized to give TCEP-25 (51.3 mg, 17.2% yield) as white solid. MS[M+H]⁺=399.25, exact mass calc. for C₁₇H₂₃N₂O₇P is 398.12. ¹H NMR (400 MHz, Deuterium Oxide): δ 8.60 (dd, J=5.9, 1.6 Hz, 1H), 8.45 (td, J=8.0, 1.6 Hz, 1H), 7.92 (d, J=8.3 Hz, 1H), 7.87 (ddd, J=7.5, 5.9, 1.3 Hz, 1H), 4.88 (s, 2H), 4.39 (s, 2H), 2.84-2.68 (m, 6H), 2.51-2.41 (m, 6H).

### Example 25: Synthesis of TCEP-26

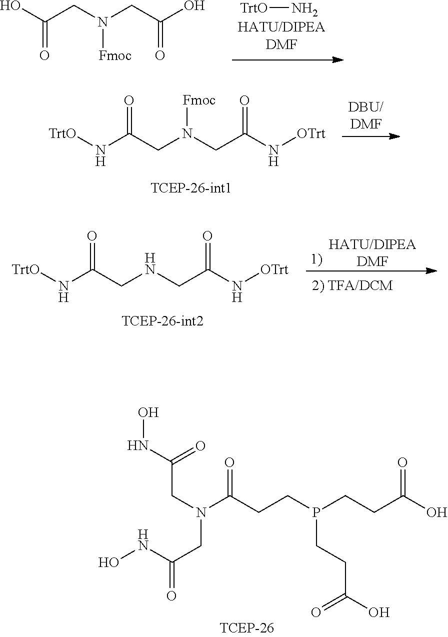

1. TCEP-26-int1

To a solution of Fmoc-iminodiacetic acid (1.8 g, 5.0 mmol, 1.0 eq, Bidepharm) in DMF (30 mL) was added HATU (4.3 g, 11.0 mmol, 2.2 eq) followed by DIPEA (2.0 mmol, 4.0 eq) under N₂ atmosphere. The mixture was stirred for 30 min and O-Tritylhydroxylamine (3.0 g, 11.0 mmol, 2.2 eq) was added. The reaction was stirred at room temperature for 4 h. The reaction mixture was poured into water (200 mL). The precipitate was collected by filtration and the filter cake was dried over vacuum to give TCEP-23-int1 (4.0 g, 92.0%), as white solid.

2. TCEP-26-int2

To a solution of TCEP-26-int1 (2.0 g, 2.3 mmol, 1.0 eq) in DMF (10 mL) was added DBU (2 mL).

The mixture was stirred for 1 h at room temperature, then poured into ice-water (100 mL), extracted with EtOAc (50 mL*3). The combined organic layer was washed with brine, dried over Na₂SO₄ and filtered. The filtrate was concentrated and purified by flash column (EtOAc/petroleum ether=0˜50%, v/v) to give TCEP-26-int2 (1.2 g, 80%).

### 3. TCEP-26

The compound was synthesized as the synthesis procedure A-1 wherein TCEP-26-int2 was amine reagent, yielding TCEP-26 (31.5 mg, 21.3% yield) as white solid. MS[M+H]+=396.17, exact mass calc. for C₁₃H₂₂N₃O₉P is 395.11. ¹H NMR (400 MHz, Deuterium Oxide) δ 4.13 (s, 2H), 3.97 (s, 2H), 2.85-2.77 (m, 4H), 2.54-2.47 (m, 6H), 2.18-2.08 (m, 2H).

### Example 26: Synthesis of TCEP-28

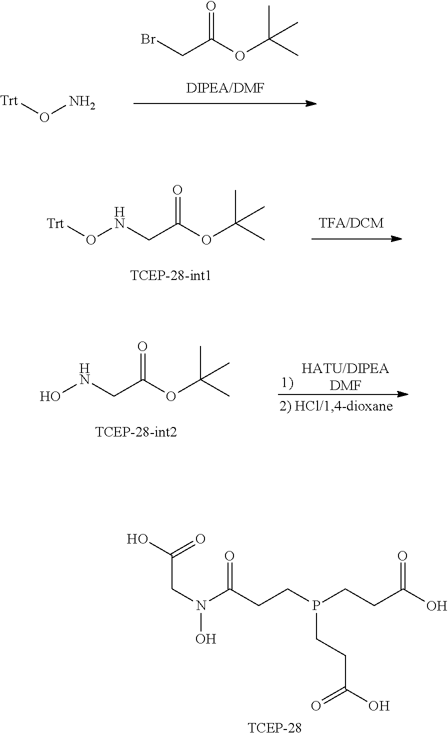

1. TCEP-28-int1

To a solution of O-Tritylhydroxylamine (1.4 g, 5.0 mmol, 1.0 eq) in DMF (15 mL) was added DIPEA (1.7 mL, 10 mmol, 2 eq) followed by tert-Butyl bromoacetate (1.0 g, 5.0 mmol, 1.0 eq, Adamas). The mixture was stirred for 16 h at 50° C. The reaction mixture was poured into water (100 mL), extracted with EtOAc (30 mL*3). The organic layer was washed with brine (30 mL), dried over Na₂SO₄ and filtered. The filtrate was concentrated and purified by flash column, to give TCEP-28-int1 (1.4 g, 70%), as white solid.

2. TCEP-28-int2

To a solution of TCEP-28-int1 (1.4 g, 3.6 mmol, 1.0 eq) in DCM (15 mL) was added TFA (1.5 mL). The mixture was stirred for 2 h at room temperature. The reaction mixture was concentrated and purified by flash column, to give TCEP-28-int2 (380 mg, 71.8%), as colorless oil.

### 3. TCEP-28

To a solution of TCEP (286.6 mg, 1.0 mmol, 2.0 eq.) in DMF (3 mL) was added HATU (190 mg, 0.5 mmol, 1 eq) followed by DIPEA (2.0 mmol, 4.0 eq) under N₂ atmosphere. The mixture was stirred for 30 min and TCEP-28-int2 (73.5 mg, 0.5 mmol, 1.0 eq) was added. The reaction was stirred at room temperature for 2 h. The reaction mixture was purified by RP-HPLC using a C18 column yielding the desired product. The product was dissolved in HCl/1,4-dioxane (3 mL, Adamas). The mixture was stirred for 1 h, LCMS showed reaction was completed. The mixture was concentrated and the residue was taken up by distilled water, lyophilized to give TCEP-28 (43.6 mg, 27.1% yield) as white solid. MS[M−H]=322.16, exact mass calc. for C₁₁H₁₈NO₈P is 323.08. ¹H NMR (400 MHz, Deuterium Oxide): δ 4.36 (s, 2H), 2.86-2.77 (m, 6H), 2.56-2.48 (m, 6H).

### Example 27: Synthesis of TCEP-30

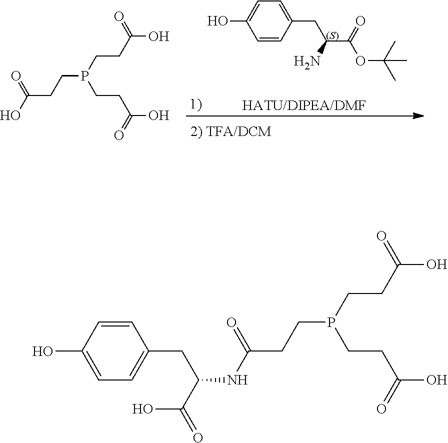

TCEP-30 was synthesized as the synthesis procedure A-1 wherein [tert-Butyl L-tyrosinate, Adamas) was amine reagent, yielding TCEP-30 (25.3 mg, 12.25%) as white solid. MS[M+H]⁺=414.23, exact mass calc. for C₁₈H₂₄NO₈P is 413.12. ¹H NMR (400 MHz, Deuterium Oxide): δ 7.24-7.14 (m, 2H), 6.91-6.82 (m, 2H), 4.67 (dd, J=10.3, 4.7 Hz, 1H), 3.26 (dd, J=14.0, 4.7 Hz, 1H), 2.92-2.64 (m, 7H), 2.57-2.30 (m, 6H).

### Example 28: Synthesis of TCEP-31

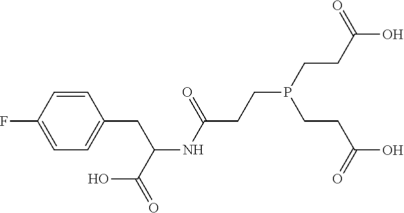 [SMILES: O=C(O)CCP(CCC(=O)O)CCC(=O)NC(Cc1ccc(F)cc1)C(=O)O]

TCEP-31 was synthesized as the synthesis procedure A wherein (DL-3-(4-Fluorophenyl)alanine, Bidepharm) was amine reagent, yielding TCEP-31 (27.1 mg, 13.10%) as white solid. MS[M+H]⁺=416.01, exact mass calc. for C₁₈H₂₃NO₇P is 415.12. ¹H NMR (400 MHz, Deuterium Oxide): δ 7.36-7.26 (m, 2H), 7.12 (t, J=8.8 Hz, 2H), 4.70 (dd, J=10.1, 4.8 Hz, 1H), 3.31 (dd, J=14.0, 4.9 Hz, 1H), 2.95 (dd, J=14.0, 10.1 Hz, 1H), 2.90-2.65 (m, 6H), 2.56-2.40 (m, 6H).

### Example 29: Synthesis of TCEP-32

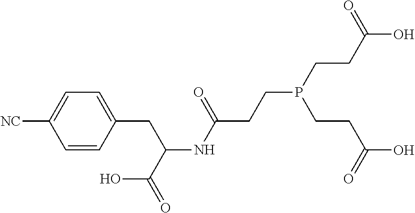 [SMILES: N#Cc1ccc(CC(NC(=O)CCP(CCC(=O)O)CCC(=O)O)C(=O)O)cc1]

TCEP-32 was synthesized as the synthesis procedure A wherein (DL4-Cyanophenylalanine, Bidepharm) was amine reagent, yielding TCEP-32 (18.5 mg, 8.76%) as white solid. MS[M+H]⁺=423.24, exact mass calc. for C₁₉H₂₃N₂O₇P is 422.12. ¹H NMR (400 MHz, Deuterium Oxide): δ 7.77 (d, J=8.1 Hz, 2H), 7.49 (d, J=8.1 Hz, 2H), 4.77-4.71 (m, 1H), 3.42 (dd, J=14.0, 5.0 Hz, 1H), 3.05 (dd, J=14.0, 10.0 Hz, 1H), 2.91-2.70 (m, 6H), 2.68-2.39 (m, 6H).

### Example 30: Synthesis of TCEP-33

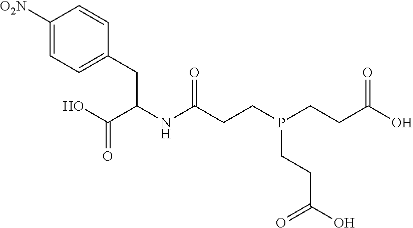 [SMILES: O=C(O)CCP(CCC(=O)O)CCC(=O)NC(Cc1ccc([N+](=O)[O-])cc1)C(=O)O]

TCEP-33 was synthesized as the synthesis procedure A wherein (DL-4-nitro-phenylalanine, Bidepharm) was amine reagent, yielding TCEP-33 (20.7 mg, 9.37%) as white solid. MS[M+H]⁺=443.24, exact mass calc. for C₁₉H₂₃N₂O₉P is 442.11. ¹H NMR (400 MHz, Deuterium Oxide): δ 8.22 (d, J=8.2 Hz, 2H), 7.54 (d, J=8.2 Hz, 2H), 4.84-4.80 (m, 1H), 3.47 (dd, J=14.0, 4.8 Hz, 1H), 3.11 (dd, J=13.9, 10.3 Hz, 1H), 2.91-2.73 (m, 6H), 2.58-2.38 (m, 6H).

### Example 31: Synthesis of TCEP-34

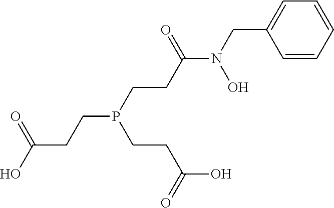 [SMILES: O=C(O)CCP(CCC(=O)O)CCC(=O)N(O)Cc1ccccc1]

TCEP-34 was synthesized as the synthesis procedure A wherein (N-Benzylhydroxylamine hydrochloride, Bidepharm) was amine reagent, yielding TCEP-34 (15.7 mg, 8.85%) as white solid. MS[M+H]⁺=356.05, exact mass calc. for C₁₆H₂₂NO₆P is 355.12. ¹H NMR (400 MHz, Deuterium Oxide): δ 7.55-7.34 (m, 5H), 4.84 (s, 2H), 3.14 (dt, J=19.8, 6.9 Hz, 2H), 2.92 (dt, J=18.2, 7.0 Hz, 4H), 2.62 (dq, J=14.5, 7.3 Hz, 6H).

### Example 32: Synthesis of TCEP-35

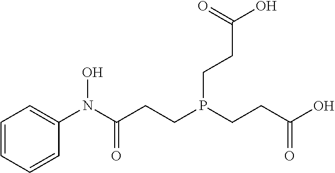 [SMILES: O=C(O)CCP(CCC(=O)O)CCC(=O)N(O)c1ccccc1]

TCEP-35 was synthesized as the synthesis procedure A wherein (N-Phenylhydroxylamine, Bidepharm) was amine reagent, yielding TCEP-35 (17.1 mg, 10.0%) as white solid. MS[M+H]⁺=341.97, exact mass calc. for C₁₅H₂NO₆P is 341.10. ¹H NMR (400 MHz, Deuterium Oxide): δ 7.57-6.88 (m, 5H), 3.27-3.22 (m, 1H), 2.92-2.78 (m, 3H), 2.65-2.53 (m, 6H), 2.29-1.98 (m, 2H).

### Example 33: Synthesis of TCEP-37

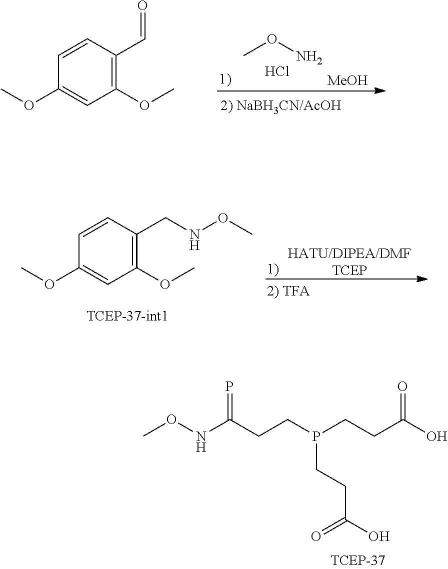

1. TCEP-37-int1

To a solution of 2,4-Dimethoxybenzaldehyde (1.66 g, 10.0 mmol, 1.0 eq, Adamas) in MeOH (25 mL) was added O-methylhydroxylamine hydrochloride (1.66 g, 20.0 mmol, 2.0 eq, Bidepharm). The resulting mixture was stirred for 16 h at room temperature, LCMS showed reaction was completed. The mixture was concentrated and the residue was taken up by AcOH (20 mL), NaBH₃CN was added and stirred for 5 h at room temperature. LCMS showed reaction was completed. The reaction mixture was concentrated and residue was poured into ice-water (200 mL) extracted with EtOAc (50 mL*3). The combined organic layer was dried over Na₂SO₄ and filtered. The filtrate was concentrated over vacuum and purified with flash column (EtOAc/petroleum ether=0˜50%) to give product TCEP-37-int1 (N-(2,4-dimethoxybenzyl)-O-methyl hydroxylamine, 1.5 g, 76.1%, colorless oil).

### 2. TCEP-37

TCEP-37 was synthesized as the synthesis procedure A-1 wherein TCEP-73-int1 was amine reagent, yielding TCEP-37 (12.8 mg, 9.14%) as white solid. MS[M+H]⁺=280.18, exact mass calc. for C₁₀H₁₈NO₆P is 279.09. ¹H NMR (400 MHz, Deuterium Oxide): δ (s, 3H), 2.89 (dt, J=18.4, 7.1 Hz, 4H), 2.72 (dd, J=18.1, 6.6 Hz, 2H), 2.61 (dq, J=13.9, 6.7 Hz, 6H).

### Examples I.1-I.36: Preparation of Antibody-[MC-VC-PAB-MMAE]₂ by TCEP or the Reductant Described Above

(1) ZnCl₂ (0.24 mM) and the reductant (0.02 mM) were added to a solution of a monoclonal antibody (0.012 mM, in MES buffer, pH6.7, 20 mM) and the reaction mixture was allowed to stay at 4° C. for 4 h, 8 h, or 12 h;
(2) EDTA (0.6 mM) was added to trap Zn²⁺; MC-VC-PAB-MMAE (0.06 mM) in DMA was introduced and the reaction was continued at 24° C. for 30 min when the reductant is TCEP, and the reaction time is 1 h when the reductant is the other reductants described in examples I.4-I.36;
(3) cysteine (0.08 mM) was added to deplete excessive MC-VC-PAB-MMAE; The reaction mixture was subjected to purification using a desalting column.

Adjust the kinds of the reductant and/or the molar ratio of antibody/reductant, which are shown in table I-1. The buffer system is MOPS buffer and the pH value is 7.4 in example I.10.

### Comparative Examples I.1-I.12: Preparation of Antibody-[MC-VC-PAB-MMAE]₂ by TCEP or the Reductant Described Above without the Transition Metal Ions

The preparation of comparative examples I.1-I.3 is similar to example I.1 and the preparation of comparative examples I.4-I.12 is similar to example I.4, but the concentration of the transition metal ions is 0. The parameters of the comparative examples I.1-I.12 are shown in table I-1.

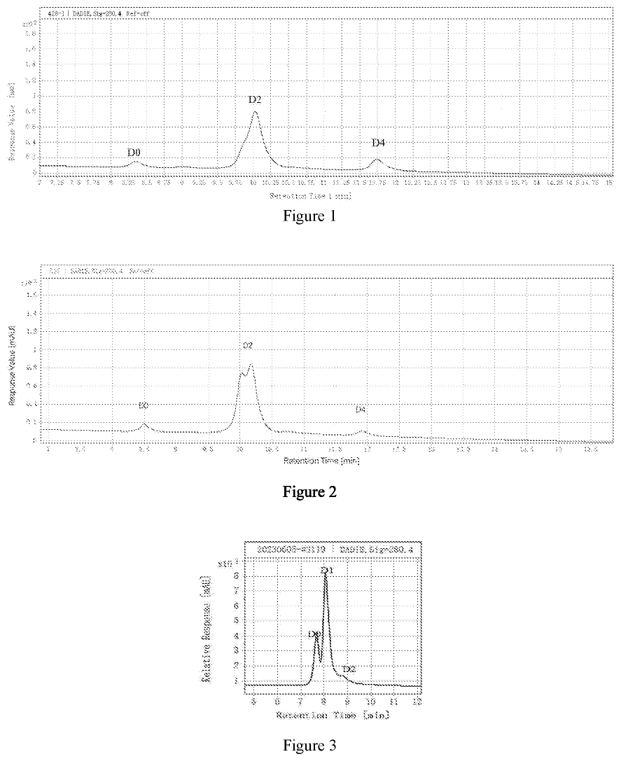

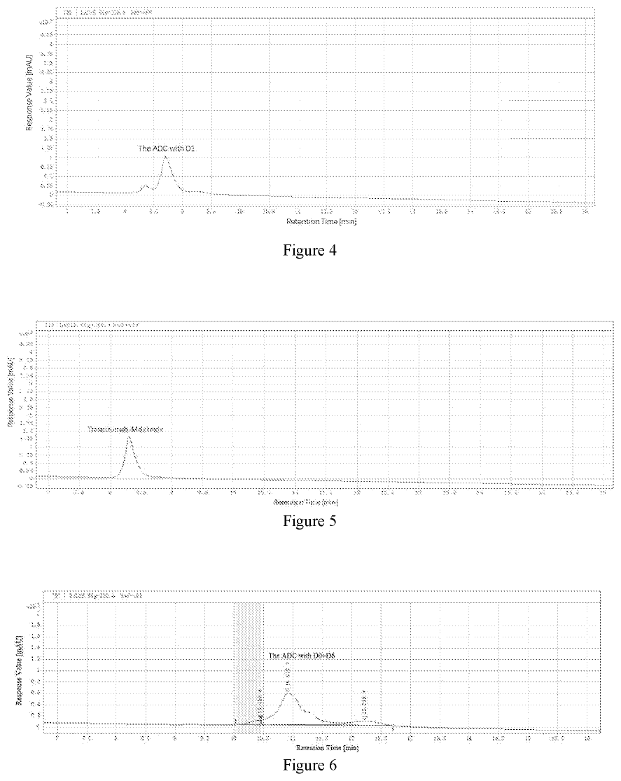

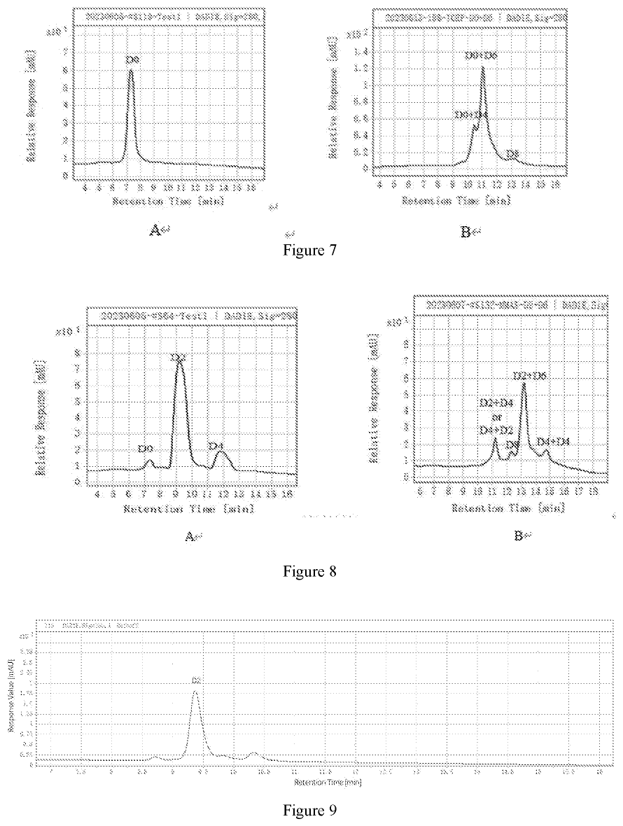

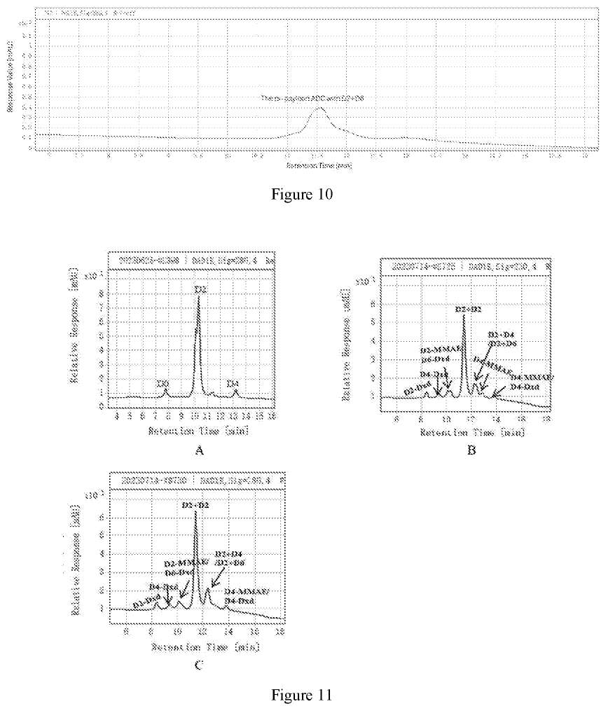

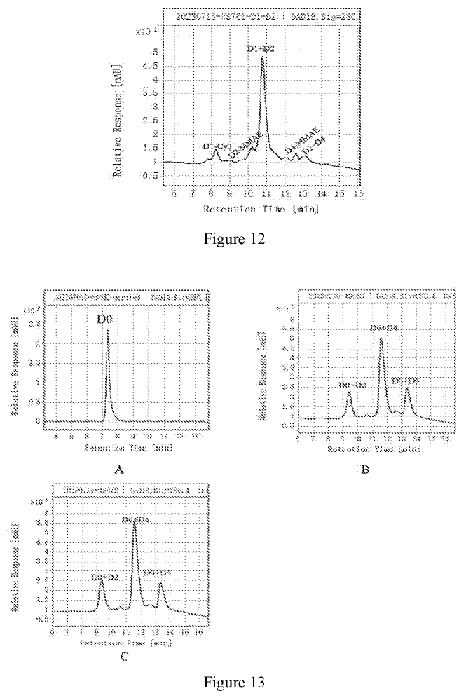

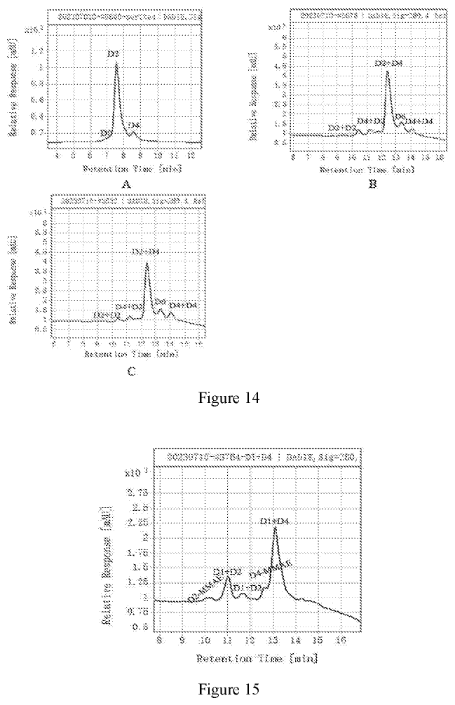

The chromatographic peak areas of examples I.1-I.36 and comparative examples I.1-I.12 were showed in Table I-2 and the homogeneity assays results were shown in Table I-1. The chromatograms of E I.1 and E I.4 were shown in FIGS. 1-2, respectively.

TABLE I-1

| | | | | The | | | | | |
|---|---|---|---|---|---|---|---|---|---|
| | | | Antibody/ | time in | | | | | |
| | | | reductant | step (1) | D0 | D2 | D4 | D6 | D8 |
| No | Antibody | Reductant | (molar ratio) | (h) | (wt %) | (wt %) | (wt %) | (wt %) | (wt %) |
| E I.1 | Trastuzumab | TCEP | 1:1.67 | 4 | 5.22 | 80.6 | 14.17 | 0 | 0 |
| E I.2 | Sacituzumab | TCEP | 1:1.67 | 4 | 7.77 | 76.5 | 15.74 | 0 | 0 |
| E I.3 | Belantamab | TCEP | 1:1.67 | 4 | 8.14 | 82.17 | 9.69 | 0 | 0 |
| E I.4 | Trastuzumab | TCEP-NO | 1:1.67 | 4 | 4.55 | 91.09 | 4.36 | 0 | 0 |
| E I.5 | Sacituzumab | TCEP-NO | 1:1.67 | 4 | 5.98 | 84.97 | 9.06 | 0 | 0 |
| E I.6 | Belantamab | TCEP-NO | 1:1.67 | 4 | 1.77 | 89.2 | 9.04 | 0 | 0 |
| E I.7 | Trastuzumab | TCEP-3NO | 1:1.67 | 8 | 2.87 | 91.47 | 5.67 | 0 | 0 |
| E I.8 | Sacituzumab | TCEP-3NO | 1:1.67 | 8 | 2.85 | 87.86 | 9.29 | 0 | 0 |
| E I.9 | Belantamab | TCEP-3NO | 1:1.67 | 8 | 1.52 | 87.62 | 10.86 | 0 | 0 |
| E I.10 | Trastuzumab | TCEP-CO | 1:1.67 | 12 | 4.87 | 90.51 | 4.63 | 0 | 0 |
| E I.11 | Sacituzumab | TCEP-CO | 1:1.67 | 12 | 9.91 | 81.26 | 8.83 | 0 | 0 |
| E I.12 | Belantamab | TCEP-CO | 1:1.67 | 12 | 3.94 | 85.44 | 10.62 | 0 | 0 |
| E I.13 | Trastuzumab | TCEP-1 | 1:1.3 | 4 | 3.47 | 90.75 | 5.78 | 0 | 0 |
| E I.14 | Trastuzumab | TCEP-2 | 1:1.2 | 4 | 6.73 | 83.46 | 9.82 | 0 | 0 |
| E I.15 | Trastuzumab | TCEP-3 | 1:1.2 | 4 | 11.83 | 80.00 | 8.17 | 0 | 0 |
| E I.16 | Trastuzumab | TCEP-4 | 1:0.9 | 4 | 11.83 | 81.08 | 7.09 | 0 | 0 |
| E I.17 | Trastuzumab | TCEP-5 | 1:1.6 | 4 | 11.82 | 77.63 | 10.56 | 0 | 0 |
| E I.18 | Trastuzumab | TCEP-6 | 1:1.6 | 4 | 2.67 | 94.25 | 3.08 | 0 | 0 |
| E I.19 | Trastuzumab | TCEP-7 | 1:0.9 | 4 | 13.08 | 76.81 | 10.11 | 0 | 0 |
| E I.20 | Trastuzumab | TCEP-8 | 1:2.2 | 4 | 10.37 | 78.93 | 10.70 | 0 | 0 |
| E I.21 | Trastuzumab | TCEP-9 | 1:1.4 | 4 | 9.95 | 78.49 | 11.56 | 0 | 0 |
| E I.22 | Trastuzumab | TCEP-10 | 1:1.8 | 4 | 8.99 | 82.27 | 8.74 | 0 | 0 |
| E I.23 | Trastuzumab | TCEP-15 | 1:1 | 4 | 7.80 | 79.66 | 12.54 | 0 | 0 |
| E I.24 | Trastuzumab | TCEP-18 | 1:1 | 4 | 10.27 | 77.66 | 12.07 | 0 | 0 |
| E I.25 | Trastuzumab | TCEP-19 | 1:1.4 | 4 | 6.66 | 88.03 | 5.31 | 0 | 0 |
| E I.26 | Trastuzumab | TCEP-20 | 1:1.6 | 4 | 3.87 | 91.57 | 4.55 | 0 | 0 |
| E I.27 | Trastuzumab | TCEP-21 | 1:1.4 | 4 | 6.22 | 87.28 | 6.50 | 0 | 0 |
| E I.28 | Trastuzumab | TCEP-23 | 1:1.2 | 4 | 7.10 | 87.62 | 5.28 | 0 | 0 |
| E I.29 | Trastuzumab | TCEP-24 | 1:1.2 | 4 | 5.15 | 85.25 | 9.60 | 0 | 0 |
| E I.30 | Trastuzumab | TCEP-25 | 1:1.7 | 4 | 10.14 | 81.34 | 8.52 | 0 | 0 |
| E I.31 | Trastuzumab | TCEP-26 | 1:1.1 | 4 | 6.24 | 86.89 | 6.87 | 0 | 0 |
| E I.32 | Trastuzumab | TCEP-28 | 1:1.7 | 4 | 8.23 | 84.97 | 6.80 | 0 | 0 |
| E I.33 | Trastuzumab | TCEPA | 1:2.3 | 4 | 10.11 | 79.17 | 10.71 | 0 | 0 |
| E I.34 | Trastuzumab | TCEP-34 | 1:1.3 | 4 | 2.68 | 91.99 | 5.33 | 0 | 0 |
| E I.35 | Trastuzumab | TCEP-35 | 1:1.6 | 4 | 1.69 | 93.05 | 5.26 | 0 | 0 |
| E I.36 | Trastuzumab | TCEP-37 | 1:1.2 | 4 | 8.75 | 84.20 | 7.05 | 0 | 0 |
| C I.1 | Trastuzumab | TCEP | 1:1.67 | 4 | 32.5 | 48.98 | 18.52 | 0 | 0 |
| C I.2 | Sacituzumab | TCEP | 1:1.67 | 4 | 32.82 | 49.08 | 18.1 | 0 | 0 |
| C I.3 | Belantamab | TCEP | 1:1.67 | 4 | 19.12 | 48.43 | 32.45 | 0 | 0 |
| C I.4 | Trastuzumab | TCEP-NO | 1:1.67 | 4 | 69.85 | 30.15 | 0 | 0 | 0 |
| C I.5 | Sacituzumab | TCEP-NO | 1:1.67 | 4 | 58.11 | 37.6 | 4.29 | 0 | 0 |
| C I.6 | Belantamab | TCEP-NO | 1:1.67 | 4 | 38.79 | 48.82 | 12.39 | 0 | 0 |
| C I.7 | Trastuzumab | TCEP-3NO | 1:1.67 | 8 | 52.13 | 42.34 | 5.53 | 0 | 0 |
| C I.8 | Sacituzumab | TCEP-3NO | 1:1.67 | 8 | 45.05 | 45.92 | 9.04 | 0 | 0 |
| C I.9 | Belantamab | TCEP-3NO | 1:1.67 | 8 | 38.56 | 49.01 | 12.43 | 0 | 0 |
| C I.10 | Trastuzumab | TCEP-CO | 1:1.67 | 12 | — | — | — | — | — |
| C I.11 | Sacituzumab | TCEP-CO | 1:1.67 | 12 | 42.92 | 48.2 | 8.88 | 0 | 0 |
| C I.12 | Belantamab | TCEP-CO | 1:1.67 | 12 | 42.54 | 50 | 7.46 | 0 | 0 |

“E” was short for Example, and “C” was short for comparative example in the application.

TABLE I-2

| | D0 area | D2 area | D4 area | D6 area | D8 area |
|---|---|---|---|---|---|
| No. | (mAU) | (mAU) | (mAU) | (mAU) | (mAU) |
| E I.1 | 94.43 | 1458.08 | 256.34 | 0 | 0 |
| E I.2 | 147.85 | 1455.71 | 299.51 | 0 | 0 |
| E I.3 | 188.52 | 1903.05 | 224.42 | 0 | 0 |
| E I.4 | 82.96 | 1660.76 | 79.49 | 0 | 0 |
| E I.5 | 199.19 | 2830.35 | 301.79 | 0 | 0 |
| E I.6 | 72.62 | 3659.55 | 370.88 | 0 | 0 |
| E I.7 | 72.77 | 2319.40 | 143.77 | 0 | 0 |
| E I.8 | 59.03 | 1819.69 | 192.41 | 0 | 0 |
| E I.9 | 28.12 | 1621.01 | 200.91 | 0 | 0 |
| E I.10 | 64.96 | 1207.37 | 61.76 | 0 | 0 |
| E I.11 | 220.25 | 1805.97 | 196.24 | 0 | 0 |
| E I.12 | 68.26 | 1480.19 | 183.98 | 0 | 0 |
| E I.13 | 104.31 | 2727.01 | 173.65 | 0 | 0 |
| E I.14 | 224.87 | 2790.48 | 328.26 | 0 | 0 |
| E I.15 | 433.92 | 2934.07 | 299.50 | 0 | 0 |
| E I.16 | 351.19 | 2407.35 | 210.45 | 0 | 0 |
| E I.17 | 370.99 | 2437.44 | 331.46 | 0 | 0 |
| E I.18 | 85.70 | 3020.38 | 98.56 | 0 | 0 |
| E I.19 | 429.40 | 2521.30 | 331.74 | 0 | 0 |
| E I.20 | 355.85 | 2709.58 | 367.44 | 0 | 0 |
| E I.21 | 310.79 | 2452.59 | 361.29 | 0 | 0 |
| E I.22 | 326.26 | 2984.49 | 317.00 | 0 | 0 |
| E I.23 | 350.22 | 2224.94 | 217.82 | 0 | 0 |
| E I.24 | 213.84 | 1616.62 | 251.17 | 0 | 0 |
| E I.25 | 205.37 | 2712.80 | 163.62 | 0 | 0 |
| E I.26 | 125.91 | 2975.92 | 147.99 | 0 | 0 |
| E I.27 | 213.10 | 2990.48 | 222.68 | 0 | 0 |
| E I.28 | 242.87 | 2995.65 | 180.57 | 0 | 0 |
| E I.29 | 145.96 | 2416.65 | 272.05 | 0 | 0 |
| E I.30 | 320.23 | 2568.42 | 268.96 | 0 | 0 |
| E I.31 | 189.08 | 2633.57 | 208.35 | 0 | 0 |
| E I.32 | 256.41 | 2647.65 | 211.84 | 0 | 0 |
| E I.33 | 345.83 | 2707.44 | 366.40 | 0 | 0 |
| E I.34 | 44.87 | 1539.09 | 89.14 | 0 | 0 |
| E I.35 | 25.93 | 1429.45 | 80.79 | 0 | 0 |
| E I.36 | 162.55 | 1564.22 | 131.05 | 0 | 0 |
| C I.1 | 2383.50 | 3592.12 | 1358.23 | 0 | 0 |
| C I.2 | 1171.93 | 1752.54 | 646.31 | 0 | 0 |
| C I.3 | 861.72 | 2182.69 | 1462.49 | 0 | 0 |
| C I.4 | 2827.45 | 1220.44 | 0 | 0 | 0 |
| C I.5 | 2168.81 | 1403.33 | 160.11 | 0 | 0 |
| C I.6 | 1305.11 | 1642.58 | 416.87 | 0 | 0 |
| C I.7 | 1739.08 | 1412.48 | 184.48 | 0 | 0 |
| C I.8 | 1433.10 | 1460.77 | 287.57 | 0 | 0 |
| C I.9 | 772.24 | 981.52 | 248.93 | 0 | 0 |
| C I.10 | / | / | / | / | / |
| C I.11 | 1253.73 | 1407.96 | 259.39 | 0 | 0 |
| C I.12 | 875.73 | 1029.31 | 153.57 | 0 | 0 |

As the results shown in table I-1, with a conjugation process using the same steps without the addition of the transition metal ions in step (1) as a negative control (C I.1-C I.12), the disclosure successfully demonstrated that combination of the transition metal ions and reductants is responsible for higher level of D2 in the resultant ADCs. By using the process of the present disclosure to produce antibody-drug conjugates, the homogeneity of the antibody-drug conjugates is dramatically higher.

Further, as the results of examples I.4-I.36, the compounds in the present application could increase the homogeneity of the ADC with D2 compared with the traditional method using TCEP without Zn²⁺, wherein, the selective reduction ability of TCEO-6 is best, with a D2 content of up to 94.25%. Meanwhile, the selective reduction ability of TCEP-NO, TCEP-3NO, TCEP-CO, TCEP-1, TCEP-19, TCEP-20, TECP-21, TCEP-23, TCEP-24, TCEP-26, TCEP-28, TCEP-34, TCEP-35 and TCEP-37 is also wonderful, with a D2 content of up to 84%, 87%, even to 90% or 93%.

### Examples I.37-I.63 and Comparative Examples I.13-I.14: Preparation of Trastuzumab-[MC-VC-PAB-MMAE]₂ Conjugate with Different Molar Ratio of the ZnCl₂ and the Reductant

When the reductant is TCEP, the preparation of Trastuzumab-[MC-VC-PAB-MMAE]₂ conjugate is similar to example I.1, when the reductant is TCEP-NO (the preparation of Trastuzumab-[MC-VC-PAB-MMAE]₂ conjugate is similar to example I.4, but those adjust the dosage of ZnC₂ in step (1). The dosage of ZnC₂ and the molar ratio of the ZnC₂ and the reductant are described in table I-3.

The homogeneity assays results of examples I.37-I.63 and comparative examples I.13-I.14 were shown in Table I-3 and those chromatographic peak areas were showed in Table I-4.

TABLE I-3

| | | Zn²⁺/ | | | | | | |
|---|---|---|---|---|---|---|---|---|
| | Zn²⁺ | reductant | | D0 | D1 | D2 | D4 | D6 |
| No | (mM) | (Molar ratio) | Reductant | (wt %) | (wt %) | (wt %) | (wt %) | (wt %) |
| E I.37 | 0.008 | 0.4:1 | TCEP | 27.12 | 0 | 59.13 | 13.75 | 0 |
| E I.38 | 0.02 | 1:1 | TCEP | 21.73 | 0 | 64.68 | 13.59 | 0 |
| E I.39 | 0.04 | 2:1 | TCEP | 12.63 | 0 | 71.36 | 16.01 | 0 |
| E I.40 | 0.08 | 4:1 | TCEP | 7.49 | 0 | 75.48 | 17.02 | 0 |
| E I.41 | 0.12 | 6:1 | TCEP | 8.28 | 0 | 77.93 | 13.79 | 0 |
| E I.42 | 0.16 | 8:1 | TCEP | 8.28 | 0 | 77.39 | 14.34 | 0 |
| E I.43 | 0.20 | 10:1 | TCEP | 7.21 | 0 | 79.13 | 13.66 | 0 |
| E I.44 | 0.28 | 14:1 | TCEP | 8.97 | 0 | 80.75 | 10.28 | 0 |
| E I.45 | 0.32 | 16:1 | TCEP | 5.44 | 0 | 80.75 | 13.81 | 0 |
| E I.46 | 0.6 | 30:1 | TCEP | 8.55 | 0 | 81.60 | 9.85 | 0 |
| E I.47 | 1.4 | 70:1 | TCEP | 10.16 | 0 | 82.26 | 7.59 | 0 |
| E I.48 | 2.5 | 125:1 | TCEP | 10.55 | 15.34 | 68.47 | 5.65 | 0 |
| C I.13 | 5 | 250:1 | TCEP | 64.17 | 14.17 | 21.66 | 0 | 0 |
| E I.49 | 0.008 | 0.4:1 | TCEP-NO | 48.78 | 0 | 41.35 | 9.87 | 0 |
| E I.50 | 0.02 | 1:1 | TCEP-NO | 39.19 | 0 | 55.53 | 5.28 | 0 |
| E I.51 | 0.04 | 2:1 | TCEP-NO | 3.15 | 0 | 78.12 | 18.74 | 0 |
| E I.52 | 0.08 | 4:1 | TCEP-NO | 8.57 | 0 | 82.55 | 8.88 | 0 |
| E I.53 | 0.12 | 6:1 | TCEP-NO | 4.19 | 0 | 87.32 | 8.48 | 0 |
| E I.54 | 0.16 | 8:1 | TCEP-NO | 5.16 | 0 | 88.52 | 6.33 | 0 |
| E I.55 | 0.2 | 10:1 | TCEP-NO | 6.09 | 0 | 87.83 | 6.08 | 0 |
| E I.56 | 0.28 | 14:1 | TCEP-NO | 5.45 | 0 | 89.64 | 4.91 | 0 |
| E I.57 | 0.32 | 16:1 | TCEP-NO | 6.37 | 0 | 89.59 | 4.04 | 0 |
| E I.58 | 0.48 | 24:1 | TCEP-NO | 4.44 | 0 | 87.03 | 8.53 | 0 |
| E I.59 | 0.6 | 30:1 | TCEP-NO | 4.99 | 0 | 90.38 | 4.63 | 0 |
| E I.60 | 1.2 | 60:1 | TCEP-NO | 8.91 | 0 | 86.52 | 4.57 | 0 |
| E I.61 | 1.4 | 70:1 | TCEP-NO | 8.29 | 0 | 82.00 | 9.71 | 0 |
| E I.62 | 2.5 | 125:1 | TCEP-NO | 7.47 | 0 | 81.22 | 11.32 | 0 |
| E I.63 | 4 | 200:1 | TCEP-NO | 14.31 | 15.11 | 63.75 | 6.83 | 0 |
| C I.14 | 5 | 250:1 | TCEP-NO | 32.03 | 14.64 | 53.33 | 0 | 0 |

TABLE I-4

| | D0 area | D1 area | D2 area | D4 area | D6 area |
|---|---|---|---|---|---|
| No. | (mAU) | (mAU) | (mAU) | (mAU) | (mAU) |
| E I.37 | 1180.82 | 0 | 2574.55 | 598.68 | 0 |
| E I.38 | 1063.49 | 0 | 3165.51 | 665.11 | 0 |
| E I.39 | 303.49 | 0 | 1714.74 | 384.71 | 0 |
| E I.40 | 127.04 | 0 | 1280.25 | 288.68 | 0 |
| E I.41 | 330.65 | 0 | 3111.54 | 550.41 | 0 |
| E I.42 | 148.85 | 0 | 1391.22 | 257.79 | 0 |
| E I.43 | 132.95 | 0 | 1459.12 | 251.88 | 0 |
| E I.44 | 318.12 | 0 | 2863.78 | 364.58 | 0 |
| E I.45 | 98.41 | 0 | 1460.79 | 249.83 | 0 |
| E I.46 | 314.63 | 0 | 3003.97 | 362.76 | 0 |
| E I.47 | 375.71 | 0 | 3043.14 | 280.79 | 0 |
| E I.48 | 283.31 | 412.04 | 1839.41 | 151.91 | 0 |
| C I.13 | 573.75 | 126.72 | 193.71 | 0 | 0 |
| E I.49 | 1778.68 | 0 | 1507.75 | 359.89 | 0 |
| E I.50 | 1310.97 | 0 | 1857.57 | 176.62 | 0 |
| E I.51 | 53.43 | 0 | 1325.03 | 317.86 | 0 |
| E I.52 | 340.43 | 0 | 3279.20 | 352.75 | 0 |
| E I.53 | 95.71 | 0 | 1994.64 | 193.71 | 0 |
| E I.54 | 120.75 | 0 | 2071.42 | 148.13 | 0 |
| E I.55 | 144.34 | 0 | 2081.71 | 144.11 | 0 |
| E I.56 | 209.08 | 0 | 3438.85 | 188.36 | 0 |
| E I.57 | 108.83 | 0 | 1530.67 | 69.02 | 0 |
| E I.58 | 205.09 | 0 | 4020.10 | 394.02 | 0 |
| E I.59 | 97.92 | 0 | 1775.44 | 91.01 | 0 |
| E I.60 | 268.18 | 0 | 2604.17 | 137.55 | 0 |
| E I.61 | 165.98 | 0 | 1642.53 | 194.56 | 0 |
| E I.62 | 160.09 | 0 | 1741.30 | 242.64 | 0 |
| E I.63 | 202.56 | 213.79 | 902.11 | 96.69 | 0 |
| C I.14 | 259.90 | 118.82 | 432.84 | 0 | 0 |

As show in table I-3, when the reductant is TCEP, the results showed the content of D2 increases as the molar ratio of Zn²⁺ and TCEP increases from 0.4:1 to 12:1. After that, the content of D2 ratio reaches a plateau, when the molar ratio of Zn²⁺ and TCEP is 250:1, the content of D2 is lower than that of the molar ratio of the Zn²⁺/TCEP-NO ranging from 0.4:1 to 125:1. Those results indicated that the molar ratio of Zn²⁺ and TCEP plays a key role in determining the content of D2 and the selective reduction. When the molar ratio of Zn²⁺ and TCEP is 0.4:1 to 200:1, the content of D2 is up to 60%, 65%, even to 70%, 75%, 80% or 83%.

When the reductant is TCEP-NO, by adding the transition metal ions, the content of D2 increases. D2 ratio increases as Zn²⁺/TCEP-NO molar ratio increases from 0.4 to 6. After that, D2 ratio reaches a plateau, when the molar ratio of Zn²⁺/TCEP-NO is up to 200:1 and 250:1, the content of D2 is lower than that of Zn²⁺/TCEP-NO molar ratio ranging from 2:1 to 125:1. This indicates the transition metal ions, especially the Zn²⁺/TCEP-NO ratio, plays a key role in determining the D2 ratio and the reduction selectivity.

### Examples I.64-I.71: Preparation of Trastuzumab-[MC-VC-PAB-MMAE]₂ Conjugate with Different Molar Ratio of the Antibody and the Reductant

When the reductant is TCEP, the preparation of Trastuzumab-[MC-VC-PAB-MMAE]₂ conjugate is similar to example I.1. When the reductant is not TCEP, the preparation of Trastuzumab-[MC-VC-PAB-MMAE]₂ conjugate is similar to example I.4. But those adjust the dosage of the antibody in step (1) or the incubation time in step (1). The dosage of antibody and the molar ratio of the antibody and the reductant are shown in table I-5.

The homogeneity assays results of examples I.64-I.71 were shown in Table I-5 and those chromatographic peak areas were showed in Table I-6.

TABLE I-5

| | | | Antibody/ | Time | | | | |
|---|---|---|---|---|---|---|---|---|
| | | Antibody | reductant | in step | D0 | D2 | D4 | D6 |
| No. | Reductant | (mM) | (Molar Ratio) | (1)(h) | (%) | (%) | (%) | (%) |
| E I.64 | TCEP | 0.017 | 1:1.2 | 4 | 9.27 | 82.64 | 8.09 | 0 |
| E I.65 | TCEP | 0.01 | 1:2.0 | 1 | 2.72 | 78.12 | 19.16 | 0 |
| E I.66 | TCEP | 0.008 | 1:2.5 | 1 | 2.75 | 68.75 | 28.50 | 0 |
| E I.67 | TCEP | 0.0067 | 1:3.0 | 1 | 1.94 | 56.33 | 39.47 | 2.26 |
| E I.68 | TCEP-NO | 0.011 | 1:1.8 | 4 | 3.07 | 89.77 | 7.16 | 0 |
| E I.69 | TCEP-NO | 0.01 | 1:2.0 | 1 | 3.21 | 84.98 | 11.83 | 0 |
| E I.70 | TCEP-NO | 0.008 | 1:2.5 | 1 | 0 | 74.07 | 25.93 | 0 |
| E I.71 | TCEP-NO | 0.0067 | 1:3.0 | 1 | 0 | 56.05 | 39.22 | 4.73 |

TABLE I-6

| | D0 area | D2 area | D4 area | D6 area |
|---|---|---|---|---|
| No. | (mAU) | (mAU) | (mAU) | (mAU) |
| E I.64 | 287.52 | 2564.00 | 251.06 | 0 |
| E I.65 | 53.01 | 1523.90 | 373.85 | 0 |
| E I.66 | 75.07 | 1876.68 | 777.92 | 0 |
| E I.67 | 48.23 | 1398.04 | 979.55 | 56.01 |
| E I.68 | 93.17 | 2726.49 | 217.49 | 0 |
| E I.69 | 68.87 | 1825.70 | 254.14 | 0 |
| E I.70 | 0 | 1565.49 | 547.93 | 0 |
| E I.71 | 0 | 1378.54 | 964.55 | 116.42 |

As the results shown in the table T-5, using the TCEP as reductant, when the molar ratio of antibody/TCEP is 1:1 to 1:3, the content of the ADC with D2 is up to 56%, 70, even to 80%. When the molar ratio of antibody/TCEP is 1:2 and 1:3, the reduction time is shortened to 1 h and the content of D2 is greater than 56%, even to 70% and 78%. As the results shown in table I-5, in example E I.16, E I.19 and E I.23-24, using the TCEO-NO as reductant, when the molar ratio of antibody and TCEP-NO is 1:0.9 to 1:3.0, the content of the ADC with D2 is up to 70%, 75, even to 80%, 85% or 90%. When the molar ratio of antibody/TCEP-NO is 1:2 and 1:2.5, the reduction time is shortened to 1 h and the content of D2 is greater than 80%.

### Examples I.72-I.101 and Comparative Examples I.15-I.22: Preparation of Trastuzumab-[MC-VC-PAB-MMAE]₂ Conjugate in Different Buffer System

When the reductant is TCEP, the preparation of Trastuzumab-[MC-VC-PAB-MMAE]₂ conjugate is the similar to example I.1. When the reductant is not TCEP, the preparation of Trastuzumab-[MC-VC-PAB-MMAE]₂ conjugate is similar to example I.4 But those adjust the buffer system (the buffer system are commercially available from Macklin) which are shown in table I-7.

The homogeneity assay results of examples I.72-I.101 and comparative examples I.15-I.22 were showed in Table I-7 and the chromatographic peak areas were showed in table I-8.

TABLE I-7

| | | The buffer | pH | | | | | |
|---|---|---|---|---|---|---|---|---|
| No. | Reductant | system | value | D0(wt %) | D1(wt %) | D2(wt %) | D4(wt %) | D6(wt %) |
| E I.72 | TCEP | MOPS buffer | 6.7 | 2.6 | 0 | 79.47 | 17.93 | 0 |
| E I.73 | TCEP | MOPS buffer | 7.0 | 3.66 | 0 | 80.03 | 16.3 | 0 |
| E I.74 | TCEP | MOPS buffer | 7.4 | 1.8 | 0 | 75.37 | 22.84 | 0 |
| E I.75 | TCEP | Bis-Tris buffer | 6.7 | 6.25 | 0 | 81.57 | 12.18 | 0 |
| E I.76 | TCEP | PIPES buffer | 6.7 | 8.41 | 0 | 81.08 | 10.51 | 0 |
| E I.77 | TCEP | BES buffer | 6.7 | 3.47 | 0 | 77.61 | 18.92 | 0 |
| E I.78 | TCEP | MES buffer | 6.7 | 2.64 | 0 | 76.79 | 20.56 | 0 |
| E I.79 | TCEP | HEPES buffer | 6.7 | 15.42 | 0 | 77.8 | 6.78 | 0 |
| E I.80 | TCEP | DIPSO buffer | 7.4 | 10.48 | 0 | 66.33 | 23.19 | 0 |
| E I.81 | TCEP | MOPSO buffer | 7.4 | 12.08 | 0 | 66.25 | 21.67 | 0 |
| E I.82 | TCEP | TES buffer | 7.4 | 15.96 | 0 | 64.79 | 19.25 | 0 |
| E I.83 | TCEP | ACES buffer | 7.4 | 14.81 | 0 | 60.34 | 22.81 | 2.04 |
| E I.84 | TCEP | MES buffer | 5.8 | 18.45 | 5.69 | 64.47 | 11.39 | 0 |
| E I.85 | TCEP | MES buffer | 6.4 | 9.45 | 0 | 79.90 | 10.65 | 0 |
| C I.15 | TCEP | PB buffer | 5.8 | 76.09 | 0 | 23.91 | 0 | 0 |
| C I.16 | TCEP | PB buffer | 6.2 | 77.93 | 0 | 22.07 | 0 | 0 |
| C I.17 | TCEP | PB buffer | 6.7 | 80.3 | 0 | 19.7 | 0 | 0 |
| C I.18 | TCEP | PB buffer | 7.0 | 75.07 | 0 | 24.93 | 0 | 0 |
| C I.19 | TCEP | PB buffer | 7.4 | 62.8 | 0 | 35.85 | 1.34 | 0 |
| C I.20 | TCEP | ADA buffer | 6.7 | 40.06 | 0 | 51.99 | 7.95 | 0 |
| C I.21 | TCEP | MOBS buffer | 7.4 | 31.01 | 0 | 53.63 | 15.36 | 0 |
| C I.22 | TCEP | TAPSO buffer | 7.4 | 25.42 | 0 | 50.24 | 22.07 | 2.27 |
| E I.86 | TCEP-NO | Bis-Tris buffer | 6.7 | 5.76 | 0 | 87.99 | 6.25 | 0 |
| E I.87 | TCEP-NO | PIPES buffer | 6.7 | 5.83 | 0 | 88.16 | 6.01 | 0 |
| E I.88 | TCEP-NO | MOPS buffer | 6.7 | 4.23 | 0 | 89.95 | 5.83 | 0 |
| E I.89 | TCEP-NO | BES buffer | 6.7 | 2.71 | 0 | 89.57 | 7.72 | 0 |
| E I.90 | TCEP-NO | HEPES buffer | 6.7 | 3.59 | 0 | 91.75 | 4.65 | 0 |
| E I.91 | TCEP-NO | ADA buffer | 6.7 | 39.6 | 0 | 51.8 | 8.6 | 0 |
| E I.92 | TCEP-NO | PB | 6.7 | 42.99 | 0 | 54.23 | 2.77 | 0 |
| E I.93 | TCEP-NO | DIPSO buffer | 7.4 | 34.18 | 0 | 53.49 | 12.33 | 0 |
| E I.94 | TCEP-NO | MOBS buffer | 7.4 | 31.2 | 0 | 56.64 | 12.16 | 0 |
| E I.95 | TCEP-NO | MOPSO buffer | 7.4 | 19.71 | 0 | 70.93 | 9.36 | 0 |
| E I.96 | TCEP-NO | TES buffer | 7.4 | 11.6 | 0 | 84.7 | 3.7 | 0 |
| E I.97 | TCEP-NO | ACES buffer | 7.4 | 23.33 | 0 | 68.1 | 8.57 | 0 |
| E I.98 | TCEP-NO | TAPSO buffer | 7.4 | 7.98 | 0 | 88.41 | 3.62 | 0 |
| E I.99 | TCEP-NO | MES buffer | 5.8 | 13.40 | 0 | 74.51 | 12.09 | 0 |
| E I.100 | TCEP-NO | MES buffer | 6.4 | 7.14 | 0 | 85.48 | 7.38 | 0 |
| E I.101 | TCEP-NO | MES buffer | 6.7 | 6.18 | 0 | 87.57 | 6.25 | 0 |

TABLE I-8

| | D0 area | D1 area | D2 area | D4 area | D6 area |
|---|---|---|---|---|---|
| Example | (mAU) | (mAU) | (mAU) | (mAU) | (mAU) |
| E I.72 | 53.76 | 0 | 1643.14 | 370.73 | 0 |
| E I.73 | 71.57 | 0 | 1564.88 | 318.73 | 0 |
| E I.74 | 45.82 | 0 | 1918.38 | 581.34 | 0 |
| E I.75 | 271.43 | 0 | 3542.49 | 528.96 | 0 |
| E I.76 | 260.68 | 0 | 2513.15 | 325.77 | 0 |
| E I.77 | 60.45 | 0 | 1352.11 | 329.62 | 0 |
| E I.78 | 59.17 | 0 | 1721.14 | 460.82 | 0 |
| E I.79 | 306.30 | 0 | 1545.39 | 134.68 | 0 |
| E I.80 | 242.21 | 0 | 1533.01 | 535.96 | 0 |
| E I.81 | 498.54 | 0 | 2734.11 | 894.31 | 0 |
| E I.82 | 796.27 | 0 | 3232.46 | 960.41 | 0 |
| E I.83 | 542.95 | 0 | 2212.12 | 836.24 | 74.79 |
| E I.84 | 460.59 | 141.66 | 1607.05 | 284.02 | 0 |
| E I.85 | 291.28 | 0 | 2463.98 | 328.53 | 0 |
| C I.15 | 1951.07 | 0 | 613.09 | 0 | 0 |
| C I.16 | 3419.34 | 0 | 968.37 | 0 | 0 |
| C I.17 | 556.45 | 0 | 136.51 | 0 | 0 |
| C I.18 | 2711.30 | 0 | 900.39 | 0 | 0 |
| C I.19 | 2776.63 | 0 | 1585.07 | 59.25 | 0 |
| C 1.20 | 1386.71 | 0 | 1799.68 | 275.20 | 0 |
| C I.21 | 1045.91 | 0 | 1808.85 | 518.07 | 0 |
| C I.22 | 1269.53 | 0 | 2509.10 | 1102.23 | 113.37 |
| E I.86 | 217.39 | 0 | 3320.81 | 235.88 | 0 |
| E I.87 | 232.06 | 0 | 3509.16 | 239.22 | 0 |
| E I.88 | 138.30 | 0 | 2940.91 | 190.61 | 0 |
| E I.89 | 64.55 | 0 | 2133.53 | 183.89 | 0 |
| E 1.90 | 56.84 | 0 | 1452.78 | 73.63 | 0 |
| E I.91 | 1238.97 | 0 | 1620.67 | 269.07 | 0 |
| E I.92 | 1963.91 | 0 | 2477.39 | 126.54 | 0 |
| E I.93 | 785.65 | 0 | 1229.51 | 283.41 | 0 |
| E I.94 | 1070.04 | 0 | 1942.53 | 417.04 | 0 |
| E I.95 | 322.41 | 0 | 1160.27 | 153.11 | 0 |
| E I.96 | 517.67 | 0 | 3779.89 | 165.12 | 0 |
| E I.97 | 832.57 | 0 | 2430.27 | 305.84 | 0 |
| E I.98 | 302.63 | 0 | 3352.86 | 137.28 | 0 |
| E I.99 | 482.75 | 0 | 2683.96 | 435.30 | 0 |
| E I.100 | 284.24 | 0 | 3401.30 | 293.64 | 0 |
| E I.101 | 224.25 | 0 | 3175.51 | 226.59 | 0 |

As the results shown in Table I-7, the buffer system affected the reduction kinetics and selectivity. The results showed that the buffer system and the pH value thereof impacted the D2 ratio and the homogeneity of conjugates.

When using reductant TCEP, MOPS buffer, Bis-Tris buffer, PIPES buffer, BES buffer, MES buffer, HEPES buffer, DIPSO buffer, MOPSO buffer, TES buffer, and ACES buffer helped to increase the D2 ratio to at least 60%, even to more than 65%, 70%, 75% and 80%.

When using reductant TCEP-NO, D2 ratio was up to 50% in ADA buffer, PB, DIPSO buffer MOBS buffer, and ACES buffer. D2 ratio was at least 70% in buffer system Bis-Tris buffer, PIPES buffer, MOPS buffer, BES buffer, HEPES buffer, MOPSO buffer, TES buffer, and TAPSO buffer.

### Examples I.102-I.123: Preparation of Trastuzumab-[MC-VC-PAB-MMAE]₂ Conjugate (the Reduction Time or (Temperature in Step (1) is Different)

When the reductant is TCEP, the preparation of Trastuzumab-[MC-VC-PAB-MMAE]₂ conjugate is the similar to example I.1. When the reductant is not TCEP, the preparation of Trastuzumab-[MC-VC-PAB-MMAE]₂ conjugate is similar to example I.4. But those adjust the reduction time or temperature in step (1) which are shown in table I-9.

The homogeneity assay results of examples 0.102-5.123 were showed in Table 0-9 and the chromatographic peak areas were showed in table I-10.

TABLE I-9

| | | temperature | Time | | | | |
|---|---|---|---|---|---|---|---|
| | | in step | in step | D0 | D2 | D4 | D6 |
| No. | Reductant | (1) (° C.) | (1)(h) | (%) | (%) | (%) | (%) |
| E I.102 | TCEP | 15 | 4 | 7.87 | 79.88 | 12.24 | 0 |
| E I.103 | TCEP | 24 | 4 | 6.66 | 78.73 | 14.61 | 0 |
| E I.104 | TCEP | 37 | 4 | 9.09 | 75.89 | 15.02 | 0 |
| E I.105 | TCEP | 4 | 1 | 17.36 | 75.30 | 7.33 | 0 |
| E I.106 | TCEP | 4 | 2 | 15.32 | 76.50 | 8.17 | 0 |
| E I.107 | TCEP | 4 | 3 | 9.47 | 80.64 | 9.90 | 0 |
| E I.108 | TCEP | 4 | 4 | 7.38 | 83.34 | 9.27 | 0 |
| E I.109 | TCEP | 4 | 5 | 8.03 | 81.64 | 10.33 | 0 |
| E I.110 | TCEP | 4 | 6 | 9.98 | 80.53 | 9.49 | 0 |
| E I.111 | TCEP | 4 | 8 | 5.99 | 83.28 | 10.73 | 0 |
| E I.112 | TCEP-NO | 15 | 4 | 8.88 | 83.16 | 7.96 | 0 |
| E I.113 | TCEP-NO | 24 | 4 | 9.32 | 81.24 | 9.45 | 0 |
| E I.114 | TCEP-NO | 37 | 4 | 9.29 | 80.73 | 9.98 | 0 |
| E I.115 | TCEP-NO | 4 | 0.25 | 15.98 | 80.25 | 3.77 | 0 |
| E I.116 | TCEP-NO | 4 | 0.5 | 10.89 | 84.77 | 4.34 | 0 |
| E I.117 | TCEP-NO | 4 | 0.75 | 9.63 | 85.52 | 4.85 | 0 |
| E I.118 | TCEP-NO | 4 | 1 | 7.86 | 86.76 | 5.38 | 0 |
| E I.119 | TCEP-NO | 4 | 2 | 7.36 | 87.41 | 5.23 | 0 |
| E I.120 | TCEP-NO | 4 | 3 | 6.89 | 87.46 | 5.66 | 0 |
| E I.121 | TCEP-NO | 4 | 4 | 7.41 | 87.29 | 5.30 | 0 |
| E I.122 | TCEP-NO | 4 | 5 | 6.57 | 87.86 | 5.58 | 0 |
| E I.123 | TCEP-NO | 4 | 6 | 6.54 | 87.19 | 6.27 | 0 |

TABLE I-10

| | D0 area | D2 area | D4 area | D6 area |
|---|---|---|---|---|
| No. | (mAU) | (mAU) | (mAU) | (mAU) |
| E I.102 | 308.93 | 3134.31 | 480.32 | 0 |
| E I.103 | 238.10 | 2815.36 | 522.61 | 0 |
| E I.104 | 289.13 | 2414.44 | 477.86 | 0 |
| E I.105 | 508.99 | 2207.41 | 214.92 | 0 |
| E I.106 | 349.05 | 1742.73 | 186.20 | 0 |
| E I.107 | 287.87 | 2452.49 | 301.00 | 0 |
| E I.108 | 240.58 | 2715.29 | 302.09 | 0 |
| E I.109 | 171.58 | 1743.83 | 220.72 | 0 |
| E I.110 | 378.17 | 3051.60 | 359.47 | 0 |
| E I.111 | 212.87 | 2961.93 | 381.67 | 0 |
| E I.112 | 332.71 | 3116.86 | 298.30 | 0 |
| E I.113 | 354.14 | 3087.96 | 359.03 | 0 |
| E I.114 | 368.62 | 3204.64 | 396.22 | 0 |
| E I.115 | 599.85 | 3012.32 | 141.52 | 0 |
| E I.116 | 363.94 | 2833.25 | 145.01 | 0 |
| E I.117 | 363.05 | 3224.31 | 182.86 | 0 |
| E I.118 | 274.03 | 3025.67 | 187.52 | 0 |
| E I.119 | 191.88 | 2278.38 | 136.44 | 0 |
| E I.120 | 286.35 | 3636.54 | 235.19 | 0 |
| E I.121 | 266.17 | 3136.98 | 190.56 | 0 |
| E I.122 | 274.98 | 3679.63 | 233.64 | 0 |
| E I.123 | 245.87 | 3279.29 | 235.75 | 0 |

As shown in example I.1, and examples I.102-I.104, using the TCEP as reductant, the content of D2 is up to 75%, even to 80% when the reductant temperature in step (1) is from 4° C. to 37° C. As shown in examples I.105-I.111, using the TCEP as reductant, the content of D2 is up to 75%, 80%, even to 83% when the reductant time in step (1) is from l h to 8 h. The content of D2 increases when the reduction time in step (1) is from 1 h to 4 h, and it reaches a plateau after 4 h.

As the results shown in examples I.112-I.123, using TCEP-NO as reductant, when the reduction temperature is 4-37° C. and the reduction time is 0.25 h to 37 h, the content of the ADC with D2 is up to 80%, the content of D2 increases as the reduction time of step (1) from 0.25 h to 1 h, and reaches plateau after I h, indicating a very fast reaction kinetics.

### Example I.124: Preparation of Trastuzumab-[MC-VC-PAB-MMAE]₂ Conjugate by the Engineered antibody

The preparation of Trastuzumab-[MC-VC-PAB-MMAE]₂ conjugate is similar to the preparation of ADC of example I.4, but it used the engineered antibody.

The engineered antibody is the mutant of trastuzumab by replacing disulfide bonds in-between heavy-light chain through cysteine to serine mutation (Order from Biointron).

The homogeneity assay result and the chromatographic peak areas of example I.124 were showed in table I-11.

TABLE I-11

| | | | | | D0 | D2 | D4 |
|---|---|---|---|---|---|---|---|
| | The engineered | | | | area | area | area |
| No. | antibody | D0(%) | D2(%) | D4(%) | (mAU) | (mAU) | (mAU) |
| E I.124 | cysteine to | 0 | 95.85 | 4.15 | 0 | 1993.32 | 86.35 |
| | serine mutation | | | | | | |

As the results shown in the table I-11, the content of D2 prepared by the engineered antibody is as high as 96%. Those results indicated that this method is also applied to antibodies with simple mutations and might have even better reduction selectivity in some mutant antibodies.

### Examples I.125-I-126: preparation of Trastuzumab-[Maleimide-PEG4-N3-DBCO-Cy3]1 Conjugate (The ADC with D1)

1. Synthesis of dibromomaleimide-PEG4-N3

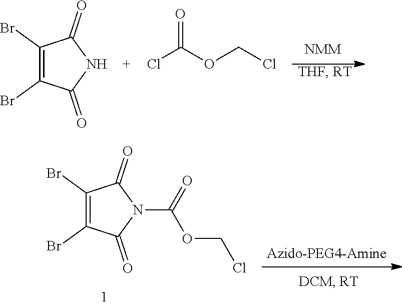 [SMILES: O=C1NC(=O)C(Br)=C1Br ; O=C(Cl)OCCl ; O=C(OCCl)N1C(=O)C(Br)=C(Br)C1=O]

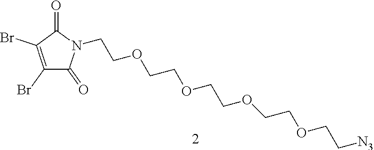 [SMILES: [N-]=[N+]=NCCOCCOCCOCCOCCN1C(=O)C(Br)=C(Br)C1=O]

To a solution of 3,4-dibromomaleimide (127 mg, 0.5 mmol, 1 eq) and N-methylmorpholine (0.22 mL, 2 mmol, 4 eq) in THF (3.5 mL), chloromethyl chloroformate (0.18 mL, 2 mmol, 4 eq) was added and the mixture was stirred for 20 min at room temperature. Then DCM (10 mL) was added, the organic phase was washed with H₂O, dried over MgSO₄ and the solvent removed in vacuo to yield the title product 1 (139 mg, 0.4 mmol, 80%).

A solution of Azido-PEG4-Amine (105 mg, 0.4 mmol, 1 eq) in dichloromethane (2 mL) was added to a stirred solution of product 1 (139 mg, 0.4 mmol, 1 eq) in dichloromethane (2 mL).

After 30 minutes, dichloromethane (6 mL) was added and the solution washed with a 0.68 M acetate buffer pH 5 (10 mL), water (1 mL), and dried with MgSO₄. Concentration in vacuo followed by purification by column chromatography (100% EtOAc as the mobile phase) yielded dibromomaleimide-PEG4-N3 as a pale-yellow oil (150 mg, 0.3 mmol, 75%).

### 2. Preparation of Trastuzumab-Maleimide-[PEG4-N3-DBCO-Cy3]₁

(1) incubating reductant TCEP/TCEP-NO (0.02 mM) and trastuzumab (0.012 mM) in the presence of ZnCl₂ (0.24 mM) in MES (20 mM, pH6.7), and the reaction mixture was allowed to stay at 4° C. for 4 h;
(2) introducing EDTA (0.6 mM) and dibromomaleimide-PEG4-N3 (0.013 mM) to react with reduced thiol groups resulted from step (1) at 24° C. for 3 h, then recovering the product using a desalting column to afford Trastuzumab-[Maleimide-PEG4-N3]₁;
(3) incubating Trastuzumab-[Maleimide-PEG4-N3]₁ and DBCO-Cy3 (0.02 mM) in MES (20 mM, pH6.7) at 25° C. for 8 h, then recovering Trastuzumab-Maleimide-PEG4-N3-DBCO-Cy3 using a desalting column.

The chromatographic peak area and the homogeneity assay result of examples I.125-I.126 were shown in Table I-12.

TABLE I-12

| | | D0 Area | D1 Area | D2 Area | D0 | D1 | D2 | |
|---|---|---|---|---|---|---|---|---|
| No. | Reductant | (mAU) | (mAU) | (mAU) | (wt %) | (wt %) | (wt %) | Figure |
| E I.125 | TCEP | 243.05 | 916.03 | 19.72 | 20.62 | 77.71 | 1.67 | 3 |
| E I.126 | TCEP-NO | 197.64 | 1502.28 | 96.85 | 11.0 | 83.61 | 5.39 | 4 |

As shown in table I-12, the results demonstrated Maleimide-PEG4-N3-DBCO-Cy3 was successfully linked to Trastuzumab. Further, the content of the ADC with D1 is generally up to 77.71% using TCEP, and D1 ratio was up to 83.61% using TCEP-NO.

### Example I.127: preparation of Trastuzumab-[Maleimide][MC-GGFG-DXd]₆ (the ADC with D0+D6)

(1) incubating the first reductant TCEP-NO (0.02 mM) and trastuzumab (0.012 mM) in the presence of ZnCl₂ (0.24 mM) in MES (20 mM, pH6.7) at 4° C. for 4 h;
(2) introducing EDTA (0.6 mM) and dibromomaleimide (0.013 mM) to react with reduced thiol groups resulted from step (1), the reaction condition was 24° C., 3 h, then recovering the product using a desalting column to afford Trastuzumab-[Maleimide]₁;
(3) introducing Trastuzumab-[Maleimide]₁ and TCEP (0.08 mM) in MES (20 mM, pH6.7), the reaction temperature was 25° C. and the reaction time was 12 h;
(4) introducing MC-GGFG-DXd (0.14 mM) to solution from step (3), and the reaction mixture was allowed to stay at 24° C. for 1 h, then recovering Trastuzumab-[Maleimide]₁[MC-GGFG-DXd]₆ using a desalting column.

### Example I.128: Preparation of Trastuzumab-[Maleimide]₁[MC-VC-PAB-MMAE]₆ (the ADC with D0+D6)

the method is the similar to example I.127. The differences are that the reductant in example I.128 is TCEP in step (1) and the second linker-payload is MC-VC-PAB-MMAE in step (4) in example 1.128.

The homogeneity assay results and the chromatographic peak area and of examples I.127-I.128 were shown in Table I-13 and Table I-14.

TABLE I-13

| No. | FIG. | Reductant | D0(%) | / | / |
|---|---|---|---|---|---|
| E I.127-step (2) | 5 | TCEP-NO | 100 | | |
| E I.128-step (2) | 7A | TCEP | 100 | / | / |
| No. | FIG. | Reductant | D0 + D4 (%) | D0 + D6 (%) | D8 (%) |
| E I.127-step (4) | 6 | TCEP-NO | 4.17 | 84.68 | 11.16 |
| E I.128-step (4) | 7B | TCEP | 19.33 | 73.50 | 7.17 |

TABLE I-14

| | D0 area | | |
|---|---|---|---|
| No. | (mAU) | / | / |
| E I.127-step(2) | 1547.94 | / | / |
| E I.128-step (2) | 1441.26 | / | / |
| | D0 + D4 area | D0 + D6 area | D8 (%) area |
| No. | (mAU) | (mAU) | (mAU) |
| E I.127-step(4) | 85.08 | 1727.80 | 227.71 |
| E I.128-step (4) | 1278.64 | 4861.13 | 474.45 |

The results demonstrated that the bi-payload conjugate was successfully synthesized. Further, the homogeneity of the ADC with D0+D6 was generally up to 84.68% using TCEP-NO, and the content of the ADC with D0+D6 is generally up to 73.50% using TCEP. It showed that the method of the present application could modify the antibody with site-specific and prepare the different kinds of ADCs with improving the homogeneity.

### Example I.129: Preparation of Bi-Payload Conjugate Trastuzumab-[MC-VC-PAB-MMAE]₂[MC-GGFG-DXd]₆ (The ADC with D2+D6)

(1) incubating the first reductant TCE (0.02 mM) and trastuzumab (0.012 mM) in the presence of ZnCl₂ (0.24 mM) in MES (20 mM, pH6.7) at 4° C. for 4 h;
(2) introducing EDTA (0.6 mM) and an excess amount of MC-VC-PAB-MMAE (0.06 mM) to react with reduced thiol groups resulted from step (1), the reaction temperature was 24° C. and the reaction time was 1 h, then recovering the product using a desalting column to afford Trastuzumab-[MC-VC-PAB-MMAE]₂;
(3) introducing Trastuzumab-[MC-VC-PAB-MMAE]₂ and TCEP (0.08 mM) in MES (20 mM, pH6.7), the reaction temperature was 25° C. and the reaction time was 8 h;
(4) introducing MC-GGFG-DXd (0.14 mM) to solution from step (3), and the reaction mixture was allowed to stay at 24° C. for 1 h, then recovering the resultant Trastuzumab-[MC-VC-PAB-MMAE]₂[MC-GGFG-DXd]₆ using a desalting column.

### Example I.130: preparation of bi-payload conjugate Trastuzumab-[MC-MMAF]₂[MC-GGFG-DXd]₆ (The ADC with D2+D6)

The method is similar to example I.129. The differences are the reductant is TCEP-NO in step (1) and the linker-payload is MC-MMAF in step (2).

The homogeneity assay results and the chromatographic peak area of examples I.129-I.130 were shown in Table I-15 and Table I-16.

TABLE I-15

| | | D0 | D2 | D4 | | |
|---|---|---|---|---|---|---|
| No. | Reductant | (wt %) | (wt %) | (wt %) | / | Figure |
| E I.129-step (2) | TCEP | 3.49 | 83.29 | 13.22 | / | 8A |
| E I.130-step (2) | TCEP-NO | 3.01 | 87.17 | 9.81 | | 9 |
| | | D2 + D4/ | D8 | D2 + | D4 + | |
| No. | Reductant | D4 + D2(%) | (%) | D6 (%) | D4 (%) | Figure |
| E I.129-step (4) | TCEP | 16.91 | 3.19 | 72.43 | 7.47 | 8B |
| | | D2 + D4/ | D2 + D6 | D4 + D4/ | | |
| No. | Reductant | D4 + D2 (%) | (%) | D0 + D8(%) | / | Figure |
| E I.130-step (4) | TCEP-NO | 7.49 | 81.31 | 11.2 | 1 | 10 |

TABLE I-16

| | D0 area | D2 area | D4 area | |
|---|---|---|---|---|
| No. | (mAU) | (mAU) | (mAU) | / |
| E I.129-step (2) | 146.92 | 3510.41 | 3.49 | / |
| E I.130-step (2) | 93.85 | 2715.10 | 305.66 | / |
| | D2 + D4/ | | D2 + | D4 + |
| | D4 + D2 area | D8 area | D6 area | D4 area |
| No. | (mAU) | (mAU) | (mAU) | (mAU) |
| E I.129-step (4) | 272.26 | 51.43 | 1166.44 | 120.34 |
| | D2 + D4/ | D2 + | D4 + D4/ | |
| | D4 + D2 area | D6 area | D0 + D8 area | |
| No. | (mAU) | (mAU) | (mAU) | / |
| E I.130-step (4) | 77.54 | 841.80 | 115.95 | / |

As shown in table I-15, the result demonstrated that the content of the ADC with D2+D6 is generally up to 72.43% using TCEP and the content of the ADC with D2+D6 is generally up to 81.31%, using TCEP-NO which indicated the process of method is benefit for site-specific modifying the antibody with D2+D6 and improving the homogeneity.

### Examples I.131-I.132: Preparation of Trastuzumab-[MC-VC-PAB-MMAE]₂[MC-GGFG-DXd]₂ (The ADC with D2+D2)

(1) incubating the first reductant TCEP-NO (0.02 mM) and trastuzumab (0.012 mM) in the presence of an effective amount of ZnCl₂ (0.12 mM) in BES (20 mM, pH7.0), The incubation temperature is 4° C. and the incubation time is 4 h;
(2) introducing EDTA (0.6 mM) and MC-VC-PAB-MMAE (0.048 mM) to react with reduced thiol groups resulted from step (1), the reaction temperature is 24° C. and the reaction time is 1 h, then recovering the product using a desalting column to afford Trastuzumab-[MC-VC-PAB-MMAE]₂;
(3) incubating ZnCl₂ (1.2 mM), the second reductant TCEP-3 (0.0144 mM)/TCEP-6 (0.0216 mM) and the product from step (2) in BES buffer (pH7.0, 20 mM) and the reaction mixture was allowed to stay at 4° C. for 4 h;
(4) introducing EDTA (3 mM) to trap Zn²⁺, and introducing MC-GGFG-DXd (0.1 mM) to react with the reduced thiol groups resulted from step (3), the reaction temperature is 24° C. and the reaction time is 1 h;
(5) the reaction mixture was subjected to purification using a desalting column.

The homogeneity assay result and the chromatographic peak area of examples I.131-I.132 were shown in Table I-17 and Table I-18.

TABLE I-17

| No. | Figure | Reductant | D0(%) | D2 (%) | D4(%) | / | / | / | / |
|---|---|---|---|---|---|---|---|---|---|
| E I.131- | 11A | TCEP-NO | 4.99 | 90.38 | 4.63 | / | / | / | / |
| step (2) | | | | | | | | | |
| No. | Figure | Reductant | D2 | D4 | D2 | D2 | | D4 | |
| | | | (DXd) | (DXd) | (MMAE)/ | (MMAE) + | D2 + D4/ | (MMAE) | D4 + |
| | | | (%) | (%) | D6(DXd)% | D2 (DXd) (%) | D2 + D6(%) | (%) | D4 (%) |
| E I.131- | 11B | TCEP-3 | 4.32 | 2.83 | 9.59 | 68.47 | 10.80 | 1.73 | 2.26 |
| step (5) | | | | | | | | | |
| E I.132- | 11C | TCEP-6 | 4.95 | 2.06 | 6.77 | 70.55 | 12.50 | / | 3.18 |
| step (5) | | | | | | | | | |

TABLE I-18

| | D0 area | D2 area | D4 area | | | | |
|---|---|---|---|---|---|---|---|
| No. | (mAU) | (mAU) | (mAU) | / | / | / | / |
| E I. 131-step (2) | 97.92 | 1775.44 | 91.01 | / | / | / | / |
| | | | D2 | D2 (MMAE) + | | | |
| | D2 | D4 | (MMAE)/ | D2 | D2 + D4/ | D4 | |
| | (DXd) | (DXd) | D6(DXd) | (DXd) | D2 + D6 | (MMAE) | D4 + D4 |
| | area | area | area | area | area | area | area |
| No | (mAU) | (mAU) | (mAU) | (mAU) | (mAU) | (mAU) | (mAU) |
| E I.131-step (5) | 45.37 | 29.72 | 100.84 | 719.67 | 113.55 | 18.13 | 23.76 |
| E I.132-step (5) | 64.81 | 26.88 | 88.54 | 922.91 | 163.50 | / | 41.62 |

In step (3) of examples I.131 and 1.132, one of the interchain disulfide bonds in the ADC with D2 was reduced. As shown in table I-17, the results demonstrate that the content of the ADC with D2+D2 is generally up to 68% or 70%, which indicated the process of method was benefit for site-specific modifying the antibody with D2+D2 and improving the homogeneity.

### Example I.133: preparation of Trastuzumab-[Maleimide-PEG4-N3-DBCO-Cy3]1[MC-VC-PAB-MMAE]₂ (the ADC with D1+D2)

(1) incubating the first reductant TCEP-NO (0.02 mM) and trastuzumab (0.012 mM) in the presence of an effective amount of ZnCl₂ (0.24 mM) in BES (20 mM, pH7.0), The incubation temperature is 4° C. and the incubation time is 4 h;
(2) introducing EDTA (0.6 mM) and dibromomaleimide-PEG4-N3 (0.013 mM) to react with reduced thiol groups resulted from step (1), the reaction temperature is 25° C. and the reaction time is 1 h, then recovering the product using a desalting column to afford Trastuzumab-[Maleimide-PEG4-N3]2;
(3) incubating Trastuzumab-[Maleimide-PEG4-N3]₁ from step (2) and DBCO-Cy3 (0.02 mM) in MES (20 mM, pH6.7), the reaction temperature is 25° C. and the reaction time is 6 h, then recovering Trastuzumab-[Maleimide-PEG4-N3-DBCO-Cy3]₁ using a desalting column.
(4) incubating ZnCl₂ (1.2 mM), the second reductant TCEP-3 (0.0144 mM) and the product prepared from step (3) in BES buffer (pH7.0, 20 mM) and the reaction mixture was allowed to stay at 4° C. for 4 h;
(5) introducing EDTA (3 mM) to trap Zn²⁺, and introducing MC-VC-PAB-MMAE (0.1 mM) to react with the reduced thiol groups resulted from step (4), the reaction temperature is 25C and the reaction time is 1 h;
(6) the reaction mixture was subjected to purification using a desalting column.

The homogeneity assay result and the chromatographic peak area of example I.133 were shown in Table I-19 and Table I-20.

TABLE I-19

| No. | Figure | D1(%) | D2 (%) | D1 + D2 (%) | D4% | D2 + D4 (%) |
|---|---|---|---|---|---|---|
| E I.133 | 12 | 6.87 | 3.04 | 83.21 | 3.06 | 3.82 |

TABLE I-20

| | D1 area | D2 area | D1 + D2 area | D4 area | D2 + D4 area |
|---|---|---|---|---|---|
| No. | (mAU) | (mAU) | (mAU) | (mAU) | (mAU)) |
| E I.133 | 63.60 | 28.13 | 769.99 | 28.29 | 35.30 |

As shown in table I-19, the results demonstrate that the content of the ADC with D1+D2 is generally up to 80% or 83%, which indicated the process of method was benefit for site-specific modifying the antibody with D1+D2 and improving the homogeneity.

### Examples I.134-I.135: Preparation of Trastuzumab-[Maleimide]₁[MC-VC-PAB-MMAE]₄ (the ADC with D0+D4)

(1) incubating the first reductant TCEP-NO (0.02 mM) and trastuzumab (0.012 mM) in the presence of an effective amount of ZnCl₂ (0.24 mM) in MES (20 mM, pH6.7), The incubation temperature is 4° C. and the incubation time is 4 h;
(2) introducing EDTA (0.6 mM) and dibromomaleimide (0.013 mM) to react with reduced thiol groups resulted from step (1), the reaction temperature is 24° C. and the reaction time is 3 h, then recovering the product using a desalting column to afford Trastuzumab-[Maleimide]₁;
(3) incubating ZnCl₂ (0.36 mM), the second reductant TCEP-3 (0.036 mM) or TCEP-6 (0.048 mM) and the product from step (2) in BES buffer (pH7.0, 20 mM) and the reaction mixture was allowed to stay at 4° C. for 16 h;
(4) introducing EDTA (0.6 mM) to trap Zn²⁺, and introducing MC-VC-PAB-MMAE (0.1 mM) to react with the reduced thiol groups resulted from step (4), the reaction temperature is 24° C. and the reaction time is 1 h;
(5) the reaction mixture was subjected to purification using a desalting column.

The homogeneity assay result and the chromatographic peak area of examples I.134 and I.135 were shown in Table I-21 and Table I-22.

TABLE I-21

| No. | FIG. | Reductant | D0(%) | / | / |
|---|---|---|---|---|---|
| E I.134-step (2) | 13A | TCEP-NO | 100 | / | / |
| | | | D0 + D2 | D0 + D4 | D0 + D6 |
| No. | FIG. | / | (%) | (%) | (%) |
| E I.134-step (5) | 13B | TCEP-3 | 18.96 | 61.27 | 19.77 |
| E I.135-step (5) | 13C | TCEP-6 | 21.23 | 61.85 | 16.92 |

TABLE I-22

| | D0 area | | |
|---|---|---|---|
| No. | (mAU) | / | / |
| E I.134-step (2) | 3861.77 | / | / |
| | D0 + D2 area | D0 + D4 area | D0 + D6 area |
| No. | (mAU) | (mAU) | (mAU) |
| E I.134-step (5) | 318.31 | 1028.36 | 331.83 |
| E I.135-step (5) | 420.07 | 1223.85 | 334.85 |

In step (3) of examples I.134-I.135, two of the interchain disulfide bonds in the ADC with D2 was reduced. As shown in table I-21, the results demonstrate that the content of the ADC with D0+D4 is generally up to 55% or 61%, which indicated the process of method was benefit for site-specific modifying the antibody with D0+D4 and improving the homogeneity.

### Examples I.136-I.137: Preparation of Trastuzumab-[MC-GGFG-DXd]₂[MC-VC-PAB-MMAE]₄ (the ADC with D2+D4)

(1) incubating the first reductant TCEP-NO (0.02 mM) and trastuzumab (0.012 mM) in the presence of an effective amount of ZnCl₂ (0.24 mM) in BES (20 mM, pH7.0), The incubation temperature is 4° C. and the incubation time is 4 h;
(2) introducing EDTA (0.6 mM) and MC-GGFG-DXd (0.072 mM) to react with reduced thiol groups resulted from step (1), the reaction temperature is 24° C. and the reaction time is 1 h, then recovering the product using a desalting column to afford Trastuzumab-[MC-GGFG-DXd]₂;
(3) incubating ZnCl₂ (0.36 mM), the second reductant TCEP-3 (0.036 mM) or TCEP-6 (0.048 mM) and the product from step (2) in BES buffer (pH7.0, 20 mM) and the reaction mixture was allowed to stay at 4° C. for 16 h;
(4) introducing EDTA (0.6 mM) to trap Zn²⁺, and introducing MC-VC-PAB-MMAE (0.1 mM) to react with the reduced thiol groups resulted from step (4), the reaction temperature is 24° C. and the reaction time is 1 h;
(5) the reaction mixture was subjected to purification using a desalting column.

The homogeneity assay result and the chromatographic peak area of examples I.136 and I.137 were shown in Table I-23 and Table I-24.

TABLE I-23

| No. | Figure | Reductant | D0(%) | D2 (%) | / | D4 (%) | / |
|---|---|---|---|---|---|---|---|
| E I.136-step (2) | 14A | TCEP-NO | 3.99 | 87.26 | / | 8.75 | / |
| No. | Figure | Reductant | D2 + D2 (%) | D4 + D2 (%) | D2 + D4 (%) | D6 (%) | D4 + D4 (%) |
| E I.136-step (5) | 14B | TCEP-3 | 5.34 | 4.01 | 79.67 | 6.78 | 4.21 |
| E I.137-step (5) | 14C | TCEP-6 | 3.03 | 3.53 | 79.37 | 6.93 | 7.14 |

TABLE I-24

| | D0 area | D2 area | | D4 area | |
|---|---|---|---|---|---|
| No. | (mAU) | (mAU) | / | (mAU) | / |
| E I.136-step (2) | 111.89 | 2446.07 | / | 245.36 | / |
| | D2 + | D4 + | D2 + | | D4 + |
| | D2 area | D2 area | D4 area | D6 area | D4 area |
| No. | (mAU) | (mAU) | (mAU) | (mAU) | (mAU) |
| E I.136-step (5) | 51.55 | 38.67 | 769.19 | 65.42 | 40.60 |
| E I.137-step (5) | 24.85 | 28.95 | 650.66 | 56.79 | 58.50 |

As shown in table I-23, the results demonstrate that the content of the ADC with D2+D4 is generally up to 70%, 75%, even to 78% or 80%, which indicated the process of method was benefit for site-specific modifying the antibody with D2+D4 and improving the homogeneity.

### Example I.138: Preparation of Trastuzumab-[Maleimide-PEG4-N3-DBCO-Cy3]₁[MC-VC-PAB-MMAE]₄ (the ADC with D1+D4)

(1) incubating the first reductant TCEP-NO (0.02 mM) and trastuzumab (0.012 mM) in the presence of an effective amount of ZnCl₂ (0.24 mM) in BES (20 mM, pH7.0), The incubation temperature is 4° C. and the incubation time is 4 h;
(2) introducing EDTA (0.6 mM) and dibromomaleimide-PEG4-N3 (0.013 mM) to react with reduced thiol groups resulted from step (1), the reaction temperature is 25° C. and the reaction time is 6 h, then recovering the product using a desalting column to afford Trastuzumab-Maleimide-PEG4-N3;
(3) incubating Trastuzumab-Maleimide-PEG4-N3 and DBCO-Cy3 (0.02 mM) in MES (20 mM, pH6.7), the reaction temperature is 25° C. and the reaction time is 6 h, then recovering Trastuzumab-Maleimide-PEG4-N3-DBCO-Cy3 using a desalting column.
(4) incubating ZnCl₂ (0.36 mM), the second reductant TCEP-3 (0.0408 mM) and the product from step (3) in BES buffer (pH7.0, 20 mM) and the reaction mixture was allowed to stay at 4° C. for 16 h; (5) introducing EDTA (0.6 mM) to trap Zn²⁺, and introducing MC-VC-PAB-MMAE (0.1 mM) to react with the reduced thiol groups resulted from step (4), the reaction temperature is 24° C. and the reaction time is 1 h;
(6) the reaction mixture was subjected to purification using a desalting column.

The homogeneity assay result and the chromatographic peak area of example I.138 were shown in Table I-25 and Table I-26.

TABLE I-25

| No. | Figure | Reductant | D2 (%) | D1 + D2 (%) | D4 (%) | D1 + D4 (%) |
|---|---|---|---|---|---|---|
| E I.138 | 15 | TCEP-3 | 3.09 | 23.96 | 2.45 | 70.49 |

TABLE I-26

| | D2 area | D1 + D2 area | D4 area | D1 + D4 area |
|---|---|---|---|---|
| No. | (mAU) | (mAU) | (mAU) | (mAU) |
| E I.138 | 11.28 | 87.34 | 8.93 | 256.96 |

As shown in table I-25, the results demonstrate that the content of the ADC with D1+D4 is generally up to 60%, 65%, even to 70%, which indicated the process of method was benefit for site-specific modifying the antibody with D1+D4 and improving the homogeneity.

### Example I.139: Preparation of Trastuzumab-[MC-VC-PAB-MMAE]₂[MC-GGFG-DXd]₂[MC-MMAF] (the ADC with D2+D2+D2)

(1) incubating the first reductant TCEP-NO (0.02304 mM) and trastuzumab (0.012 mM) in the presence of an effective amount of ZnCl₂ (0.36 mM) in BES (20 mM, pH7.0), The incubation temperature is 4° C. and the incubation time is 3 h;
(2) introducing EDTA (0.72 mM) and MC-VC-PAB-MMAE (0.06 mM) to react with reduced thiol groups resulted from step (1), the reaction temperature is 24° C. and the reaction time is 1 h, then recovering the product using a desalting column to afford Trastuzumab-[MC-VC-PAB-MMAE]₂;
(3) incubating ZnCl₂ (1.2 mM), the second reductant TCEP-3 (0.02472 mM) and the product from step (2) in BES buffer (pH7.0, 20 mM) and the reaction mixture was allowed to stay at 4° C. for 2.5 h;
(4) introducing EDTA (2.4 mM) to trap Zn²⁺, and introducing MC-GGFG-DXd (0.048 mM) to react with the reduced thiol groups resulted from step (3), the reaction temperature is 24° C. and the reaction time is 1 h, the reaction mixture was subjected to purification using a desalting column and AKTA with HIC chromatography;
(5) incubating ZnCl₂ (1.2 mM), the second reductant TCEP-3 (0.0168 mM) and the product from step (4) in BES buffer (pH7.0, 20 mM) and the reaction mixture was allowed to stay at 4° C. for 6 h;
(6) introducing EDTA (2.4 mM) to trap Zn²⁺, and introducing MC-MMAF (0.048 mM) to react with the reduced thiol groups resulted from step (5), the reaction temperature is 24° C. and the reaction time is 1 h, the reaction mixture was subjected to purification using a desalting column.

The homogeneity assay result and the chromatographic peak area of example I.139 were shown in Table I-27 and Table I-28.

TABLE I-27

| No. | Figure | D0(%) | D2 (%) | D4(%) | / | / | / |
|---|---|---|---|---|---|---|---|
| E I.139-step (2) | 42A | 2.95 | 86.90 | 10.15 | / | / | / |
| | | D2 | D2 | | | D4 | |
| | | (DXd) | (MMAE) | D2 + D2 | D2 + D4 | (MMAE) | D4 + D2 |
| No. | Figure | (%) | (%) | (%) | (%) | (%) | (%) |
| E I.139-before | 42B | 1.93 | 15.45 | 61.97 | 11.78 | 4.89 | 3.99 |
| AKTA in step (4) | | | | | | | |
| E I.139- After | 42C | / | 10.79 | 89.22 | / | / | / |
| AKTA in step (4) | | | | | | | |
| | | D2 + D2 | D2 + D2 + | D2 + D2 + | | | |
| No. | Figure | (%) | D2 (%) | D4 (%) | / | / | / |
| E139-step (6) | 42D | 6.48 | 79.84 | 13.69 | / | / | / |

TABLE I-28

| | D0 area | D2 area | D4 area | | | |
|---|---|---|---|---|---|---|
| No. | (mAU) | (mAU) | (mAU) | / | / | / |
| E I. 139-step (2) | 94.55 | 2782.26 | 325.03 | / | / | / |
| | D2 | D2 | | | D4 | |
| | (DXd) | (MMAE) | D2 + D2 | D2 + D4 | (MMAE) | D4 + D2 |
| | area | area | area | area | area | area |
| No. | (mAU) | (mAU) | (mAU) | (mAU) | (mAU) | (mAU) |
| E I.139-before | 30.15 | 240.98 | 966.78 | 183.83 | 76.21 | 62.17 |
| AKTA in step (4) | | | | | | |
| E I.139- After | 1 | 57.32 | 474.15 | / | / | / |
| AKTA in step (4) | | | | | | |
| | D2 + D2 | D2 + D2 + | D2 + D2 + | / | / | / |
| | area | D2 area | D4 area | | | |
| No. | (mAU) | (mAU) | (mAU) | | | |
| E139-step (6) | 20.86 | 257.09 | 44.07 | | / | / |

As shown in table I-27, the results demonstrate that the content of the ADC with D2+D2+D2 is generally up to 75% or 80%, which indicated the process of method was benefit for site-specific modifying the antibody with D2+D2+D2 and improving the homogeneity.

### Example II.1: Preparation of Antibody-[MC-VC-PAB-MMAE]₆ (The ADC with D6)

(1) TCEP (0.048 mM) and ZnCl₂ (0.024 mM) were added to a solution of Trastuzumab (0.012 mM) in BES buffer (20 mM, pH7.0) and the reaction mixture was vortexed for mixing, then the reaction mixture was incubated at 4° C. for 18 h;
(2) EDTA (0.6 mM) and MC-VC-PAB-MMAE (0.08 mM) in DMA was introduced and the reaction was incubated at room temperature for 1 h;
(3) The reaction mixture was subjected to purification using a desalting column.

### Examples II.2-II.3: Preparation of Antibody-[MC-VC-PAB-MMAE]₆ (The ADC with D6)

The method of example II.2-II.3 is similar to example II.1, and the difference is that the antibody is Sacituzumab/Belantamab, the buffer system is MES buffer and the molar ratio of Zn²⁺/TCEP is 0.87:1.

### Example II.4.: Preparation of Antibody-[MC-VC-PAB-MMAE]₆ (The ADC with D6)

(1) ZnCl₂ (0.024 mM) and reductant TCEP-NO-Trtyl (0.048 mM) were added to a solution of a monoclonal antibody Trastuzumab (0.012 mM, in BES buffer, pH7.0, 20 mM) and the reaction mixture was allowed to stay at 4° C. for 18 h;
(2) EDTA-2Na (0.6 mM) and MC-VC-PAB-MMAE (0.096 mM) in DMA was introduced and the reaction was continued at room temperature for 1 h;
(3) The reaction mixture was subjected to purification using a desalting column.

### Examples II.5-II.29 and Comparative Example II.1: Preparation of Antibody-[MC-VC-PAB-MMAE]₆ (The ADC with D6)

The method of examples II.5-II.29 and comparative example II.1 was the same as example II.4, and the differences were the kinds of reductant and the antibody, the molar ratio of the reductant and the antibody, and/or the molar ratio of the molar ratio of the Zn²⁺ and the reductant, which are shown in Table II-1. Meanwhile, and the incubation time in step (1) is 16 h in examples II.27-II.29.

TABLE II-1

| | | Reductant | Reductant/mAb | ZnCl₂ | ZnCl₂/Reductant | Antibody |
|---|---|---|---|---|---|---|
| No. | Reductant | (mM) | (Molar Ratio) | (mM) | (Molar Ratio) | (mAb) |
| E II.1 | TCEP | 0.048 | 4:1 | 0.024 | 0.5:1 | Trastuzumab |
| E II.2 | TCEP | 0.048 | 4:1 | 0.042 | 0.87:1 | Sacituzumab |
| E II.3 | TCEP | 0.048 | 4:1 | 0.042 | 0.87:1 | Belantamab |
| E II.4 | TCEP-NO-Trtyl | 0.048 | 4:1 | 0.096 | 2:1 | Trastuzumab |
| E II.5 | TCEP-NO | 0.048 | 4:1 | 0.096 | 2:1 | Trastuzumab |
| E II.6 | TCEP-1 | 0.054 | 4.5:1 | 0.024 | 0.44:1 | Trastuzumab |
| E II.7 | TCEP-2 | 0.144 | 12:1 | 0.024 | 0.17:1 | Trastuzumab |
| E II.8 | TCEP-3 | 0.120 | 10:1 | 0.024 | 0.20:1 | Trastuzumab |
| E II.9 | TCEP-4 | 0.120 | 10:1 | 0.024 | 0.20:1 | Trastuzumab |
| E II.10 | TCEP-5 | 0.144 | 12:1 | 0.024 | 0.25:1 | Trastuzumab |
| E II.11 | TCEP-6 | 0.103 | 8.6:1 | 0.024 | 0.23:1 | Trastuzumab |
| E II.12 | TCEP-7 | 0.036 | 3:1 | 0.012 | 0.34:1 | Trastuzumab |
| E II.13 | TCEP-8 | 0.048 | 4:1 | 0.024 | 0.50:1 | Trastuzumab |
| E II.14 | TCEP-9 | 0.048 | 4:1 | 0.024 | 0.50:1 | Trastuzumab |
| E II.15 | TCEP-10 | 0.048 | 4:1 | 0.024 | 0.50:1 | Trastuzumab |
| E II.16 | TCEP-15 | 0.048 | 4:1 | 0.024 | 0.50:1 | Trastuzumab |
| E II.17 | TCEP-18 | 0.054 | 4.5:1 | 0.024 | 0.44:1 | Trastuzumab |
| E II.18 | TCEP-19 | 0.077 | 6.4:1 | 0.024 | 0.31:1 | Trastuzumab |
| E II.19 | TCEP-20 | 0.103 | 8.6:1 | 0.024 | 0.23:1 | Trastuzumab |
| E II.20 | TCEP-23 | 0.077 | 6.4:1 | 0.024 | 0.31:1 | Trastuzumab |
| E II.21 | TCEP-24 | 0.046 | 3.8:1 | 0.024 | 0.53:1 | Trastuzumab |
| E II.22 | TCEP-25 | 0.103 | 8.6:1 | 0.024 | 0.23:1 | Trastuzumab |
| E II.23 | TCEP-26 | 0.096 | 8:1 | 0.024 | 0.25:1 | Trastuzumab |
| E II.24 | TCEP-28 | 0.103 | 8.6:1 | 0.024 | 0.23:1 | Trastuzumab |
| E II.25 | TCEPA | 0.077 | 6.4:1 | 0.024 | 0.31:1 | Belantamab |
| E II.26 | TCEPA | 0.042 | 3.5:1 | 0.024 | 0.57:1 | Sacituzumab |
| E II.27 | TCEP-30 | 0.077 | 6.4:1 | 0.024 | 0.31:1 | Trastuzumab |
| E II.28 | TCEP-31 | 0.077 | 6.4:1 | 0.024 | 0.31:1 | Trastuzumab |
| E II.29 | TCEP-33 | 0.077 | 6.4:1 | 0.024 | 0.31:1 | Trastuzumab |
| C II.1 | TCEP | 0.048 | 4:1 | 0 | 0:1 | Trastuzumab |

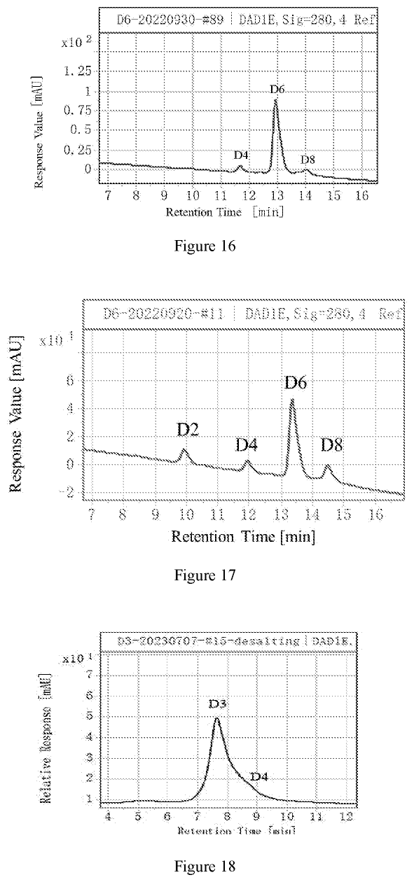

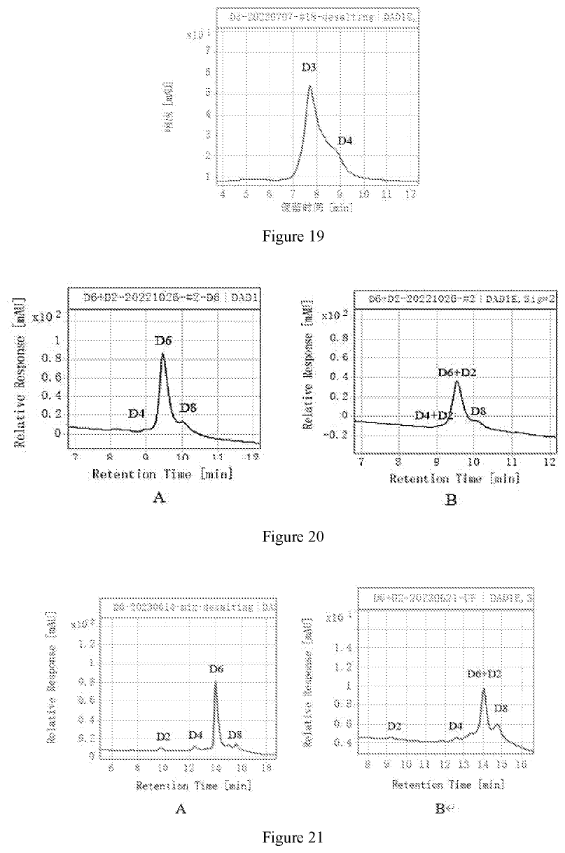

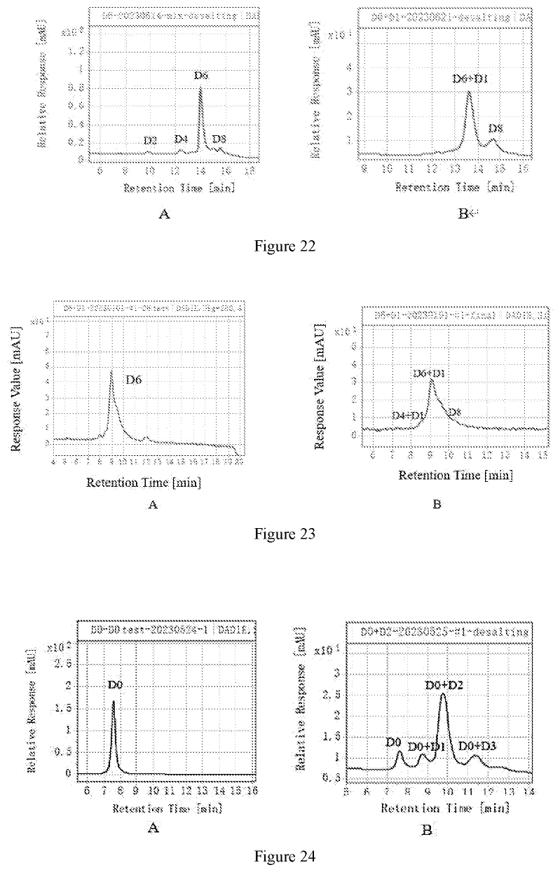

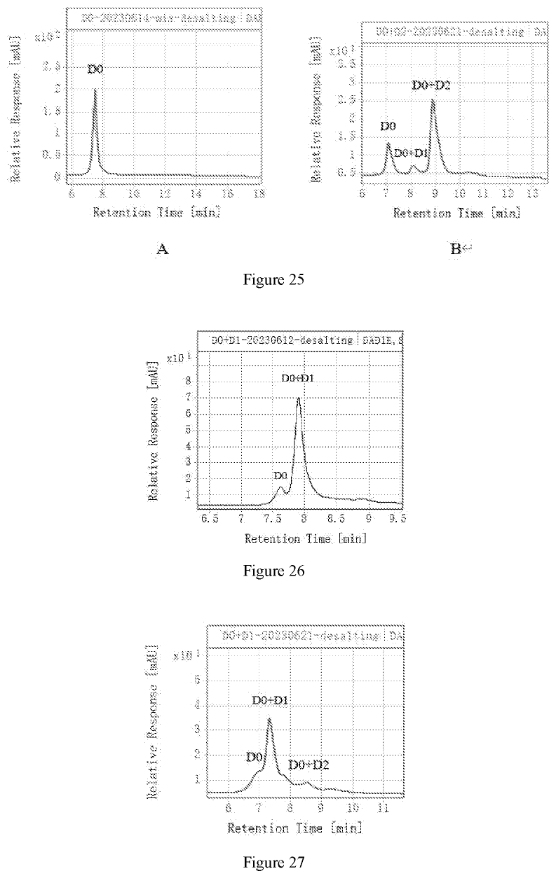

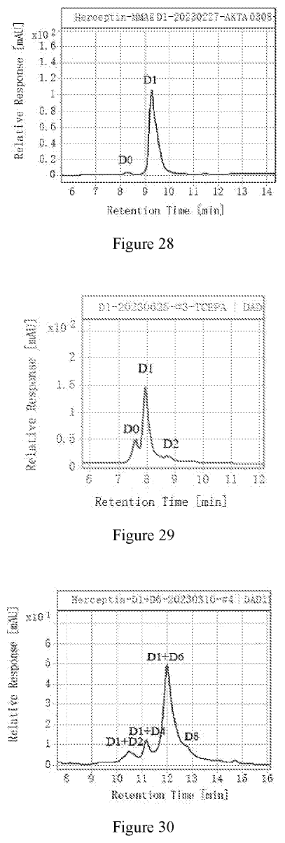

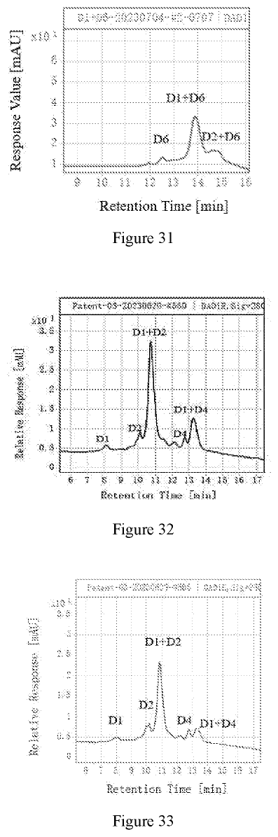

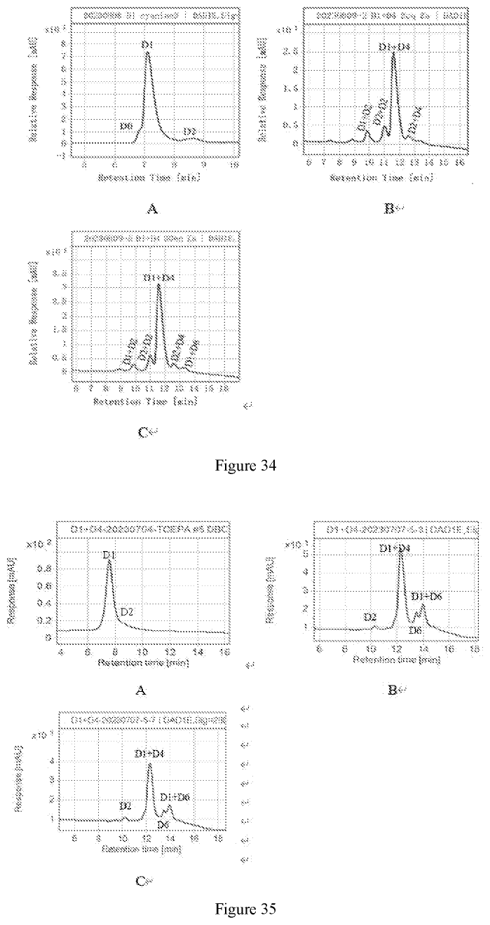

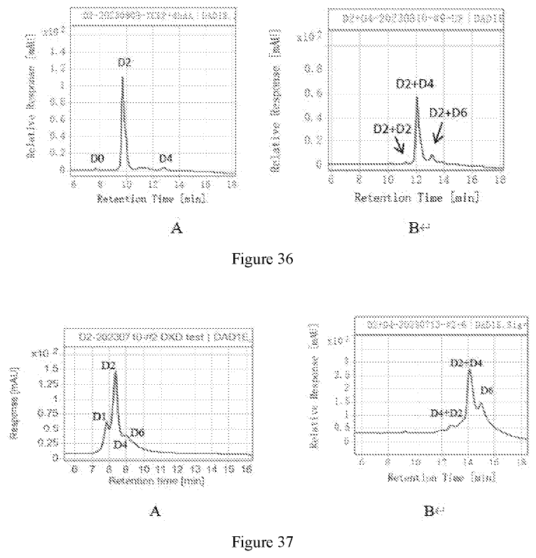

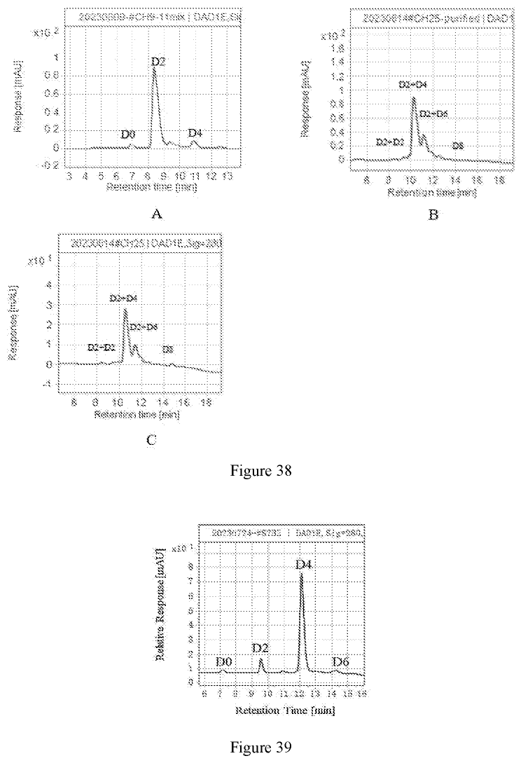

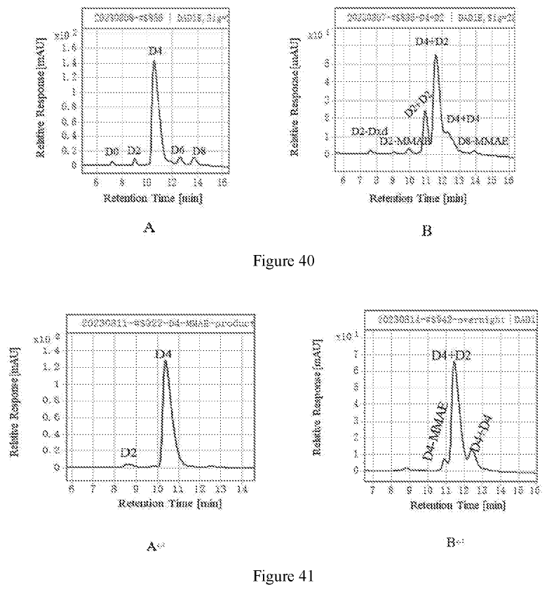

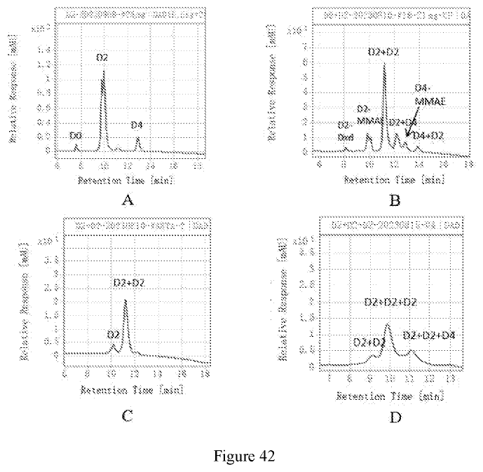

The homogeneity assay result and the chromatographic peak area of examples II.1-II.29 and comparative example II.1 were shown in Table II-2 and Table II-3. The chromatograms of E II.1 and E II.4 were shown in FIGS. 16-17, respectively.

TABLE II-2

| NO. | Reductant | D0 (%) | D2 (%) | D4 (%) | D6 (%) | D8 (%) |
|---|---|---|---|---|---|---|
| E II.1 | TCEP | 0 | 0 | 7 | 90 | 3 |
| E II.2 | TCEP | 0 | 1 | 3 | 85 | 11 |
| E II.3 | TCEP | 0 | 1 | 7 | 82 | 10 |
| E II.4 | TCEP-NO- | 0 | 11 | 9 | 66 | 14 |
| | Trtyl | | | | | |
| E II.5 | TCEP-NO | 0 | 4 | 16 | 70 | 10 |
| E II.6 | TCEP1 | 0 | 0 | 3.75 | 90.59 | 5.66 |
| E II.7 | TCEP-2 | 0 | 1.19 | 2.81 | 93.02 | 2.98 |
| E II.8 | TCEP-3 | 0 | 0 | 3.09 | 95.52 | 1.39 |
| E II.9 | TCEP-4 | 0 | 0 | 2.78 | 88.40 | 8.82 |
| E II.10 | TCEP-5 | 0 | 0 | 2.27 | 88.04 | 9.69 |
| E II.11 | TCEP-6 | 0 | 3.11 | 5.47 | 82.56 | 8.86 |
| E II.12 | TCEP-7 | 0 | 1.4 | 2.502 | 83.08 | 13.07 |
| E II.13 | TCEP-8 | 0 | 2.79 | 3.91 | 89.59 | 3.71 |
| E II.14 | TCEP-9 | 0 | 13.36 | 5.52 | 69.33 | 11.79 |
| E II.15 | TCEP-10 | 0 | 0 | 1.96 | 90.39 | 7.65 |
| E II.16 | TCEP-15 | 0 | 1.50 | 6.41 | 89.06 | 3.04 |
| E II.17 | TCEP-18 | 0 | 3.26 | 5.68 | 80.77 | 10.30 |
| E II.18 | TCEP-19 | 0 | 5.13 | 5.76 | 79.65 | 9.47 |
| E II.19 | TCEP-20 | 0 | 2.40 | 3.30 | 74.78 | 19.53 |
| E II.20 | TCEP-23 | 0 | 2.12 | 4.86 | 85.68 | 7.35 |
| E II.21 | TCEP-24 | 0 | 1.91 | 1.65 | 81.56 | 14.87 |
| E II.22 | TCEP-25 | 0 | 0.75 | 4.28 | 91.37 | 3.61 |
| E II.23 | TCEP-26 | 0 | 0 | 3.03 | 90.67 | 6.30 |
| E II.24 | TCEP-28 | 0 | 0 | 3.20 | 92.75 | 4.06 |
| E II.25 | TCEPA | 0 | 6.77 | 11.44 | 71.23 | 10.57 |
| E II.26 | TCEPA | 0 | 0 | 0 | 77.51 | 22.49 |
| E II.27 | TCEP-30 | 0 | 1.11 | 3.38 | 92.55 | 2.97 |
| E II.28 | TCEP-31 | 0 | 1.54 | 5.39 | 93.07 | 0 |
| E II.29 | TCEP-33 | 0 | 2.20 | 10.16 | 87.65 | 0 |
| C II.1 | TCEP | 0 | 0 | 9 | 10 | 81 |

TABLE II.3

| | | D0 | D2 | D4 | D6 | D8 |
|---|---|---|---|---|---|---|
| | | area | area | area | area | area |
| No | Reductant | (mAU) | (mAU) | (mAU) | (mAU) | (mAU) |
| E II.1 | TCEP | 0 | 0 | 117.31 | 1615.81 | 57.90 |
| E II.2 | TCEP | 0 | 41.35 | 117.75 | 2892.94 | 373.01 |
| E II.3 | TCEP | 0 | 48.40 | 239.30 | 2769.41 | 343.21 |
| E II.4 | TCEP-NO-Trtyl | 0 | 173.47 | 130.24 | 1020.62 | 216.72 |
| E II.5 | TCEP-NO | 0 | 67.46 | 248.72 | 1100.50 | 163.18 |
| E II.6 | TCEP1 | 0 | 0 | 61.87 | 1495.92 | 93.52 |
| E II.7 | TCEP-2 | 0 | 16.49 | 39.14 | 1294.70 | 41.50 |
| E II.8 | TCEP-3 | 0 | 0 | 59.67 | 1846.44 | 26.93 |
| E II.9 | TCEP-4 | 0 | 0 | 32.93 | 1047.19 | 104.47 |
| E II.10 | TCEP-5 | 0 | 0 | 24.50 | 949.40 | 104.51 |
| E II.11 | TCEP-6 | 0 | 49.02 | 86.22 | 1301.25 | 139.59 |
| E II.12 | TCEP-7 | 0 | 11.49 | 21.28 | 706.55 | 111.14 |
| E II.13 | TCEP-8 | 0 | 34.60 | 48.48 | 1111.56 | 46.07 |
| E II.14 | TCEP-9 | 0 | 140.00 | 57.87 | 726.63 | 123.52 |
| E II.15 | TCEP-10 | 0 | 0 | 23.60 | 1090.96 | 92.33 |
| E II.16 | TCEP-15 | 0 | 21.79 | 93.15 | 1294.14 | 44.11 |
| E II.17 | TCEP-18 | 0 | 49.98 | 86.98 | 1237.78 | 157.83 |
| E II.18 | TCEP-19 | 0 | 106.00 | 118.99 | 1646.13 | 195.70 |
| E II.19 | TCEP-20 | 0 | 28.50 | 39.22 | 890.01 | 232.40 |
| E II.20 | TCEP-23 | 0 | 40.19 | 92.21 | 1626.44 | 139.47 |
| E II.21 | TCEP-24 | 0 | 24.48 | 21.15 | 1043.94 | 190.35 |
| E II.22 | TCEP-25 | 0 | 13.07 | 74.87 | 1599.53 | 63.24 |
| E II.23 | TCEP-26 | 0 | 0 | 35.56 | 1064.41 | 73.96 |
| E II.24 | TCEP-28 | 0 | 0 | 55.87 | 1621.69 | 70.92 |
| E II.25 | TCEPA | 0 | 114.08 | 192.80 | 1201.07 | 178.20 |
| E II.26 | TCEPA | 0 | 0 | 0 | 1297.68 | 376.46 |
| E II.27 | TCEP-30 | 0 | 13.777 | 42.06 | 1153.36 | 37.035 |
| E II.28 | TCEP-31 | 0 | 12.59 | 44.213 | 763.08 | 0 |
| E II.29 | TCEP-33 | 0 | 14.215 | 65.785 | 567.537 | 0 |
| C II.1 | TCEP | 0 | 0 | 155.05 | 185.00 | 1427.70 |

As shown in the above table, linker-payloads (MC-VC-PAB-MMAE) were successfully linked to the antibody, which indicated ADCs of examples II. 1-II.29 prepared by the reductant in the present application were successfully synthesized.

Further, as the results of example [II.4-II.29], the compounds in the present application could increase the homogeneity of the ADC with D6 compared with the traditional method using TCEP without Zn²⁺, wherein, the selective reduction ability of TCEP-3 is best, with a D6 content of up to 95.52%. Meanwhile, the selective reduction ability of TCEP-1, TCEP-2, TCEP-10, TCEP-25, TCEP-26, TCEP-28, TCEP-30, TCEP-31 and TCEP-33 is also wonderful, with a D6 content of up to 90%.

According to the results of examples II.1-II.3 and II.25-II.26, the reductants in the present application are suitable for preparing the ADC with different antibody.

### Examples II.30-II.60 Preparation of Antibody-[MC-VC-PAB-MMAE]₆ Conjugate (the Molar Ratio of Reductant and the Antibody and/or the Reduction Time in Step (1) is (are) Different)

When the reductant is TCEP, the method is the similar to example II.1, and when the reductant is not TCEP, the method is the similar to example 4:1. The differences are the concentration of ZnC₂ and reductant in step (1) and/or the reduction time in step (1), Which are shown in Table II-4. Meanwhile, the antibody is belantamab in example II.31-II.32.

TABLE II-4

| | | | | | | Reductant |
|---|---|---|---|---|---|---|
| | | Reductant/mAb | ZnCl₂/mAb | ZnCl₂/Reductant | | time in step |
| No. | Reductant | (Molar Ratio) | (Molar Ratio) | (Molar Ratio) | mAb (mM) | (1) (h) |
| E II.30 | TCEP | 3:1 | 4:1 | 1.33:1 | 0.012 | 18 |
| E II.31 | TCEP | 3.2:1 | 12:1 | 3.75:1 | 0.012 | 18 |
| E II.32 | TCEP | 5:1 | 4:1 | 0.80:1 | 0.012 | 18 |
| E II.33 | TCEP | 6:1 | 4:1 | 0.67:1 | 0.012 | 18 |
| E II.34 | TCEP | 8:1 | 2:1 | 0.25:1 | 0.012 | 6 |
| E II.35 | TCEP | 9:1 | 2:1 | 0.22:1 | 0.012 | 6 |
| E II.36 | TCEP | 10:1 | 2:1 | 0.20:1 | 0.012 | 6 |
| E II.37 | TCEP | 11:1 | 2:1 | 0.18:1 | 0.012 | 6 |
| E II.38 | TCEP | 12:1 | 2:1 | 0.17:1 | 0.012 | 6 |
| E II.39 | TCEP | 13:1 | 2:1 | 0.15:1 | 0.012 | 6 |
| E II.40 | TCEPA | 2.8:1 | 2:1 | 0.71:1 | 0.012 | 18 |
| E II.41 | TCEPA | 3.5:1 | 2:1 | 0.57:1 | 0.012 | 18 |
| E II.42 | TCEPA | 3.8:1 | 2:1 | 0.52:1 | 0.012 | 18 |
| E II.43 | TCEPA | 4:1 | 2:1 | 0.5:1 | 0.012 | 18 |
| E II.44 | TCEPA | 4.2:1 | 2:1 | 0.47:1 | 0.012 | 18 |
| E II.45 | TCEPA | 4.4:1 | 2:1 | 0.45:1 | 0.012 | 18 |
| E II.46 | TCEPA | 4.6:1 | 2:1 | 0.43:1 | 0.012 | 18 |
| E II.47 | TCEPA | 3.8:1 | 1:1 | 0.26:1 | 0.012 | 18 |
| E II.48 | TCEPA | 4:1 | 1:1 | 0.25:1 | 0.012 | 18 |
| E II.49 | TCEPA | 4.2:1 | 1:1 | 0.23:1 | 0.012 | 18 |
| E II.50 | TCEPA | 4.4:1 | 1:1 | 0.22:1 | 0.012 | 18 |
| E II.51 | TCEPA | 4.6:1 | 1:1 | 0.21:1 | 0.012 | 18 |
| E II.52 | TCEP-NO | 5:1 | 1:1 | 0.20:1 | 0.012 | 6 |
| E II.53 | TCEP-NO | 6:1 | 1:1 | 0.16:1 | 0.012 | 6 |
| E II.54 | TCEP-NO | 7:1 | 1:1 | 0.14:1 | 0.012 | 6 |
| E II.55 | TCEP-NO | 8:1 | 1:1 | 0.125:1 | 0.012 | 6 |
| E II.56 | TCEP-NO | 9:1 | 1:1 | 0.11:1 | 0.012 | 6 |
| E II.57 | TCEP-NO | 10:1 | 1:1 | 0.1:1 | 0.012 | 6 |
| E II.58 | TCEP-NO | 11:1 | 1:1 | 0.09:1 | 0.012 | 6 |
| E II.59 | TCEP-NO | 12:1 | 1:1 | 0.083:1 | 0.012 | 6 |
| E II.60 | TCEP-NO | 13:1 | 1:1 | 0.077:1 | 0.012 | 6 |

The homogeneity assay result and the chromatographic peak area of examples II.30-II.60 were shown in Table II-5 and Table II-6.

TABLE II-5

| | | Reductant/mAb | Reductant time | | | | | |
|---|---|---|---|---|---|---|---|---|
| No. | Reductant | (Molar Ratio) | in step (1) (h) | D0 (%) | D2 (%) | D4 (%) | D6 (%) | D8 (%) |
| E II.30 | TCEP | 3:1 | 18 | 0 | 12.75 | 32.22 | 55.03 | 0 |
| E II.31 | TCEP | 3.2:1 | 18 | 0 | 2.87 | 11.09 | 80.33 | 5.71 |
| E II.32 | TCEP | 5:1 | 18 | 0 | 0 | 6.27 | 82.40 | 11.32 |
| E II.33 | TCEP | 6:1 | 18 | 0 | 0 | 4.67 | 80.91 | 14.42 |
| E II.34 | TCEP | 8:1 | 6 | 0 | 3.50 | 5.64 | 87.41 | 3.45 |
| E II.35 | TCEP | 9:1 | 6 | 0 | 3.47 | 5.11 | 87.20 | 4.22 |
| E II.36 | TCEP | 10:1 | 6 | 0 | 1.72 | 4.09 | 89.34 | 4.86 |
| E II.37 | TCEP | 11:1 | 6 | 0 | 1.57 | 3.27 | 89.24 | 5.93 |
| E II.38 | TCEP | 12:1 | 6 | 0 | 1.54 | 2.99 | 89.07 | 6.41 |
| E II.39 | TCEP | 13:1 | 6 | 0 | 4.43 | 4.96 | 83.50 | 7.12 |
| E II.40 | TCEPA | 2.8:1 | 18 | 0 | 11.11 | 13.35 | 75.54 | 0 |
| E II.41 | TCEPA | 3.5:1 | 18 | 0 | 5.35 | 8.07 | 84.71 | 1.87 |
| E II.42 | TCEPA | 3.8:1 | 18 | 0 | 5 | 10 | 81 | 4 |
| E II.43 | TCEPA | 4:1 | 18 | 0 | 4 | 10 | 81 | 5 |
| E II.44 | TCEPA | 4.2:1 | 18 | 0 | 3 | 6 | 83 | 8 |
| E II.45 | TCEPA | 4.4:1 | 18 | 0 | 3 | 6 | 83 | 7 |
| E II.46 | TCEPA | 4.6:1 | 18 | 0 | 3 | 7 | 82 | 8 |
| E II.47 | TCEPA | 3.8:1 | 18 | 0 | 5 | 24 | 70 | 1 |
| E II.48 | TCEPA | 4:1 | 18 | 0 | 4 | 17 | 76 | 3 |
| E II.49 | TCEPA | 4.2:1 | 18 | 0 | 3 | 15 | 78 | 4 |
| E II.50 | TCEPA | 4.4:1 | 18 | 0 | 4 | 17 | 75 | 4 |
| E II.51 | TCEPA | 4.6:1 | 18 | 0 | 3 | 12 | 79 | 6 |
| E II.52 | TCEP-NO | 5:1 | 6 | 0 | 23.69 | 32.30 | 44.01 | 0 |
| E II.53 | TCEP-NO | 6:1 | 6 | 0 | 13.74 | 21.65 | 64.60 | 0 |
| E II.54 | TCEP-NO | 7:1 | 6 | 0 | 11.16 | 22.20 | 66.64 | 0 |
| E II.55 | TCEP-NO | 8:1 | 6 | 0 | 4.69 | 5.88 | 81.04 | 8.40 |
| E II.56 | TCEP-NO | 9:1 | 6 | 0 | 3.81 | 5.02 | 82.00 | 9.17 |
| E II.57 | TCEP-NO | 10:1 | 6 | 0 | 2.79 | 4.07 | 83.81 | 9.33 |
| E II.58 | TCEP-NO | 11:1 | 6 | 0 | 1.14 | 2.74 | 82.19 | 13.93 |
| E II.59 | TCEP-NO | 12:1 | 6 | 0 | 1.25 | 2.70 | 82.22 | 13.84 |
| E II.60 | TCEP-NO | 13:1 | 6 | 0 | 1.95 | 2.79 | 81.04 | 14.23 |

TABLE II-6

| | D0 area | D2 area | D4 area | D6 area | D8 area |
|---|---|---|---|---|---|
| Example | (mAU) | (mAU) | (mAU) | (mAU) | (mAU) |
| E II.30 | 0 | 328.70 | 830.70 | 1418.67 | 0 |
| E II.31 | 0 | 54.53 | 210.51 | 1525.41 | 108.48 |
| E II.32 | 0 | 0 | 211.00 | 2771.55 | 380.84 |
| E II.33 | 0 | 0 | 172.93 | 2995.57 | 533.79 |
| E II.34 | 0 | 51.36 | 82.69 | 1282.31 | 50.63 |
| E II.35 | 0 | 60.73 | 89.51 | 1526.56 | 73.88 |
| E II.36 | 0 | 28.86 | 68.75 | 1502.11 | 81.70 |
| E II.37 | 0 | 24.91 | 51.80 | 1414.32 | 93.92 |
| E II.38 | 0 | 25.71 | 50.01 | 1491.33 | 107.29 |
| E II.39 | 0 | 41.35 | 46.33 | 779.88 | 66.46 |
| E II.40 | 0 | 169.79 | 204.06 | 1154.37 | 0 |
| E II.41 | 0 | 68.99 | 104.01 | 1091.96 | 24.13 |
| E II.42 | 0 | 69.47 | 141.31 | 1160.71 | 63.87 |
| E II.43 | 0 | 65.63 | 139.04 | 1177.61 | 77.57 |
| E II.44 | 0 | 72.96 | 133.61 | 1806.36 | 175.60 |
| E II.45 | 0 | 56.21 | 115.90 | 1500.17 | 120.74 |
| E II.46 | 0 | 58.50 | 121.97 | 1505.02 | 148.16 |
| E II.47 | 0 | 100.76 | 441.85 | 1296.29 | 21.11 |
| E II.48 | 0 | 76.68 | 297.96 | 1293.10 | 43.13 |
| E II.49 | 0 | 58.15 | 269.34 | 1423.97 | 70.52 |
| E II.50 | 0 | 80.95 | 326.74 | 1456.73 | 68.24 |
| E II.51 | 0 | 61.94 | 260.44 | 1649.14 | 129.15 |
| E II.52 | 0 | 244.41 | 333.20 | 453.94 | 0 |
| E II.53 | 0 | 179.92 | 283.48 | 845.80 | 0 |
| E II.54 | 0 | 125.57 | 249.73 | 749.54 | 0 |
| E II.55 | 0 | 68.47 | 85.90 | 1184.29 | 122.69 |
| E II.56 | 0 | 76.92 | 101.16 | 1654.11 | 185.05 |
| E II.57 | 0 | 30.92 | 45.21 | 930.34 | 103.60 |
| E II.58 | 0 | 15.43 | 36.94 | 1108.89 | 187.95 |
| E II.59 | 0 | 14.05 | 30.46 | 927.85 | 156.19 |
| E II.60 | 0 | 16.76 | 24.04 | 698.34 | 122.61 |

As the results shown in table II-5, linker-payloads (MC-VC-PAB-MMAE) were successfully linked to the antibody, which indicated ADCs of examples II.30-II.60 prepared by TCEP, TCEPA or TCEP-NO were successfully synthesized.

As shown in examples II.30-II.39, using the TCEP as reductant, the content of D6 is up to 55%, 80%, even to 85%, and 87% when the molar ratio of TCEP and the antibody is from 3:1 to 13:1. When the molar ratio of TCEP and the antibody is from 8:1 to 13:1, the reduction time in step (1) is shortened to 6 h. When the molar ratio of TCEP and the antibody is from 8:1 to 12:1 and the reduction time in step (1) is 6 h, the content of D6 is up to 87% or 89%, the results showed the molar ratio of TCEP/antibody plays an important role in determining the content of D6 and the selective reduction.

According to the results of examples II.40-II.60, using the TCEP-NO or TCEPA as reductant, the highest proportion of D6 was 84.71%, when the molar ratio of the ZnCl₂ and the monoclonal antibody was 2 and the molar ratio of the reductant and the antibody was 2.8:1 to 4.6:1. And D6 was at least 40% when the molar ratio of the ZnCl₂ and the monoclonal antibody was 1 and the molar ratio of the reductant and the antibody was 3.8:1 to 13:1. The resultant ADCs with high level of D6 showed that the molar ratio of the reductant and the antibody ranging from 2.8 to 13 was benefit for improving the homogeneity of ADCs.

According to example II.52-II.60, when the molar ratio of the reductant and the antibody is from 5:1 to 13:1, the reduction time in step (1) is shortened to 6 h. When the molar ratio of the reductant and the antibody is from 6:1 to 13:1 and the reduction time in step (1) is 6 h, the content of D6 is up to 65%, 70%, even to 75% or 80%.

### Examples II.61-II.87 and Comparative Examples II.2-II.9: Preparation of Trastuzumab-[MC-VC-PAB-Mmae]ₕ conjugate (the molar ratio of Zn²⁺ And TCEP is Different and/or the Reduction Time in Step (1) is (are) Different)

When the reductant is TCEP, the method is similar to example II.1, and when the reductant is not TCEP, the method is similar to example II.4. The difference is the concentration of ZnCl₂, the concentration of TCEP and/or the reduction time in step (1). The parameters in step (1) are shown in table II-7.

The method of comparative example II.2 is the same as example II.1, comparative example II.3 is the same as example II.34, comparative example II.4 is the same as example II.36, comparative example II.5 is the same as example II.38, comparative example II.6 is the same as example II.4, comparative example II.7 is the same as example II.55, comparative example II.8 is the same as example II.57, comparative example II.9 is the same as example II.59, the difference is that the concentration of ZnCl₂ in step (1) is 0 and the reductant is TCEPA in comparative example II.6.

TABLE II-7

| | | ZnCl₂/ | | Reductant/ | |
|---|---|---|---|---|---|
| | | Reductant | | mAb | The reductant |
| | | (Molar | mAb | (Molar | time in step |
| No. | Reductant | Ratio) | (mM) | Ratio) | (1) (h) |
| C II.2 | TCEP | 0 | 0.012 | 4:1 | 18 |
| C II.3 | TCEP | 0 | 0.012 | 8:1 | 6 |
| C II.4 | TCEP | 0 | 0.012 | 10:1 | 6 |
| C II.5 | TCEP | 0 | 0.012 | 12:1 | 6 |
| E II.61 | TCEP | 0.25:1 | 0.012 | 4:1 | 18 |
| E II.62 | TCEP | 0.5:1 | 0.012 | 4:1 | 18 |
| E II.63 | TCEP | 1:1 | 0.012 | 4:1 | 18 |
| E II.64 | TCEP | 2:1 | 0.012 | 4:1 | 18 |
| E II.65 | TCEP | 3:1 | 0.012 | 4:1 | 18 |
| E II.66 | TCEP | 4:1 | 0.012 | 4:1 | 18 |
| E II.67 | TCEP | 7.5:1 | 0.012 | 4:1 | 18 |
| E II.68 | TCEP | 12:1 | 0.012 | 5:1 | 18 |
| E II.69 | TCEP | 27.27:1 | 0.012 | 5:1 | 18 |
| E II.70 | TCEP | 0.11:1 | 0.012 | 9:1 | 6 |
| E II.71 | TCEP | 0.22:1 | 0.012 | 9:1 | 6 |
| E II.72 | TCEP | 0.44:1 | 0.012 | 9:1 | 6 |
| E II.73 | TCEP | 0.66:1 | 0.012 | 9:1 | 6 |
| E II.74 | TCEP | 0.88:1 | 0.012 | 9:1 | 6 |
| E II.75 | TCEP | 1.67:1 | 0.012 | 9:1 | 6 |
| C II.6 | TCEPA | 0 | 0.012 | 4:1 | 18 |
| C II.7 | TCEP-NO | 0 | 0.012 | 8:1 | 6 |
| C II.8 | TCEP-NO | 0 | 0.012 | 10:1 | 6 |
| C II.9 | TCEP-NO | 0 | 0.012 | 12:1 | 6 |
| E II.76 | TCEPA | 0.25:1 | 0.012 | 4 | 18 |
| E II.77 | TCEPA | 0.5:1 | 0.012 | 4 | 18 |
| E II.78 | TCEPA | 1:1 | 0.012 | 4 | 18 |
| E II.79 | TCEPA | 2:1 | 0.012 | 4 | 18 |
| E II.80 | TCEPA | 3:1 | 0.012 | 4 | 18 |
| E II.81 | TCEPA | 4:1 | 0.012 | 4 | 18 |
| E II.82 | TCEPA | 7:1 | 0.012 | 4 | 18 |
| E II.83 | TCEPA | 7.5:1 | 0.012 | 4 | 18 |
| E II.84 | TCEPA | 15:1 | 0.012 | 3.2:1 | 18 |
| E II.85 | TCEPA | 30:1 | 0.012 | 3.2:1 | 18 |
| E II.86 | TCEP-NO | 0.22:1 | 0.012 | 9:1 | 4 |
| E II.87 | TCEP-NO | 3.33:1 | 0.012 | 9:1 | 4 |

mAb was short for monoclonal antibody.

The homogeneity assay result and the chromatographic peak area of examples II.61-II.87 and comparative examples II.2-II.9 were shown in Table II-8 and Table II-9.

TABLE II-8

| | | ZnCl₂/Reductant | | | | | |
|---|---|---|---|---|---|---|---|
| No. | Reductant | (Molar Ratio) | D0 (%) | D2 (%) | D4 (%) | D6 (%) | D8 (%) |
| C II.2 | TCEP | 0 | 0 | 0 | 9 | 10 | 81 |
| C II.3 | TCEP | 0 | 0 | 2.64 | 10.01 | 17.10 | 70.25 |
| C II.4 | TCEP | 0 | 0 | 1.62 | 6.89 | 11.75 | 79.75 |
| C II.5 | TCEP | 0 | 0 | 3.83 | 4.70 | 10.45 | 81.02 |
| E II.61 | TCEP | 0.25:1 | 0 | 0 | 12 | 81 | 7 |
| E II.62 | TCEP | 0.5:1 | 0 | 2 | 5 | 86 | 7 |
| E II.63 | TCEP | 1:1 | 0 | 2 | 5 | 85 | 8 |
| E II.64 | TCEP | 2:1 | 0 | 0 | 4.40 | 86.59 | 9.01 |
| E II.65 | TCEP | 3:1 | 0 | 0 | 4 | 83 | 13 |
| E II.66 | TCEP | 4:1 | 0 | 0 | 6 | 81 | 13 |
| E II.67 | TCEP | 7.5:1 | 0 | 0 | 5 | 75 | 20 |
| E II.68 | TCEP | 12:1 | 0 | 1.88 | 7.48 | 85.81 | 4.83 |
| E II.69 | TCEP | 27.27:1 | 0 | 2.99 | 7.75 | 77.99 | 11.27 |
| E II.70 | TCEP | 0.11:1 | 0 | 2.09 | 5.40 | 90.48 | 2.03 |
| E II.71 | TCEP | 0.22:1 | 0 | 2.57 | 4.79 | 89.99 | 2.65 |
| E II.72 | TCEP | 0.44:1 | 0 | 2.58 | 5.83 | 89.20 | 2.39 |
| E II.73 | TCEP | 0.66:1 | 0 | 2.86 | 6.09 | 89.11 | 1.95 |
| E II.74 | TCEP | 0.88:1 | 0 | 2.55 | 5.82 | 89.59 | 2.03 |
| E II.75 | TCEP | 1.67:1 | 0 | 3.23 | 6.04 | 87.19 | 3.54 |
| C II.6 | TCEPA | 0 | 0 | 2 | 18 | 20 | 60 |
| C II.7 | TCEP-NO | 0 | 0 | 0 | 24.55 | 30.92 | 44.54 |
| C II.8 | TCEP-NO | 0 | 0 | 0 | 6.97 | 17.14 | 75.89 |
| C II.9 | TCEP-NO | 0 | 0 | 0 | 0 | 11.56 | 88.44 |
| E II.76 | TCEPA | 0.25:1 | 0 | 3 | 12 | 79 | 6 |
| E II.77 | TCEPA | 0.5:1 | 0 | 3 | 6 | 83 | 8 |
| E II.78 | TCEPA | 1:1 | 0 | 3 | 5 | 82 | 10 |
| E II.79 | TCEPA | 2:1 | 0 | 3 | 6 | 83 | 8 |
| E II.80 | TCEPA | 3:1 | 0 | 0 | 5 | 83 | 12 |
| E II.81 | TCEPA | 4:1 | 0 | 0 | 6 | 81 | 13 |
| E II.82 | TCEPA | 7:1 | 0 | 0 | 5 | 79 | 16 |
| E II.83 | TCEPA | 7.5:1 | 0 | 0 | 5.29 | 79.31 | 15.40 |
| E II.84 | TCEPA | 15:1 | 0 | 2.94 | 6.64 | 85.07 | 5.36 |
| E II.85 | TCEPA | 30:1 | 0 | 2.08 | 7.43 | 78.90 | 11.59 |
| E II.86 | TCEP-NO | 0.22:1 | 0 | 4.12 | 5.79 | 77.34 | 12.75 |
| E II.87 | TCEP-NO | 3.33:1 | 4.81 | 2.83 | 4.49 | 63.04 | 24.83 |

TABLE II-9

| | | D0 area | D2 area | D4 area | D6 area | D8 area |
|---|---|---|---|---|---|---|
| Example | Reductant | (mAU) | (mAU) | (mAU) | (mAU) | (mAU) |
| C II.2 | TCEP | 0 | 0 | 155.05 | 185.00 | 1427.70 |
| C II.3 | TCEP | 0 | 33.17 | 125.95 | 215.22 | 884.11 |
| C II.4 | TCEP | 0 | 20.30 | 86.36 | 147.32 | 1000.25 |
| C II.5 | TCEP | 0 | 29.97 | 36.72 | 81.71 | 633.61 |
| E II.61 | TCEP | 0 | 0 | 193.00 | 1373.82 | 124.17 |
| E II.62 | TCEP | 0 | 37.42 | 106.78 | 1634.81 | 130.69 |
| E II.63 | TCEP | 0 | 32.76 | 83.23 | 1524.30 | 149.14 |
| E II.64 | TCEP | 0 | 0 | 85.02 | 1674.75 | 174.27 |
| E II.65 | TCEP | 0 | 0 | 82.86 | 1688.64 | 260.04 |
| E II.66 | TCEP | 0 | 0 | 114.29 | 1597.47 | 258.67 |
| E II.67 | TCEP | 0 | 0 | 104.58 | 1642.59 | 452.71 |
| E II.68 | TCEP | 0 | 30.09 | 119.57 | 1371.69 | 77.27 |
| E II.69 | TCEP | 0 | 48.03 | 124.29 | 1250.95 | 180.80 |
| E II.70 | TCEP | 0 | 25.65 | 66.39 | 1111.66 | 24.96 |
| E II.71 | TCEP | 0 | 31.48 | 58.60 | 1100.43 | 32.36 |
| E II.72 | TCEP | 0 | 30.23 | 68.29 | 1045.27 | 28.01 |
| E II.73 | TCEP | 0 | 42.08 | 89.67 | 1311.86 | 28.66 |
| E II.74 | TCEP | 0 | 32.69 | 74.56 | 1146.98 | 25.99 |
| E II.75 | TCEP | 0 | 52.84 | 98.74 | 1425.09 | 57.86 |
| C II.6 | TCEPA | 0 | 34.16 | 281.08 | 321.24 | 948.75 |
| C II.7 | TCEP-NO | 0 | 0 | 235.56 | 296.71 | 427.38 |
| C II.8 | TCEP-NO | 0 | 0 | 78.41 | 192.84 | 853.64 |
| C II.9 | TCEP-NO | 0 | 0 | 0 | 90.26 | 690.72 |
| E II.76 | TCEPA | 0 | 44.90 | 202.90 | 1298.74 | 95.35 |
| E II.77 | TCEPA | 0 | 62.69 | 108.15 | 1595.32 | 156.96 |
| E II.78 | TCEPA | 0 | 57.78 | 98.60 | 1581.70 | 182.49 |
| E II.79 | TCEPA | 0 | 57.81 | 89.45 | 1311.02 | 124.66 |
| E II.80 | TCEPA | 0 | 0 | 87.59 | 1379.68 | 201.42 |
| E II.81 | TCEPA | 0 | 0 | 100.12 | 1491.45 | 244.26 |
| E II.82 | TCEPA | 0 | 0 | 90.18 | 1352.22 | 272.18 |
| E II.83 | TCEPA | 0 | 0 | 90.18 | 1352.22 | 262.65 |
| E II.84 | TCEPA | 0 | 18.30 | 41.27 | 529.21 | 33.32 |
| E II.85 | TCEPA | 0 | 11.38 | 40.75 | 432.58 | 63.53 |
| E II.86 | TCEP-NO | 0 | 74.66 | 104.84 | 1401.10 | 230.97 |
| E II.87 | TCEP-NO | 64.03 | 37.72 | 59.70 | 838.96 | 330.37 |

As the results shown in table II-8, linker-payloads (MC-VC-PAB-MMAE) were successfully linked to Trastuzumab, which indicated ADCs of examples II.61-II.87 prepared with TCEP/TCEPA/TCEP-NO and Zn²⁺ were successfully synthesized.

According to the results of table II-8, The proportion of D6 in comparative examples II.2-II.9 was only 11%, 17%, 20% or 31%, but the proportion of D6 in Example II.61-II.87 was at least 60%. It showed that Zn²⁺ was important to improve the homogeneity of ADCs with D6. Further, the molar ratio of Zn²⁺ and the reductant ranging from 0.05 to 30 was benefit for improving the homogeneity of ADCs.

### Examples II.88-II.110 and Comparative Examples II.10-II.11: Preparation of Trastuzumab-[MC-VC-PAB-MMAE]₆ Conjugate by Using the Different Buffer System

When the reductant is TCEP, the method is similar to example II.1, and when the reductant is not TCEP, the method is similar to example II.79. The difference is the molar ratio of the TCEPA and the antibody and/or and the buffer system which are shown in table II-10.

TABLE II-10

| | | | Reductant/mAb | ZnCl₂/mAb | |
|---|---|---|---|---|---|
| | | The buffer | (Molar | (Molar | |
| No. | Reductant | system | Ratio) | Ratio) | pH |
| E II.88 | TCEP | Bis-Tris | 4:1 | 2:1 | 6.7 |
| | | buffer | | | |
| E II.89 | TCEP | PIPES buffer | 4:1 | 2:1 | 6.7 |
| E II.90 | TCEP | MOPS buffer | 4:1 | 2:1 | 6.7 |
| E II.91 | TCEP | BES buffer | 4:1 | 2:1 | 6.7 |
| E II.92 | TCEP | HEPES buffer | 4:1 | 2:1 | 6.7 |
| E II.93 | TCEP | DIPSO buffer | 4:1 | 2:1 | 6.7 |
| E II.94 | TCEP | MOBS buffer | 4:1 | 2:1 | 7.4 |
| E II.95 | TCEP | MOPSO buffer | 4:1 | 2:1 | 7.4 |
| E II.96 | TCEP | TES buffer | 4:1 | 2:1 | 7.4 |
| E II.97 | TCEP | ACES buffer | 4:1 | 2:1 | 7.4 |
| E II.98 | TCEP | TAPSO buffer | 4:1 | 2:1 | 7.4 |
| C II.10 | TCEP | PB | 4:1 | 2:1 | 6.7 |
| C II.11 | TCEP | ADA buffer | 4:1 | 2:1 | 6.7 |
| E II.99 | TCEPA | MOBS buffer | 4:1 | 2:1 | 7.4 |
| E II.100 | TCEPA | TAPSO buffer | 4:1 | 2:1 | 7.4 |
| E II.101 | TCEPA | DIPSO buffer | 4:1 | 2:1 | 7.4 |
| E II.102 | TCEPA | MOPSO buffer | 4:1 | 2:1 | 7.4 |
| E II.103 | TCEPA | ACES buffer | 4:1 | 2:1 | 7.4 |
| E II.104 | TCEPA | Bis-Tris | 3.5:1 | 2:1 | 6.7 |
| | | buffer | | | |
| E II.105 | TCEPA | PIPES buffer | 3.5:1 | 2:1 | 6.7 |
| E II.106 | TCEPA | HEPES buffer | 3.5:1 | 2:1 | 7.0 |
| E II.107 | TCEPA | MOPS buffer | 3.5:1 | 2:1 | 7.0 |
| E II.108 | TCEPA | TES buffer | 3.5:1 | 2:1 | 7.4 |
| E II.109 | TCEPA | PB | 3.5:1 | 2:1 | 6.7 |
| E II.110 | TCEPA | ADA buffer | 3.5:1 | 2:1 | 6.7 |

The homogeneity assay result and the chromatographic peak area of examples II.88-II.110 and comparative examples II.10-II.11 were shown in Table II-I11 and Table II-12.

TABLE II-11

| N0. | Reductant | D0 (%) | D2 (%) | D4 (%) | D6 (%) | D8 (%) |
|---|---|---|---|---|---|---|
| E II.88 | TCEP | 0 | 5 | 11 | 79 | 5 |
| E II.89 | TCEP | 0 | 11 | 31 | 56 | 2 |
| E II.90 | TCEP | 0 | 2 | 7 | 83 | 8 |
| E II.91 | TCEP | 0 | 4 | 7 | 84 | 5 |
| E II.92 | TCEP | 0 | 1 | 4 | 82 | 13 |
| E II.93 | TCEP | 0 | 0 | 2 | 87 | 11 |
| E II.94 | TCEP | 0 | 0 | 3 | 88 | 9 |
| E II.95 | TCEP | 0 | 0 | 4 | 87 | 9 |
| E II.96 | TCEP | 0 | 2 | 5 | 86 | 7 |
| E II.97 | TCEP | 0 | 0 | 5 | 87 | 8 |
| E II.98 | TCEP | 0 | 1 | 4 | 88 | 7 |
| C II.10 | TCEP | 2 | 36 | 46 | 16 | 0 |
| C II.11 | TCEP | 0 | 8 | 33 | 47 | 12 |
| E II.99 | TCEPA | 0 | 2 | 5 | 85 | 8 |
| E II.100 | TCEPA | 0 | 0 | 4 | 87 | 9 |
| E II.101 | TCEPA | 0 | 0 | 4 | 88 | 8 |
| E II.102 | TCEPA | 0 | 3 | 5 | 85 | 7 |
| E II.103 | TCEPA | 0 | 0 | 7 | 86 | 7 |
| E II.104 | TCEPA | 0.96 | 3.89 | 4.05 | 84.65 | 6.46 |
| E II.105 | TCEPA | 1.09 | 11.27 | 14.10 | 71.45 | 2.09 |
| E II.106 | TCEPA | 0 | 1.22 | 4.83 | 86.55 | 7.40 |
| E II.107 | TCEPA | 1.04 | 4.67 | 5.54 | 83.54 | 5.21 |
| E II.108 | TCEPA | 0 | 3.70 | 3.90 | 88.11 | 4.28 |
| E II.109 | TCEPA | 1.41 | 8.83 | 20.36 | 66.55 | 2.85 |
| E II.110 | TCEPA | 0 | 7.59 | 17.42 | 52.10 | 22.89 |

TABLE II-12

| | D0 area | D2 area | D4 area | D6 area | D8 area |
|---|---|---|---|---|---|
| Example | (mAU) | (mAU) | (mAU) | (mAU) | (mAU) |
| E II.88 | 0 | 162.79 | 368.48 | 2676.72 | 174.34 |
| E II.89 | 0 | 349.55 | 933.80 | 1708.83 | 45.39 |
| E II.90 | 0 | 45.91 | 152.32 | 1822.70 | 176.29 |
| E II.91 | 0 | 55.00 | 96.42 | 1127.59 | 70.88 |
| E II.92 | 0 | 41.06 | 120.56 | 2431.45 | 388.07 |
| E II.93 | 0 | 0 | 42.22 | 1500.75 | 187.84 |
| E II.94 | 0 | 0 | 75.16 | 2139.05 | 224.92 |
| E II.95 | 0 | 0 | 72.62 | 1676.02 | 166.04 |
| E II.96 | 0 | 44.00 | 114.85 | 1907.43 | 165.15 |
| E II.97 | 0 | 0 | 107.31 | 1670.01 | 148.17 |
| E II.98 | 0 | 32.23 | 97.78 | 2195.59 | 183.24 |
| C II.10 | 64.37 | 994.97 | 1262.14 | 434.27 | 0 |
| C II.11 | 0 | 289.15 | 1111.14 | 1594.53 | 419.56 |
| E II.99 | 0 | 37.16 | 88.19 | 1474.36 | 127.80 |
| E II.100 | 0 | 0 | 82.19 | 1623.61 | 158.78 |
| E II.101 | 0 | 0 | 64.66 | 1612.94 | 151.08 |
| E II.102 | 0 | 50.17 | 91.17 | 1516.96 | 133.57 |
| E II.103 | 0 | 0 | 114.57 | 1472.38 | 125.46 |
| E II.104 | 16.06 | 64.98 | 67.75 | 1415.64 | 108.00 |
| E II.105 | 20.38 | 210.38 | 263.25 | 1334.13 | 38.98 |
| E II.106 | 0 | 14.77 | 58.28 | 1045.22 | 89.38 |
| E II.107 | 15.14 | 68.05 | 80.68 | 1216.98 | 75.85 |
| E II.108 | 0 | 56.24 | 59.23 | 1337.44 | 65.07 |
| E II.109 | 26.50 | 166.02 | 382.87 | 1251.44 | 53.50 |
| E II.110 | 0 | 94.55 | 216.94 | 648.74 | 285.06 |

As the results shown in table II-11, linker-payloads (MC-VC-PAB-MMAE) were successfully linked to Trastuzumab, which indicated ADCs in different buffers were successfully synthesized.

According to the results of table IT-11, the results showed the types of the buffer system will impact the content of D6 by impacting the reduction kinetics and selectivity. When using TCEP, the buffer systems of examples II.88-II.98 are useful to increase the content of D6. When using TCEPA, the proportion of D6 was more than 50% in examples II.99-II.110. Further, the proportion of D6 was more than 70% in examples II.99-II. 108, and the highest proportion of D6 was 88%.

### Examples II.111-II.119: Preparation of Trastuzumab-[MC-VC-PAB-MMAE]₆ Conjugate at Different pH Value

When the reductant is TCEP, the method is similar to example II.1, and when the reductant is not TCEP, the method is similar to example II.79. The differences are the pH value of the buffer system, the molar ratio of TCEPA and the antibody, and/or the kinds of the buffer system which are shown in table II-13.

TABLE II-13

| | | The buffer | Reductant/mAb | |
|---|---|---|---|---|
| No. | Reductant | system | (Molar Ratio) | pH |
| E II.111 | TCEP | BES buffer | 4:1 | 6.4 |
| E II.112 | TCEP | BES buffer | 4:1 | 6.7 |
| E II.113 | TCEP | BES buffer | 4:1 | 7.0 |
| E II.114 | TCEP | BES buffer | 4:1 | 7.4 |
| E II.115 | TCEPA | BES buffer | 3.5:1 | 6.4 |
| E II.116 | TCEPA | BES buffer | 3.5:1 | 6.7 |
| E II.117 | TCEPA | BES buffer | 3.5:1 | 7.4 |
| E II.118 | TCEPA | MES buffer | 3.5:1 | 5.8 |
| E II.119 | TCEPA | MES buffer | 3.5:1 | 6.1 |

The homogeneity assay result and the chromatographic peak area of examples II.111-II.119 were shown in Table II-14 and Table II-15.

TABLE II-14

| | | D0 | D2 | D4 | D6 | D8 |
|---|---|---|---|---|---|---|
| No. | Reductant | (wt %) | (wt %) | (wt %) | (wt %) | (wt %) |
| E II.111 | TCEP | 0 | 4 | 10 | 79 | 7 |
| E II.112 | TCEP | 0 | 0 | 7 | 89 | 4 |
| E II.113 | TCEP | 0 | 0 | 6 | 90 | 4 |
| E II.114 | TCEP | 0 | 0 | 5 | 90 | 5 |
| E II.115 | TCEPA | 0 | 6.21 | 8.05 | 80.67 | 5.07 |
| E II.116 | TCEPA | 0 | 5.34 | 13.90 | 77.02 | 3.75 |
| E II.117 | TCEPA | 0 | 2.73 | 14.61 | 79.28 | 3.38 |
| E II.118 | TCEPA | 1.43 | 14.67 | 25.41 | 58.49 | 0 |
| E II.119 | TCEPA | 0 | 13.45 | 24.28 | 60.53 | 1.74 |

TABLE II-15

| | D0 area | D2 area | D4 area | D6 area | D8 area |
|---|---|---|---|---|---|
| Example | (mAU) | (mAU) | (mAU) | (mAU) | (mAU) |
| E II.111 | 0 | 75.41 | 204.40 | 1543.27 | 136.51 |
| E II.112 | 0 | 0 | 95.27 | 1197.38 | 57.48 |
| E II.113 | 0 | 0 | 117.31 | 1,615.81 | 57.90 |
| E II.114 | 0 | 0 | 75.86 | 1323.11 | 71.83 |
| E II.115 | 0 | 90.90 | 117.88 | 1181.50 | 74.28 |
| E II.116 | 0 | 87.14 | 226.95 | 1257.33 | 61.16 |
| E II.117 | 0 | 30.46 | 163.08 | 884.84 | 37.72 |
| E II.118 | 19.89 | 203.88 | 353.10 | 812.67 | 0 |
| E II.119 | 0 | 251.03 | 453.24 | 1129.75 | 32.50 |

As the results shown in table II-14, linker-payloads (MC-VC-PAB-MMAE) were successfully linked to Trastuzumab, which indicated ADCs with buffers pH value in a range from 5.8 to 7.4 were successfully synthesized.

### Examples II.120-II.126: Preparation of Trastuzumab-[MC-VC-PAB-MMAE]₆ Conjugate at Different Concentration of the Buffer System

When the reductant is TCEP, the method is similar to example II.1, and when the reductant is TCEPA, the method is similar to example II.79. The differences are the concentration of the buffer system and/or the molar ratio of TCEPA and the antibody which are shown in table II-16.

TABLE II-16

| | | The buffer | The concentration of | Reductant/mAb |
|---|---|---|---|---|
| No. | Reductant | system | the buffer system | (Molar Ratio) |
| E II.120 | TCEP | BES buffer | 20 mM | 4:1 |
| E II.121 | TCEP | BES buffer | 40 mM | 4:1 |
| E II.122 | TCEP | BES buffer | 60 mM | 4:1 |
| E II.123 | TCEP | BES buffer | 80 mM | 4:1 |
| E II.124 | TCEPA | BES buffer | 40 mM | 3.5:1 |
| E II.125 | TCEPA | BES buffer | 60 mM | 3.5:1 |
| E II.126 | TCEPA | BES buffer | 80 mM | 3.5:1 |

The homogeneity assay result and the chromatographic peak area of examples II.120-II.126 were shown in Table II-17 and Table II-18.

TABLE II-17

| | | D0 | D2 | D4 | D6 | D8 |
|---|---|---|---|---|---|---|
| No. | Figure | (wt %) | (wt %) | (wt %) | (wt %) | (wt %) |
| E II.120 | Reductant | 0 | 0 | 4 | 86 | 10 |
| E II.121 | TCEP | 0 | 0 | 6 | 85 | 9 |
| E II.122 | TCEP | 0 | 3 | 9 | 81 | 7 |
| E II.123 | TCEP | 0 | 4 | 14 | 76 | 6 |
| E II.124 | TCEP | 0 | 5.83 | 6.32 | 84.06 | 3.79 |
| E II.125 | TCEPA | 1.70 | 5.14 | 3.72 | 85.50 | 3.95 |
| E II.126 | TCEPA | 0 | 7.10 | 6.95 | 83.64 | 2.31 |

TABLE II-18

| | D0 area | D2 area | D4 area | D6 area | D8 area |
|---|---|---|---|---|---|
| Example | (mAU) | (mAU) | (mAU) | (mAU) | (mAU) |
| E II.120 | 0 | 0 | 90.61 | 1817.77 | 204.37 |
| E II.121 | 0 | 0 | 92.33 | 1220.21 | 130.48 |
| E II.122 | 0 | 52.85 | 170.32 | 1588.88 | 140.58 |
| E II.123 | 0 | 64.07 | 250.68 | 1334.35 | 115.92 |
| E II.124 | 0 | 84.54 | 91.60 | 1218.01 | 54.90 |
| E II.125 | 20.27 | 61.08 | 44.24 | 1017.04 | 46.96 |
| E II.126 | 0 | 103.73 | 101.52 | 1222.05 | 33.75 |

As show in table II-17, the results showed the concentration of the buffer systems of examples II.120-II.126 are useful to increase the content of D6.

### Examples II.127-II.143: Preparation of Trastuzumab-[MC-VC-PAB-MMAE]₆ Conjugate (the Reduction Time and/or the Reductant Temperature in Step (1) is Different)

When the reductant is TCEP, the method is similar to example II.1, and when the reductant is TCEPA, the method is similar to example II.79, the differences are the reduction time and/or the reductant temperature in step (I), the reductant, the molar ratio of the reductant and the antibody and/or the molar ratio of the ZnCl₂ and the antibody which are shown in Table II-19.

TABLE II-19

| | | The reduction | The reduction | Reductant/mAb | ZnCl₂/Reductant |
|---|---|---|---|---|---|
| | | temperature in | time in step | (Molar | (Molar |
| No. | Reductant | step (1) (° C.) | (1) (h) | Ratio) | Ratio) |
| E II.127 | TCEP | 4 | 4 | 9:1 | 0.22:1 |
| E II.128 | TCEP | 4 | 6 | 9:1 | 0.22:1 |
| E II.129 | TCEP | 4 | 8 | 9:1 | 0.22:1 |
| E II.130 | TCEP | 4 | 10 | 9:1 | 0.22:1 |
| E II.131 | TCEP | 0 | 18 | 4:1 | 4:1 |
| E II.132 | TCEP | 10 | 18 | 3.8:1 | 2:1 |
| E II.133 | TCEP | 15 | 18 | 3.8:1 | 4:1 |
| E II.134 | TCEP | 25 | 18 | 4:1 | 0.5:1 |
| E II.135 | TCEP-NO | 4 | 4 | 9:1 | 0.22:1 |
| E II.136 | TCEP-NO | 4 | 4 | 10:1 | 0.1:1 |
| E II.137 | TCEP-NO | 4 | 6 | 10:1 | 0.1:1 |
| E II.138 | TCEP-NO | 4 | 8 | 10:1 | 0.1:1 |
| E II.139 | TCEP-NO | 4 | 10 | 10:1 | 0.1:1 |
| E II.140 | TCEPA | 4 | 22 | 3.5:1 | 0.57:1 |
| E II.141 | TCEPA | 10 | 16 | 3.2:1 | 0.63:1 |
| E II.142 | TCEPA | 15 | 16 | 3.5:1 | 0.57:1 |
| E II.143 | TCEPA | 25 | 6 | 3.9:1 | 0.51:1 |

The homogeneity assay result and the chromatographic peak area of examples II.127-II.143 were shown in Table II-20 and Table II-21.

TABLE II-20

| | | The reduction | | | | | |
|---|---|---|---|---|---|---|---|
| | | temperature and | | | | | |
| No. | Reductant | time in step (1) | D0 (%) | D2 (%) | D4 (%) | D6 (%) | D8 (%) |
| E II.127 | TCEP | 4° C., 4 h | 0 | 3.83 | 9.73 | 86.44 | 0 |
| E II.128 | TCEP | 4° C., 6 h | 0 | 2.39 | 5.87 | 90.62 | 1.13 |
| E II.129 | TCEP | 4° C., 8 h | 0 | 2.09 | 4.66 | 91.65 | 1.60 |
| E II.130 | TCEP | 4° C., 10 h | 0 | 1.88 | 4.00 | 91.34 | 2.79 |
| E II.131 | TCEP | 0° C., 18 h | 0 | 1.37 | 4.48 | 88.17 | 5.99 |
| E II.132 | TCEP | 10° C., 18 h | 0 | 2.28 | 9.34 | 88.38 | 0 |
| E II.133 | TCEP | 15° C., 18 h | 0 | 2.06 | 9.48 | 86.32 | 2.14 |
| E II.134 | TCEP | 25° C., 18 h | 1.05 | 1.47 | 12.27 | 79.69 | 5.52 |
| E II.135 | TCEP-NO | 4° C., 4 h | 0 | 4.12 | 5.79 | 77.34 | 12.75 |
| E II.136 | TCEP-NO | 4° C., 4 h | 0 | 3.49 | 9.03 | 85.31 | 2.17 |
| E II.137 | TCEP-NO | 4° C., 6 h | 0 | 2.00 | 5.23 | 88.91 | 3.86 |
| E II.138 | TCEP-NO | 4° C., 8 h | 0 | 0 | 3.19 | 88.59 | 8.22 |
| E II.139 | TCEP-NO | 4° C., 10 h | 0 | 0 | 2.09 | 85.57 | 12.34 |
| E II.140 | TCEPA | 4° C., 22 h | 0 | 4.42 | 7.84 | 87.75 | 0 |
| E II.141 | TCEPA | 10° C., 16 h | 0 | 2.82 | 8.48 | 84.42 | 4.28 |
| E II.142 | TCEPA | 15° C., 16 h | 0 | 5.35 | 8.07 | 84.71 | 1.87 |
| E II.143 | TCEPA | 25° C., 6 h | 1.05 | 2.63 | 5.76 | 75.35 | 15.22 |

TABLE II-21

| | D0 area | D2 area | D4 area | D6 area | D8 area |
|---|---|---|---|---|---|
| Example | (mAU) | (mAU) | (mAU) | (mAU) | (mAU) |
| E II.127 | 0 | 48.71 | 123.55 | 1098.12 | 0 |
| E II.128 | 0 | 32.40 | 79.41 | 1226.98 | 15.23 |
| E II.129 | 0 | 22.68 | 50.73 | 996.84 | 17.40 |
| E II.130 | 0 | 25.49 | 54.22 | 1238.66 | 37.78 |
| E II.131 | 0 | 179.67 | 307.09 | 1400.99 | 68.54 |
| E II.132 | 0 | 26.32 | 107.77 | 1020.16 | 0 |
| E II.133 | 0 | 31.54 | 145.26 | 1322.88 | 32.79 |
| E II.134 | 14.33 | 20.03 | 167.27 | 1086.53 | 75.31 |
| E II.135 | 0 | 74.66 | 104.84 | 1401.10 | 230.97 |
| E II.136 | 0 | 51.13 | 132.11 | 1248.66 | 31.73 |
| E II.137 | 0 | 22.82 | 59.68 | 1014.39 | 44.08 |
| E II.138 | 0 | 0 | 43.33 | 1203.77 | 111.73 |
| E II.139 | 0 | 0 | 24.75 | 1013.74 | 146.21 |
| E II.140 | 0 | 44.94 | 79.74 | 892.72 | 0 |
| E II.141 | 0 | 22.92 | 69.08 | 687.46 | 34.86 |
| E II.142 | 0 | 68.99 | 104.01 | 1091.96 | 24.13 |
| E II.143 | 4.07 | 10.21 | 22.39 | 292.94 | 59.16 |

As shown in table II.20, using TCEP, the results showed the content of D6 is up to 86%, even to 90% when the molar ratio of TCEP and the antibody is 9:1 and the reductant time in step (1) is from 4 h to 10 h, which indicates that increasing the molar ratio of TCEP and the antibody, the method displayed here is with less reduction time cost. Meanwhile, the content of D6 is up to 80%, 85%, even to 88% when the reductant temperature in step (1) is from 0° C. to 25° C.

As shown in examples II.135-II.140, the results showed the content of D6 is up to 75%, even to 80, 85 or 88% when the molar ratio of the reductant and the antibody is 3.5:1, 9:1 or 10:1 and the reductant time in step (1) is from 4 h to 22 h, which also indicates that increasing the molar ratio of the reductant and the antibody, the method displayed here is with less reduction time cost. Meanwhile, the results showed the content of D6 is up to 75%, 85%, even to 87% when the reductant temperature in step (1) is from 4° C. to 25° C..

### Example II.144: Preparation of Trastuzumab-[Bismaleimide-DBCO]₃ Conjugate

### 1. Synthesis of Bismaleimide-DBCO (a Thiobridge)

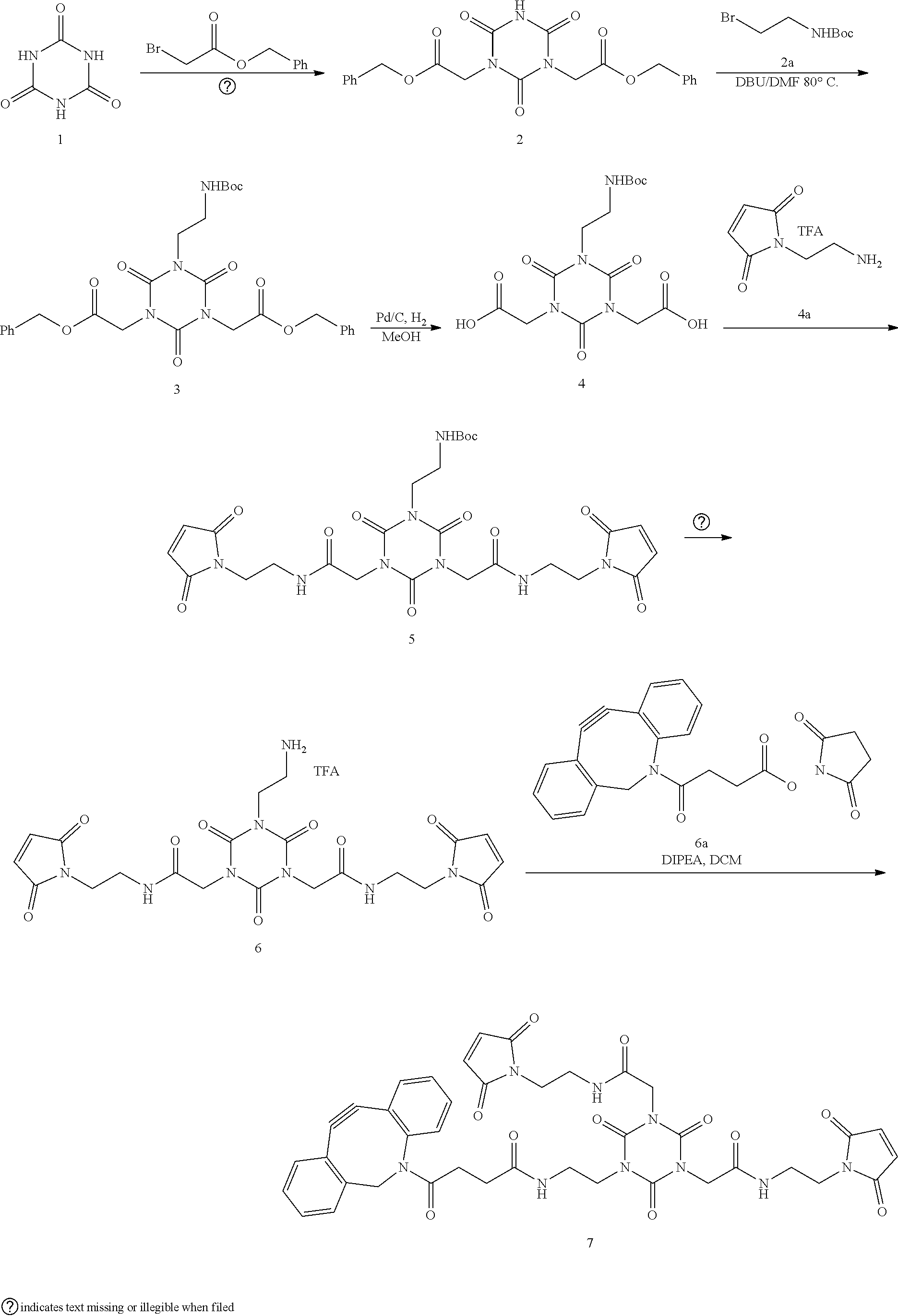

Intermediate 2: To a mixture of 1 (6.5 g, 50.0 mmol, 1.0 eq) in DMF (100 mL) was added DBU (30.7 g, 0.2 mol, 4.0 eq). The mixture was stirred for 1 h at 80° C., and Benzyl 2-bromoacetate (25.4 g, 0.11 mol, 2.2 eq). The resulting mixture was stirred for 16 h at 80° C. The mixture was poured into ice-water (600 mL), extracted with EtOAc (200 mL*3). The combined organic layer was washed with brine (200 mL), dried over Na₂SO₄ and filtered. The filtrate was concentrated, and purified by flash column (EtOAc/petroleum ether=0˜40%) to give product 2 (4.5 g, 21.0%) as white solid.

Intermediate 3: To a mixture of intermediate 2 (4.5 g, 10.58 mmol, 1.0 eq) in DMF (50 mL) was added DBU (3.22 g, 21.16 mmol, 2.0 eq). The mixture was stirred for 0.5 h at 80° C., and tert-Butyl N-(2-bromoethyl) carbamate (3.56 g, 15.87 mmol, 1.5 eq). The resulting mixture was stirred for 6 h at 80° C. TLC showed compound 1 was consumed completely. The mixture was poured into ice-water (300 mL), extracted with EtOAc (100 mL*3). The combined organic layer was washed with brine (100 mL), dried over Na₂SO₄ and filtered. The filtrate was concentrated, and purified by flash column (EtOAc/petroleum ether=0˜40%) to give product 3 (4.8 g, 79.8%) as white solid.

Intermediate 4: To a mixture of intermediate 3 (4.8 g, 8.44 mmol, 1.0 eq) in THF (50 mL) was added Pd/C (500 mg, 10% Pd/C, wetted with ca. 55% Water). The mixture was degassed 3 times and purged with H₂. The resulting mixture was stirred for 2 h at room temperature under H₂ atmosphere. TLC showed intermediate 3 was consumed completely. The mixture was filtered through a Celite pad and filtrate was concentrated to give product 4 (3.0 g, 91.5%) as white solid.

Intermediate 5: To a mixture of intermediate 4 (50 mg, 1.00 mmol, 1.0 eq) in DMF (5 mL) was added HATU (0.92 g, 2.4 mmol, 2.4 eq) and DIPEA (0.65 g, 5.0 mmol, 5.0 eq). The mixture was stirred for 0.5 h at room temperature, and 4a (0.51 g, 2.0 mmol, 2.0 eq) was added. The resulting mixture was stirred for 2 h at room temperature. LCMS showed intermediate 4 was consumed completely. The mixture was quenched by adding HCl (0.5M, 2 mL), and purified by RP-column (water/MeCN=10˜70%), and the eluent was lyophilized to give product 5 (0.51 g, 80.3%) as white solid.

Intermediate 6: To a mixture of intermediate 5 (100 mg, 0.16 mmol, 1.0 eq) in DCM (2 mL) was added TFA (100 uL). The mixture was stirred for 1 hours at room temperature. LCMS showed intermediate 5 was consumed completely. The mixture was concentrated and the residue was taken up by water (10 mL), then lyophilized to give product 6 (100 mg, 97.8%) as white solid.

Compound 7 (Bismaleimide-DBCO): To a mixture of intermediate 6 (50 mg, 93.9 umol, 1.0 eq) in DCM (2 mL) was added 6a (37.8 mg, 93.9 umol, 1.0 eq) followed by DIPEA (17 uL, 93.9 umol, 1.0 eq). The mixture was stirred for 1 h at room temperature. LCMS showed intermediate 6 was consumed completely. The mixture was concentrated and the residue was purified by prep-HPLC (water/MeCN=10˜50%), and the eluent was lyophilized to give product 7 (28.8 mg, 3.41%) as white solid. MS(M+H)⁺=820.19, exact mass calc. for C₄₀H₃₇N₉O₁₁ is 819.26. ¹H NMR (400 MHz, DMSO-d6): δ 8.17 (t, J=6.0 Hz, 2H), 7.85 (d, J=6.4 Hz, 1H), 7.64 (dd, J=18.4, 7.3 Hz, 2H), 7.47 (d, J=10.3 Hz, 3H), 7.38-7.24 (m, 3H), 6.96 (s, 4H), 5.02 (d, J=14.0 Hz, 1H), 4.22 (s, 4H), 3.68 (t, J=6.6 Hz, 2H), 3.65-3.58 (m, 4H), 3.43 (t, J=6.2 Hz, 3H), 3.24-3.20 (m, 3H), 3.16-3.10 (m, 5H).

### 2. Preparation of Trastuzumab-[Bismaleimide-DBCO]₃

(1) TCEP (0.048 mM) and ZnCl₂ (0.024 mM) were added to a solution of Trastuzumab (0.012 mM) in BES buffer (20 mM, pH7.0) and the reaction mixture was vortexed for mixing, then the reaction mixture was incubated at 4° C. for 18 h;
(2) introducing EDTA (0.6 mM) and Bismaleimide-DBCO (0.045 mM) to react with reduced thiol groups resulted from step (1) at room temperature for 1 h, then recovering Trastuzumab-[Bismaleimide-DBCO]₃ using a desalting column.

### Example II.145: Preparation of Trastuzumab-[Bismaleimide-DBCO]₃ Conjugate

(1) ZnCl₂ (0.024 mM) and reductant TCEPA (0.048 mM) were added to a solution of a monoclonal antibody Trastuzumab (0.012 mM, in BES buffer, pH7.0, 20 mM) and the reaction mixture was allowed to stay at 4° C. for 18 h;
(2) EDTA-2Na (0.6 mM) and Bismaleimide-DBCO (0.045 mM) in DMA was introduced and the reaction was continued at room temperature for 1 h, then recovering Trastuzumab-[Bismaleimide-DBCO]₃ using a desalting column.

The homogeneity assay result and the chromatographic peak area and of examples II.144-II.145 were shown in Table II-22 and Table II-23.

TABLE II-22

| No. | Figure | D0 (%) | D1 (%) | D3 (%) | D4 (%) |
|---|---|---|---|---|---|
| E II.144 | 18 | 0 | 0 | 86.55 | 13.45 |
| E II 145 | 19 | 0 | 0 | 82.93 | 17.07 |

TABLE II-23

| | D0 area | D1 area | D3 area | D4 area |
|---|---|---|---|---|
| No. | (mAU) | (mAU) | (mAU) | (mAU) |
| E II.144 | 0 | 0 | 1995.26 | 310.08 |
| E II 145 | 0 | 0 | 2248.78 | 462.98 |

As shown in the above table, the result demonstrated that the content of Trastuzumab-[Bismaleimide-DBCO]₃ was generally up to 82% or 86%, which indicated the process of method was benefit for site-specific modifying the antibody with D3 and improving the homogeneity.

### Example II.146: Preparation of Trastuzumab-[MC-GGFG-DXd]₆[MC-VC-PAB-MMAE]₂ (The ADC with D6+D2)

(1) ZnCl₂ (0.048 mM) and reductant TCEP (0.048 mM) were added to a solution of a monoclonal antibody Trastuzumab (0.012 mM) in BES buffer (pH7.0, 20 mM) and the reaction mixture was allowed to stay at 4° C. for 18 h;
(2) EDTA-2Na (0.6 mM) and MC-GGFG-DXd (0.1 mM) in DMA was introduced and the reaction was continued at room temperature for 1 h;
(3) The resulting product and the second reductant TCEP (0.02 mM) were incubating at room temperature for 3 h; a second linker-payload MC-VC-PAB-MMAE (0.05 mM) was added and the reaction was continued at room temperature for 2 h.
(4) purifying the resultant product using a desalting column

### Example II.147: Preparation of Trastuzumab-[MC-VC-PAB-MMAE]₆ [MC-GGFG-DXd]₂ (The ADC with D6+D2)

(1) ZnCl₂ (0.024 mM) and reductant TCEPA (0.048 mM) were added to a solution of a monoclonal antibody Trastuzumab (0.012 mM) in BES buffer (pH7.0, 20 mM) and the reaction mixture was allowed to stay at 4° C. for 18 h;
(2) EDTA-2Na (0.6 mM) and MC-VC-PAB-MMAE (0.08 mM) in DMA was introduced and the reaction was continued at room temperature for 1 h;
(3) The resulting product of step (2) and the second reductant TCEP (0.02 mM) were incubating at 37° C. for 3 h; a second linker-payload MC-GGFG-DXd (0.05 mM) added and the reaction was continued at room temperature for 2 h.
(4) purifying the resultant product using a desalting column The homogeneity assay result and the chromatographic peak area and of examples II.146-II.147 were shown in Table II-24 and Table II-25.

TABLE II-24

| NO. | Figure | D2 (%) | D4 (%) | D6 (%) | D8 (%) |
|---|---|---|---|---|---|
| E II.146- | 20A | 0 | 2.24 | 88.82 | 8.93 |
| step (2) | | | | | |
| E II.147- | 21A | 4.48 | 4.97 | 86.49 | 4.06 |
| step (2) | | | | | |

| | | | D4 + | D6 + | |
|---|---|---|---|---|---|
| NO. | Figure | / | D2 (%) | D2 (%) | D8 (%) |
| E II.146- | 20B | / | 3.58 | 87.10 | 9.32 |
| step (4) | | | | | |

| | | | | D6 + | |
|---|---|---|---|---|---|
| NO. | Figure | D2 (%) | D4(%) | D2 (%) | D8 (%) |
| E II.147- | 21B | 3.67 | 3.88 | 84.81 | 7.65 |
| step (4) | | | | | |

TABLE II-25

| | D2 area | D4 area | D6 area | D8 area |
|---|---|---|---|---|
| NO. | (mAU) | (mAU) | (mAU) | (mAU) |
| E II.146- | 0 | 40.552 | 1605.450 | 161.482 |
| step (2) | | | | |
| E II.147- | 72.86 | 80.77 | 1405.26 | 65.97 |
| step (2) | | | | |

| | | D4 + | D6 + | |
|---|---|---|---|---|
| | | D2 area | D2 area | D8 area |
| NO. | / | (mAU) | (mAU) | (mAU) |
| E II.146- | / | 45.033 | 1094.874 | 117.143 |
| step (4) | | | | |

| | | | D6 + | |
|---|---|---|---|---|
| | D2 area | D4 area | D2 area | D8 area |
| NO. | (mAU) | (mAU) | (mAU) | (mAU) |
| E II.147- | 3.72 | 3.94 | 86.11 | 7.77 |
| step (4) | | | | |

As shown in the above table, the result demonstrated that the content of the ADC with D6+D2 was generally up to 84.81% or 87.10%, which indicated the process of method was benefit for site-specific modifying the antibody with D6+D2 and improving the homogeneity.

### Example II.148: Preparation of Trastuzumab-[MC-VC-PAB-MMAE]₆[Maleimide-PEG4-N3-DBCO-Cy3]₁ (The ADC with D6+D1)

(1) ZnCl₂ (0.0408 mM) and TCEP (0.024 mM) were added toa solution of a monoclonal antibody Trastuzumab (0.012 mM) in BES buffer (pH7.0, 20 mM) and the reaction mixture was allowed to stay at 4° C. for 16 h;
(2) EDTA-2Na (0.6 mM) and MC-VC-PAB-MMAE (0.1 mM) in DMA was introduced and the reaction was continued at room temperature for 1 h, then recovering the product using a desalting column;
(3) The resulting product and reductant TCEP (0.09 mM) were incubating at room temperature for 20 h; the second thiobridge reagent with the reactive group Dibromomaleimide-PEG4-N3 (0.012 mM) was added and the reaction was continued at room temperature for 2 h; then the second linker-payload DBCO-Cy3 (0.05 mM) was added and the reaction was continued at room temperature for 4 h.
(4) purifying the resultant product using a desalting column.

### Example II.149: Preparation of Trastuzumab-[MC-GGFG-DXd]₆[Maleimide-PEG4-N3-DBCO-Cy3]₁ (The ADC with D6+D1)

(1) ZnCl₂ (0.024 mM) and reductant TCEPA (0.048 mM) were added to a solution of a monoclonal antibody Trastuzumab (0.012 mM) in BES buffer (pH7.0, 20 mM) and the reaction mixture was allowed to stay at 4° C. for 18 h;
(2) EDTA-2Na (0.6 mM) and MC-GGFG-DXd (0.08 mM) in DMA was introduced and the reaction was continued at room temperature for 1 h, then recovering the product using a desalting column;
(3) The resulting product of step (2) and the second reductant TCEP (0.02 mM) were incubating at room temperature for 2 h; a thio-bridging reagent [Dibromomaleimide-PEG4-N3](0.012 mM) added and the reaction was continued at room temperature for 2 h; then a second linker-payload [DBCO-Cy3](0.05 mM) added and the reaction was continued at room temperature for 4 h.
(4) purifying the resultant product using a desalting column.

The homogeneity assay result and the chromatographic peak area and of examples II.148-II.149 were shown in Table II-26 and Table II-27.

TABLE II-26

| NO. | Figure | D2 (%) | D4 (%) | D6 (%) | D8 (%) |
|---|---|---|---|---|---|
| E II.148- | 22A | 4.48 | 4.97 | 86.49 | 4.06 |
| step (2) | | | | | |
| E II.149- | 23A | 0 | 4.58 | 92.20 | 3.23 |
| step (2) | | | | | |

| | | D4 + | D6 + | | |
|---|---|---|---|---|---|
| NO. | Figure | D1 (%) | D1 (%) | D8 (%) | / |
| E II.148- | 22B | 1 | 89.89 | 10.11 | / |
| step (4) | | | | | |
| E II.149- | 23B | 4.26 | 92.25 | 3.50 | / |
| step (4) | | | | | |

TABLE II-27

| | D2 (%) area | D4 (%) area | D6 (%) area | D8 (%) area |
|---|---|---|---|---|
| No. | (mAU) | (mAU) | (mAU) | (mAU) |
| E II.148- | 72.86 | 80.77 | 1405.26 | 65.97 |
| step (2) | | | | |
| E II.149- | 0 | 81.80 | 1739.35 | 64.51 |
| step (2) | | | | |

| | D4 + | D6 + | | |
|---|---|---|---|---|
| | D1 area | D1 area | D8 area | |
| NO. | (mAU) | (mAU) | (mAU) | / |
| E II.148- | / | 575.00 | 64.69 | / |
| sep (4) | | | | |
| E II.149- | 46.43 | 1006.05 | 38.16 | / |
| step (4) | | | | |

As shown the above table, the result demonstrated that the content of the ADC with D6+D1 was generally up to 89.89% or 92.25%, which indicated the process of method was benefit for site-specific modifying the antibody with D6+D1 and improving the homogeneity.

### Example II.150: Preparation of Trastuzumab-[Maleimide]₆[MC-VC-PAB-MMAE]₂ (The ADC with D0+D2)

(1) TCEP (0.048 mM) and ZnCl₂ (0.024 mM) were added to a solution of Trastuzumab (0.012 mM) in BES buffer (20 mM, pH7.0) and the reaction mixture was vortexed for mixing, then the reaction mixture was incubated at 4° C. for 18 h;
(2) introducing EDTA (0.6 mM) and (2-Aminoethyl) maleimide (0.1 mM) to react with reduced thiol groups resulted from step (1) at room temperature for 1 h, then recovering the product using a desalting column to afford Trastuzumab-[Maleimide]₆;
(3) Trastuzumab-[Maleimide]₆ and reductant TCEP (0.02 mM) were incubating at room temperature for 18 h; a second linker-payload MC-VC-PAB-MMAE (0.05 mM) was added and the reaction was continued at room temperature for 2 h.
(4) purifying the resultant product using a desalting column.

### Example II.151: Preparation of Trastuzumab-[Maleimide]₆[MC-VC-PAB-MMAE]₂ (The ADC with D0+D2)

The method is similar to example II.150. The differences are that the reductant is TCEPA in step (1) and the incubation temperature of Trastuzumab-[Maleimide]₆ and reductant TCEP is 37C in step (3).

The homogeneity assay result and the chromatographic peak area and of examples II.150-II.151 were shown in Table II-28 and Table II-29.

TABLE II-28

| NO. | Figure | D0(%) | / | / | / |
|---|---|---|---|---|---|
| E II.150- | 24A | 100 | / | / | / |
| step (2) | | | | | |
| E II.151- | 25A | 100 | / | / | / |
| step (2) | | | | | |

| | | | D0 + | D0 + | D0 + |
|---|---|---|---|---|---|
| NO. | Figure | D0 | D1 (%) | D2 (%) | D3 (%) |
| E II.150- | 24B | 11.66 | 6.31 | 70.92 | 11.11 |
| step (4) | | | | | |
| E II.151- | 25B | 24.30 | 5.18 | 70.53 | 0 |
| step (4) | | | | | |

TABLE II-29

| | D0 area | | | |
|---|---|---|---|---|
| NO. | (mAU) | / | / | / |
| E II.150-step (2) | 3091.23 | / | / | / |
| E II.151-step (2) | 3362.90 | / | / | / |

| | | D0 + | D0 + | D0 + |
|---|---|---|---|---|
| | D0 area | D1 area | D2 area | D3 area |
| NO. | (mAU) | (mAU) | (mAU) | (mAU) |
| E II.150-step (4) | 92.167 | 49.884 | 560.664 | 87.845 |
| E II.151-step (4) | 143.74 | 30.61 | 417.28 | 0 |

As shown the above table, the result demonstrated that the content of the ADC with D0+D2 was generally up to 70%, which indicated the process of method was benefit for site-specific modifying the antibody with D0+D2 and improving the homogeneity.

### Example II.152: Preparation of Trastuzumab-[Maleimide]₆[Maleimide-PEG4-N3-DBCO-Cy3]₁ (The ADC with D0+D1)

(1) TCEP (0.048 mM) and ZnCl₂ (0.024 mM) were added to a solution of Trastuzumab (0.012 mM) in BES buffer (20 mM, pH7.0) and the reaction mixture was vortexed for mixing, then the reaction mixture was incubated at 4° C. for 18 h;
(2) introducing EDTA (0.6 mM) and (2-Aminoethyl) maleimide (0.1 mM) to react with reduced thiol groups resulted from step (1) at room temperature for Ih, then recovering the product using a desalting column to afford Trastuzumab-[Maleimide]₆;
(3) Trastuzumab-[Maleimide]₂ and the second reductant TCEP (0.02 mM) were incubating at room temperature for 18 h. the second thiobridge reagent with the reactive group dibromomaleimide-PEG4-N3 (0.013 mM) to react with reduced thiol groups, the reaction temperature is 24° C. and the reaction time is 3 h;
(4) incubating resulting product and DBCO-Cy3 (0.02 mM) in MES (20 mM, pH6.7), the reaction temperature is 25° C. and the reaction time is 8 h, then recovering Trastuzumab-[Maleimide]₆[Maleimide-PEG4-N3-DBCO-Cy3] using a desalting column.

### Example II.153: Preparation of Trastuzumab-[Maleimide]₆[Maleimide-PEG4-N3-DBCO-Cy3]₁ (The ADC with D0+D1)

The method is similar to example II.152. The differences are that the reductant is TCEPA in step (1) and the incubation temperature of Trastuzumab-[Maleimide]₂ and TCEP is 37° C. in step (3).

The homogeneity assay result and the chromatographic peak area and of examples II.152-II.153 were shown in Table II-30 and Table II-31.

TABLE II-30

| | | | D0 + | D0 + |
|---|---|---|---|---|
| No. | Figure | D (%) | D1 (%) | D2 (%) |
| E II.152 | 26 | 7.04 | 92.96 | 0 |
| E II.153 | 27 | 17.00 | 79.40 | 3.60 |

TABLE II-31

| | D0 area | D0 + | D0 + |
|---|---|---|---|
| No. | (mAU) | D1 area (mAU) | D2 area (mAU) |
| E II.152 | 43.473 | 574.038 | 0 |
| E II.153 | 109.38 | 510.84 | 23.15 |

As shown the above table, the result demonstrated that the content of the ADC with D0+D1 was generally up to 80% or 92.96%, which indicated the process of method was benefit for site-specific modifying the antibody with D0+D1 and improving the homogeneity.

### Example III.1: Preparation of Trastuzumab-[MC-VC-PAB-MMAE]₂ conjugates (The ADC with D2)

(1) ZnCl₂ (0.012 mM) and TCEP (0.048 mM) were added to a solution of a monoclonal antibody Trastuzumab (0.012 mM) in BES buffer (pH7.0, 20 mM) and the reaction mixture was allowed to stay at 4° C. for 18 h;
(2) DHAA (0.096) was added and vortexed for mixing. The mixture was then incubated in darkness, the reaction condition showed table 8 below;
(3) optionally, purification with a desalting column to remove excess DHAA;
(4) EDTA (0.06 mM) and MC-VC-PAB-MMAE (0.048 mM) in DMA were added and the mixture was then incubated at room temperature for 1 h.
(5) The reaction mixture was subjected to purification using a de-salting column.

### Examples III.2-III.53: Preparation of Trastuzumab-[MC-VC-PAB-MMAE]₂ Conjugates (The ADC with D2)

The method is similar to example III. 1, and the differences are the reductant and/or the parameters in step (1) and in step (2) which are shown in table III-1 and Table III-2. Meanwhile, the oxidation time in step (2) is 2 h in examples III.40-III.53.

TABLE III-1

| | | DHAA/antibody | | Zn²⁺/antibody | The temperature, time |
|---|---|---|---|---|---|
| | | (molar | Antibody | (molar | and purification |
| No. | Reductant | ratio) | (mM) | ratio) | of oxidation |
| E III.2 | TCEP | 4:1 | 0.012 | 1:1 | RT, 2 h |
| E III.3 | TCEP | 4.5:1 | 0.012 | 1:1 | RT, 2 h |
| E III.4 | TCEP | 6:1 | 0.012 | 1:1 | RT, 2 h |
| E III.5 | TCEP | 8:1 | 0.012 | 1:1 | RT, 2 h |
| E III.6 | TCEP | 8:1 | 0.012 | 1:1 | 4° C., 5 h |
| E III.7 | TCEP | 8:1 | 0.012 | 1:1 | RT, 2 h, purify the |
| | | | | | oxidated products |
| | | | | | using a de-salting |
| | | | | | column |
| E III.8 | TCEP | 10:1 | 0.012 | 1:1 | RT, 2 h |
| E III.9 | TCEP | 10:1 | 0.012 | 1:1 | 4° C., 5 h |
| E III.10 | TCEP | 10:1 | 0.012 | 1:1 | RT, 2 h, purify the |
| | | | | | oxidated products |
| | | | | | using a de-salting |
| | | | | | column |
| E III.11 | TCEP | 12:1 | 0.012 | 1:1 | RT, 2 h |
| E III.12 | TCEP | 14:1 | 0.012 | 1:1 | RT, 2 h |
| E III.13 | TCEP | 15:1 | 0.012 | 1:1 | RT, 2 h |
| E III.14 | TCEP | 8:1 | 0.024 | 2:1 | RT, 2 h |
| E III.15 | TCEP | 15:1 | 0.024 | 2:1 | RT, 2 h |
| E III.16 | TCEPA | 6:1 | 0.012 | 1:1 | RT, 2 h |
| E III.17 | TCEPA | 8:1 | 0.012 | 1:1 | RT, 2 h |
| E III.18 | TCEPA | 10:1 | 0.012 | 1:1 | RT, 2 h |
| E III.19 | TCEPA | 12:1 | 0.012 | 1:1 | RT, 2 h |
| E III.20 | TCEPA | 14:1 | 0.012 | 1:1 | RT, 2 h |
| E III.21 | TCEPA | 8:1 | 0.012 | 1:1 | RT, 2 h and |
| | | | | | purification |
| | | | | | after oxidation |
| E III.22 | TCEPA | 8:1 | 0.012 | 1:1 | 4° C., 5 h |
| E III.23 | TCEPA | 10:1 | 0.012 | 1:1 | RT, 2 h and |
| | | | | | purification |
| | | | | | after oxidation |
| E III.24 | TCEPA | 10:1 | 0.012 | 1:1 | 4° C., 5 h |

TABLE III-2

| | | Reductant/antibody | DHAA/antibody | | The reduction |
|---|---|---|---|---|---|
| | | (molar | (molar | Antibody | time in step |
| No. | Reductant | ratio) | ratio) | (mM) | (1) (h) |
| E III.25 | TCEP | 6:1 | 16:1 | 0.012 | 18 |
| E III.26 | TCEP | 10:1 | 18:1 | 0.012 | 18 |
| E III.27 | TCEP | 4:1 | 7:1 | 0.012 | 4 |
| E III.28 | TCEP | 4:1 | 9:1 | 0.012 | 4 |
| E III.29 | TCEP | 4:1 | 11:1 | 0.012 | 4 |
| E III.30 | TCEP | 6:1 | 12:1 | 0.012 | 4 |
| E III.31 | TCEP | 6:1 | 14:1 | 0.012 | 4 |
| E III.32 | TCEP | 6:1 | 16:1 | 0.012 | 4 |
| E III.33 | TCEP | 10:1 | 14:1 | 0.012 | 4 |
| E III.34 | TCEP | 10:1 | 16:1 | 0.012 | 4 |
| E III.35 | TCEP | 10:1 | 18:1 | 0.012 | 4 |
| E III.36 | TCEP | 10:1 | 20:1 | 0.012 | 4 |
| E III.37 | TCEP | 10:1 | 22:1 | 0.012 | 4 |
| E III.38 | TCEP | 10:1 | 24:1 | 0.012 | 4 |
| E III.39 | TCEP | 5:1 | 12:1 | 0.012 | 4 |
| E III.40 | TCEP-NO | 4:1 | 4:1 | 0.012 | 2 |
| E III.41 | TCEP-NO | 4:1 | 4:1 | 0.012 | 4 |
| E III.42 | TCEP-NO | 4:1 | 6:1 | 0.012 | 4 |
| E III.43 | TCEP-NO | 4:1 | 8:1 | 0.012 | 2 |
| E III.44 | TCEP-NO | 4:1 | 8:1 | 0.012 | 4 |
| E III.45 | TCEP-NO | 4:1 | 10:1 | 0.012 | 4 |
| E III.46 | TCEP-NO | 6:1 | 7:1 | 0.012 | 4 |
| E III.47 | TCEP-NO | 6:1 | 10:1 | 0.012 | 4 |
| E III.48 | TCEP-NO | 6:1 | 13:1 | 0.012 | 4 |
| E III.49 | TCEP-NO | 6:1 | 16:1 | 0.012 | 4 |
| E III.50 | TCEP-NO | 10:1 | 10:1 | 0.012 | 4 |
| E III.51 | TCEP-NO | 10:1 | 14:1 | 0.012 | 4 |
| E III.52 | TCEP-NO | 10:1 | 18:1 | 0.012 | 4 |
| E III.53 | TCEP-NO | 10:1 | 22:1 | 0.012 | 4 |

### Comparative Examples III.1-III.3: Preparation of Trastuzumab-[MC-VC-PAB-MMAE]₂ Conjugate (the Molar Ratio of and Zn²⁺ and TCEP is Different)

The method of comparative examples III. 1 is the same as example III.29, comparative examples III.2 is the same as example III.32, comparative examples III.3 is the same as example III.35, the difference is that the concentration of ZnCl₂ in step (1) is 0.

The homogeneity assay result and the chromatographic peak area and of examples III.1-III.53 and comparative examples III. 1-III.3 were shown in Table III-3-Table III-6.

TABLE III-3

| | | | The temperature, time | | | | | |
|---|---|---|---|---|---|---|---|---|
| | | DHAA/antibody | and purification of | D0 | D2 | D4 | D6 | D8 |
| No. | Reductant | (molar ratio) | oxidation | (%) | (%) | (%) | (%) | (%) |
| E III.1 | TCEP | 8:1 | 25° C., 1 h | 5.26 | 92.06 | 2.68 | 0 | 0 |
| E III.2 | TCEP | 4:1 | RT, 2 h | 1.25 | 63.55 | 22.44 | 12.75 | 0 |
| E III.3 | TCEP | 4.5:1 | RT, 2 h | 2.13 | 76.49 | 14.67 | 6.71 | 0 |
| E III.4 | TCEP | 6:1 | RT, 2 h | 4.79 | 93.22 | 1.98 | 0 | 0 |
| E I5I.5 | TCEP | 8:1 | RT, 2 h | 6.46 | 93.54 | 0 | 0 | 0 |
| E III.6 | TCEP | 8:1 | 4° C., 5 h | 5.74 | 87.37 | 4.80 | 2.10 | 0 |
| E III.7 | TCEP | 8:1 | RT, 2 h, purify the | 5.03 | 94.97 | 0 | 0 | 0 |
| | | | oxidated products using | | | | | |
| | | | a de-salting column | | | | | |
| E III.8 | TCEP | 10:1 | RT, 2 h | 8.57 | 91.43 | 0 | 0 | 0 |
| E III.9 | TCEP | 10:1 | 4° C., 5 h | 7.95 | 90.08 | 1.97 | 0 | 0 |
| E III.10 | TCEP | 10:1 | RT, 2 h, purify the | 6.20 | 93.8 | 0 | 0 | 0 |
| | | | oxidated products using | | | | | |
| | | | a de-salting column | | | | | |
| E III.11 | TCEP | 12:1 | RT, 2 h | 10.05 | 89.95 | 0 | 0 | 0 |
| E III.12 | TCEP | 14:1 | RT, 2 h | 11.19 | 88.81 | 0 | 0 | 0 |
| E III.13 | TCEP | 15:1 | RT, 2 h | 10.64 | 89.36 | 0 | 0 | 0 |
| E III.14 | TCEP | 8:1 | RT, 2 h | 2.97 | 76.43 | 16.20 | 4.40 | 0 |
| E III.15 | TCEP | 15:1 | RT, 2 h | 8.06 | 89.19 | 2.75 | 0 | 0 |
| E III.16 | TCEPA | 6:1 | RT, 2 h | 3.93 | 93.07 | 3.00 | 0 | 0 |
| E III.17 | TCEPA | 8:1 | RT, 2 h | 6.82 | 93.18 | 0 | 0 | 0 |
| E III.18 | TCEPA | 10:1 | RT, 2 h | 7.64 | 92.36 | 0 | 0 | 0 |
| E III.19 | TCEPA | 12:1 | RT, 2 h | 10.10 | 89.90 | 0 | 0 | 0 |
| E III.20 | TCEPA | 14:1 | RT, 2 h | 12.23 | 87.78 | 0 | 0 | 0 |
| E III.21 | TCEPA | 8:1 | RT, 2 h and purification | 4.67 | 95.33 | 0 | 0 | 0 |
| | | | after oxidation | | | | | |
| E III.22 | TCEPA | 8:1 | 4° C., 5 h | 6.36 | 86.09 | 4.64 | 2.91 | 0 |
| E III.23 | TCEPA | 10:1 | RT, 2 h and purification | 5.95 | 94.05 | 0 | 0 | 0 |
| | | | after oxidation | | | | | |
| E III.24 | TCEPA | 10:1 | 4° C., 5 h | 7.43 | 88.09 | 4.48 | 0 | 0 |

TABLE III 4

| | | D0 area | D2 area | D4 area | D6 area | D8 area |
|---|---|---|---|---|---|---|
| No. | Reductant | (mAU) | (mAU) | (mAU) | (mAU) | (mAU) |
| E III.1 | TCEP | 174.13 | 3047.34 | 88.64 | 0 | 0 |
| E III.2 | TCEP | 25.68 | 1302.07 | 459.84 | 261.32 | 0 |
| E III.3 | TCEP | 62.58 | 2245.29 | 430.55 | 197.02 | 0 |
| E III.4 | TCEP | 149.56 | 2909.57 | 61.93 | 0 | 0 |
| E I5I.5 | TCEP | 227.39 | 3294.35 | 0 | 0 | 0 |
| E III.6 | TCEP | 134.90 | 2054.05 | 112.76 | 49.35 | 0 |
| E III.7 | TCEP | 101.86 | 1921.93 | 0 | 0 | 0 |
| E III.8 | TCEP | 220.34 | 2350.36 | 0 | 0 | 0 |
| E III.9 | TCEP | 173.44 | 1964.29 | 42.90 | 0 | 0 |
| E III.10 | TCEP | 131.88 | 1995.79 | 0 | 0 | 0 |
| E III.11 | TCEP | 251.05 | 2246.2 | 0 | 0 | 0 |
| E III.12 | TCEP | 257.04 | 2040.49 | 0 | 0 | 0 |
| E III.13 | TCEP | 514.93 | 4324.3 | 0 | 0 | 0 |
| E III.14 | TCEP | 90.57 | 2331.72 | 494.31 | 134.19 | 0 |
| E III.15 | TCEP | 206.53 | 2284.57 | 70.51 | 0 | 0 |
| E III.16 | TCEPA | 86.52 | 2047.25 | 66.03 | 0 | 0 |
| E III.17 | TCEPA | 182.21 | 2489.28 | 0 | 0 | 0 |
| E III.18 | TCEPA | 187.54 | 2266.04 | 0 | 0 | 0 |
| E III.19 | TCEPA | 242.95 | 2163.03 | 0 | 0 | 0 |
| E III.20 | TCEPA | 276.04 | 1981.32 | 0 | 0 | 0 |
| E III.21 | TCEPA | 70.83 | 1446.61 | 0 | 0 | 0 |
| E III.22 | TCEPA | 149.46 | 2023.43 | 109.15 | 68.30 | 0 |
| E III.23 | TCEPA | 88.50 | 1398.65 | 0 | 0 | 0 |
| E III.24 | TCEPA | 158.79 | 1883.54 | 95.79 | 0 | 0 |

TABLE III-5

| | | | | The reduction | | | | |
|---|---|---|---|---|---|---|---|---|
| | | Reductant/antibody | DHAA/antibody | time in step | D0 | D2 | D4 | D6 |
| No. | Reductant | (molar ratio) | (molar ratio) | (1) (h) | (%) | (%) | (%) | (%) |
| E III.25 | TCEP | 6:1 | 16:1 | 18 | 10.59 | 89.41 | 0 | 0 |
| E III.26 | TCEP | 10:1 | 18:1 | 18 | 7.33 | 92.67 | 0 | 0 |
| E III.27 | TCEP | 4:1 | 7:1 | 4 | 6.28 | 80.22 | 13.51 | 0 |
| E III.28 | TCEP | 4:1 | 9:1 | 4 | 9.03 | 86.18 | 4.79 | 0 |
| E III.29 | TCEP | 4:1 | 11:1 | 4 | 11.51 | 86.42 | 2.08 | 0 |
| E III.30 | TCEP | 6:1 | 12:1 | 4 | 6.44 | 87.12 | 6.45 | 0 |
| E III.31 | TCEP | 6:1 | 14:1 | 4 | 9.12 | 88.57 | 2.31 | 0 |
| E III.32 | TCEP | 6:1 | 16:1 | 4 | 10.44 | 88.16 | 1.41 | 0 |
| E III.33 | TCEP | 10:1 | 14:1 | 4 | 3.04 | 81.78 | 15.18 | 0 |
| E III.34 | TCEP | 10:1 | 16:1 | 4 | 4.35 | 91.25 | 4.40 | 0 |
| E III.35 | TCEP | 10:1 | 18:1 | 4 | 7.33 | 92.67 | 0 | 0 |
| E III.36 | TCEP | 10:1 | 20:1 | 4 | 11.79 | 88.21 | 0 | 0 |
| E III.37 | TCEP | 10:1 | 22:1 | 4 | 8.37 | 91.63 | 0 | 0 |
| E III.38 | TCEP | 10:1 | 24:1 | 4 | 10.08 | 89.92 | 0 | 0 |
| E III.39 | TCEP | 5:1 | 12:1 | 1 | 11.04 | 88.96 | 0 | 0 |
| C III.1 | TCEP | 4:1 | 11:1 | 4 | 83.25 | 13.09 | 3.66 | 0 |
| C III.2 | TCEP | 6:1 | 16:1 | 4 | 92.13 | 7.87 | 0 | 0 |
| C III.3 | TCEP | 10:1 | 18:1 | 4 | 29.82 | 35.56 | 30.80 | 3.82 |
| E III.40 | TCEP-NO | 4:1 | 4:1 | 2 | 7.24 | 81.27 | 11.49 | 0 |
| E III.41 | TCEP-NO | 4:1 | 4:1 | 4 | 22.43 | 71.72 | 5.85 | 0 |
| E III.42 | TCEP-NO | 4:1 | 6:1 | 4 | 26.93 | 73.07 | 0 | 0 |
| E III.43 | TCEP-NO | 4:1 | 8:1 | 2 | 12.42 | 85.32 | 2.26 | 0 |
| E III.44 | TCEP-NO | 4:1 | 8:1 | 4 | 32.55 | 67.45 | 0 | 0 |
| E III.45 | TCEP-NO | 4:1 | 10:1 | 4 | 33.57 | 66.43 | 0 | 0 |
| E III.46 | TCEP-NO | 6:1 | 7:1 | 4 | 2.34 | 60.64 | 33.30 | 3.72 |
| E III.47 | TCEP-NO | 6:1 | 10:1 | 4 | 4.50 | 87.68 | 7.82 | 0 |
| E III.48 | TCEP-NO | 6:1 | 13:1 | 4 | 6.19 | 92.05 | 1.77 | 0 |
| E III.49 | TCEP-NO | 6:1 | 16:1 | 4 | 8.49 | 91.51 | 0 | 0 |
| E III.50 | TCEP-NO | 10:1 | 10:1 | 4 | 2.23 | 71.71 | 26.06 | 0 |
| E III.51 | TCEP-NO | 10:1 | 14:1 | 4 | 4.48 | 91.94 | 3.58 | 0 |
| E III.52 | TCEP-NO | 10:1 | 18:1 | 4 | 7.36 | 92.64 | 0 | 0 |
| E III.53 | TCEP-NO | 10:1 | 22:1 | 4 | 8.06 | 91.94 | 0 | 0 |

TABLE III-6

| | | D0 area | D2 area | D4 area | D6 area |
|---|---|---|---|---|---|
| No. | Reductant | (mAU) | (mAU) | (mAU) | (mAU) |
| E III.25 | TCEP | 192.89 | 1629.29 | 0 | 0 |
| E III.26 | TCEP | 126.81 | 1602.36 | 0 | 0 |
| E III.27 | TCEP | 111.91 | 1429.97 | 240.77 | 0 |
| E III.28 | TCEP | 135.18 | 1290.04 | 71.73 | 0 |
| E III.29 | TCEP | 195.56 | 1468.31 | 35.26 | 0 |
| E III.30 | TCEP | 108.52 | 1468.32 | 108.62 | 0 |
| E III.31 | TCEP | 163.30 | 1586.74 | 41.42 | 0 |
| E III.32 | TCEP | 192.89 | 1629.29 | 25.99 | 0 |
| E III.33 | TCEP | 47.33 | 1273.63 | 236.49 | 0 |
| E III.34 | TCEP | 66.61 | 1397.14 | 67.28 | 0 |
| E III.35 | TCEP | 126.81 | 1602.36 | 0 | 0 |
| E III.36 | TCEP | 182.13 | 1362.81 | 0 | 0 |
| E III.37 | TCEP | 120.49 | 1319.15 | 0 | 0 |
| E III.38 | TCEP | 149.14 | 1330.53 | 0 | 0 |
| E III.39 | TCEP | 684.71 | 5514.97 | 0 | 0 |
| C III.1 | TCEP | 1467.41 | 230.71 | 64.56 | 0 |
| C III.2 | TCEP | 1289.75 | 110.22 | 0 | 0 |
| C III.3 | TCEP | 394.72 | 470.61 | 407.68 | 50.49 |
| E III.40 | TCEP-NO | 98.44 | 1105.12 | 156.29 | 98.44 |
| E III.41 | TCEP-NO | 356.04 | 1138.46 | 92.91 | 0 |
| E III.42 | TCEP-NO | 448.21 | 1216.33 | 0 | 0 |
| E III.43 | TCEP-NO | 185.18 | 1272.41 | 33.73 | 0 |
| E III.44 | TCEP-NO | 524.39 | 1086.64 | 0 | 0 |
| E III.45 | TCEP-NO | 520.21 | 1029.58 | 0 | 0 |
| E III.46 | TCEP-NO | 35.94 | 932.25 | 511.96 | 57.23 |
| E III.47 | TCEP-NO | 67.06 | 1307.57 | 116.61 | 0 |
| E III.48 | TCEP-NO | 108.31 | 1611.96 | 30.94 | 0 |
| E III.49 | TCEP-NO | 143.80 | 1550.64 | 0 | 0 |
| E III.50 | TCEP-NO | 29.05 | 933.13 | 339.08 | 0 |
| E III.51 | TCEP-NO | 66.67 | 1368.72 | 53.31 | 0 |
| E III.52 | TCEP-NO | 95.51 | 1202.40 | 0 | 0 |
| E III.53 | TCEP-NO | 119.06 | 1357.55 | 0 | 0 |

As the results shown in table III-3-III-6, linker-payloads (MC-VC-PAB-MMAE) were successfully linked to Trastuzumab, which indicated ADCs with D2 were successfully synthesized.

As shown in examples III.25-III.53, the content of D6 is up to 60%, 70%, 80%, even to 85%, and 90% when the molar ratio of TCEP and the antibody is from 4:1 to 10:1. Meanwhile, the reduction time in step (1) is shortened to 4 h or 1 h, which is with less reduction time cost. The results also showed the molar ratio of DHAA and antibody plays an important role in determining the content of D2 and the selective oxidation. The content of D2 is up to 60%, 70%, 80%, 85%, even to 90% or 95% when the molar ratio of DHAA and the antibody is 4:1 to 24:1.

The molar ratio of Zn²⁺ and the antibody also impacts the content of D2 and the selective oxidation. As shown in comparative examples III.1-I1I.3, when the concentration of Zn²⁺ is 0, the content of D2 is as low as 7.87%. It is helpful to improve the homogeneity of the ADC with D2 that the molar ratio of Zn²⁺ and the antibody is 1:1 or 2:1.

The proportion of D6 was up to 95% when performing purification after oxidation, which indicated that the purification could improve the homogeneity of ADCs with D2 significantly.

These results clearly indicated that the process of the method was benefit for conjugates with D2 with significantly improved homogeneity.

### Examples III.54-III.60: Preparation of Trastuzumab-[MC-VC-PAB-MMAE]₂ Conjugate (the Oxidation Temperature and/or Time in Step (2) is Different)

The method is similar to example III.1, and the differences are the oxidation temperature, time in step (2), the kinds of the reductant, and/or the molar ratio of the reductant and the antibody which are shown in table III-7.

The homogeneity assay result and the chromatographic peak area of examples III.54-III.60 were shown in Table III-7-Table II-8.

TABLE III-7

| | | | The oxidation | | | | | |
|---|---|---|---|---|---|---|---|---|
| | | Reductant/antibody | temperature and | | | | | |
| No. | Reductant | (molar ratio) | time in step (2) | D0(%) | D2(%) | D4(%) | D6(%) | D8(%) |
| E III.54 | TCEP | 4:1 | 25° C., 6 h | 5.52 | 92.28 | 2.20 | 0 | 0 |
| E.III.55 | TCEP | 4:1 | 4° C., 24 h | 6.81 | 93.19 | 0 | 0 | 0 |
| E III.56 | TCEP | 4:1 | 4° C., 48 h | 7.77 | 92.24 | 0 | 0 | 0 |
| E III.57 | TCEP | 4:1 | 37° C., 1 h | 5.28 | 73.18 | 21.54 | 0 | 0 |
| E III.58 | TCEPA | 3.2:1 | 37° C., 1 h | 2.86 | 80.80 | 15.20 | 1.14 | 0 |
| E III.59 | TCEPA | 3.5:1 | 4° C., 24 h | 2.69 | 87.97 | 9.34 | 0 | 0 |
| E III.60 | TCEPA | 3.5:1 | 4° C., 48 h | 4.18 | 95.83 | 0 | 0 | 0 |

TABLE III-8

| | | D0 | D2 | D4 | D6 | D8 |
|---|---|---|---|---|---|---|
| | | area | area | area | area | area |
| No. | Reductant | (mAU) | (mAU) | (mAU) | (mAU) | (mAU) |
| E III.54 | TCEP | 89.54 | 1497.41 | 35.72 | 0 | 0 |
| E.III.55 | TCEP | 193.69 | 2651.78 | 0 | 0 | 0 |
| E III.56 | TCEP | 142.27 | 1689.89 | 0 | 0 | 0 |
| E III.57 | TCEP | 65.815 | 911.888 | 268.448 | 0 | 0 |
| E III.58 | TCEPA | 50.53 | 1428.62 | 268.76 | 20.09 | 0 |
| E III.59 | TCEPA | 67.66 | 2213.14 | 235.04 | 0 | 0 |
| E III.60 | TCEPA | 77.57 | 1780.35 | 0 | 0 | 0 |

As shown in above table, the results showed the content of D6 is up to 70%, even to 90% when the oxidation temperature in step (2) is from 4° C. to 37° C., and the oxidation time in step (2) is from 1 h to 48 h.

### Examples III.61-III.92 and comparative Examples III.4-III.5: Preparation of Trastuzumab-[MC-VC-PAB-MMAE]₂ Conjugate by Using the Different Buffer System

The method of examples and comparative example is similar to example III. 1, and the differences is the buffer system or the reductant which is shown in Table III-9.

Meanwhile, the oxidation time in step (2) is 2 h in examples III.61-III.92 and comparative examples 4-5. The molar ratio of the reductant and the antibody is 3.5:1 in examples III.77-III.92 and comparative example III.5.

The homogeneity assay result and the chromatographic peak area of examples III.61-III.92 were shown in Table III-9-Table III-10.

TABLE III-9

| No. | Reductant | The buffer system | D0(%) | D2(%) | D4(%) | D6(%) | D8(%) |
|---|---|---|---|---|---|---|---|
| E III.61 | TCEP | Bis-Tris, pH 6.7 | 4.68 | 83.54 | 11.78 | 0 | 0 |
| E III.62 | TCEP | PIPES, pH 6.7 | 20.77 | 75.71 | 3.51 | 0 | 0 |
| E III.63 | TCEP | PB, pH 6.7 | 26.00 | 74.00 | 0 | 0 | 0 |
| E III.64 | TCEP | HEPES, pH 7.0 | 9.80 | 90.20 | 0 | 0 | 0 |
| E III.65 | TCEP | MOPS, pH 7.0 | 12.48 | 81.43 | 6.09 | 0 | 0 |
| E III.66 | TCEP | DIPSO, pH 7.4 | 5.23 | 91.09 | 3.68 | 0 | 0 |
| E III.67 | TCEP | MOBS, pH 7.4 | 7.89 | 92.11 | 0 | 0 | 0 |
| E III.68 | TCEP | MOPSO, pH 7.4 | 7.45 | 88.78 | 3.77 | 0 | 0 |
| E III.69 | TCEP | TES, pH 7.4 | 9.20 | 86.09 | 4.71 | 0 | 0 |
| E III.70 | TCEP | ACES, pH 7.4 | 6.64 | 93.36 | 0 | 0 | 0 |
| E III.71 | TCEP | TAPSO, pH 7.4 | 8.16 | 88.92 | 2.93 | 0 | 0 |
| E III.72 | TCEP | MES, pH 5.8 | 8.43 | 91.57 | 0 | 0 | 0 |
| E III.73 | TCEP | MES, pH 6.1 | 6.47 | 93.53 | 0 | 0 | 0 |
| E III.74 | TCEP | BES, pH 6.4 | 8.10 | 91.90 | 0 | 0 | 0 |
| E III.75 | TCEP | BES, pH 7.4 | 4.08 | 95.92 | 0 | 0 | 0 |
| E III.76 | TCEP | BES, pH 7.4 | 7.78 | 92.22 | 0 | 0 | 0 |
| C III.4 | TCEP | ADA, pH 6.7 | 67.96 | 25.96 | 6.08 | 0 | 0 |
| E III.77 | TCEPA | Bis-Tris, pH 6.7 | 5.51 | 87.96 | 6.53 | 0 | 0 |
| E III.78 | TCEPA | PIPES, pH 6.7 | 4.46 | 79.81 | 15.73 | 0 | 0 |
| E III.79 | TCEPA | PB, pH 6.7 | 38.34 | 61.67 | 0 | 0 | 0 |
| E III.80 | TCEPA | HEPES, pH 7.0 | 6.06 | 91.51 | 2.43 | 0 | 0 |
| E III.81 | TCEPA | MOPS, pH 7.0 | 6.36 | 87.38 | 6.26 | 0 | 0 |
| E III.82 | TCEPA | DIPSO, pH 7.4 | 7.19 | 92.81 | 0 | 0 | 0 |
| E III.83 | TCEPA | MOBS, pH 7.4 | 6.18 | 93.83 | 0 | 0 | 0 |
| E III.84 | TCEPA | MOPSO, pH 7.4 | 5.70 | 92.18 | 2.12 | 0 | 0 |
| E III.85 | TCEPA | TES, pH 7.4 | 5.66 | 91.75 | 2.59 | 0 | 0 |
| E III.86 | TCEPA | ACES, pH 7.4 | 7.71 | 92.29 | 0 | 0 | 0 |
| E III.87 | TCEPA | TAPSO, pH 7.4 | 4.77 | 92.26 | 2.98 | 0 | 0 |
| E III.88 | TCEPA | MES pH 5.8 | 3.48 | 60.48 | 32.66 | 3.39 | 0 |
| E III.89 | TCEPA | MES pH 6.1 | 4.12 | 73.51 | 22.37 | 0 | 0 |
| E III.90 | TCEPA | BES, pH 6.4 | 3.57 | 76.45 | 19.98 | 0 | 0 |
| E III.91 | TCEPA | BES, pH 6.7 | 1.79 | 82.25 | 14.77 | 1.20 | 0 |
| E III.92 | TCEPA | BES, pH 7.4 | 3.05 | 94.48 | 2.48 | 0 | 0 |
| C III.5 | TCEPA | ADA, pH 6.7 | 86.47 | 13.53 | 0 | 0 | 0 |

TABLE III-10

| | | D0 | D2 | D4 | D6 | D8 |
|---|---|---|---|---|---|---|
| | | area | area | area | area | area |
| No. | Reductant | (mAU) | (mAU) | (mAU) | (mAU) | (mAU) |
| E III.61 | TCEP | 96.13 | 1716.94 | 242.18 | 0 | 0 |
| E III.62 | TCEP | 500.82 | 1825.44 | 84.72 | 0 | 0 |
| E III.63 | TCEP | 549.77 | 1565.08 | 0 | 0 | 0 |
| E III.64 | TCEP | 162.92 | 1499.80 | 0 | 0 | 0 |
| E III.65 | TCEP | 307.20 | 2004.26 | 150.01 | 0 | 0 |
| E III.66 | TCEP | 97.54 | 1698.24 | 68.61 | 0 | 0 |
| E III.67 | TCEP | 163.97 | 1914.69 | 0 | 0 | 0 |
| E III.68 | TCEP | 141.26 | 1684.33 | 71.57 | 0 | 0 |
| E III.69 | TCEP | 178.35 | 1668.89 | 91.26 | 0 | 0 |
| E III.70 | TCEP | 96.95 | 1362.93 | 0 | 0 | 0 |
| E III.71 | TCEP | 177.70 | 1937.39 | 63.76 | 0 | 0 |
| E III.72 | TCEP | 153.04 | 1662.23 | 0 | 0 | 0 |
| E III.73 | TCEP | 145.54 | 2104.43 | 0 | 0 | 0 |
| E III.74 | TCEP | 78.98 | 896.45 | 0 | 0 | 0 |
| E III.75 | TCEP | 75.54 | 1776.57 | 0 | 0 | 0 |
| E III.76 | TCEP | 140.76 | 1667.58 | 0 | 0 | 0 |
| C III.4 | TCEP | 1580.01 | 603.49 | 141.28 | 0 | 0 |
| E III.77 | TCEPA | 104.03 | 1660.98 | 123.34 | 0 | 0 |
| E III.78 | TCEPA | 99.11 | 1772.13 | 349.34 | 0 | 0 |
| E III.79 | TCEPA | 773.08 | 1243.59 | 0 | 0 | 0 |
| E III.80 | TCEPA | 120.80 | 1824.45 | 48.43 | 0 | 0 |
| E III.81 | TCEPA | 137.48 | 1888.12 | 135.32 | 0 | 0 |
| E III.82 | TCEPA | 137.48 | 1773.54 | 0 | 0 | 0 |
| E III.83 | TCEPA | 105.96 | 1609.91 | 0 | 0 | 0 |
| E III.84 | TCEPA | 107.92 | 1745.03 | 40.04 | 0 | 0 |
| E III.85 | TCEPA | 118.00 | 1912.94 | 53.99 | 0 | 0 |
| E III.86 | TCEPA | 151.24 | 1811.29 | 0 | 0 | 0 |
| E III.87 | TCEPA | 114.53 | 2217.68 | 71.53 | 0 | 0 |
| E III.88 | TCEPA | 55.52 | 965.40 | 521.37 | 54.05 | 0 |
| E III.89 | TCEPA | 72.21 | 1287.04 | 391.65 | 0 | 0 |
| E III.90 | TCEPA | 28.38 | 607.86 | 158.83 | 0 | 0 |
| E III.91 | TCEPA | 43.22 | 1990.82 | 357.41 | 29.07 | 0 |
| E III.92 | TCEPA | 59.44 | 1842.92 | 48.31 | 0 | 0 |
| C III.5 | TCEPA | 1490.69 | 233.17 | 0 | 0 | 0 |

As shown in above table, the results showed the types and the pH value of the buffer system will impact the content of D2 by impacting the reduction kinetics and selectivity. The buffer systems of examples III.61-III.92 are useful to increase the content of D2, and the pH value of the buffer system is from 5.8 to 7.4.

### Example III.93: Preparation of Trastuzumab-[Maleimide-PEG4-N3-DBCO-MMAE]₁ (the ADC with D1)

(1) TCEP (0.048 mM) and ZnCl₂ (0.012 mM) were added to a solution of Trastuzumab (0.012 mM) in BES buffer (20 mM, pH7.0) and the reaction mixture was vortexed for mixing, then the reaction mixture was incubated at 4° C. for 18 h;
(2) Adding DHAA (0.066 mM) to selectively re-oxidize the reduced thiol groups in Fab region resulted from step (1) at 25° C. for 2 h;
(3) Introducing EDTA (0.6 mM) and a first thiobridge reagent dibromomaleimide-PEG4-N3 (0.013 mM) to react with reduced thiol groups resulted from step (3), the reaction temperature is 25° C. and the reaction time is 1 h, then recovering the product using a desalting column to afford Trastuzumab-[Maleimide-PEG4-N3]₁;
(4) incubating Trastuzumab-[Maleimide-PEG4-N3]₁ and DBCO-MMAE (0.02 mM) in BES buffer (20 mM, pH7.0), the reaction temperature is 25° C. and the reaction time is 8 h.
(5) The reaction mixture was subjected to purification using a de-salting column and AKTA with HIC chromatography.

### Example III.94: Preparation of Trastuzumab-[Maleimide-PEG4-N3-DBCO-Cy3]₁ (the ADC with D1)

The method is similar to example III.93, the differences are that the reductant is TCEPA, the concentration of DHAA is 0.096 mM in step (2) and the first linker-payload is DBCO-Cy3. Meanwhile, in step (5) of example III.94, the reaction mixture was subjected to purification using a de-salting column.

The homogeneity assay result and the chromatographic peak area of examples III.93-III.94 were shown in Table III-11.

TABLE III-11

| | | | | | D0 | D1 | D2 |
|---|---|---|---|---|---|---|---|
| | | | | | area | area | area |
| No. | Figure | D0(%) | D1(%) | D2(%) | (mAU) | (mAU) | (mAU) |
| E III.93 | 28 | 2.42 | 97.58 | / | 60.439 | 2437.577 | / |
| E III.94 | 29 | 13.07 | 83.81 | 3.13 | 244.79 | 1570.13 | 58.54 |

As shown in the above table, the result demonstrated that the content of the ADC with D1 was generally up to 83.81% or 97.58%, which indicated the process of method was benefit for site-specific modifying the antibody with D1 and improving the homogeneity.

### Example III.95: Preparation of Trastuzumab-[Maleimide-PEG4-N3-DBCO-MMAE]₁[MC-GGFG-DXd]₆ (the ADC with D1+D6)

(1) introducing Trastuzumab-[Maleimide-PEG4-N3-DBCO-MMAE]₁ prepared from example III.93 (0.008 mM) and TCEP (0.08 mM) in BES buffer (20 mM, pH7.0), the reaction temperature is 37° C. and the reaction time is 16 h;
(2) introducing MC-GGFG-DXd (0.14 mM) to solution from step (1), and the reaction mixture was allowed to stay at 24° C. for 1 h, then recovering Trastuzumab-[Maleimide-PEG4-N3-DBCO-MMAE]₁[MC-GGFG-DXd]₆ using a desalting column.

### Example III.96: Preparation of Trastuzumab-[Maleimide-PEG4-N3-DBCO-Cy3]₁[MC-VC-PAB-MMAE]₆ (the ADC with D1+D6)

(1) introducing Trastuzumab-[Maleimide-PEG4-N3-DBCO-Cy3]₁ prepared from example III.94 (0.008 mM) and TCEPA (0.08 mM) in BES buffer (20 mM, pH7.0), the reaction temperature is 24° C. and the reaction time is 24 h;
(2) introducing MC-VC-PAB-MMAE (0.14 mM) to solution from step (1), and the reaction mixture was allowed to stay at 24° C. for 1 h, then recovering Trastuzumab-[Maleimide-PEG4-N3-DBCO-Cy3]₁[MC-VC-PAB-MMAE]₆ using a desalting column.

The homogeneity assay result and the chromatographic peak area of examples III.95-III.96 were shown in Table III-12 and Table III-13.

TABLE III-12

| | | D1 + | D1 + | D1 + | |
|---|---|---|---|---|---|
| No. | Figure | D2(%) | D4 (%) | D6 (%) | D8% |
| E III.95 | 30 | 5.66 | 5.98 | 82.42 | 5.94 |

| | | | D1 + | D2 + | |
|---|---|---|---|---|---|
| No. | Figure | D6(%) | D6 (%) | D6 (%) | / |
| E III.96 | 31 | 7.79 | 88.02 | 4.20 | / |

TABLE III-13

| | D1 + | D1 + | D1 + | |
|---|---|---|---|---|
| | D2 area | D4 area | D6 area | D8 area |
| No. | (mAU) | (mAU) | (mAU) | (mAU) |
| E III.95 | 80.903 | 85.513 | 1178.764 | 84.982 |

| | | D1 + | D2 + | |
|---|---|---|---|---|
| | D6 area | D6 area | D6 area | |
| No. | (mAU) | (mAU) | (mAU) | / |
| E III.96 | 42.48 | 480.13 | 22.90 | / |

As shown in the above table, the result demonstrated that the content of the ADC with D1+D6 was generally up to 82.42% or 88.02%, which indicated the process of method was benefit for site-specific modifying the antibody with D1+D6 and improving the homogeneity.

### Example III.97: Preparation of Trastuzumab-[Maleimide-PEG4-N3-DBCO-Cy3]₁[MC-VC-PAB-MMAE]₂ (the ADC with D1+D2)

(1) TCEP (0.048 mM) and ZnCl₂ (0.012 mM) were added to a solution of Trastuzumab (0.012 mM) in BES buffer (20 mM, pH7.0) and the reaction mixture was vortexed for mixing, then the reaction mixture was incubated at 4° C. for 18 h;
(2) Adding DHAA (0.096 mM) to selectively re-oxidize the reduced thiol groups in Fab region resulted from step (1) at 25° C. for 2 h;
(3) Introducing EDTA (0.6 mM) and a first thiobridge reagent dibromomaleimide-PEG4-N3 (0.013 mM) to react with the reduced thiol groups resulted from step (3), the reaction temperature is 25° C. and the reaction time is 1 h, then recovering the product using a desalting column to afford Trastuzumab-[Maleimide-PEG4-N3]₁;
(4) incubating Trastuzumab-[Maleimide-PEG4-N3]₁ (0.013 mM), the reaction temperature is 25C and the reaction time is 1 h, and then DBCO-Cy3 (0.02 mM) in BES buffer (20 mM, pH7.0), the reaction temperature is 25° C. and the reaction time is 8 h;
(5) The reaction mixture was subjected to purification using a de-salting column;
(6) incubating ZnCl₂ (0.8 mM), the second reductant TCEP (0.011 mM) and the product from step (5) (0.008 mM) in BES buffer (pH7.0, 20 mM) and the reaction mixture was allowed to stay at 4° C. for 4 h;
(7) introducing EDTA (3 mM) to trap Zn²⁺, and introducing MC-VC-PAB-MMAE (0.048 mM) to react with the reduced thiol groups resulted from step (6), the reaction temperature is 25° C. and the reaction time is 2 h;
(8) the reaction mixture was subjected to purification using a desalting column.

### Example III.98: Preparation of Trastuzumab-[Maleimide-PEG4-N3-DBCO-Cy3]₁[MC-VC-PAB-MMAE]₂ (the ADC with D1+D2)

(1) incubating ZnCl₂ (0.8 mM), the second reductant TCEP-6 (0.0144 mM) and Trastuzumab-[Maleimide-PEG4-N3-DBCO-Cy3]₁ prepared from example III.94 (0.008 mM) in BES buffer (pH7.0, 20 mM) and the reaction mixture was allowed to stay at 4° C. for 4 h;
(2) introducing EDTA (3 mM) to trap Zn²⁺, and introducing MC-VC-PAB-MMAE (0.048 mM) to react with the reduced thiol groups resulted from step (2), the reaction temperature is 25° C. and the reaction time is 2 h;
(3) the reaction mixture was subjected to purification using a desalting column.

The homogeneity assay result and the chromatographic peak area of examples III.97-III.98 were shown in Table III-14 and Table III-15.

TABLE III-14

| | | | | D1 + | | D1 + |
|---|---|---|---|---|---|---|
| No. | Figure | D1(%) | D2 (%) | D2 (%) | D4(%) | D4(%) |
| E III.97 | 32 | 2.84 | 2.39 | 71.97 | 2.49 | 20.31 |
| E III.98 | 33 | 3.72 | 7.92 | 73.80 | 4.06 | 10.51 |

TABLE III-15

| | | | D1 + | | D1 + |
|---|---|---|---|---|---|
| | D1area | D2 area | D2 area | D4 area | D4 area |
| No. | (mAU) | (mAU) | (mAU) | (mAU) | (mAU) |
| E III.97 | 24.67 | 20.78 | 625.96 | 21.64 | 176.67 |
| E III.98 | 22.23 | 47.30 | 440.97 | 24.26 | 62.79 |

With step (6) in example III.97 and with step (1) in example III.98, one of the interchain disulfide bonds in the ADC with D1 was reduced. As shown in the above table, the result demonstrated that the content of the ADC with D1+D2 was generally up to 71.97% or 73.8%, which indicated the process of method was benefit for site-specific modifying the antibody with D1+D2 and improving the homogeneity.

### Examples III.99-III.100: preparation of Trastuzumab-[Maleimide-PEG4-N3-DBCO-Cy3]₁[MC-VC-PAB-MMAE]₄ (the ADC with D1+D4)

(1) TCEP (0.048 mM) and ZnCl₂ (0.024 mM) were added to a solution of Trastuzumab (0.012 mM) in BES buffer (20 mM, pH7.0) and the reaction mixture was vortexed for mixing, then the reaction mixture was incubated at 4° C. for 18 h;
(2) Adding DHAA (0.12 mM) to selectively re-oxidize the reduced thiol groups in Fab region resulted from step (1) at 25° C. for 2 h;
(3) Introducing EDTA (0.6 mM) and a mixture of a first thiobridge reagent dibromomaleimide-PEG4-N3 (0.028 mM) and DBCO-Cy3 (0.033 mM) to react with the reduced thiol groups resulted from step (2), the reaction temperature is 25° C. and the reaction time is 2 h, then recovering the product using a desalting column to afford Trastuzumab-[Maleimide-PEG4-N3-DBCO-Cy3]₁;
(4) incubating ZnCl₂ (example III.99: 0.024 mM; example III.100: 0.36 mM), the second reductant TCEP (0.024 mM) and the product from step (3) (0.008 mM) in BES buffer (pH7.0, 20 mM) and the reaction mixture was allowed to stay at 4° C. for 16 h;
(5) introducing EDTA (3 mM) to trap Zn²⁺, and introducing MC-VC-PAB-MMAE (0.048 mM) to react with the reduced thiol groups resulted from step (4), the reaction temperature is 25° C. and the reaction time is 2 h;
(6) the reaction mixture was subjected to purification using a desalting column.

### Examples III.101-III.102: Preparation of Trastuzumab-[Maleimide-PEG4-N3-DBCO-Cy3]1[MC-VC-PAB-MMAE]₄ (the ADC with D1+D4)

(1) TCEPA (0.048 mM) and ZnCl₂ (0.012 mM) were added to a solution of Trastuzumab (0.012 mM) in BES buffer (20 mM, pH7.0) and the reaction mixture was vortexed for mixing, then the reaction mixture was incubated at 4° C. for 14 h;
(2) Adding DHAA (0.096 mM) to selectively re-oxidize the reduced thiol groups mainly in Fab region resulted from step (1) at 25° C. for 2 h;
(3) Introducing EDTA (0.6 mM) and a first thio-bridging reagent dibromomaleimide-PEG4-N3 (0.013 mM) to react with the reduced thiol groups resulted from step (3), the reaction temperature is 25° C. and the reaction time is 1.5 h, then recovering the product using a desalting column to afford Trastuzumab-[Maleimide-PEG4-N3]₁;
(4) incubating Trastuzumab-[Maleimide-PEG4-N3]₁ (0.013 mM), the incubation temperature is 25° C. and the incubation time is 8 h, and then, introducing DBCO-Cy3 (0.02 mM) in BES buffer (20 mM, pH7.0), the reaction temperature is 25° C. and the reaction time is 8 h;
(5) The reaction mixture was subjected to purification using a de-salting column;
(6) incubating ZnCl₂ (0.8 mM), the second reductant TCEP-3 (example II.101: 0.0384 mM) or TCEP-6 (example III.102: 0.0336 mM) and the product from step (5) in BES buffer (pH7.0, 20 mM) and the reaction mixture was allowed to stay at 4° C. for 16 h;
(7) introducing EDAT (3 mM) to trap Zn²⁺, and introducing MC-VC-PAB-MMAE (0.08 mM) to react with the reduced thiol groups resulted from step (6), the reaction temperature is 25° C. and the reaction time is 2 h;
(8) the reaction mixture was subjected to purification using a desalting column.

The homogeneity assay result and the chromatographic peak area of examples III.99-III.102 were shown in Table III-16 and Table III-17.

TABLE III-16

| No. | Figure | Reductant | D0 | D1 | D2 (%) | / | / |
|---|---|---|---|---|---|---|---|
| E III.99-step (3) | 34A | TCEP | 5.54 | 87.75 | 6.71 | / | / |
| E III. 101-step (5) | 35A | TCEPA | 0 | 89.43 | 10.57 | / | / |
| No. | Figure | Reductant | D1 + D2(%) | D2 + D2 (%) | D1 + D4 (%) | D2 + D4(%) | D1 + D6 (%) |
| E III.99-step (6) | 34B | TCEP | 8.81 | 7.74 | 79.18 | 4.26 | / |
| E III.100-step (6) | 34C | TCEP | 5.40 | 6.94 | 81.17 | 3.77 | 2.72 |
| No. | Figure | Reductant | D2(%) | D4 (%) | D1 + D4 (%) | D6% | D1 + D6 (%) |
| E III.101-step (8) | 35B | TCEP-3 | 2.01 | / | 84.48 | 2.83 | 10.69 |
| E III. 102-step (8) | 35C | TCEP-6 | 3.74 | / | 84.17 | 2.38 | 9.71 |

TABLE III-17

| | D0 area | D1 area | D2 area | | |
|---|---|---|---|---|---|
| No. | (mAU) | (mAU) | (mAU) | / | / |
| E III.99-step (3) | 111.12 | 1760.94 | 134.65 | / | / |
| E III.101-step (5) | 0 | 2870.206 | 339.348 | / | / |

| | D1 + | D2 + | D1 + | D2 + | D1 + |
|---|---|---|---|---|---|
| | D2 area | D2 area | D4 area | D4 area | D6 area |
| No. | (mAU) | (mAU) | (mAU) | (mAU) | (mAU) |
| E III.99-step (6) | 69.09 | 60.70 | 620.80 | 33.43 | / |
| E III.100-step (6) | 52.36 | 67.33 | 787.43 | 36.60 | 26.35 |
| | | | D1 + | | D1 + |
| | D2 area | D4 area | D4 area | D6 area | D6 area |
| No. | (mAU) | (mAU) | (mAU) | (mAU) | (mAU) |
| E III.101-step (8) | 32.11 | / | 1351.22 | 45.27 | 170.93 |
| E III.102-step (8) | 40.55 | / | 913.68 | 25.88 | 105.46 |

With step (4) in example 99 and with step (6) in example 101, two of the interchain disulfide bonds in the ADC with D1 were reduced. As shown in the above table, the result demonstrated that the content of the ADC with D1+D4 was generally up to 80%, which indicated the process of method was benefit for site-specific modifying the antibody with D1+D4 and improving the homogeneity.

### Example III.103: Preparation of Trastuzumab-[MC-VC-PAB-MMAE]₂[MC-GGFG-DXd]₄ Conjugate (The ADC with D2+D4)

(1) TCEP (0.0672 mM) and ZnCl₂ (0.024 mM) were added to a solution of Trastuzumab (0.012 mM) in BES buffer (20 mM, pH7.0) and the reaction mixture was vortexed for mixing, then the reaction mixture was incubated at 4° C. for 16 h;
(2) Adding DHAA (0.12 mM) to selectively re-oxidize the reduced thiol groups in Fab region resulted from step (1) at 25° C. for 2 h;
(3) Introducing EDTA (0.6 mM) and MC-VC-PAB-MMAE (0.048 mM) in DMA to react with remained thiol groups from step (2) and the reaction was incubated at room temperature for 1 h:
(4) The reaction mixture was subjected to purification using a de-salting column:
(5) incubating ZnCl₂ (0.552 mM), the second reductant TCEP (1.2 mM) and the product from step (4) in BES buffer (pH7.0, 20 mM) and the reaction mixture was allowed to stay at 4° C. for 2.5 h;
(6) introducing EDTA (3 mM) to trap Zn²⁺, and introducing MC-GGFG-DXd (0.048 mM) to react with the reduced thiol groups resulted from step (5), the reaction temperature is 25° C. and the reaction time is 2 h;
(7) the reaction mixture was subjected to purification using a desalting column.

### Example III.104: Preparation of Trastuzumab-[MC-GGFG-DXd]₂[MC-VC-PAB-MMAE]₄ (The ADC with D2+D4)

(1) TCEPA (0.048 mM) and ZnCl₂ (0.012 mM) were added to a solution of Trastuzumab (0.012 mM) in BES buffer (20 mM, pH7.0) and the reaction mixture was vortexed for mixing, then the reaction mixture was incubated at 4° C. for 16 h;
(2) Adding DHAA (0.096 mM) to selectively re-oxidize the reduced thiol groups in Fab region resulted from step (1) at 25° C. for 2 h;
(3) Introducing EDTA (0.6 mM) and MC-GGFG-DXd (0.048 mM) in DMA to react with remained thiol groups from step (2) and the reaction was incubated at room temperature for 1.5 h;
(4) The reaction mixture was subjected to purification using a de-salting column;
(5) incubating ZnCl₂ (0.8 mM), the second reductant TCEP-3 mM (0.072) and the product from step (4) in BES buffer (pH7.0, 20 mM) and the reaction mixture was allowed to stay at 4° C. for 16 h;
(6) introducing EDAT (3 mM) to trap Zn²⁺, and introducing MC-VC-PAB-MMAE (0.08 mM) to react with the reduced thiol groups resulted from step (5), the reaction temperature is 25° C. and the reaction time is 2 h;
(7) the reaction mixture was subjected to purification using a desalting column.

### Examples III.105-106: Preparation of Trastuzumab-[MC-VC-PAB-MMAE]₂[MC-GGFG-DXd]₄ Conjugate (The ADC with D2+D4)

(1) TCEPA (0.042 mM) and ZnCl₂ (0.024 mM) were added to a solution of Trastuzumab (0.012 mM) in BES buffer (20 mM, pH7.0) and the reaction mixture was vortexed for mixing, then the reaction mixture was incubated at 4° C. for 16 h;
(2) Adding DHAA (0.12 mM) to selectively re-oxidize the reduced thiol groups in Fab region resulted from step (1) at 25° C. for 2 h;
(3) Introducing EDTA (0.12 mM) and MC-VC-PAB-MMAE (0.048 mM) in DMA to react with remained thiol groups from step (2) and the reaction was incubated at room temperature for 1 h;
(4) The reaction mixture was subjected to purification using a de-salting column:
(5) incubating ZnCl₂ (example III. 105: 0.36 mM; example III. 106: 0.024 mM), the second reductant TCEP-6 (example III.105: 0.042 mM; example III.106: 0.054 mM) and the product from step (4) in BES buffer (pH7.0, 20 mM) and the reaction mixture was allowed to stay at Y° C. for 1 h;
(6) introducing EDAT (1.2 mM) to trap Zn²⁺, and introducing MC-GGFG-DXd (0.096 mM) to react with the reduced thiol groups resulted from step (5), the reaction temperature is 25° C. and the reaction time is 2 h;
(7) the reaction mixture was subjected to purification using a desalting column.

The homogeneity assay result and the chromatographic peak area of examples III.103-III.106 were shown in Table III-18 and Table III-19.

TABLE III-18

| No. | Figure | D0(%) | D2 (%) | D4 (%) | D6 (%) |
|---|---|---|---|---|---|
| E III.103-step (4) | 36A | 1.29 | 95.09 | 3.62 | 0 |
| E III.105-step (4) | 38A | 2.60 | 91.38 | 6.02 | 0 |
| E III.106-step (4) | / | 2.59 | 91.25 | 6.16 | 0 |
| No. | Figure | D1(%) | D2 (%) | D4 (%) | D6 (%) |
| E III.104-step (4) | 37A | 11.30 | 85.66 | 2.54 | 0.51 |

| | | D2 + | D2 + | D2 + | |
|---|---|---|---|---|---|
| No. | Figure | D2(%) | D4(%) | D6 (%) | D8 |
| E III.103-step (7) | 36B | 1.42 | 91.27 | 7.31 | / |
| E III.105-step (7) | 38B | 1.5 | 81.7 | 14.2 | 2.6 |
| E III.106-step (7) | 38C | 4.41 | 62.84 | 29.88 | 2.87 |

| | | D4(Dxd) + | D2(DXd) + | | |
|---|---|---|---|---|---|
| | | D2 (MMAE) | D4 (MMAE) | | |
| No. | Figure | (%) | (%) | D6 (%) | / |
| E III.104-step (7) | 37B | 2.58 | 82.23 | 15.19 | / |

TABLE III-19

| | D0 area | D2 area | D4 area | D6 area |
|---|---|---|---|---|
| No. | (mAU) | (mAU) | (mAU) | (mAU) |
| E III.103-step (4) | 27.54 | 2024.82 | 77.10 | 0 |
| E III.105-step (4) | 66.75 | 2346.46 | 154.6 | 0 |
| E III.106-step (4) | 68.28 | 2401.81 | 162.01 | 0 |
| | D1 area | D2 area | D4 area | D6 area |
| No. | (mAU) | (mAU) | (mAU) | (mAU) |
| E III.104-step (4) | 225.459 | 1709.11 | 50.67 | / |
| | D2 + | D2 + | D2 + | |
| | D2 area | D4 area | D6 area | D8 area |
| No. | (mAU) | (mAU) | (mAU) | (mAU) |
| E III.103-step (7) | 15.85 | 1015.92 | 81.39 | / |
| E III.105-step (7) | 40.5 | 2212.1 | 385.9 | 69.7 |
| E III.106-step (7) | 86.40 | 1229.97 | 584.75 | 56.11 |

| | D2(Dxd) + | D4(DXd) + | | |
|---|---|---|---|---|
| | D4 (MMAE) | 2 (MMAE) | D6 area | |
| No. | area (mAU) | area (mAU) | (mAU) | / |
| E III.104-step (7) | 431.04 | 13.51 | 79.64 | / |

As shown in the above table, the result demonstrated that the content of the ADC with D2D4 was generally up to 80% or 90%, which indicated the process of method was benefit for site-specific modifying the antibody with D2+D4 and improving the homogeneity.

As shown in examples III.105-III.106, the result demonstrated that the selective reduction ability of the reductant increases in step (5) when the molar ratio of the transition metal ions and the reductant increases in step (5).

### Example V.1: Preparation of Trastuzumab-[MC-VC-PAB-MMAE]₄ Conjugate (The ADC with D4)

(1) ZnCl₂ (0.0096 mM) and TCEP-3 (0.048 mM) were added to a solution of a monoclonal antibody Trastuzumab (0.012 mM, in BES buffer, pH7.0, 20 mM) and the reaction mixture was allowed to stay at 4° C. for 16 h;
(2) MC-VC-PAB-MMAE (0.072 mM) in DMA was introduced and the reaction was continued at room temperature for 2 h;
(3) The reaction mixture was subjected to purification using a desalting column.

### Examples V.2-V.12: Preparation of Trastuzumab-[MC-VC-PAB-MMAE]₄ conjugate (The ADC with D4)

The method is similar to Example V.1. The difference is the concentration of ZnCl₂ which is shown in Table V-1.

The homogeneity assay result and the chromatographic peak area of examples V.1-V.12 were shown in Table V-1 and Table V-2. The chromatogram of example V.1 was shown in FIG. 39.

TABLE V-1

| | Reductant/ZnCl₂ | ZnCl₂/Antibody | | | | |
|---|---|---|---|---|---|---|
| NO. | (Malor ratio) | (Malor ratio) | D0 (%) | D2 (%) | D4 (%) | D6 (%) |
| E V.1 | 1:0.2 | 0.80:1 | 2.45 | 8.78 | 85.25 | 3.52 |
| E V.2 | 1:0.25 | 1.00:1 | 0 | 10.11 | 85.55 | 4.34 |
| E V.3 | 1:0.3 | 1.2:1 | 0 | 9.81 | 87.75 | 2.44 |
| E V.4 | 1:0.375 | 1.5:1 | 0 | 10.90 | 86.80 | 2.30 |
| E V.5 | 1:0.425 | 1.7:1 | 0 | 8.61 | 88.67 | 2.72 |
| E V.6 | 1:0.5 | 2:1 | 0 | 8.48 | 89.91 | 1.61 |
| E V.7 | 1:0.575 | 2.3:1 | 0 | 7.21 | 91.02 | 1.77 |
| E V.8 | 1:0.625 | 2.5:1 | 0 | 6.92 | 90.98 | 2.10 |
| E V.9 | 1:0.675 | 2.7:1 | 0 | 7.43 | 90.47 | 2.11 |
| E V.10 | 1:0.75 | 3:1 | 3.19 | 7.58 | 89.23 | 0 |
| E V.11 | 1:0.875 | 3.5:1 | 1.64 | 7.32 | 89.34 | 1.70 |
| E V.12 | 1:1 | 4:1 | 2.22 | 8.98 | 87.44 | 1.36 |

TABLE V-2

| | D0 area | D2 area | D4 area | D6 area |
|---|---|---|---|---|
| NO. | (mAU) | (mAU) | (mAU) | (mAU) |
| E V.1 | 35.22 | 126.19 | 1224.91 | 50.54 |
| E V.2 | 0 | 277.83 | 2351.98 | 119.33 |
| E V.3 | 0 | 307.74 | 2752.01 | 76.46 |
| E V.4 | 0 | 362.98 | 2891.51 | 76.61 |
| E V.5 | 0 | 277.16 | 2853.71 | 87.44 |
| E V.6 | 0 | 267.83 | 2840.25 | 50.88 |
| E V.7 | 0 | 179.76 | 2268.89 | 44.00 |
| E V.8 | 0 | 172.95 | 2274.73 | 52.59 |
| E V.9 | 0 | 188.80 | 2299.60 | 53.51 |
| E V.10 | 62.28 | 147.82 | 1741.24 | 0 |
| E V.11 | 38.78 | 172.99 | 2111.87 | 40.25 |
| E V.12 | 47.73 | 193.01 | 1880.58 | 29.32 |

As the results shown in above table, the linker-payloads (MC-VC-PAB-MMAE) were successfully linked to Trastuzumab, which indicated the ADC with D4 was successfully synthesized.

According to the results of table V-1, D4 was at least 85% when the molar ratio of the ZnCl₂ and the monoclonal antibody was 0.8:1 to 4:1 and the molar ratio of the reductant and the ZnCl₂ is 1:0.2 to 1:1. Further, the highest proportion of D4 was 91.02%, when the molar ratio of the ZnCl₂ and the monoclonal antibody was 2.3:1.

### Example V.13: Preparation of Trastuzumab-[MC-VC-PAB-MMAE]₄ Conjugate (The ADC with D4)

(1) ZnCl₂ (0.0276 mM) and TCEP-3 (0.054 mM) were added to a solution of a monoclonal antibody Trastuzumab (0.012 mM, in Bis-Tris, pH6.7, 20 mM) and the reaction mixture was allowed to stay at 4° C. for 16 h;
(2) MC-VC-PAB-MMAE (0.084 mM) in DMA was introduced and the reaction was continued at room temperature for 2 h;
(3) The reaction mixture was subjected to purification using a desalting column.

### Examples V.14-V.28 and Comparative Example V.1: Preparation of Trastuzumab-[MC-VC-PAB-MMAE]₄ Conjugate (The ADC with D4) with Different Buffer System

The method is similar to Example V.13. The difference is the buffer system which is shown in Table V-3. Meanwhile, the reaction time is 3.5 h in step (1) and the concentration of MC-VC-PAB-MMAE is 0.072 mM in example V.25-V.27.

The homogeneity assay result and the chromatographic peak area of examples V.13-V.28 and comparative example V.1 were shown in Table V-3 and Table V-4.

TABLE V-3

| | Buffer | | | | | |
|---|---|---|---|---|---|---|
| NO. | system | D0 (%) | D2 (%) | D4 (%) | D6 (%) | D8(%) |
| E V.13 | Bis-Tris, | 2.01 | 4.26 | 90.61 | 3.12 | 0 |
| | pH 6.7 | | | | | |
| E V.14 | PIPES, pH 6.7 | 4.29 | 9.15 | 83.92 | 2.65 | 0 |
| E V.15 | HEPES, pH 7.0 | 0 | 3.56 | 93.14 | 3.30 | 0 |
| E V.16 | MOPS, pH 7.0 | 1.92 | 6.82 | 87.86 | 3.40 | 0 |
| E V.17 | MOBS, pH 7.4 | 0 | 3.42 | 92.92 | 3.66 | 0 |
| E V.18 | DIPSO, pH 7.4 | 0 | 3.98 | 93.12 | 2.90 | 0 |
| E V.19 | MOPSO, pH 7.4 | 0 | 3.96 | 92.90 | 3.14 | 0 |
| E V.20 | TES, pH 7.4 | 0 | 4.77 | 92.84 | 2.39 | 0 |
| E V.21 | TAPSO, PH 7.4 | 0 | 4.24 | 93.71 | 2.05 | 0 |
| E V.22 | ACES, pH 7.4 | 0 | 4.53 | 92.87 | 2.60 | 0 |
| E V.23 | BES, pH 7.0 | 1.22 | 4.46 | 92.35 | 1.97 | 0 |
| E V.24 | MES, pH 7.0 | 2.15 | 5.87 | 88.03 | 3.95 | 0 |
| E V.25 | MES, pH 5.8 | 9.72 | 8.33 | 70.27 | 9.22 | 2.46 |
| E V.26 | MES, pH 6.4 | 8.70 | 8.36 | 76.53 | 5.29 | 1.11 |
| E V.27 | MES, pH 6.7 | 4.83 | 4.91 | 83.90 | 4.80 | 1.57 |
| E V.28 | PB, PH 6.7 | 3.71 | 16.18 | 76.60 | 3.50 | 0 |
| C V.1 | ADA, PH 6.7 | 0 | 0 | 0 | 0 | 0 |

TABLE V-4

| | D0 area | D2 area | D4 area | D6 area | D8 area |
|---|---|---|---|---|---|
| NO. | (mAU) | (mAU) | (mAU) | (mAU) | (mAU) |
| E V.13 | 48.90 | 103.66 | 2204.81 | 75.94 | 0 |
| E V.14 | 92.53 | 197.36 | 1810.95 | 57.17 | 0 |
| E V.15 | 0 | 85.33 | 2231.28 | 78.99 | 0 |
| E V.16 | 40.42 | 143.71 | 1851.25 | 71.61 | 0 |
| E V.17 | 0 | 197.42 | 5366.75 | 211.50 | 0 |
| E V.18 | 0 | 91.96 | 2151.17 | 66.98 | 0 |
| E V.19 | 0 | 103.02 | 2419.05 | 81.76 | 0 |
| E V.20 | 0 | 98.05 | 1908.79 | 49.17 | 0 |
| E V.21 | 0 | 97.09 | 2145.12 | 47.01 | 0 |
| E V.22 | 0 | 111.90 | 2294.99 | 64.23 | 0 |
| E V.23 | 27.72 | 101.05 | 2093.07 | 44.71 | 0 |
| E V.24 | 53.79 | 146.70 | 2198.19 | 98.57 | 0 |
| E V.25 | 257.73 | 220.91 | 1863.77 | 244.66 | 65.29 |
| E V.26 | 264.90 | 254.52 | 2329.71 | 161.03 | 33.88 |
| E V.27 | 135.66 | 137.99 | 2358.62 | 134.96 | 44.01 |
| E V.28 | 82.13 | 358.15 | 1695.19 | 77.54 | 0 |
| C V.1 | 0 | 0 | 0 | 0 | 0 |

As shown in the above table, when the buffer system is ADA system, the reaction is largely unsuccessful. The content of D4 is greater than 80% when the buffer system is Bis-Tris buffer, PIPES buffer, MOPS buffer, BES buffer, HEPES buffer, MOPSO buffer, MOBS buffer, TES buffer, DIPSO buffer, MES buffer, ACES buffer or TAPSO buffer.

### Examples V.29-V.39: Preparation of Trastuzumab-[MC-VC-PAB-MMAE]₄ Conjugate (The ADC with D34) with Different Reductant Time and/or Temperature in Step (1)

The method is similar to Example V.1. The difference is the reductant time and/or temperature in step (1) which is shown in Table V-5. Meanwhile, the molar ratio of the reductant and the antibody is 4.5:1, and the molar ratio of the ZnCl₂ and the antibody is 2.3:1 in examples V.29-V.39.

The homogeneity assay result and the chromatographic peak area of examples V.29-V.39 were shown in Table V-5 and Table V-6.

TABLE V-5

| | Temperature | Time | | | | | | |
|---|---|---|---|---|---|---|---|---|
| | in step (1) | in step | | | | | | |
| NO. | (° C.) | (1) (h) | D0 (%) | D2 (%) | D3(%) | D4 (%) | D6 (%) | D8(%) |
| E V.29 | 4 | 2 | 6.23 | 13.85 | 0 | 76.35 | 3.57 | 0 |
| E V.30 | 4 | 4 | 3.22 | 7.84 | 0 | 81.44 | 3.06 | 4.44 |
| E V.31 | 4 | 6 | 2.09 | 6.88 | 0 | 88.50 | 2.54 | 0 |
| E V.32 | 4 | 8 | 1.53 | 5.23 | 0 | 89.99 | 3.25 | 0 |
| E V.33 | 4 | 12 | 1.72 | 4.96 | 0 | 90.26 | 3.06 | 0 |
| E V.34 | 4 | 16 | 1.22 | 4.46 | 0 | 92.35 | 1.97 | 0 |
| E V.35 | 25 | 1 | 2.30 | 11.67 | 0 | 80.96 | 5.06 | 0 |
| E V.36 | 25 | 2 | 1.53 | 8.49 | 0 | 78.87 | 7.48 | 3.64 |
| E V.37 | 25 | 4 | 1.75 | 6.63 | 1.19 | 79.35 | 8.72 | 2.36 |
| E V.38 | 37 | 1 | 1.98 | 9.97 | 0 | 73.35 | 11.59 | 3.13 |
| E V.39 | 37 | 2 | 2.54 | 6.82 | 0 | 67.16 | 17.70 | 5.79 |

TABLE V-6

| | D0 | D2 | D3 | D4 | D6 | D8 |
|---|---|---|---|---|---|---|
| | area | area | area | area | area | area |
| NO. | (mAU) | (mAU) | (mAU) | (mAU) | (mAU) | (mAU) |
| E V.29 | 114.03 | 253.61 | 0 | 1398.31 | 65.44 | 0 |
| E V.30 | 78.30 | 190.61 | 0 | 1980.28 | 74.295 | 107.995 |
| E V.31 | 45.98 | 151.75 | 0 | 1951.39 | 55.95 | 0 |
| E V.32 | 37.04 | 126.82 | 0 | 2180.76 | 78.86 | 0 |
| E V.33 | 39.07 | 112.81 | 0 | 2051.16 | 69.60 | 0 |
| E V.34 | 27.72 | 101.05 | 0 | 2093.07 | 44.71 | 0 |
| E V.35 | 47.42 | 240.42 | 0 | 1667.54 | 104.23 | 0 |
| E V.36 | 39.18 | 217.76 | 0 | 2023.33 | 191.96 | 93.25 |
| E V.37 | 40.47 | 153.28 | 27.54 | 1834.47 | 201.66 | 54.46 |
| E V.38 | 43.70 | 220.57 | 0 | 1622.90 | 256.37 | 69.15 |
| E V.39 | 36.34 | 97.59 | 0 | 961.52 | 253.33 | 82.88 |

As shown in above table, the content of D4 is up to 67%, even to 80, 85 or 90% when the reductant time is 2 h to 18 h and the reduction temperature is 4° C. to 37° C. Further, the content of D4 increases as the reaction time from 2 h to 6 h and reaches plateau after 6 h.

Examples V.40-V.43: Preparation of Trastuzumab-[MC-VC-PAB-MMAE]₄ Conjugate (The ADC with D4) with Different Molar Ratio of the Linker-Payload and the Antibody in Step (1)

The method is similar to Example V.1. The difference is the molar ratio of the linker-payload and the antibody in step (1) which is shown in Table V-7. Meanwhile, the molar ratio of the reductant and the antibody is 4.5:1, and the molar ratio of the ZnCl₂ and the antibody is 2.3:1 in examples V.40-V.43.

The homogeneity assay result and the chromatographic peak area of examples V.40-V.43 were shown in Table V-7 and Table V-8.

TABLE V-7

| | The linker | | | | | |
|---|---|---|---|---|---|---|
| | payload/ | | | | | |
| | antibody | | | | | |
| NO. | (molar ratio) | D0 (%) | D2 (%) | D4 (%) | D6 (%) | D8(%) |
| E V.40 | 7:1 | 1.84 | 7.93 | 87.87 | 2.37 | 0 |
| E V.41 | 10:1 | 1.48 | 5.00 | 87.45 | 6.08 | 0 |
| E V.42 | 12:1 | 1.35 | 5.09 | 89.98 | 3.58 | 0 |
| E V.43 | 15:1 | 1.57 | 6.26 | 87.80 | 4.37 | 0 |

TABLE V-8

| | D0 area | D2 area | D4 area | D6 area | D8 area |
|---|---|---|---|---|---|
| NO. | (mAU) | (mAU) | (mAU) | (mAU) | (mAU) |
| E V.40 | 32.88 | 142.09 | 1574.90 | 42.40 | 0 |
| E V.41 | 34.45 | 116.70 | 2042.50 | 141.97 | 0 |
| E V.42 | 19.63 | 73.79 | 1305.63 | 51.99 | 0 |
| E V.43 | 27.29 | 108.89 | 1526.57 | 76.02 | 0 |

According to the results of the above table, D4 was at least 87% when the molar ratio of the linker-payload and the antibody in step (1) was 7:1 to 15:1.

### Example V.44: Preparation of Belantamab-[MC-VC-PAB-MMAE]₄ Conjugate (The ADC with D4)

(1) ZnCl₂ (0.0276 mM) and TCEP-3 (0.06 mM) were added to a solution of a monoclonal antibody Belantamab (0.012 mM, in BES buffer, pH7.0, 20 mM) and the reaction mixture was allowed to stay at 4° C. for 16 h;
(2) MC-VC-PAB-MMAE (0.072 mM) in DMA was introduced and the reaction was continued at room temperature for 2 h;
(3) The reaction mixture was subjected to purification using a desalting column.

The homogeneity assay result and the chromatographic peak area of example V.44 were shown in Table V-9.

TABLE-9

| | | | | | D2 Area | D4 Area | D6 Area | D8 Area |
|---|---|---|---|---|---|---|---|---|
| NO. | D2 (%) | D4 (%) | D6 (%) | D8(%) | (mAU) | (mAU) | (mAU) | (mAU) |
| E V.44 | 3.65 | 82.64 | 8.99 | 4.72 | 107.20 | 2424.66 | 263.74 | 138.51 |

According to the results of the above table, the content of D4 was at least 82% when the antibody is Belantamab. The result demonstrated that the method of the present application is suitable for different antibodies.

### Example V.45: Preparation of Trastuzumab-[MC-VC-PAB-MMAE]₄ Conjugate (The ADC with D4)

(1) ZnCl₂ (0.024 mM) and TCEP (0.0576 mM) were added to a solution of a monoclonal antibody Belantamab (0.012 mM, in BES buffer, pH7.0, 20 mM) and the reaction mixture was allowed to stay at 4° C. for 16 h;
(2) MC-VC-PAB-MMAE (0.144 mM) in DMA was introduced and the reaction was continued at room temperature for 1 h;
(3) The reaction mixture was subjected to purification using a desalting column.

The homogeneity assay result and the chromatographic peak area of example V.55 were shown in Table V-10.

TABLE-10

| | | | | D2 | D4 | D6 |
|---|---|---|---|---|---|---|
| | | | | Area | Area | Area |
| NO. | D2 (%) | D4 (%) | D6 (%) | (mAU) | (mAU) | (mAU) |
| E V.55 | 4.39 | 87.63 | 7.98 | 98.66 | 1967.44 | 179.10 |

According to the results of the above table, the content of D4 was 87.63% when the reductant is TCEP. The result demonstrated that the method of the present application is suitable for different reductants.

### Example V.46: Preparation of Trastuzumab-[MC-VC-PAB-MMAE]₄[MC-GGFG-DXd]₂ Conjugate (The ADC with D4+D2)

(1) ZnCl₂ (0.08 mM) and TCEP-3 (0.15 mM) were added to a solution of a monoclonal antibody Trastuzumab (0.034 mM, in BES buffer, pH7.0, 20 mM) and the reaction mixture was allowed to stay at 4° C. for 16 h;
(2) MC-VC-PAB-MMAE (0.20 mM) in DMA was introduced and the reaction was continued at room temperature for 2 h. The reaction mixture was subjected to purification using a desalting column.
(3) introducing EDTA (0.55 mM) and MC-GGFG-Dxd (0.10 mM), the reaction time is 1.5 h and the reaction temperature is room temperature.
(4) The reaction mixture was subjected to purification using a desalting column.

### Example V.47: preparation of Trastuzumab-[MC-VC-PAB-MMAE]₄[MC-GGFG-DXd]₂ (the ADC with D4+D2)

(1) incubating TCEP-3 (0.048 mM) and trastuzumab (0.012 mM) in the presence of an effective amount of ZnCl₂ (0.0276 mM) in BES (20 mM, pH7.0), The incubation temperature is 4° C. and the incubation time is 16 h;
(2) introducing MC-VC-PAB-MMAE (0.12 mM) to react with reduced thiol groups resulted from step (1), the reaction temperature is room temperature and the reaction time is 2 h. then recovering the product using a desalting column and AKTA with HIC chromatography to afford Trastuzumab-[MC-VC-PAB-MMAE]₄;
(3) incubating ZnCl₂ (0.18 mM), the second reductant TCEP-3 (0.024 mM) and the product from step (2) in BES buffer (pH7.0, 20 mM) and the reaction mixture was allowed to stay at 4° C. overnight;
(4) introducing EDTA (1.2 mM) and introducing MC-GGFG-DXd (0.096 mM) to react with the reduced thiol groups resulted from step (3), the reaction temperature is room temperature and the reaction time is 2 h;
(5) the reaction mixture was subjected to purification using a desalting column.

The homogeneity assay result and the chromatographic peak area of examples V.46-V.47 were shown in Table V-11 and Table V-12.

TABLE V-11

| No. | Figure | D0(%) | D2 (%) | D4 (%) | D6 (%) | D8 (%) | / |
|---|---|---|---|---|---|---|---|
| E V.46-step (2) | 40A | 1.42 | 2.62 | 88.60 | 3.22 | 4.14 | / |
| E V.47-step (2) | 41A | 0 | 2.64 | 97.36 | 0 | 0 | / |
| No. | Figure | D2 (DXd) (%) | D2 (MMAE) (%) | D2 + D2 (%) | D4 + D2 (%) | D4 + D4 (%) | D8 (%) |
| E V.46-step (4) | 40B | 1.50 | 2.75 | 21.99 | 68.08 | 4.08 | 1.61 |
| No. | Figure | / | / | D4 (%) | D4 + D2 (%) | D4 + D4 (%) | D8 (%) |
| E V.47-step (5) | 41B | / | / | 2.27 | 89.47 | 8.26 | / |

TABLE V-12

| | D0 area | D2 area | D4 area | D6 area | D8 area | |
|---|---|---|---|---|---|---|
| No. | (mAU) | (mAU) | (mAU) | (mAU) | (mAU) | / |
| E V.46-step (2) | 85.69 | 158.47 | 5362.04 | 195.15 | 250.80 | / |
| E V.47-step (2) | / | 101.92 | 3752.98 | 0 | 0 | / |
| | D2 (DXd) | D2 (MMAE) | D2 + D2 area | D4 + D2 area | D4 + D4 area | D8 area |
| No. | area (mAU) | area (mAU) | (mAU) | (mAU) | (mAU) | (mAU) |
| E V.46-step (4) | 25.02 | 45.86 | 367.30 | 1137.32 | 68.22 | 26.85 |
| | | | D4 area | D4 + D2 area | D4 + D4 area | D8 area |
| No. | / | / | (mAU) | (mAU) | (mAU) | (mAU) |
| E V.47-step (5) | / | / | 42.26 | 1666.99 | 153.89 | / |

As shown in the above table, the result demonstrated that the content of the ADC with D4+D2 was generally up to 68% or 89%, which indicated the process of method was benefit for site-specific modifying the antibody with D4+D2 and improving the homogeneity.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications, variations, improvements, and substantial equivalents.

1. A method for programmatically managing antibody disulfide bonds site-specific modification, comprising step,
(R1) contacting a first reductant or salt thereof and an antibody in a buffer system in the presence of transition metal ions, to reduce at least one of the interchain disulfide bond of the antibody.

2. The method of claim 1, which characterized in that, in step (R1), one of the interchain disulfide bond of the antibody is reduced, optionally, one of the interchain disulfide bond in the hinge region of the antibody is reduced.

3. The method of claim 1, which characterized in that, in step (R1), three of the interchain disulfide bonds of the antibody are reduced, optionally, two of the interchain disulfide bonds in the Fab region and one of the interchain disulfide bond in the hinge region of the antibody are reduced.

4. The method of claim 1, which characterized in that, the method comprises step (O1),
(O1) introducing an oxidant to selectively re-oxidize the reduced thiol groups resulted from step (R1).

5. The method of claim 4, which characterized in that, in step (O1), the oxidant re-oxidizes the reduced thiol groups in Fab region of the antibody, optionally, four of the reduced thiol groups are re-oxidized.

6. The method of claim 4, which characterized in that, the method comprises step (R2),
(R2) incubating a second reductant or salt thereof in the buffer system to reduce one of the interchain disulfide bond of the antibody resulted from step (O1).

7. The method of claim 1, which characterized in that, the method further comprises the following step,
(C1) introducing an amount of metal chelators and at least equimolecular proportion of a first conjugating group to react with the reduced thiol groups resulted from step (R1) calculated as the molar amount of the antibody, wherein, the first conjugating group is a first end capping reagent, a first linker-payload or a first thiobridge reagent, optionally, the first thiobridge reagent bears the first linker-payload or reactive groups;
(C1) introducing an amount of metal chelators and at least equimolecular proportion of a first conjugating group to react with the reduced thiol groups resulted from step (O1), calculated as the molar amount of the antibody, wherein, the first conjugating group is a first end capping reagent, a first linker-payload or a first thiobridge reagent, optionally, the first thiobridge reagent bears the first linker-payload or reactive groups, the step (O1) is that introducing an oxidant to selectively re-oxidize the reduced thiol groups resulted from step (R1);
or,
(C1) introducing an amount of metal chelators and at least equimolecular proportion of a first conjugating group to react with the reduced thiol groups resulted from step (R2), calculated as the molar amount of the antibody, wherein, the first conjugating group is a first end capping reagent, a first linker-payload or a first thiobridge reagent, optionally, the first thiobridge reagent bears the first linker-payload or reactive groups, the step (R2) is that incubating a second reductant or salt thereof in the buffer system to reduce one of the interchain disulfide bond of the antibody resulted from step (O1).

8. The method of claim 3, which characterized in that, the method further comprises the following step,
(C2) incubating at least equimolecular proportion of the first conjugating group to react with the reduced thiol groups resulted from step (R1), calculated as the molar amount of antibody; then,
optionally, introducing the metal chelators and the oxidant, or
optionally, introducing the metal chelators and the first conjugating group.

9. The method of claim 7 or 8, which characterized in that, the method further comprises the following steps,
(R3) incubating the second reductant or salt thereof in the buffer system to reduce the interchain disulfide bonds of the antibody resulted from step (C1) or (C2), optionally, introducing the transition metal ions;
(C3) introducing at least equimolecular proportion of a second conjugating group to react with the reduced thiol groups resulted from step (R3), calculated as the molar amount of antibody, optionally, introducing the metal chelator, wherein, the second conjugating group is a second end capping reagent, a second linker-payload or a second thiobridge reagent, optionally, the second thiobridge reagent bears the second linker-payload or reactive groups.

10. The method of claim 9, which characterized in that, in step (R3), one, two or three of the interchain disulfide bonds of the antibody are reduced.

11. The method of claim 7, which characterized in that, in step (C1), one, two, three or six of the first conjugating groups are covalently linked to the reduced thiol groups resulted from step (R1).

12. The method of claim 7, which characterized in that, in step (C1), one or two of the first conjugating groups are covalently linked to the remaining thiol groups resulted from step (O1).

13. The method of claim 7, which characterized in that, in step (C1), two or four of the first conjugating groups are covalently linked to the reduced thiol groups resulted from step (R2).

14. The method of claim 8, which characterized in that, in step (C2), two, three, four or six of the first conjugating groups are covalently linked to the reduced thiol groups resulted from step (R1).

15. The method of claim 9, which characterized in that, in step (C3), one, two, three, four or six of the second conjugating groups are covalently linked to the reduced thiol groups resulted from step (R3).

16. The method of claim 6, which characterized in that, the reductants, including the first reductant and the second reductant, are independently selected from the group consisting of tris (2-carboxyethyl) phosphine (TCEP), or a compound having formula (I) or a salt, solvate, stereoisomer thereof; wherein 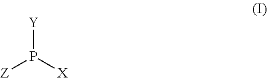
in formula (I),
X, Y and Z independently covalently connect the phosphorus atom through P-C bond, which is P-C(sp³) or P-C(sp²);
X is of formula (III): 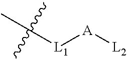
L₁ is selected from the group consisting of —CH(R₁)-, —C(CH₃)(R₁)-, —CH(R₁)CH(R₂)—, —CH(R₁)CH(R₂)CH(R₃)-, aryl group which is independently unsubstituted or substituted with group containing at least a coordinating atom selected from N, O and S, and heteroaryl group which is independently unsubstituted or substituted with group containing at least a coordinating atom selected from O and S;
R₁, R₂ and R₃ independently are H, C₁-C₅ alkyl group, C₁-C₅ hydroxyalkyl group, C₁-C₅ carboxy alkyl group, C₁-C₅ hydroxylamine alkyl group, C₁-C₅ N-hydroxy amide alkyl group, aryl group or heteroaryl group; or
R₂ or R3 forms a 5-6 membered unsubstituted or substituted ring with L₂;
A is not present or present, when A is present, A is —C(O)—, or —C(O) J-;
J is organic group comprising amino or imino group and carbonyl group at the same time, of which the amino or imino group forms amide group with —C(O), the carboxyl group unlinked or covalently linked to L₂;
L₂ is not present or present, L₂ works as transition metal chelator motif and is —N(R₄)(R₅) or hydroxy;
R₄ and R₅ independently are hydrogen, C₀-C₅ hydroxyalkyl group, C₁-C₅ alkyl group, C₁-C₅ alkoxy group, —(CH₂)n¹(OCH₂CH₂O)n²CH(R₆)CO(R₇), unsubstituted or substituted 5-6 membered saturated heterocyclic group, unsubstituted or substituted arylalkyl group, unsubstituted or substituted aryl alkoxy group, unsubstituted or substituted aryl group, unsubstituted or substituted heteroaryl group, unsubstituted or substituted heteroaryl alkyl group, R₄ or R₅ forms a 5-6 membered unsubstituted or substituted ring with R₂ or R₃;
R₆ is hydrogen, amino, C₁-C₅ alkyl, C₁-C₅ hydroxyalkyl group, C₁-C₅ carboxy alkyl group, aryl group, unsubstituted or substituted arylalkyl group, C₁-C₅ N-hydroxy amide alkyl group, heteroaryl group or heteroaryl alkyl group;
R₇ is hydroxy, C₁-C₅ alkoxy group, —NH(CH₂CONH)n³OH;
n¹, n² and n³ independently are the number 0, 1, 2, 3, 4;
R₄ and R₅ are not hydroxy at the same time;
Y is same as X,
Z is same as X, or
Y and Z independently are 5-6 membered unsubstituted or substituted saturated heterocyclic group, C₁-C₅ alkyl group, C₁-C₅ hydroxyalkyl group, aryl group, C₁-C₅ carboxy alkyl group, 5-6 membered unsubstituted or substituted cycloalkyl group, or 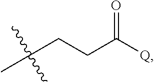
—C(O)Q is ester group, imide group or amide group,
X, Y and Z are not —CH₂CH₂C(O)OH at the same time.

17. The method of claim 16, which characterized in that, in formula (I),
L₁ is unsubstituted phenyl group or phenyl group substituted with hydroxy or carboxy group, in ortho or meta position, and the phenyl group connected to A in ortho, meta or para position,
A is —C(O)—;
L₂ is —N(R₄)(R₅) or hydroxy; and
R₄ is hydrogen, R₅ is hydroxy.

18. (canceled)

19. The method of claim 16, which characterized in that, in formula (I),
L₁ is phenyl group which is unsubstituted or substituted with hydroxy, halogen, carboxyl, sulfonyl, amino, methoxy or ethoxy in ortho, meta or para position, or L₁ is unsubstituted or substituted 4-pyridyl group or unsubstituted or substituted 4-quinolyl group; and
A and L₂ are not present.

20. (canceled)

21. The method of claim 19, which characterized in that, in formula (I),
L₁ is

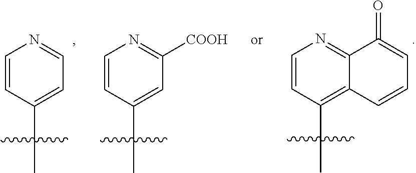

22. The method of claim 16, which characterized in that, in formula (I),
L₁ is —CH(R₁)CH(R₂)—,
R₁ and R₂ independently are H, methyl group, isopropyl group, hydroxymethyl group, hydroxyethyl group, carboxy methyl group, carboxy ethyl group, N-hydroxy ethyl amide group, phenyl group, 2-pyridyl group, 4-pyridyl group or 4-imidazole group, or R₂ forms a 5-6 membered unsubstituted or substituted ring with L₂.

23. The method of claim 22, which characterized in that, in formula (I),
L₁ is —CH(R₁)CH(R₂)—;
R₁ is H, and R₂ forms a 5-6 membered unsubstituted or substituted ring with R₄ of L₂; and
L₂ is —N(R₄)(R₅), R₅ is hydroxy.

24. (canceled)

25. The method of claim 22, which characterized in that, in formula (I),
L₁ is —CH(R₁)CH(R₂)—,
R₁ is H,
R₂ is methyl group, hydroxymethyl group, hydroxyethyl group, carboxy ethyl group, phenyl group, N-hydroxy ethyl amide group, 2-pyridyl group, 4-pyridyl group or 4-imidazole group,
A is —C(O)—;
L₂ is —N(R₄)(R₅);
R₄ is hydrogen, unsubstituted or substituted 5-6 membered saturated heterocyclic group,
R₅ is hydroxy;
or R₄ and R₅ form a 5-6 membered unsubstituted or substituted ring.

26. The method of claim 22, which characterized in that, in formula (I),
R₄ is

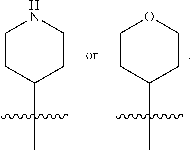 [SMILES: *C1CCNCC1 ; *C1CCOCC1]

27. (canceled)

28. The method of claim 25, which characterized in that, in formula (I),
L₂ is

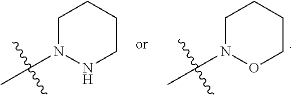 [SMILES: *N1CCCCN1 ; *N1CCCCO1]

29. (canceled)

30. The method of claim 16, which characterized in that, in formula (I),
L₁ is —CH(R₁)CH(R₂)—,
R₁ is methyl group, isopropyl group, carboxy ethyl group or N-hydroxy ethyl amide group,
R₂ is H,
A is —C(O)—;
L₂ is —N(R₄)(R₅); and
R₄ is hydrogen, and R₅ is hydroxy.

31. (canceled)

32. The method of claim 16, which characterized in that, in formula (I),
L₁ is —CH(R₁)CH(R₂)—;
R₁ and R₂ independently are H;
L₂ is —N(R₄)(R₅);
R₄ is hydrogen, C₁-C₅ alkyl group, —(CH₂)n¹(OCH₂CH₂O)n²CH(R₆)CO(R₇), unsubstituted or substituted 5-6 membered saturated heterocyclic group, unsubstituted or substituted arylalkyl group, unsubstituted or substituted aryl group, unsubstituted or substituted heteroaryl alkyl group, or R₄ and R5 form a 5-6 membered unsubstituted or substituted ring;
R₅ is hydroxy,
R₆ is hydrogen, amino, C₁-C₅ alkyl, C₁-C₅ hydroxyalkyl group, C₁-C₅ carboxy alkyl group, aryl group, C₁-C₅ N-hydroxy amide alkyl group, heteroaryl group or heteroaryl alkyl group;
R₇ is hydroxy, C₁-C₅ alkoxy group, —NH(CH₂CONH)n³OH; and
n¹, n² and n³ independently are the number 0, 1, 2, 3, 4.

33. The method of claim 32, which characterized in that, in formula (I),
R₄ is hydrogen, methyl group, —(CH₂)n¹(OCH₂CH₂O)n²CH(R₆)CO(R₇), 5-6 membered saturated heterocyclic group which comprises a heteroatom N or O, benzyl group, benzyl group which is substituted with hydroxy on the phenyl ring, phenyl which is unsubstituted or substituted with hydroxy, halogen or carboxyl group, heteroaryl alkyl group which comprises a heteroatom N, or R₄ and R₅ form a 5-6 membered ring;
R₅ is hydroxy,
R₆ is hydrogen, C₁-C₅ alkyl, C₁-C₅ hydroxyalkyl group, or heteroaryl alkyl group;
R₇ is hydroxy, C₁-C₅ alkoxy group, —NH(CH₂CONH)n³OH; and
n¹, n² and n³ independently are the number 0, 1, 2, 3, 4.

34. The method of claim 32, which characterized in that, in formula (I),
R₄ is 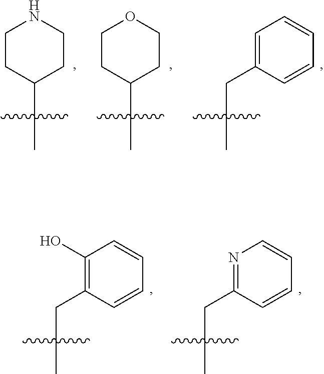
hydrogen or —(CH₂)n¹(OCH₂CH₂O)n²CH(R₆)CO(R₇),
R₅ is hydroxy,
R₆ is hydrogen, methyl group, hydroxymethyl group or 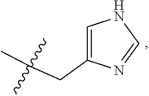 [SMILES: *Cc1c[nH]cn1]
R₇ is hydroxy or —NH(CH₂CONH)n³OH; and
n¹, n² and n³ independently are the number 0.

35. The method of claim 16, which characterized in that, in formula (I),
L₁ is —CH(R₁)CH(R₂)—;
R₁ and R₂ independently are H;
L₂ is —N(R₄)(R₅);
R₄ and R₅ are independently —(CH₂)n¹(OCH₂CH₂O)n²CH(R₆)CO(R₇) or unsubstituted or substituted heteroaryl alkyl group,
R₆ is hydrogen, amino, C₁-C₅ alkyl, C₁-C₅ hydroxyalkyl group, C₁-C₅ carboxy alkyl group, aryl group, C₁-C₅ N-hydroxy amide alkyl group, heteroaryl group or heteroaryl alkyl group;
R₇ is hydroxy, C₁-C₅ alkoxy group, —NH(CH₂CONH)n³OH; and
n¹, n² and n³ independently are the number 0, 1, 2, 3, 4.

36. The method of claim 35, which characterized in that, in formula (I),
R₄ and R₅ are independently —(CH₂)n¹(OCH₂CH₂O)n²CH(R₆)CO(R₇) or 6 membered heteroaryl alkyl group,
R₆ is hydrogen,
R₇ is hydroxy, C₁-C₅ alkoxy group, —NH(CH₂CONH)n³OH; and
n¹, n² and n³ independently are the number 0, 1, 2, 3, 4.

37. The method of claim 36, which characterized in that, in formula (I),
R₄ and R₅ are independently —(CH₂)n¹(OCH₂CH₂O)n²CH(R₆)CO(R₇) or 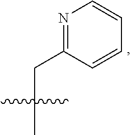 [SMILES: *Cc1ccccn1]
R₆ is hydrogen,
R₇ is hydroxy or —NH(CH₂CONH)n³OH; and
n¹, n² and n³ independently are the number 0.

38. The method of claim 16, which characterized in that, in formula (I),
L₁ is —CH(R₁)CH(R₂)—;
R₁ and R₂ independently are H;
L₂ is —N(R₄)(R₅);
R₄ is hydrogen, C₀-C₅ hydroxyalkyl group, C₁-C₅ alkyl group, unsubstituted or substituted C₁-C₅ alkoxy group, —(CH₂)n¹(OCH₂CH₂O)n²CH(R₆)CO(R₇), unsubstituted or substituted arylalkyl group, unsubstituted or substituted aryl alkoxy group, unsubstituted or substituted aryl group, unsubstituted or substituted heteroaryl group, unsubstituted or substituted heteroaryl alkyl group;
R₅ is hydrogen,
R₆ is hydrogen, amino, C₁-C₅ alkyl, C₁-C₅ hydroxyalkyl group, C₁-C₅ carboxy alkyl group, aryl group, unsubstituted or substituted arylalkyl group, C₁-C₅ N-hydroxy amide alkyl group, heteroaryl group or heteroaryl alkyl group;
R₇ is hydroxy, C₁-C₅ alkoxy group, —NH(CH₂CONH)n³OH; and
n¹, n² and n³ independently are the number 0, 1, 2, 3, 4.

39. The method of claim 38, which characterized in that, in formula (I),
R₄ is hydrogen, C₀-C₃ hydroxyalkyl group, C₁-C₃ alkoxy group, —(CH₂)n¹(OCH₂CH₂O)n²CH(R₆)CO(R₇), phenyl group which is substituted with carboxy, hydroxy, amino, halogen, pyridyl group, amino which is substituted with 2-methylpyridine, benzyl group which is substituted with carboxy, hydroxy, amino or halogen, aryl alkoxy group, pyridyl group which is substituted with carboxy, bipyridyl group, 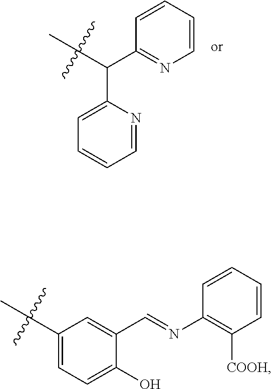 [SMILES: *C(c1ccccn1)c1ccccn1 ; *c1ccc(O)c(/C=N/c2ccccc2C(=O)O)c1]
R₅ is hydrogen,
R₆ is hydrogen, amino, C₁-C₃ alkyl, C₁-C₃ hydroxyalkyl group, C₁-C₃ carboxy alkyl group, aryl group, arylalkyl group which is unsubstituted or substituted with hydroxy group, halogen, cyano group or nitro group, C₁-C₅ N-hydroxy amide alkyl group, heteroaryl group or heteroaryl alkyl group;
R₇ is hydroxy, C₁-C₅ alkoxy group, —NH(CH₂CONH)n³OH; and
n¹, n² and n³ independently are the number 0, 1, 2, 3, 4.

40. The method of claim 39, which characterized in that, in formula (I),
R₄ is hydrogen, hydroxy, ethyl hydroxyl group, 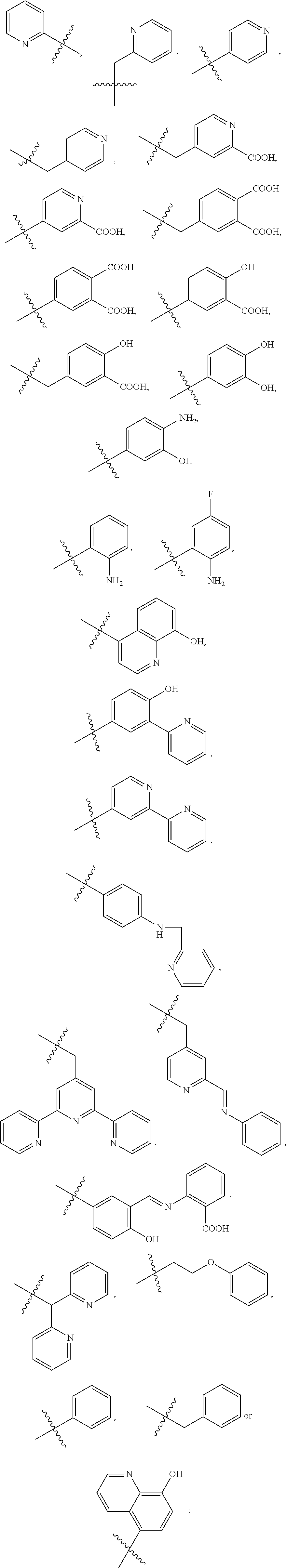
and
R₅ is hydrogen.

41. The method of claim 38, wherein in formula (I),
R₄ is —(CH₂)n¹(OCH₂CH₂O)n²CH(R₆)CO(R₇),
R₅ is hydrogen,
R₆ is hydrogen, amino, methyl, hydroxymethyl group, benzyl group, carboxy ethyl group, benzyl group substituted with —OH, F, —CN or —NO₂, N-hydroxy ethyl amide group, 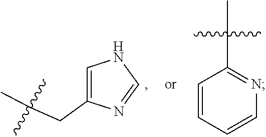 [SMILES: *Cc1c[nH]cn1 ; *c1ccccn1]
R₇ is hydroxy, —NH(CH₂CONH)n³OH;
n¹ and n³ independently are the number 0, 1, 2, 3, 4, and
n² is the number 0.

42. The method of claim 38, which characterized in that, in formula (I),
R₄ is —(CH₂)n¹(OCH₂CH₂O)n²CH(R₆)CO(R₇),
R₅ is hydrogen,
R₆ is hydrogen, amino, methyl, hydroxymethyl group, benzyl group, carboxy ethyl group, 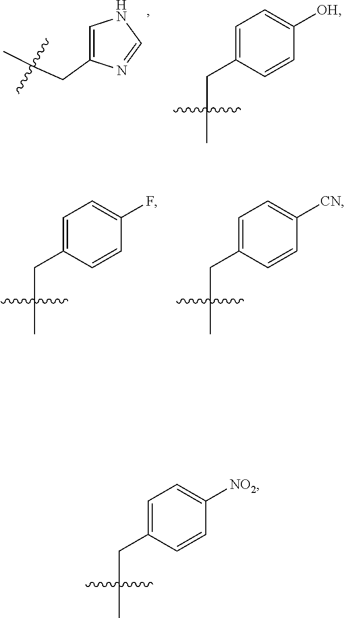 [SMILES: *Cc1c[nH]cn1 ; *Cc1ccc(O)cc1 ; *Cc1ccc(F)cc1 ; *Cc1ccc(C#N)cc1 ; *Cc1ccc([N+](=O)[O-])cc1]
N-hydroxy ethyl amide group, heteroaryl group or heteroaryl alkyl group;
R₇ is hydroxy, —NH(CH₂CONH)n³OH;
n¹ is the number 0 or 2,
n² is the number 0 or 1, and
n³ is the number 0.

43. The method of claim 16, which characterized in that, in formula (I),
J is peptide residue comprising mono amino acid residue, dipeptide, tripeptide, tetrapeptide, pentapeptide, aminopropionic acid, aminobutyric acid, amino valeric acid, aminoacid, aminoheptanoic acid, aminooctanoic acid, or NH₂(OCH₂CH₂O)n⁴CH₂COOH, n⁴ is the number of 2-10,
the amino acid is selected from the group consisting of glycine (Gly), alanine (Ala), serine (Ser), arginine (Arg), asparagine (Asn), aspartic acid (Asp), cysteine (Cys), glutamine (Gln), glutamic acid (Glu), histidine (His), isoleucine (Ile), leucine (Leu), lysine (Lys), methionine (Met), phenylalanine (Phe), proline (Pro), threonine (Thr), tryptophan (Trp), tyrosine (Tyr) and valine (Val).

44. The method of claim 43, which characterized in that, in formula (I),
J is the residue of histidine, serine, alanine, glycine, phenylalanine, asparagine, tyrosine or asparagine.

45. The method of claim 43, which characterized in that, in formula (I),
A is —C(O) J-,
J is the residue of histidine, serine, alanine, glycine, phenylalanine, asparagine, tyrosine or asparagine,
L₂ is —N(R₄)(R₅), R₄ is hydrogen, R₅ is hydroxy.

46. The method of claim 16, which characterized in that, in formula (I),
Y and Z independently are 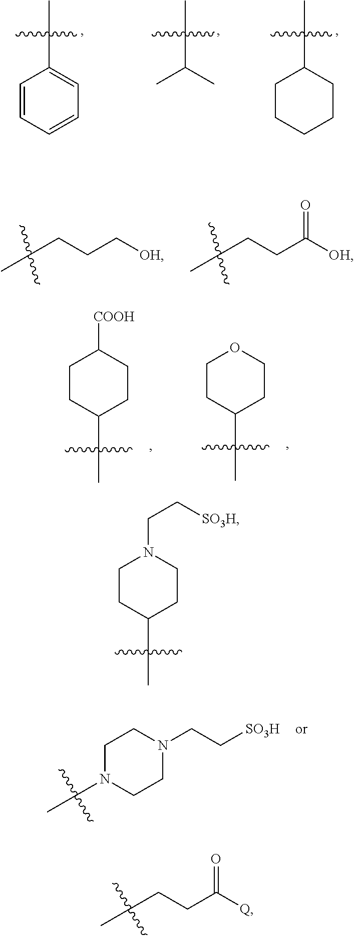
Q is —NHOH, —NHCH₂CH₂SO₃H, —N(CH₂CH₂OH)₂, —NHCH₂COOH, —NHCH₂(CH₃)COOH, —NH(CH₂CH₂O)₃CH₃.

47. The method of claim 16, which characterized in that, the reductant is selected from the group consisting of

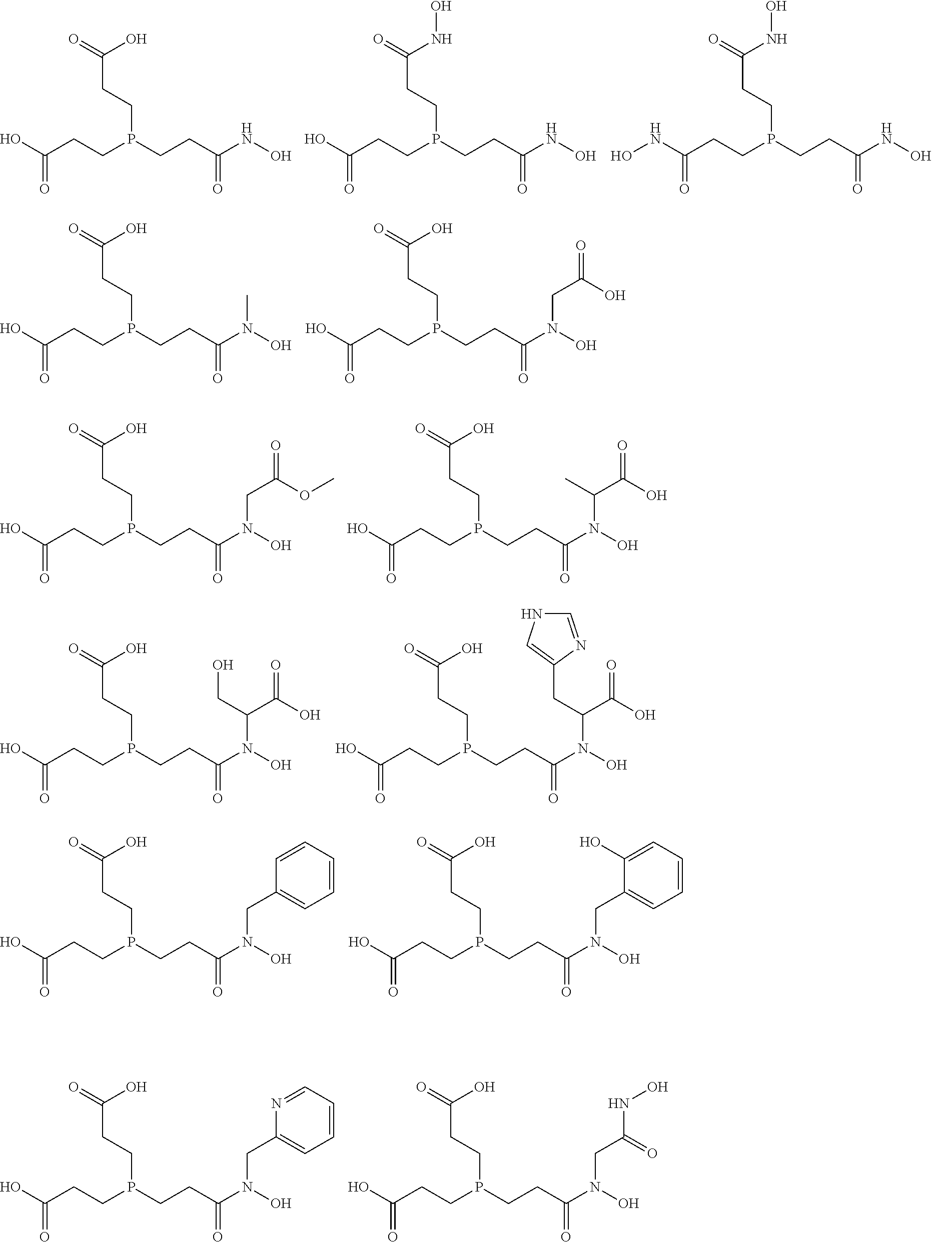

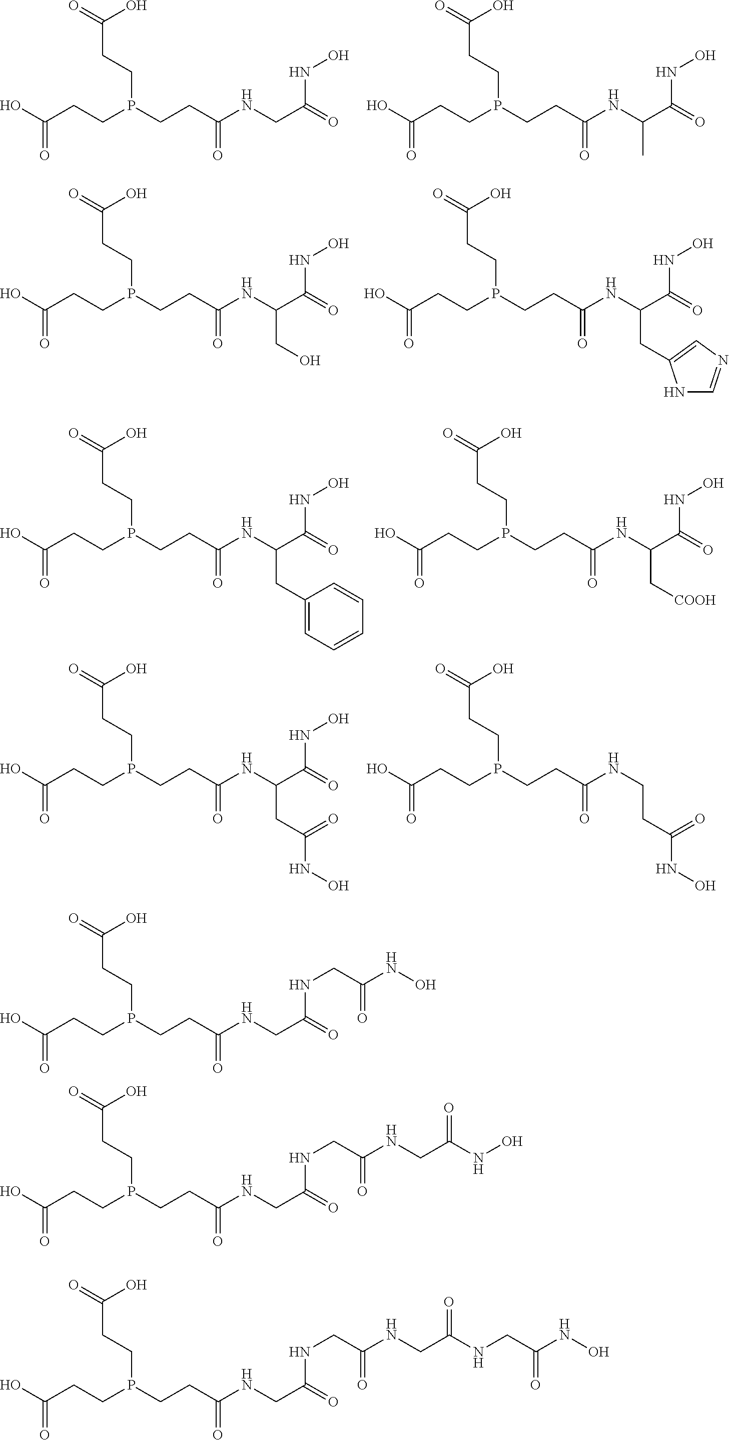

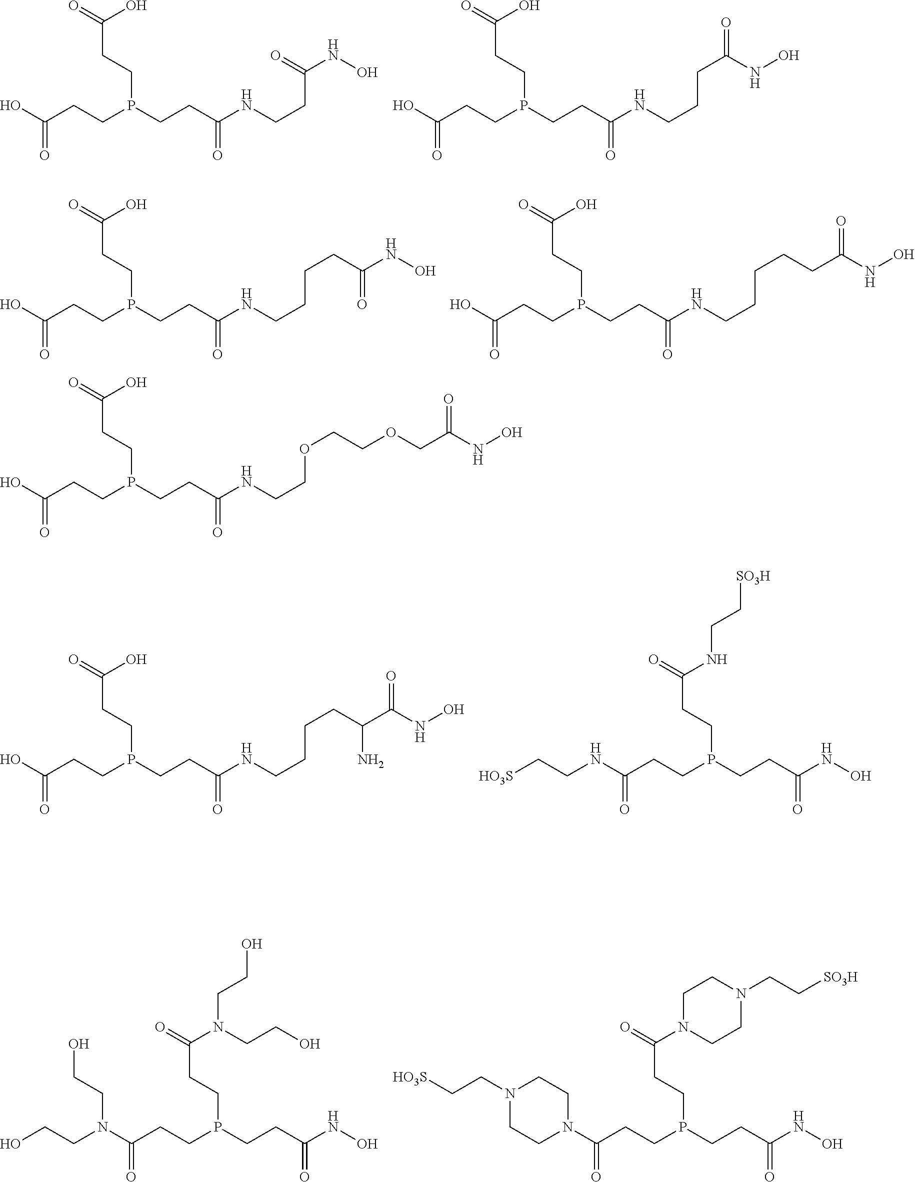

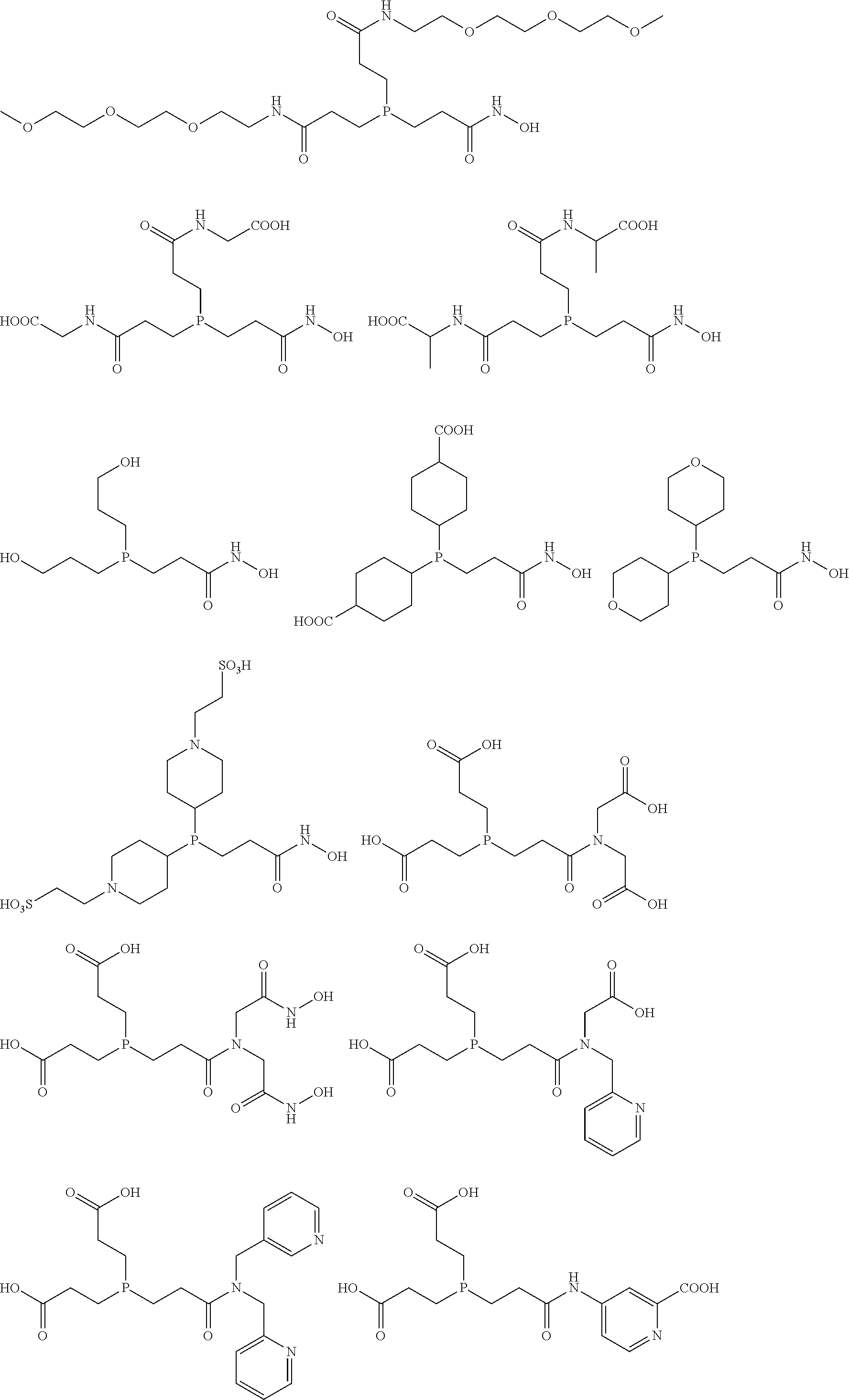

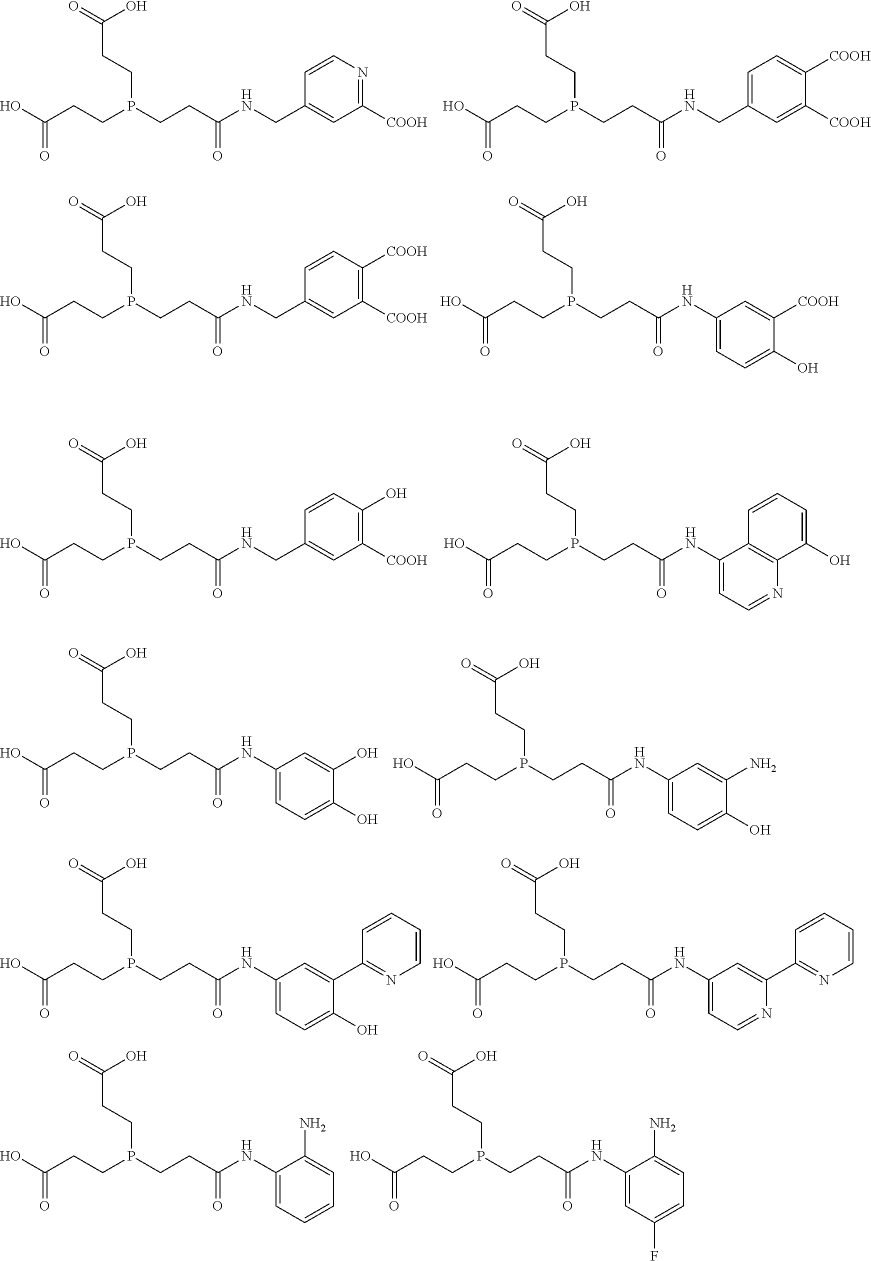

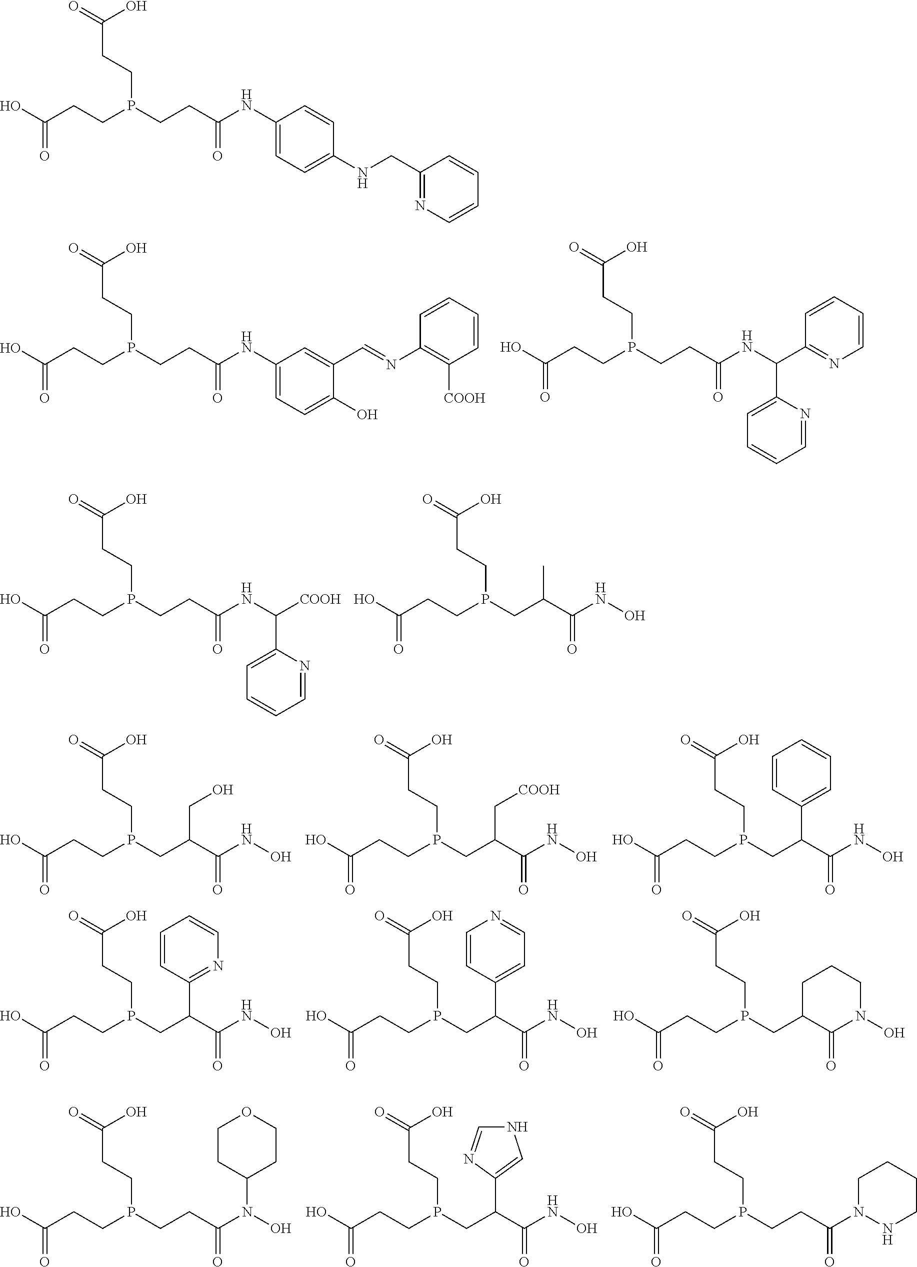

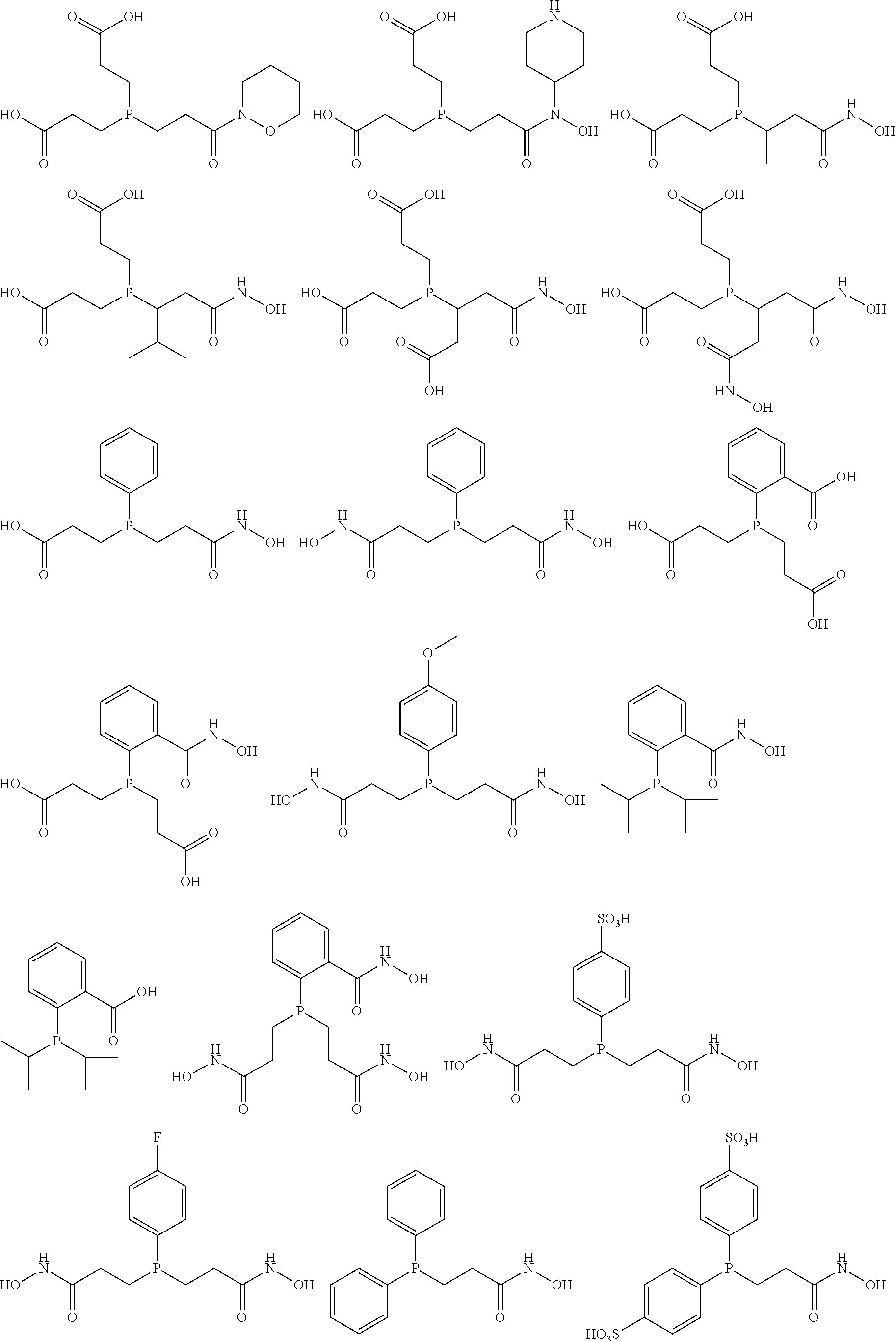

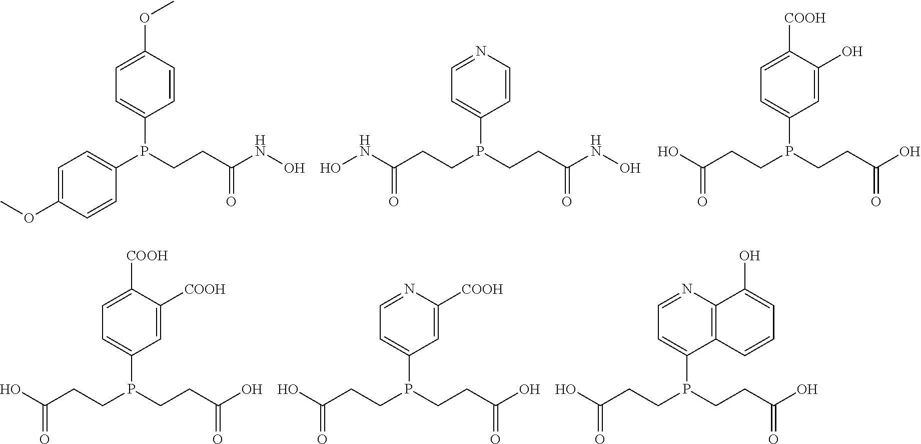

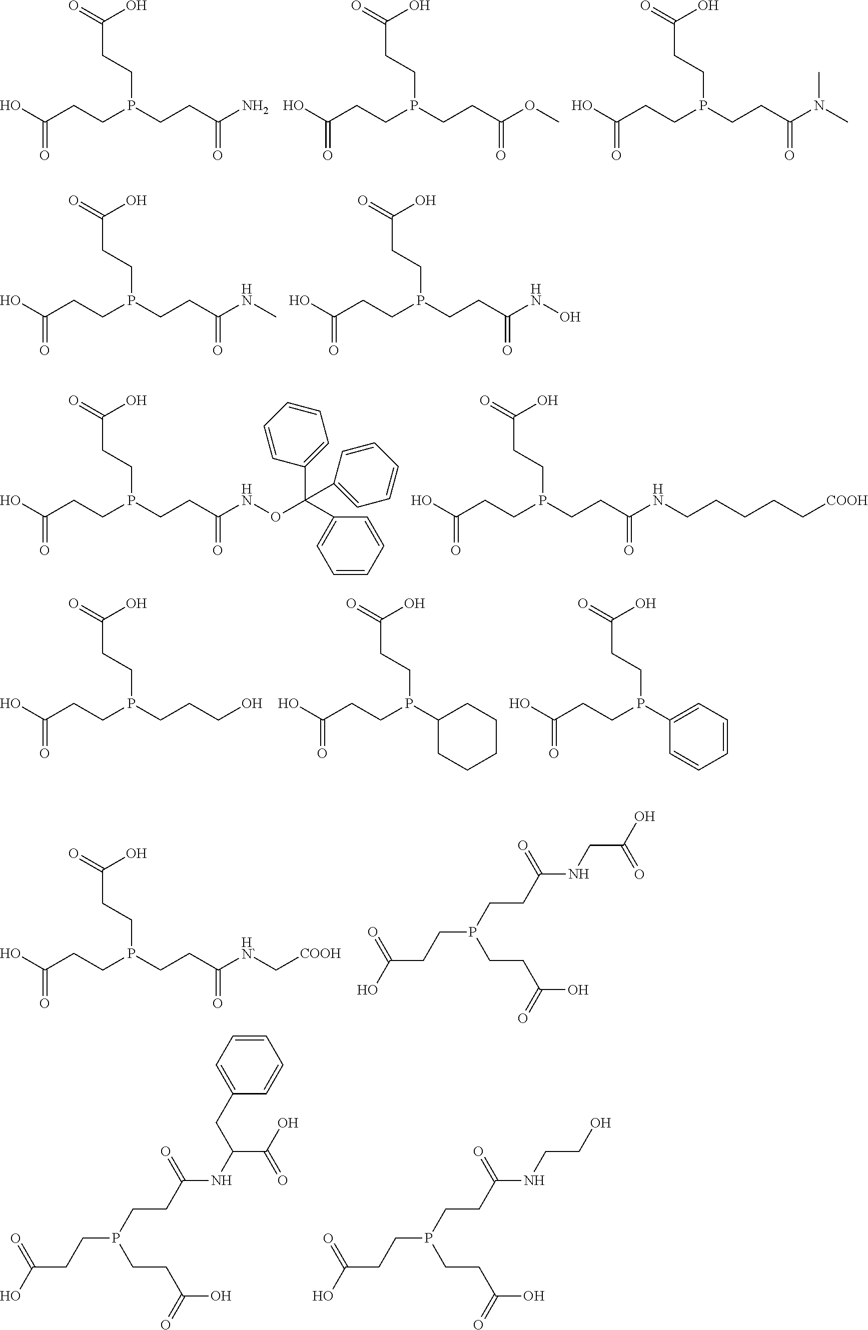

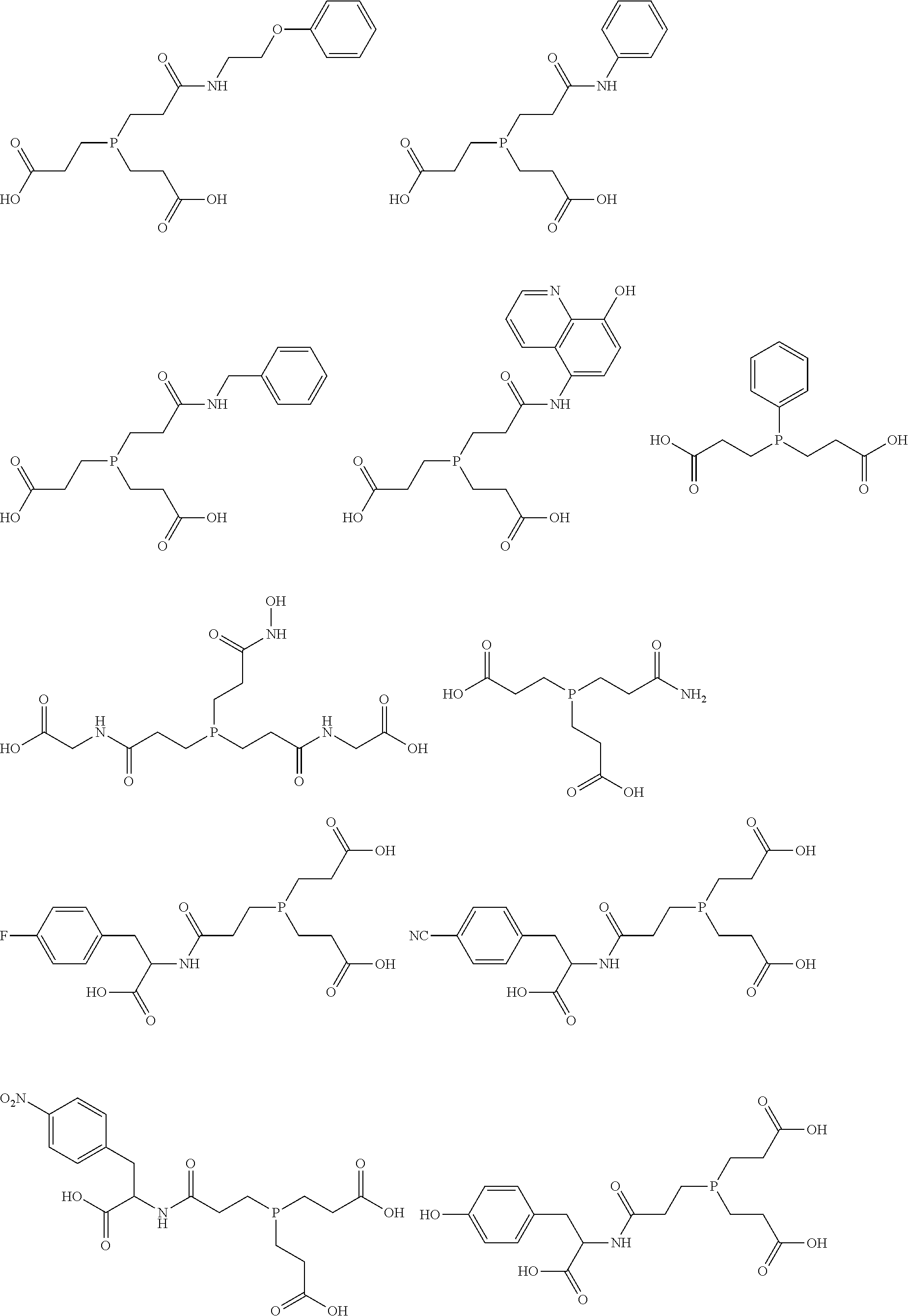

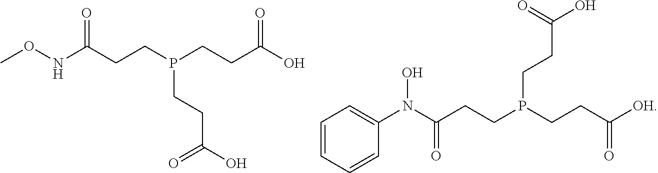 [SMILES: CONC(=O)CCP(CCC(=O)O)CCC(=O)O ; O=C(O)CCP(CCC(=O)O)CCC(=O)N(O)c1ccccc1]

48. The method of claim 9, which characterized in that, the first thiobridge reagent and the second thiobridge reagent independently contain at least two substituted groups allowing a re-bridging of the thiol groups.

49. The method of claim 48, which characterized in that, the first thiobridge reagent and the second thiobridge reagent are independently selected from the group consisting of

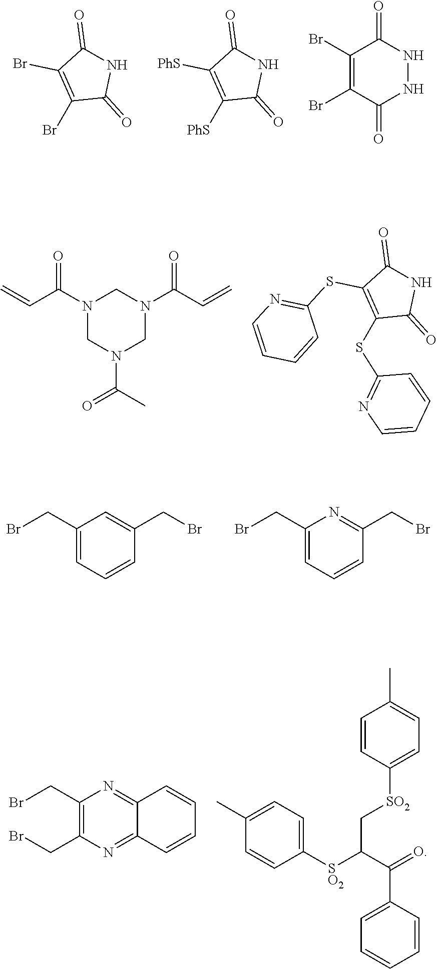

50. The method of claim 48, which characterized in that, the reactive groups independently include azido and/or dibenzocyclooctyne (DBCO).

51. The method of claim 2, which characterized in that, the molar ratio of the first reductant and the transition metal ions in step (R1) is 1:0.4 to 1:250, 1:0.4 to 1:200, 1:1 to 1:70, 1:0.4 to 1:60, 1:0.1 to 1:20, 1:6 to 1:16, 1:0.2 to 1:8, 1:0.5 to 1:8, 1:0.25 to 1:7.5, or 1:0.25 to 1:7.

52. The method of claim 51, which characterized in that, when the first reductant is TCEP, the molar ratio of the first reductant and the transition metal ions in step (R1) is 1:0.4 to 1:200, 1:0.4 to 1:70, 1:1 to 1:16, or 1:2 to 1:16.

53. The method of claim 51, which characterized in that, when the first reductant is the compound having formula (I), optionally, the first reductant is TCEP-NO, TCEP-3NO, or TCEP-CO, the molar ratio of the first reductant and the transition metal ions in step (R1) is 1:0.4 to 1:250, 1:0.4 to 1:60, 1:2 to 1:60, 1: 4 to 1:60, 1: 4 to 1:24, 1: 2 to 1:12, or 1:6 to 1:16.

54. The method of claim 2, which characterized in that, the molar ratio of the first reductant and the antibody is 3:1 to 0.5:1, optionally, the molar ratio of the first reductant and the antibody is 3:1 to 1:1, more optionally, the molar ratio of the first reductant and the antibody is 2:1 to 1:1.

55. The method of claim 2, which characterized in that, the incubation time in step (R1) is 0.2 h to 24 h, optionally, the incubation time in step (R1) is 2 h to 16 h.

56. The method of claim 2, which characterized in that, the molar ratio of the first reductant and the antibody is 2.8:1 to 3.0:1, and the incubation time is 0.5 h to 9 h.

57. The method of claim 3, which characterized in that, the molar ratio of the first reductant and the transition metal ions in step (R1) is 1:0.05 to 1:40, 1:0.08 to 1:30, 1:0.1 to 1:30, 1:0.1 to 1:20, 1:0.5 to 8:1 or 1:0.25 to 1:7.5, optionally, the first reductant is TCEP or the compound having formula (I).

58. The method of claim 3, which characterized in that, the molar ratio of the first reductant and the antibody in step (R1) is 2.8:1 to 20:1, 3:1 to 15:1, 3:1 to 6:1, 3.5:1 to 5:1, 4:1 to 10:1, 5:1 to 13:1.

59. The method of claim 8, which characterized in that, the molar ratio of the first reductant and the transition metal ions in step (R1) is 1:0.05 to 1:40 or 1:0.5 to 1:10, optionally, the first reductant is TCEP or the compound having formula (I).

60. The method of claim 8, which characterized in that, the molar ratio of the first reductant and the antibody in step (R1) is 2.8:1 to 20:1, optionally, the molar ratio of the first reductant and the antibody in step (R1) is 3.5:1 to 10:1.

61. The method of claim 3, which characterized in that, the incubation time in step (R1) is 1 h to 24 h, 14 h to 24 h, 16 h to 20 h or 16 h to 18 h.

62. The method of claim 3, which characterized in that, in step (R1), the molar ratio of the first reductant and the antibody is 2.8 to 3.0, and the incubation time is 10 h to 24 h.

63. The method of claim 3, which characterized in that, in step (R1), the molar ratio of the first reductant and the antibody is 6:1 to 20:1, and the incubation time is 4 h to 16 h.

64. The method of claim 4, which characterized in that, in step (R1), the molar ratio of the first reductant and the antibody is 4:1 to 15:1, and the incubation time is 1 h to 16 h.

65. The method of claim 1, which characterized in that, the incubation temperature in step (R1) is 0° C. to 37° C., 0° C. to 25° C., 0° C. to 15° C., 0° C. to 10° C., or 0° C. to 5° C.

66. The method of claim 1, which characterized in that, the transition metal ions are Zn²⁺, Cd²⁺, Hg²⁺, Ni²⁺, Co²⁺ or the combination thereof, optionally, the transition metal ions are Zn²⁺.

67. The method of claim 1, which characterized in that, the buffer system is selected from the group consisting of MES buffer, Bis-Tris buffer, PIPES buffer, MOPS buffer, BES buffer, HEPES buffer, ADA buffer, PB buffer, DIPSO buffer, MOBS buffer, MOPSO buffer, TES buffer, ACES buffer, TAPSO buffer, PBS, Acetate buffer, BTP buffer, HEPPSO buffer, POPSO buffer, EPPS buffer or Tris buffer, optionally, the buffer system is selected from the group consisting of Bis-Tris buffer, MOPS buffer, BES buffer, HEPES buffer, DIPSO buffer, MOBS buffer, MOPSO buffer, TES buffer, ACES buffer, TAPSO buffer or MES buffer, more optionally, the buffer system is selected from the group consisting of Bis-Tris buffer, MOPS buffer, BES buffer, HEPES buffer, DIPSO buffer, MOBS buffer, MOPSO buffer, TES buffer, ACES buffer, TAPSO buffer, PIPES buffer or MES buffer, and/or the concertation of the buffer system is 10 mM to 100 mM, 20 mM to 80 mM, 20 mM to 40 mM, 20 mM to 60 mM, 40 mM to 80 mM, or 40 mM to 60 mM; and/or the pH value of the buffer system is 5.5 to 8, optionally, the pH value of the buffer system is 6.4 to 7.4, or 6.7 to 7.4.

68-70. (canceled)

71. The method of claim 4, which characterized in that, the oxidant is Dehydroascorbic acid (DHAA) in step (O1).

72. The method of claim 4, which characterized in that, the molar ratio of oxidant and the antibody in step (O1) is 2:1 to 25:1, 2:1 to 20:1, 4:1 to 22:1, 4:1 to 15:1, or 6:1 to 14:1.

73. The method of claim 4, which characterized in that, the oxidation reaction in step (O1) is performing, optionally in darkness, at temperature of 0° C. to 37° C., 0° C. to 30° C., 15° C. to 30° C., or 20° C. to 30° C., and/or the oxidation time is 1 h to 48 h, 1 h to 5 h, or 1 h to 3 h.

74. The method of claim 73, which characterized in that, the oxidation temperature in step (O1) is 0° C. to 25° C., 0° C. to 15° C., 0° C. to 10° C., or 0° C. to 5° C., and/or the oxidation time is 1 h to 8 h, 2 h to 5 h, or 3 h to 8 h.

75. The method of claim 9, which characterized in that, in step (R2) or (R3), the second reductant and the antibody are incubated at temperature of 0° C. to 37° C., 0° C. to 30° C., 15° C. to 30° C., or 20° C. to 30° C., and/or incubation time is 0.2 h to 24 h, 1 h to 10 h, 5 h to 0 h, 1 h to 5 h, or 1 h to 3 h.

76. The method of claim 6, which characterized in that, the molar ratio of the second reductant and the antibody in step (R2) is 1:1 to 3:1 or 1:1 to 2:1.

77. The method of claim 9, which characterized in that, in step (R3) the molar ratio of the second reductant and the transition metal ions is 1:0.05 to 1:40, and/or the molar ratio of the second reductant and the antibody is 2.5:1 to 20:1, and/or the incubation time is 1 h to 24 h.

78. The method of claim 9, which characterized in that, in step (R3), the molar ratio of the second reductant and the transition metal ions is 1:0.4 to 1:100, and/or the molar ratio of the second reductant and the antibody is 0.8:1 to 2.5:1, and/or the incubation time is 0.5 h to 24 h.

79. The method of claim 78, which characterized in that, the method further comprises the following steps,
(R4) incubating the transition metal ions and the second reductant or salt thereof in the buffer system to reduce the interchain disulfide bonds of the antibody resulted from step (C3); and
(C4) introducing the metal chelators and at least equimolecular proportion of the second conjugating group to react with the reduced thiol groups resulted from step (R4), calculated as the molar amount of antibody.

80. The method of claim 79, which characterized in that, in step (R4), the molar ratio of the third reductant and the transition metal ions is 1:0.4 to 1:100, and/or the molar ratio of the third reductant and the antibody is 0.8:1 to 2.5:1, and/or the incubation time is 0.5 h to 24 h.

81. The method of claim 7, which characterized in that, the metal chelators are Ethylenediaminetetraacetic acid disodium salt (EDTA-2Na).

82. The method of claim 9, which characterized in that, when the first thiobridge reagent bears the reactive groups, the step (C1) comprises the following step,
introducing the metal chelators and the first thiobridge reagent bearing the reactive groups to re-bridge the reduced thiol groups resulted from step (R1), (O1) or (R2), then, incubating the first linker-payload in the buffer system to react with the reactive groups of the thiobridge group;
and/or when the first thiobridge reagent bears the reactive groups, the step (C2) comprises the following step,
introducing the first thiobridge reagent bearing the reactive groups to re-bridge the reduced thiol groups resulted from step (R1), incubating the first linker-payload in the buffer system to react with the reactive groups of the thiobridge group; then,
optionally, introducing the metal chelators and the oxidant, or
optionally, introducing the metal chelators and the first conjugating group;
and/or when the second thiobridge reagent bears the reactive groups, the step (C3) comprises the following step,
introducing the second thiobridge reagent bearing the reactive groups to re-bridge the reduced thiol groups resulted from step (R3), optionally, introducing the metal chelators, then, incubating the second linker-payload in the buffer system to react with the reactive groups of the thiobridge group.

83-84. (canceled)

85. The method of claim 9, which characterized in that, the method further comprises a step of purification after step (O1), (C1), (C2) and/or (C3).

86. The method of claim 1, which characterized in that, the antibody is a monoclonal antibody, a polyclonal antibody, a mono-specific antibody or a multi-specific antibody, optionally, the antibody is IgG1 or IgG4 and/or,
a linker of the first linker-payload and the second linker payload is selected from any one of which the one terminal can be connected to the reduced thiol group of the antibody or the reactive groups of the thiobridge reagent, and the other terminal can be connected to the payload; and/or
the payload of linker-payload is selected from any one of which contains at least one substituted group allowing a connection from the payload to the linker, optionally, the payload is a cytotoxic agent, a cytokine, a nucleic acid, a radionuclide, a chemokine, an immuno(co)-stimulatory molecule, an immunosuppressive molecule, a kinase, a prodrug-converting enzyme, a RNase, a growth factor, a hormone, a coagulation factor, a fibrinolytic protein, peptides mimicking these, and fragments, fusion proteins, or derivatives thereof.

87. The method of claim 1, which characterized in that, the antibody is an engineered antibody having two amino acid substitutions of two interchain cysteines forming one interchain disulfide bond in the hinge region, optionally, the amino acid substitutions are selected from the following, cysteine to alanine, to leucine, to arginine, to lysine, to asparagines, to methionine, to aspartic acid, to phenylalanine, to praline, to glutamine, to serine, to glutamic acid, to threonine, to glycine, to tryptophan, to histidine, to tyrosine, to isoleucine or to valine, respectively, more optionally, the amino acid substitutions are selected from the following, cysteine to serine.

88.-89. (canceled)

90. A modified antibody prepared by the method of claim 1.

91. The modified antibody of claim 90, which characterized in that, the modified antibody is conjugated with one, two or three kinds of conjugating groups.

92. The modified antibody of claim 90, which characterized in that, the modified antibody is an antibody drug conjugate (ADC) with D1, D2, D3, D4, D6, D1+D3, D1+D6, D1+D2, D1+D4, D2+D3, D2+D6, D0+D3, D0+D6, D3+D1, D3+D2, D6+D2, D6+D1, D0+D1, D0+D3, D4+D1 or D4+D2.

93. A pharmaceutical composition comprising the modified antibody prepared by the method of claim 1, and at least one pharmaceutically acceptable ingredient.

94. (canceled)

95. A method of preventing or treating a disease in a subject in need thereof, comprising administrating to the subject a therapeutically effective amount of a modified antibody prepared by the method of claim 1.
